(19) United States
(12) Patent Application Publication
(10) Pub. No.: US 2012/0083501 A1
(43) Pub. Date: Apr. 5, 2012

(54) COMPOUNDS FOR TREATING NEURODEGENERATIVE DISEASES

(76) Inventors: Kevin W. Hunt, Boulder, CO (US); Tony P. Tang, Boulder, CO (US); Allen A. Thomas, Boulder, CO (US)

(21) Appl. No.: 13/244,034

(22) Filed: Sep. 23, 2011

Related U.S. Application Data
(60) Provisional application No. 61/386,296, filed on Sep. 24, 2010.

(51) Int. Cl. A61K 31/4188; C07D 491/20; A61P 25/28; A61K 31/4439; A61K 31/506; C07D 491/107; A61K 31/437

(52) U.S. Cl. 514/256; 548/301.1; 546/15; 544/230; 514/392; 514/278

(57) ABSTRACT

The invention provides novel tricyclic compounds of Formula I′ that inhibit β-secretase cleavage of APP and are useful as therapeutic agents for treating neurodegenerative diseases.

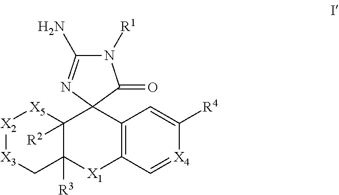

### PRIORITY OF INVENTION

This application claims priority to U.S. Provisional Application No. 61/386,296 that was filed on 24 Sep. 2010. The entire content of this provisional application is hereby incorporated herein by reference.

### BACKGROUND OF THE INVENTION

1. Field of the Invention

The present invention relates to organic compounds useful for inhibition of β-secretase enzymatic activity and the therapy and/or prophylaxis of neurodegenerative diseases associated therewith. More particularly, certain tricyclic compounds useful in the treatment and prevention of neurodegenerative diseases, such as Alzheimer's disease, are provided herein.

2. Description of the State of the Art

Alzheimer's disease (AD) is a neurological disorder thought to be primarily caused by amyloid plaques, an accumulation of abnormal protein deposits in the brain. It is believed that an increase in the production and accumulation of amyloid beta peptides (also referred to as Aβ or A-beta) in plaques leads to nerve cell death, which contributes to the development and progression of AD. Loss of nerve cells due to amyloid plaques in strategic brain areas, in turn, causes reduction in the neurotransmitters and impairment of memory. The proteins principally responsible for the plaque build up include amyloid precursor protein (APP) and presenilin I and II (PSI and PSII). Mutations in each of these three proteins have been observed to enhance proteolytic processing of APP via an intracellular pathway that produces Aβ peptides ranging from 39 to 43 amino acids. The Aβ 1-42 fragment has a particularly high propensity of forming aggregates due to two very hydrophobic amino acid residues at its C-terminus. Thus, Aβ 1-42 fragment is believed to be mainly responsible for the initiation of neuritic amyloid plaque formation in AD and is therefore actively being pursued as a therapeutic target. Anti-Aβ antibodies have been shown to reverse the histologic and cognitive impairments in mice which overexpress Aβ and are currently being tested in human clinical trials. Effective treatment requires anti-Aβ antibodies to cross the blood-brain bather (BBB), however, antibodies typically cross the BBB very poorly and accumulate in the brain in low concentration.

Different forms of APP range in size from 695-770 amino acids, localize to the cell surface, and have a single C-terminal transmembrane domain. Aβ is derived from a region of APP adjacent to and containing a portion of the transmembrane domain. Normally, processing of APP by α-secretase cleaves the midregion of the Aβ sequence adjacent to the membrane and releases a soluble, extracellular domain fragment of APP from the cell surface referred to as APP-α. APP-α is not thought to contribute to AD. On the other hand, pathological processing of APP by the proteases β-secretase (also referred to as “β-site of APP cleaving enzyme” (BACE-1), memapsin-2 and Aspartyl Protease 2 (Asp2)) followed by γ-secretase cleavage, at sites which are located N-terminal and C-terminal to the α-secretase cleavage site, respectively, produces a very different result than processing at the α site, i.e. the release of amyloidogenic Aβ peptides, in particular, Aβ 1-42. Processing at the β- and γ-secretase sites can occur in both the endoplasmic reticulum and in the endosomal/lysosomal pathway after reinternalization of cell surface APP. Dysregulation of intracellular pathways for proteolytic processing may be central to the pathophysiology of AD. In the case of amyloid plaque formation, mutations in APP, PS1 or PS2 consistently alter the proteolytic processing of APP so as to enhance Aβ 1-42 formation.

The initial processing of APP by β-secretase results in a soluble N-APP, which has recently been implicated in neuronal cell death through a pathway independent of amyloid plaque formation. N-APP is involved in normal pruning of neurons in early development in which relatively unused neurons and their nerve-fiber connections (axons) wither and degenerate. Recently, however, it has been shown that N-APP binds to and activates the apoptotic death receptor 6 (DR6) in vitro, which is expressed on axons in response to trophic factor (e.g., nerve growth factor) withdrawal resulting in axonal degeneration. The aging process can lead to a reduction in the levels of growth factors in certain areas of the brain and/or the ability to sense growth factors. This in turn would lead to the release of N-APP fragment by cleavage of APP on neuronal surfaces, activating nearby DR6 receptors to initiate the axonal shrinkage and neuronal degeneration of Alzheimer's.

See also, Rauk, Arvi. “The chemistry of Alzheimer's disease.” Chem. Soc. Rev. 38 (2009): p. 2698-2715; Vassar, Robert, Dora M. Kovacs, Riqiang Yan and Philip C. Wong. “The •-Secretase Enzyme BACE in Health and Alzheimer's disease: Regulation, Cell Biology, Function, and Therapeutic Potential. J. Neurosci. 29(41) (2009): 12787-12794; and Silvestri, Romano. “Boom in the Development of Non-Peptidic β-Secretase (BACE1) Inhibitors for the Treatment of Alzheimer's Disease.” Medicinal Research Reviews. Vol. 29, No. 2 (2009): p. 295-338.

Since β-secretase cleavage of APP is essential for both amyloid plaque formation and DR6-mediated apoptosis, it is a key target in the search for therapeutic agents for treating AD.

### SUMMARY OF THE INVENTION

In one aspect of the present invention there is provided novel compounds having the general Formula I′:

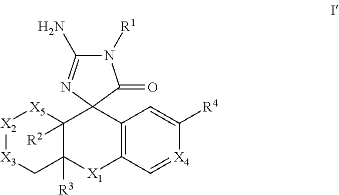

and stereoisomers, diastereomers, enantiomers, tautomers and pharmaceutically acceptable salts thereof, wherein X₁, X₂, X₃, X₄, X₅, R¹, R², R³ and R⁴ are as defined herein.

In another aspect of the invention, there are provided pharmaceutical compositions comprising compounds of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j and a pharmaceutically acceptable carrier, diluent or excipient.

In another aspect of the invention, there is provided a method of inhibiting cleavage of APP by β-secretase in a mammal comprising administering to said mammal an effective amount of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j.

In another aspect of the invention, there is provided a method for treating a disease or condition mediated by the cleavage of APP by β-secretase in a mammal, comprising administering to said mammal an effective amount of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j.

In another aspect of the invention, there is provided a use of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j in the manufacture of a medicament for the treatment of neurodegenerative diseases, such as Alzheimer's disease.

In another aspect of the invention, there is provided a use of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j in the treatment of neurodegenerative diseases, such as Alzheimer's disease.

Another aspect provides intermediates for preparing compounds of Formula I′, I, I′a, Ia, I′b, Ib, I′c, Ic, I′d, Id, I′e, Ie, If, I′g, I″g, I′″g, Ig, I′h, I″h, I′″h, Ih, I′j, I″j or I′″j. Certain compounds of Formula I′, I, I′a, Ia, I′b, Ib, I′c, Ic, I′d, Id, I′e, Ie, If, I′g, Ig, I′h, I″h, I′″h, Ih, I′j, I″j or I′″j may be used as intermediates for other compounds of Formula I′, I, I′a, Ia, I′b, Ib, I′c, Ic, I′d, Id, I′e, Ie, If, I′g, I″g, I′″g, Ig, I′h, I″h, I′″h, Ih, I′j, I″j or I′″j.

Another aspect includes processes for preparing, methods of separation, and methods of purification of the compounds described herein.

### DETAILED DESCRIPTION OF THE INVENTION

### Definitions

The term “acyl” means a carbonyl containing substituent represented by the formula —C(O)—R, in which R is hydrogen, alkyl, alkoxy, amino, a carbocycle, a heterocycle, carbocycle-substituted alkyl or heterocycle-substituted alkyl, wherein the alkyl, alkoxy, amino, carbocycle and heterocycle are as defined herein. Acyl groups include alkanoyl (e.g., acetyl), aroyl (e.g., benzoyl), and heteroaroyl.

The term “alkoxycarbonyl” means the group —C(═O)OR in which R is alkyl. A particular alkoxycarbonyl group is C₁-C₆ alkoxycarbonyl, wherein the R group is C₁-C₆ alkyl.

The term “alkyl” means a branched or unbranched, saturated or unsaturated (i.e., alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. In other examples, the alkyl group is C₁-C₂, C₁-C₃, C₁-C₄, C₁-C₅ or C₁-C₆. Unless specified otherwise, substituted alkyl groups contain one, two, three or four substituents which may be the same or different. Alkyl substituents are, unless otherwise specified, halogen, amino, hydroxyl, protected hydroxyl, mercapto, carboxy, alkoxy, nitro, cyano, amidino, guanidino, urea, oxo, sulfonyl, sulfinyl, aminosulfonyl, alkylsulfonylamino, arylsulfonylamino, aminocarbonyl, acylamino, alkoxy, acyl, acyloxy, an optionally substituted carbocycle and an optionally substituted heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyls, e.g., a methyl group substituted by the same substituents as the “substituted Cₙ-Cₘ alkyl” group. Examples of the substituted methyl group include groups such as hydroxymethyl, protected hydroxymethyl (e.g., tetrahydropyranyloxymethyl), acetoxymethyl, carbamoyloxymethyl, trifluoromethyl, chloromethyl, carboxymethyl, bromomethyl and iodomethyl.

The terms “alkenyl” and “alkynyl” also include linear or branched-chain radicals of carbon atoms.

The term “alkoxy” means the group —O(alkyl), wherein the alkyl is linear or branched-chain. The alkyl may be substituted by the same substituents as the “substituted alkyl” group. C₁-C₆ alkoxy means —O(C₁-C₆ alkyl).

The term “amidine” means the group —C(NH)—NHR in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. A particular amidine is the group —NH—C(NH)—NH₂.

The term “amino” means primary (i.e., —NH₂), secondary (i.e., —NRH) and tertiary (i.e., —NRR) amines in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. Particular secondary and tertiary amines are alkylamine, dialkylamine, arylamine, diarylamine, aralkylamine and diaralkylamine, wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and disopropylamine.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are acetyl, trifluoroacetyl, t-butyloxycarbonyl (“Boc”), benzyloxycarbonyl (“CBz”) and 9-fluorenylmethyleneoxycarbonyl (“Fmoc”). Further examples of these groups, and other protecting groups, are found in T. W. Greene, et al. Greene's Protective Groups in Organic Synthesis. New York: Wiley Interscience, 2006.

The term “aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g., Dean, J. A. Lange's Handbook of Chemistry. 15th ed. New York: McGraw-Hill Professional, 1998). A particular aryl is phenyl. Substituted phenyl or substituted aryl means a phenyl group or aryl group substituted with one, two, three, four or five substituents, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” includes, but is not limited to, a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such as 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; a mono- or di(N-(methylsulfonylamino))phenyl such as 3-(N-methylsulfonylamino))phenyl; disubstituted phenyl groups such as 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl and 2-hydroxy-4-chlorophenyl; trisubstituted phenyl groups such as 3-methoxy-4-benzyloxy-6-methylsulfonylamino and 3-methoxy-4-benzyloxy-6-phenylsulfonylamino; tetrasubstituted phenyl groups such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

The terms “carbocyclyl”, “carbocyclic”, “carbocycle” and “carbocyclo” alone and when used as a moiety in a complex group such as a carbocycloalkyl group, refer to a mono-, bi-, or tricyclic aliphatic ring having 3 to 14 carbon atoms, for example 3 to 7 carbon atoms or 3 to 6 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic, e.g., aryl groups as previously defined, for example phenyl. The terms “substituted carbocyclyl”, “carbocycle” and “carbocyclo” mean these groups substituted by the same substituents as the “substituted alkyl” group.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethypprop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. Such harsh removal conditions are also to be avoided when removing amino-protecting groups and hydroxy-protecting groups, discussed below. Particular carboxylic acid protecting groups are the alkyl (e.g., methyl, ethyl, t-butyl), allyl, benzyl and p-nitrobenzyl groups. The term “protected carboxy” refers to a carboxy group substituted with one of the above carboxy-protecting groups. Further examples are found in Greene's Protective Groups in Organic Synthesis, supra.

The terms “comprise” and “comprising” when used herein are non-limiting in scope, i.e., are intended to specify the presence of the stated features, integers, components, or steps, but do not preclude the presence or addition such features, integers, components, steps, or groups thereof.

The term “guanidine” means the group —NH—C(NH)—NHR in which R is hydrogen, alkyl, alkoxy, a carbocycle, a heterocycle, carbocycle-substituted alkyl or heterocycle-substituted alkyl, wherein the alkyl, alkoxy, carbocycle and heterocycle are as defined herein. A particular guanidine is the group —NH—C(NH)—NH₂.

The term “hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g., tert-butyldimethylsilyl (“TBS”), tert-butyldiphenylsilyl (“TBDPS”)) groups. Further examples are found in Greene's Protective Groups in Organic Synthesis, supra. The term “protected hydroxy” refers to a hydroxy group substituted with one of the above hydroxy-protecting groups.

The term “heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. The sulfur heteroatoms may optionally be oxidized (e.g., SO, SO₂), and any nitrogen heteroatom may optionally be quaternized. Typically, a 5-membered ring has 0 to 2 double bonds and 6- or 7-membered ring has 0 to 3 double bonds. In a particular embodiment, heterocyclic groups are four to seven membered cyclic groups containing one, two or three heteroatoms selected from the group consisting of nitrogen, oxygen and sulfur. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide; thiadiazolyl, in particular 1,3,4-thiadiazol-5-yl and 1,2,4-thiadiazol-5-yl; oxazolyl, for example oxazol-2-yl; and oxadiazolyl, such as 1,3,4-oxadiazol-5-yl and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl, 1,2,3-triazol-5-yl, and 1,2,4-triazol-5-yl; and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl; and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substituted heterocycle groups are substituted with hydroxyl, alkyl, alkoxy, acyl, halogen, mercapto, oxo, carboxyl, acyl, halo-substituted alkyl, amino, cyano, nitro, amidino and guanidino.

The term “heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (see Lange's Handbook of Chemistry, supra). In a particular embodiment, the heteroaryl is a 5-membered aromatic ring containing one, two or three heteroatoms selected from nitrogen, oxygen and sulfur. Included in the definition are any bicyclic groups where any of the above heteroaryl rings are fused to a benzene ring. Particular heteroaryls incorporate a nitrogen or oxygen heteroatom. In a particular embodiment, the heteroaryl is a 5-membered aromatic ring containing one, two or three heteroatoms selected from nitrogen, oxygen and sulfur. In a particular embodiment, the heteroaryl group is a 6-membered aromatic ring containing one, two or three heteroatoms selected from nitrogen, oxygen and sulfur. The following are examples of the heteroaryl groups (substituted and unsubstituted): thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. In a particular embodiment the heteroaryl group may be: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1 ,3 ,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, or 8-aminotetrazolo[1,5-b]pyridazin-6-yl. Heteroaryl groups are optionally substituted as described for heterocycles.

The term “inhibitor” means a compound which reduces or prevents the enzymatic cleavage of APP by β-secretase. Alternatively, “inhibitor” means a compound which prevents or slows the formation of beta-amyloid plaques in mammalian brain. Alternatively, “inhibitor” means a compound that prevents or slows the progression of a disease or condition associated with β-secretase enzymatic activity, e.g., cleavage of APP. Alternatively, “inhibitor” means a compound which prevents Alzheimer's disease. Alternatively, “inhibitor” means a compound which slows the progression of Alzheimer's disease or its symptoms.

The term “optionally substituted” unless otherwise specified means that a group may be unsubstituted or substituted by one or more (e.g., 0, 1, 2, 3 or 4) of the substituents listed for that group in which said substituents may be the same or different. In a particular embodiment, an optionally substituted group has 1 substituent. In another embodiment an optionally substituted group has 2 substituents. In another embodiment an optionally substituted group has 3 substituents.

The term “pharmaceutically acceptable” indicates that the substance or composition is compatible chemically and/or toxicologically, with the other ingredients comprising a formulation, and/or the mammal being treated therewith.

The term “pharmaceutically acceptable salts” include both acid and base addition salts.

The term “pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly organic non-toxic bases are isopropylamine, diethylamine, ethanolamine, trimethamine, dicyclohexylamine, choline, and caffeine.

The term “sulfanyl” means —S—R group in which R is alkyl, a carbocycle, a heterocycle, carbocycle-substituted alkyl or heterocycle-substituted alkyl, wherein the alkyl, carbocycle and heterocycle are as defined herein. Particular sulfanyl groups are alkylsulfanyl (i.e., —S-alkyl), for example methylsulfanyl; arylsulfanyl, for example phenylsulfanyl; and aralkylsulfanyl, for example benzylsulfanyl.

The term “sulfinyl” means —SO—R group in which R is hydrogen, alkyl, a carbocycle, a heterocycle, carbocycle-substituted alkyl or heterocycle-substituted alkyl, wherein the alkyl, carbocycle and heterocycle are as defined herein. Particular sulfinyl groups are alkylsulfinyl (i.e., —SO-alkyl), for example methylsulfinyl; arylsulfinyl, for example phenylsulfinyl; and aralkylsulfinyl, for example benzylsulfinyl.

The term “sulfonyl” means a —SO₂—R group in which R is hydrogen, alkyl, a carbocycle, a heterocycle, carbocycle-substituted alkyl or heterocycle-substituted alkyl wherein the alkyl, carbocycle and heterocycle are as defined herein. Particular sulfonyl groups are alkylsulfonyl (i.e., —SO₂-alkyl), for example methylsulfonyl; arylsulfonyl, for example phenylsulfonyl; and aralkylsulfonyl, for example benzylsulfonyl.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art. The “effective amount” of the compound to be administered will be governed by such considerations, and is the minimum amount necessary to inhibit cleavage of APP by J3-secretase, for example by 10% or greater in situ. In a particular embodiment an “effective amount” of the compound inhibits cleavage of APP by β-secretase by 25% or greater in situ. In a particular embodiment the effective amount inhibits cleavage of APP by β-secretase by 50% or greater in situ. In a particular embodiment the effective amount inhibits cleavage of APP by β-secretase by 70% or greater in situ. In a particular embodiment the effective amount inhibits cleavage of APP by β-secretase by 80% or greater in situ. In a particular embodiment the effective amount inhibits cleavage of APP by β-secretase by 90% or greater in situ. Such amount may be below the amount that is toxic to normal cells, or the mammal as a whole. Alternatively, an “effective amount” is the amount of compound necessary to reduce A-beta levels in plasma or cerebrospinal fluid of a mammal, for example, by 10% or greater. In a particular embodiment, an “effective amount” is the amount of compound necessary to reduce A-beta levels in plasma or cerebrospinal fluid of a mammal by 25% or greater. In a particular embodiment, an “effective amount” is the amount of compound necessary to reduce A-beta levels in plasma or cerebrospinal fluid of a mammal by 50% or greater. In a particular embodiment, an “effective amount” is the amount of compound necessary to reduce A-beta levels in plasma or cerebrospinal fluid of a mammal by 75% or greater. Alternatively, an “effective amount” of the compound may be the amount of compound necessary to slow the progression of AD or symptoms thereof.

Abbreviations are sometimes used in conjunction with elemental abbreviations and chemical structures, for example, methanol (“MeOH”), ethanol (“EtOH”) or ethyl acetate (“EtOAc”). Additional abbreviations used throughout the application may include, for example, benzyl (“Bn”), phenyl (“Ph”) and acetate (“Ac”).

Tricyclic Compounds

Provided herein are compounds, and pharmaceutical formulations thereof, that are potentially useful in the treatment of diseases, conditions and/or disorders modulated by BACE-1.

One embodiment provides compounds of Formula I′:

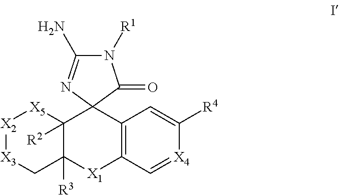

and stereoisomers, diastereomers, enantiomers, tautomers and pharmaceutically acceptable salts thereof, wherein:

X₁ is selected from O, S, S(O), SO₂, NR¹⁰ and CHR¹⁰;

X₂ is selected from CR⁵R⁶, NR⁷ and O;

X₃ is selected from CR⁸R⁹ and O;

X₄ is selected from CR¹¹ and N;

X₅ is selected from CR¹²R¹³ and O, wherein two of X₂, X₃ and X₅ must contain C;

R¹ is selected from hydrogen, alkyl, aralkyl, heteroaryl and heteroaralkyl;

R² and R³ are independently selected from hydrogen, halogen and alkyl;

R⁴ is selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁵ and R⁶ taken together form an oxo group, or

R⁵ and R⁶ together with the atom to which they are attached form a carbocycle or heterocycle;

R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁸ and R⁹ taken together form an oxo or alkenyl group, wherein the double bond of the alkenyl group is immediately attached to the carbon atom to which R⁸ and R⁹ are attached, or

R⁸ and R⁹ together with the atom to which they are attached form a carbocycle or heterocycle;

R¹⁰ is selected from hydrogen, halogen and alkyl;

R¹¹ is selected from hydrogen, halogen and alkyl; and

R¹² and R¹³ are independently selected from hydrogen and alkyl.

In certain embodiments of Formula I:

X₁ is selected from O, S, S(O), SO₂, NR¹⁰ and CHR¹⁰;

X₂ is selected from CR⁵R⁶, NR⁷ and O;

X₃ is selected from CR⁸R⁹ and O;

X₄ is selected from CR¹¹ and N;

X₅ is selected from CR¹²R¹³ and O, wherein two of X₂, X₃ and X₅ must contain C;

R¹ is selected from hydrogen, benzyl and C₁-C₃ alkyl, wherein the alkyl is optionally substituted with one or more R^{a} groups;

R² and R³ are independently selected from hydrogen, halogen and C₁-C₆ alkyl;

R⁴ is selected from hydrogen, halogen, C₁-C₆ alkyl, C₁-C₆ alkenyl, C₁-C₆ alkynyl, C₁-C₆ alkoxy, —NHC(═O)(C₁-C₆ alkyl), —C(═O)NH(C₁-C₆ alkyl), a 3 to 6 membered carbocycle, a 3 to 6 membered heterocycle, phenyl, and a 5 to 6 membered heteroaryl, wherein the alkyl, alkenyl, alkynyl, alkoxy, carbocycle, heterocycle, phenyl and heteroaryl are optionally substituted with one or more R^{b} groups;

R⁵ and R⁶ are independently selected from hydrogen, halogen, hydroxyl, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or

R⁵ and R⁶ taken together form an oxo group, or

R⁵ and R⁶ together with the atom to which they are attached form a 3 to 6 membered carbocycle or heterocycle;

R⁷ is selected from hydrogen, C₁-C₆ alkyl, C₁-C₆ alkoxycarbonyl, —C(═O)NR^{f}R^{g}, —SO₂(C₁-C₆ alkyl), a 3 to 6 membered carbocycle, a 3 to 6 membered heterocycle, phenyl, and a 5 to 6 membered heteroaryl, wherein the alkyl, alkoxycarbonyl, carbocycle, heterocycle, phenyl and heteroaryl are optionally substituted with one or more R^{b} groups;

R⁸ and R⁹ are independently selected from hydrogen, halogen, CN, C₁-C₆ alkyl, C₁-C₆ alkenyl, C₁-C₆ alkynyl, C₁-C₆ alkoxy, phenyl, a 5 to 6 membered heteroaryl and OR^{d}, wherein the alkyl, alkenyl, alkynyl, alkoxy, phenyl and heteroaryl are optionally substituted with halogen, or

R⁸ and R⁹ taken together form an oxo group or C₁-C₆ alkenyl group, wherein the double bond of the alkenyl group is immediately attached to the carbon atom to which R⁸ and R⁹ are attached, or

R⁸ and R⁹ together with the atom to which they are attached form a 3 to 6 membered carbocycle or heterocycle;

R¹⁰ is selected from hydrogen, halogen and C₁-C₆ alkyl;

R¹¹ is selected from hydrogen, halogen and C₁-C₆ alkyl, wherein the alkyl is optionally substituted with one or more R^{b} groups;

R¹² and R¹³ are independently selected from hydrogen and C₁-C₆ alkyl;

each R^{a} is independently selected from OH, OCH₃, halogen, a 5 to 6 membered heteroaryl, and a 3 to 6 membered heterocyclyl, wherein the heterocyclyl is optionally substituted with C₁-C₃ alkyl optionally substituted with oxo;

each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen;

each R^{d} is independently selected from hydrogen and C₁-C₆ alkyl, wherein the alkyl is optionally substituted with one or more R^{e} groups;

each R^{e} is independently selected from halogen and C₃-C₆ cycloalkyl; and

R^{f} and R^{g} are independently selected from hydrogen and C₁-C₆ alkyl, wherein the alkyl is optionally substituted with halogen, CN or C₁-C₆ alkoxy.

In certain embodiments of Formula I:

X₁ is selected from O and CH₂;

X₂ is selected from CR⁵R⁶, NR⁷ and O;

X₃ is CR⁸R⁹;

X₄ is CR¹¹;

X₅ is selected from CHR¹² and O, wherein one of X₂ and X₅ must contain C;

R¹ is C₁-C₃ alkyl;

R² and R³ are independently selected from hydrogen, halogen and C₁-C₆ alkyl;

R⁴ is selected from halogen, C₁-C₆ alkoxy, phenyl and a 5 to 6 membered heteroaryl, wherein the phenyl and heteroaryl are optionally substituted with one or two R^{b} groups;

R⁵ and R⁶ are independently selected from hydrogen, halogen, hydroxyl and C₁-C₆ alkoxy optionally substituted with a 3 to 6 membered carbocycle, or

R⁵ and R⁶ taken together form an oxo group, or

R⁵ and R⁶ together with the atom to which they are attached form a 3 to 6 membered heterocycle;

R⁷ is selected from hydrogen and C₁-C₆ alkyl;

R⁸ and R⁹ are independently selected from hydrogen, halogen, C₁-C₆ alkyl, C₁-C_{alkenyl, C}₁-C₆ alkynyl, and OR^{d}, or

R⁸ and R⁹ taken together form an oxo group or C₁-C₆ alkenyl group wherein the double bond of the alkenyl group is immediately attached to the carbon atom to which R⁸ and R⁹ are attached, or

R⁸ and R⁹ together with the atom to which they are attached form a 3 to 6 membered heterocycle;

R¹¹ is selected from hydrogen and halogen;

R¹² is selected from hydrogen and C₁-C₆ alkyl;

each R^{b} is independently selected from halogen, CN, C₁-C₆ alkyl and C₁-C₆ alkoxy, wherein the alkyl and alkoxy are optionally substituted with halogen;

each R^{d} is independently selected from hydrogen and C₁-C₆ alkyl, wherein the alkyl is optionally substituted with one or more R^{e} groups; and

each R^{e} is independently selected from halogen and C₃-C₆ cycloalkyl.

One embodiment provides compounds of Formula I:

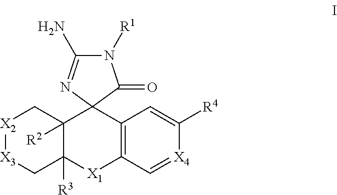

and stereoisomers, diastereomers, enantiomers, tautomers and pharmaceutically acceptable salts thereof, wherein:

X₁ is selected from O, S, S(O), SO₂, NR¹⁰ and CHR¹⁰;

X₂ is selected from CR⁵R⁶, NR⁷ and O;

X₃ is selected from CR⁸R⁹ and O, wherein at least one of X₂ or X₃ must contain C;

X₄ is selected from CR¹¹ and N;

R¹ is selected from hydrogen, alkyl, aralkyl, heteroaryl and heteroaralkyl;

R² and R³ are hydrogen or alkyl;

R⁴ is selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁵ and R⁶ taken together form an oxo group, or

R⁵ and R⁶ together with the atom to which they are attached form a carbocycle or heterocycle;

R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁸ and R⁹ taken together form an oxo or alkenyl group, wherein the double bond of the alkenyl group is immediately attached to the carbon atom to which R⁸ and R⁹ are attached, or

R⁸ and R⁹ together with the atom to which they are attached form a carbocycle or heterocycle;

R¹⁰ is selected from hydrogen, halogen and alkyl; and

R¹¹ is selected from hydrogen, halogen and alkyl.

In certain embodiments of Formula I:

X₁ is selected from O, S, S(O), SO₂, NR¹⁰ and CHR¹⁰;

X₂ is selected from CR⁵R⁶, NR⁷ and O;

X₃ is selected from CR⁸R⁹ and O, wherein at least one of X₂ or X₃ must contain C;

X₄ is selected from CR¹¹ and N;

R¹ is selected from hydrogen, benzyl and C₁-C₃ alkyl, wherein the alkyl is optionally substituted with one or more R^{a} groups;

R² and R³ are independently selected from hydrogen and C₁-C₆ alkyl;

R⁴ is selected from hydrogen, halogen, C₁-C₆ alkyl, C₁-C₆ alkenyl, C₁-C₆ alkynyl, —NHC(═O)(C₁-C₆ alkyl), —C(═O)NH(C₁-C₆ alkyl), a 3 to 6 membered carbocycle, a 3 to 6 membered heterocycle, phenyl, and a 5 to 6 membered heteroaryl, wherein the alkyl, alkenyl, alkynyl, carbocycle, heterocycle, phenyl and heteroaryl are optionally substituted with one or more R^{b} groups;

R⁵ and R⁶ are independently selected from hydrogen, halogen, OR^{c}, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or

R⁵ and R⁶ taken together form an oxo group, or

R⁵ and R⁶ together with the atom to which they are attached form a 3 to 6 membered carbocycle or heterocycle;

R⁷ is selected from hydrogen, C₁-C₆ alkyl, C₁-C₆ alkoxycarbonyl, —C(═O)NR^{f}R^{g}, —SO₂(C₁-C₆ alkyl), a 3 to 6 membered carbocycle, a 3 to 6 membered heterocycle, phenyl, and a 5 to 6 membered heteroaryl, wherein the alkyl, alkoxycarbonyl, carbocycle, heterocycle, phenyl and heteroaryl are optionally substituted with one or more R^{b} groups;

R⁸ and R⁹ are independently selected from hydrogen, halogen, CN, C₁-C₆ alkyl, C₁-C₆ alkenyl, C₁-C₆ alkynyl, C₁-C₆ alkoxy, phenyl, a 5 to 6 membered heteroaryl and OR^{d}, wherein the alkyl, alkenyl, alkynyl, alkoxy, phenyl and heteroaryl are optionally substituted with halogen, or

R⁸ and R⁹ taken together form an oxo group or C₁-C₆ alkenyl group, wherein the double bond of the alkenyl group is immediately attached to the carbon atom to which R⁸ and R⁹ are attached, or

R⁸ and R⁹ together with the atom to which they are attached form a 3 to 6 membered carbocycle or heterocycle;

R¹⁰ is selected from hydrogen, halogen and C₁-C₆ alkyl;

R¹¹ is selected from hydrogen, halogen and C₁-C₆ alkyl, wherein the alkyl is optionally substituted with one or more R^{b} groups;

each R^{a} is independently selected from OH, OCH₃, halogen, a 5 to 6 membered heteroaryl, and a 3 to 6 membered heterocyclyl, wherein the heterocyclyl is optionally substituted with C₁-C₃ alkyl optionally substituted with oxo;

each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen;

each R^{c} is independently selected from hydrogen and C₁-C₆ alkyl;

each R^{d} is independently selected from hydrogen and C₁-C₆ alkyl, wherein the alkyl is optionally substituted with one or more R^{e} groups;

each R^{e} is independently selected from halogen and C₃-C₆ cycloalkyl; and

R^{f} and R^{g} are independently selected from hydrogen and C₁-C₆ alkyl, wherein the alkyl is optionally substituted with halogen, CN or C₁-C₆ alkoxy.

In certain embodiments of Formula I:

X₁ is O;

X₂ is selected from CR⁵R⁶, NR⁷ and O;

X₃ is CR⁸R⁹;

X₄ is CH;

R¹ is C₁-C₃ alkyl;

R² and R³ are hydrogen;

R⁴ is selected from phenyl and a 5 to 6 membered heteroaryl, wherein the phenyl and heteroaryl are optionally substituted with one or more R^{b} groups;

R⁵ and R⁶ are independently selected from hydrogen and halogen, or

R⁵ and R⁶ taken together form an oxo group, or

R⁵ and R⁶ together with the atom to which they are attached form a 3 to 6 membered heterocycle;

R⁷ is selected from hydrogen and C₁-C₆ alkyl;

R⁸ and R⁹ are independently selected from hydrogen, halogen, C₁-C₆ alkyl, C₁-C₆ alkenyl, C₁-C₆ alkynyl, and OR^{d}, or

R⁸ and R⁹ taken together form an oxo group or C₁-C₆ alkenyl group wherein the double bond of the alkenyl group is immediately attached to the carbon atom to which R⁸ and R⁹ are attached, or

R⁸ and R⁹ together with the atom to which they are attached form a 3 to 6 membered heterocycle;

each R^{b} is independently selected from halogen, C₁-C₆ alkyl and C₁-C₆ alkoxy, wherein the alkyl and alkoxy are optionally substituted with halogen;

each R^{d} is independently selected from hydrogen and C₁-C₆ alkyl, wherein the alkyl is optionally substituted with one or more R^{e} groups; and

each R^{e} is independently selected from halogen and C₃-C₆ cycloalkyl.

In a particular embodiment, compounds of the invention have the stereochemical orientation represented by Formula I′a:

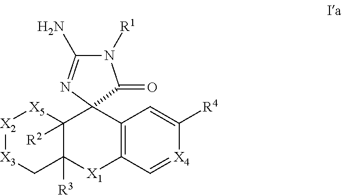

wherein X₁, X₂, X₃, X₄, X₅, R¹, R², R³ and R⁴ are as defined herein.

In a particular embodiment, compounds of the invention have the stereochemical orientation represented by Formula Ia:

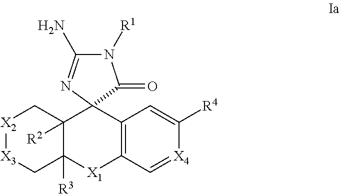

wherein X₁, X₂, X₃, X₄, R¹, R², R³ and R⁴ are as defined herein.

In a particular embodiment, compounds of the invention have the stereochemical orientation represented by Formula I′b:

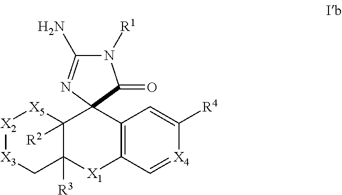

wherein X₁, X₂, X₃, X₄, X₅, R¹, R², R³ and R⁴ are as defined herein.

In a particular embodiment, compounds of the invention have the stereochemical orientation represented by Formula Ib:

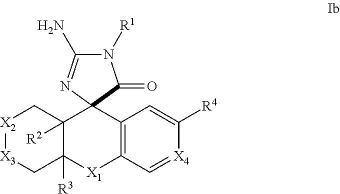

wherein X₁, X₂, X₃, X₄, R¹, R², R³ and R⁴ are as defined herein.

In a particular embodiment, compounds of the invention have the stereochemical orientation represented by Formula I′c:

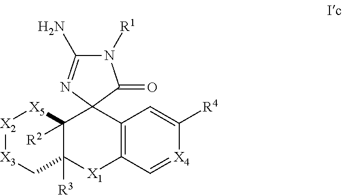

wherein X₁, X₂, X₃, X₄, X₅, R¹, R², R³ and R⁴ are as defined herein.

In a particular embodiment, compounds of the invention have the stereochemical orientation represented by Formula Ic:

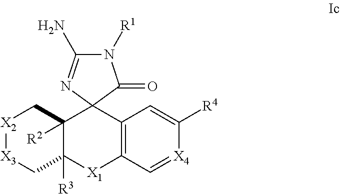

wherein X₁, X₂, X₃, X₄, R¹, R², R³ and R⁴ are as defined herein.

In a particular embodiment, compounds of the invention have the stereochemical orientation represented by Formula I′d:

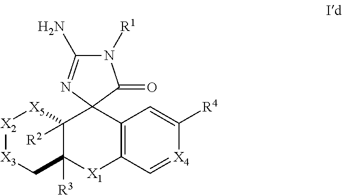

wherein X₁, X₂, X₃, X₄, X₅, R¹, R², R³ and R⁴ are as defined herein.

In a particular embodiment, compounds of the invention have the stereochemical orientation represented by Formula Id:

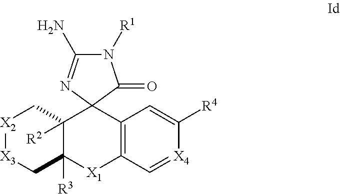

wherein X₁, X₂, X₃, X₄, R¹, R², R³ and R⁴ are as defined herein.

In a particular embodiment, compounds of the invention have the stereochemical orientation represented by Formula I′e:

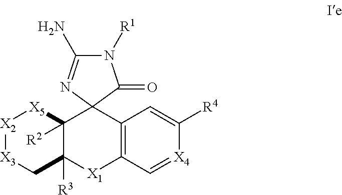

wherein X₁, X₂, X₃, X₄, X₅, R¹, R², R³ and R⁴ are as defined herein.

In a particular embodiment, compounds of the invention have the stereochemical orientation represented by Formula Ie:

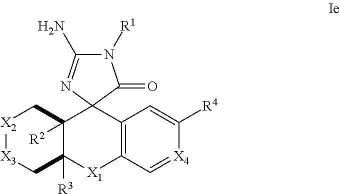

wherein X₁, X₂, X₃, X₄, R¹, R², R³ and R⁴ are as defined herein.

In a particular embodiment, compounds of the invention have the stereochemical orientation represented by Formula I′f:

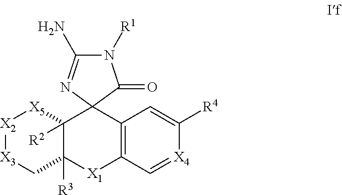

wherein X₁, X₂, X₃, X₄, X₅, R¹, R², R³ and R⁴ are as defined herein.

In a particular embodiment, compounds of the invention have the stereochemical orientation represented by Formula If:

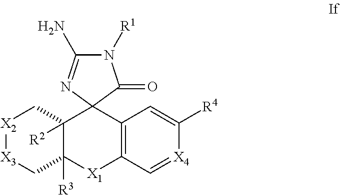

wherein X₁, X₂, X₃, X₄, R¹, R², R³ and R⁴ are as defined herein.

In a particular embodiment, compounds of the invention have the Formula I′g:

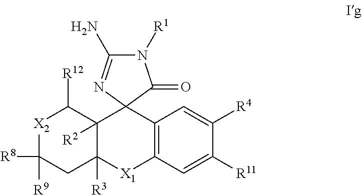

wherein X₁, X₂, R¹, R², R³, R⁴, R⁸, R⁹ and R¹¹ are as defined herein.

In a particular embodiment, compounds of the invention have the Formula I″g:

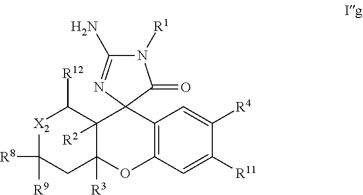

wherein X₂, R¹, R², R³, R⁴, R⁸, R⁹ and R¹¹ are as defined herein.

In a particular embodiment, compounds of the invention have the Formula I′″g:

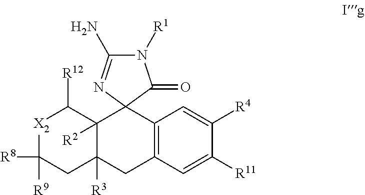

wherein X₂, R¹, R², R³, R⁴, R⁸, R⁹ and R¹¹ are as defined herein.

In a particular embodiment, compounds of the invention have the Formula Ig:

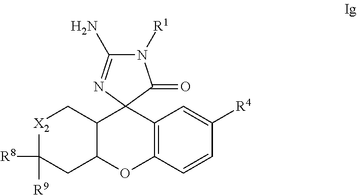

wherein X₂, R¹, R⁴, R⁸ and R⁹ are as defined herein.

In a particular embodiment, compounds of the invention have the Formula I′h:

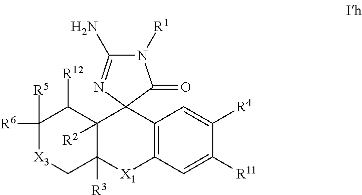

wherein X₁, X₃, R¹, R², R³, R⁴, R⁵, R⁶ and R¹¹ are as defined herein.

In a particular embodiment, compounds of the invention have the Formula I″h:

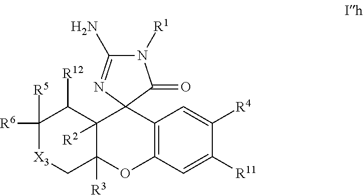

wherein X₃, R¹, R², R³, R⁴, R⁵, R⁶ and R¹¹ are as defined herein.

In a particular embodiment, compounds of the invention have the Formula I′″h:

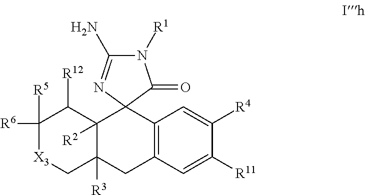

wherein X₃, R¹, R², R³, R⁴, R⁵, R⁶ and R¹¹ are as defined herein.

In a particular embodiment, compounds of the invention have the Formula Ih:

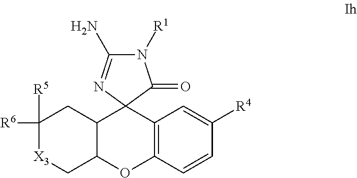

wherein X₃, R¹, R⁴, R⁵ and R⁶ are as defined herein.

In a particular embodiment, compounds of the invention have the Formula I′j:

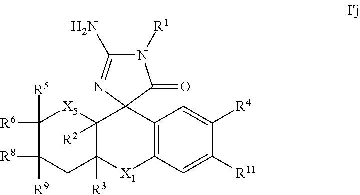

wherein X₁, X₅, R¹, R², R³, R⁴, R⁵, R⁶, R⁸, R⁹ and R¹¹ are as defined herein.

In a particular embodiment, compounds of the invention have the Formula I″j:

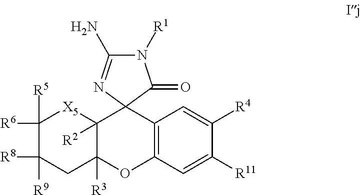

wherein X₅, R¹, R², R³, R⁴, R⁵, R⁶, R⁸, R⁹ and R¹¹ are as defined herein.

In a particular embodiment, compounds of the invention have the Formula I′″j:

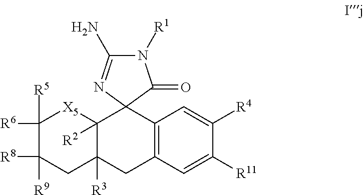

wherein X₅, R¹, R², R³, R⁴, R⁵, R⁶, R⁸, R⁹ and R¹¹ are as defined herein.

In certain embodiments, X₁ is selected from O, S, S(O), SO₂, NR¹⁰ and CHR¹⁰. In certain embodiments, X₁ is selected from O and CHR¹⁰. In certain embodiments, X₁ is O. In certain embodiments, X₁ is CHR¹⁰. In certain embodiments, R¹⁰ is hydrogen. In certain embodiments, X₁ is CH₂.

In certain embodiments, X₁ is selected from O, S, S(O), SO₂, NR¹⁰ and CHR¹⁰. In certain embodiments, X₁ is O.

In certain embodiments, X₂ is selected from CR⁵R⁶, NR⁷ and O. In certain embodiments, X₂ is CR⁵R⁶. In certain embodiments, X₂ is NR⁷. In certain embodiments, X₂ is O.

In certain embodiments, X₃ is selected from CR⁸R⁹ and O. In certain embodiments, X₃ is CR⁸R⁹. In certain embodiments, X₃ is O.

In certain embodiments, X₂ is selected from CR⁵R⁶, NR⁷ and O; X₃ is selected from CR⁸R⁹ and O; and X₅ is selected from CR¹²R¹³ and O, wherein two of X₂, X₃ and X₅ must contain C. In certain embodiments:

(i) X₂ is selected from CR⁵R⁶, NR⁷ and O; X₃ is CR⁸R⁹, and X₅ is CR¹²R¹³;

(ii) X₂ is CR⁵R⁶; X₃ is selected from CR⁸R⁹ and O; and X₅ is CR¹²R¹³; or

(iii) X₂ is CR⁵R⁶; X₃ is CR⁸R⁹; and X₅ is selected from CR¹²R¹³ and O. In certain embodiments, X₂ is selected from CR⁵R⁶, NR⁷ and O; X₃ is CR⁸R⁹; and X₅ is CR¹²R¹³. In certain embodiments, X₂ is selected from CR⁵R⁶, NR⁷ and O; X₃ is CR⁸R⁹; and X₅ is CHR¹². In certain embodiments, X₂ is CR⁵R⁶; X₃ is CR⁸R⁹ or O; and X₅ is CR¹²R¹³. In certain embodiments, X₂ is CR⁵R⁶; X₃ is CR⁸R⁹ or O; and X₅ is CHR¹². In certain embodiments, X₂ is CR⁵R⁶; X₃ is CR⁸R⁹; and X₅ is selected from CR¹²R¹³ and O. In certain embodiments, X₂ is CR⁵R⁶; X₃ is CR⁸R⁹; and X₅ is selected from CHR¹² and O.

In certain embodiments, X₂ is selected from CR⁵R⁶, NR⁷ and O, and X₃ is selected from CR⁸R⁹ and O, wherein at least one of X₂ or X₃ must contain C. In certain embodiments, X₂ is selected from CR⁵R⁶, NR⁷ and O, and X₃ is CR⁸R⁹, or X₂ is CR⁵R⁶, and X₃ is CR⁸R⁹ or O. In certain embodiments, X₂ is selected from CR⁵R⁶, NR⁷ and O, and X₃ is CR⁸R⁹. In certain embodiments, X₂ is CR⁵R⁶, and X₃ is CR⁸R⁹ or O.

In certain embodiments, X₄ is selected from CR¹¹ and N. In certain embodiments, X₄ is CH. In certain embodiments, X₄ is N.

In certain embodiments, X₅ is selected from CR¹²R¹³ and O. In certain embodiments, X₅ is CR¹²R¹³. In certain embodiments, X₅ is CHR¹². In certain embodiments, X₅ is O.

In certain embodiments, R¹ is selected from hydrogen, benzyl and C₁-C₃ alkyl, wherein the alkyl is optionally substituted with one or more R^{a} groups. In certain embodiments, each R^{a} is independently selected from OH, OCH₃, halogen, a 5 to 6 membered heteroaryl, and a 3 to 6 membered heterocyclyl, wherein the heterocyclyl is optionally substituted with C₁-C₃ alkyl optionally substituted with oxo. In certain embodiments, R¹ is selected from benzyl and C₁-C₃ alkyl, wherein the alkyl is optionally substituted with one or more R^{a} groups. In certain embodiments, R¹ is C₁-C₃ alkyl. In certain embodiments, R¹ is methyl.

In certain embodiments, R¹ is selected from hydrogen, benzyl and C₁-C₃ alkyl, wherein the alkyl is optionally substituted with one or more R^{a} groups. In certain embodiments, each R^{a} is independently selected from OH, OCH₃, halogen, a 5 to 6 membered heteroaryl, and a 3 to 6 membered heterocyclyl, wherein the heterocyclyl is optionally substituted with C₁-C₃ alkyl optionally substituted with oxo. In certain embodiments, R^{a} is a 5 to 6 membered heteroaryl, wherein the heteroaryl contains one, two or three heteroatoms selected from oxygen, nitrogen and sulfur. In certain embodiments, R^{a} is a 5 to 6 membered heteroaryl, wherein the heteroaryl is pyridinyl. In certain embodiments, R^{a} is a 3 to 6 membered heterocyclyl optionally substituted with C₁-C₃ alkyl optionally substituted with oxo, wherein the heterocyclyl contains one or two heteroatoms selected from oxygen, nitrogen and sulfur. In certain embodiments, R^{a} is a 3-6 membered heterocyclyl optionally substituted with C₁-C₃ alkyl optionally substituted with oxo, wherein the heterocyclyl is piperidinyl. In certain embodiments, R¹ is selected from hydrogen, benzyl, methyl, ethyl, —CH₂CH₂OH, —CH₂CH₂CH₂OH, —CH₂CH₂OCH₃, —CH₂CH₂CH₂OCH₃, —CH₂CF₃, pyridin-2-ylmethyl, pyridin-4-ylmethyl and (1-acetylpiperidin-4-yl)methyl. In certain embodiments, R¹ is selected from benzyl, methyl, ethyl, —CH₂CH₂OH, CH₂CH₂CH₂OH, —CH₂CH₂OCH₃, —CH₂CH₂CH₂OCH₃, —CH₂CF₃, pyridin-2-ylmethyl, pyridin-4-ylmethyl and (1-acetylpiperidin-4-yl)methyl.

In certain embodiments, R¹ is selected from hydrogen, benzyl and C₁-C₃ alkyl, wherein the alkyl is optionally substituted with one or more R^{a} groups. In certain embodiments, R^{a} is OH, OCH₃ or halogen. In certain embodiments, R¹ is selected from hydrogen, benzyl, methyl, ethyl, —CH₂CH₂OH, —CH₂CH₂CH₂OH, —CH₂CH₂OCH₃, —CH₂CH₂CH₂OCH₃ and —CH₂CF₃.

In certain embodiments, R² is hydrogen, halogen or C₁-C₆ alkyl. In certain embodiments, R² is hydrogen, halogen or C₁-C₃ alkyl. In certain embodiments, R² is hydrogen, F, methyl or ethyl.

In certain embodiments, R² is hydrogen or C₁-C₆ alkyl. In certain embodiments, R² is hydrogen or C₁-C₃ alkyl. In certain embodiments, R² is hydrogen. In certain embodiments, R² is in the (S) configuration. In certain embodiments, R² is in the (R) configuration.

In certain embodiments, R³ is hydrogen, halogen or C₁-C₆ alkyl. In certain embodiments, R³ is hydrogen or C₁-C₆ alkyl. In certain embodiments, R³ is hydrogen, halgogen or C₁-C₃ alkyl. In certain embodiments, R³ is hydrogen or C₁-C₃ alkyl. In certain embodiments, R³ is hydrogen or methyl.

In certain embodiments, R³ is hydrogen or C₁-C₆ alkyl. In certain embodiments, R³ is hydrogen or C₁-C₃ alkyl. In certain embodiments, R³ is hydrogen. In certain embodiments, R³ is in the (S) configuration. In certain embodiments, R³ is in the (R) configuration.

In certain embodiments, R² and R³ are independently selected form hydrogen, halogen and C₁-C₆ alkyl. In certain embodiments, R² is hydrogen, halogen or C₁-C₆ alkyl, and R³ is hydrogen or C₁-C₆ alkyl. In certain embodiments, R² and R³ are independently selected from hydrogen, halogen and C₁-C₃ alkyl. In certain embodiments, R² is hydrogen, halogen or C₁-C₃ alkyl, and R³ is hydrogen or C₁-C₃ alkyl. In certain embodiments, R² and R³ are hydrogen. In certain embodiments, R² is selected from hydrogen, F, methyl and ethyl, and R³ is selected from hydrogen and methyl. In certain embodiments, R² is selected from hydrogen, F, methyl and ethyl, and R³ is hydrogen. In certain embodiments, R² is hydrogen, and R³ is selected from hydrogen and methyl.

In certain embodiments, R² and R³ are hydrogen or C₁-C₆ alkyl. In certain embodiments, R² and R³ are hydrogen or C₁-C₃ alkyl. In certain embodiments, R² and R³ are hydrogen. In certain embodiments, R² and R³ are both in the (S) configuration. In certain embodiments, R² and R³ are both in the (R) configuration. In certain embodiments, R² is in the (S) configuration and R³ is in the (R) configuration. In certain embodiments, R² is in the (R) configuration and R³ is in the (S) configuration.

In certain embodiments, R⁴ is selected from Br, methoxy, 3-chloro-5-fluorophenyl, 3-chlorophenyl, 5-chloropyridin-3-yl, 2-fluoropyridin-3-yl, 5-(trifluoromethyl)pyridin-3-yl, pyrimidin-5-yl, 3-(difluoromethoxy)phenyl, 3-fluorophenyl, 5-fluoropyridin-3-yl, 3-cyanophenyl, 5-methoxypyridin-3-yl, 3-methoxyphenyl, 5-cyanopyridin-3-yl, 3-cyano-5-fluorophenyl, and 3-cyano-5-chlorophenyl.

In certain embodiments, R⁴ is selected from hydrogen, halogen, C₁-C₆ alkyl, C₁-C₆ alkenyl, C₁-C₆ alkynyl, —NHC(═O)(C₁-C₆ alkyl), —C(═O)NH(C₁-C₆ alkyl), a 3 to 6 membered carbocycle, a 3 to 6 membered heterocycle, phenyl, and a 5 to 6 membered heteroaryl, wherein the alkyl, alkenyl, alkynyl, carbocycle, heterocycle, phenyl and heteroaryl are optionally substituted with one or more R^{b} groups. In certain embodiments, R⁴ is selected from phenyl and 5 to 6 membered heteroaryl, wherein the phenyl and heteroaryl are optionally substituted with one or more R^{b} groups. In certain embodiments, each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen. In certain embodiments, each R^{b} is independently selected from halogen, CN, C₁-C₆ alkyl, and C₁-C₆ alkoxy, wherein the alkyl and alkoxy are optionally substituted with halogen. In certain embodiments, each R^{b} is independently selected from halogen, C₁-C₆ alkyl and C₁-C₆ alkoxy, wherein the alkyl and alkoxy are optionally substituted with halogen. In certain embodiments, R^{b} is selected from F, Cl, CF₃ and OCH₂F. In certain embodiments, R⁴ is phenyl, wherein the phenyl is optionally substituted with one or more R^{b} groups. In certain embodiments, R⁴ is a 5 to 6 membered heteroaryl, wherein the heteroaryl is optionally substituted with one or more R^{b} groups. In certain embodiments, R⁴ is a 5 to 6 membered heteroaryl, wherein the heteroaryl is optionally substituted with one or more R^{b} groups, and wherein the heteroaryl contains one, two, three or four heteroatoms selected from N, O and S. In certain embodiments, R⁴ is a 5 to 6 membered heteroaryl, wherein the heteroaryl is optionally substituted with one or more R^{b} groups, and wherein the heteroaryl contains one or two N heteroatoms. In certain embodiments, R⁴ is a 5 to 6 membered heteroaryl, wherein the heteroaryl is optionally substituted with one or more R^{b} groups, and wherein the heteroaryl is selected from pyridinyl and pyrimidinyl. In certain embodiments, R⁴ is selected from 3-chloro-5-fluorophenyl, 3-chlorophenyl, 5-chloropyridin-3-yl, 2-fluoropyridin-3-yl, 5-(trifluoromethyl)pyridin-3-yl, pyrimidin-5-yl, 3-(difluoromethoxy)phenyl and 3-fluorophenyl. In certain embodiments, R⁴ is selected from 3-chloro-5-fluorophenyl, 3-chlorophenyl, 3-(difluoromethoxy)phenyl and 3-fluorophenyl. In certain embodiments, R⁴ is selected from 5-chloropyridin-3-yl, 2-fluoropyridin-3-yl, 5-(trifluoromethyl)pyridin-3-yl and pyrimidin-5-yl.

In certain embodiments, each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen. In certain embodiments, each R^{b} is independently selected from halogen, CN, C₁-C₆ alkyl, and C₁-C₆ alkoxy, wherein the alkyl and alkoxy are optionally substituted with halogen. In certain embodiments, each R^{b} is independently selected from halogen, CN, C₁-C₆ alkyl and C₁-C₆ alkoxy, wherein the alkyl and alkoxy are optionally substituted with halogen. In certain embodiments, le is selected from F, Cl, CN, CF₃, OCH₂F and methoxy.

In certain embodiments, R⁴ is selected from phenyl and a 5 to 6 membered heteroaryl, wherein the phenyl and heteroaryl are optionally substituted with one or more R^{b} groups. In certain embodiments, R⁴ is selected from phenyl and a 5 to 6 membered heteroaryl, wherein the phenyl and heteroaryl are optionally substituted with one or two R^{b} groups. In certain embodiments, R⁴ is selected from 3-chloro-5-fluorophenyl, 3-chlorophenyl, 5-chloropyridin-3-yl, 2-fluoropyridin-3-yl, 5-(trifluoromethyl)pyridin-3-yl, pyrimidin-5-yl, 3-(difluoromethoxy)phenyl, 3-fluorophenyl, 5-fluoropyridin-3-yl, 3-cyanophenyl, 5-methoxypyridin-3-yl, 3-methoxyphenyl, 5-cyanopyridin-3-yl, 3-cyano-5-fluorophenyl, and 3-cyano-5-chlorophenyl.

In certain embodiments, R⁴ is phenyl, wherein the phenyl is optionally substituted with one or more R^{b} groups. In certain embodiments, R⁴ is selected from 3-chloro-5-fluorophenyl, 3-chlorophenyl, 3-(difluoromethoxy)phenyl, 3-fluorophenyl, 3-cyanophenyl, 3-methoxyphenyl, 3-cyano-5-fluorophenyl, and 3-cyano-5-chlorophenyl.

In certain embodiments, R⁴ is a 5 to 6 membered heteroaryl, wherein the heteroaryl is optionally substituted with one or more R^{b} groups. In certain embodiments, R⁴ is a 5 to 6 membered heteroaryl, wherein the heteroaryl is optionally substituted with one or more R^{b} groups, and wherein the heteroaryl contains one, two, three or four heteroatoms selected from N, O and S. In certain embodiments, R⁴ is a 5 to 6 membered heteroaryl, wherein the heteroaryl is optionally substituted with one or more R^{b} groups, and wherein the heteroaryl contains one or two N heteroatoms. In certain embodiments, R⁴ is a 5 to 6 membered heteroaryl, wherein the heteroaryl is optionally substituted with one or more R^{b} groups, and wherein the heteroaryl is selected from pyridinyl and pyrimidinyl. In certain embodiments, R⁴ is selected from 5-chloropyridin-3-yl, 2-fluoropyridin-3-yl, 5-(trifluoromethyl)pyridin-3-yl, 5-fluoropyridin-3-yl, 5-methoxypyridin-3-yl, 5-cyanopyridin-3-yl and pyrimidin-5-yl.

In certain embodiments, R⁵ and R⁶ are independently selected from hydrogen, halogen, hydroxyl, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or R⁵ and R⁶ taken together form an oxo group, or R⁵ and R⁶ together with the atom to which they are attached form a 3 to 6 membered heterocycle. In certain embodiments, R⁵ and R⁶ are independently selected from hydrogen, halogen, hydroxyl, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or R⁵ and R⁶ taken together form an oxo group, or R⁵ and R⁶ together with the atom to which they are attached form a 3 to 6 membered heterocycle.

In certain embodiments, R⁵ and R⁶ are independently selected from hydrogen, halogen, hydroxyl, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle. In certain embodiments, R⁵ and R⁶ are independently selected from hydrogen, halogen, hydroxyl and C₁-C₆ alkoxy optionally substituted with halogen or a 3 to 6 membered carbocycle. In certain embodiments, R⁵ and R⁶ are independently selected from hydrogen, halogen, hydroxyl and C₁-C₆ alkoxy optionally substituted with a 3 to 6 membered carbocycle. In certain embodiments, R⁵ and R⁶ are independently selected from hydrogen, F, OH, ethoxy and cyclopropylmethoxy.

In certain embodiments, R⁵ is hydrogen and R⁶ is selected from hydrogen, OH, ethoxy and cyclopropylmethoxy. In certain embodiments, R⁵ and R⁶ are F.

In certain embodiments, R⁵ and R⁶ are independently selected from hydrogen, halogen, OR^{c}, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or R⁵ and R⁶ taken together form an oxo group, or R⁵ and R⁶ together with the atom to which they are attached form a 3 to 6 membered heterocycle. In certain embodiments, R⁵ and R⁶ are independently selected from hydrogen, halogen, OR^{c}, CN, C₁-C₆ alkyl, phenyl, and a 5 to 6 membered heteroaryl, wherein the alkyl, phenyl and heteroaryl are optionally substituted with halogen, or R⁵ and R⁶ taken together form an oxo group, or R⁵ and R⁶ together with the atom to which they are attached form a 3 to 6 membered heterocycle. In certain embodiments, each R^{c} is independently selected from hydrogen and C₁-C₆ alkyl. In certain embodiments, R^{c} is hydrogen. In certain embodiments, R⁵ and R⁶ are independently selected from hydrogen, F and OH. In certain embodiments, R⁵ is hydrogen and R⁶ is selected from hydrogen and OH. In certain embodiments, R⁵ and R⁶ are F. In certain embodiments, R⁵ and R⁶ are taken together and form an oxo group. In certain embodiments, R⁵ and R⁶ together with the atom to which they are attached form a 3 to 6 membered heterocycle, wherein the heterocycle contains one, two or three heteroatoms selected from N, O and S. In certain embodiments, R⁵ and R⁶ together with the atom to which they are attached form a 3 to 6 membered heterocycle, wherein the heterocycle contains two O heteroatoms. In certain embodiments, R⁵ and R⁶ together with the atom to which they are attached form a 3 to 6 membered heterocycle, wherein the heterocycle is 1,3-dioxolanyl. In certain embodiments, R⁵ and R⁶ together form an oxo group or 1,3-dioxolan-2-yl. In certain embodiments, R⁵ and R⁶ together form 1,3-dioxolan-2-yl.

In certain embodiments, R⁷ is selected from hydrogen, C₁-C₆ alkyl, C₁-C₆ alkoxycarbonyl, —C(═O)NR^{f}R^{g}, —SO₂(C₁-C₆ alkyl), a 3 to 6 membered carbocycle, a 3 to 6 membered heterocycle, phenyl, and a 5 to 6 membered heteroaryl, wherein the alkyl, alkoxycarbonyl, carbocycle, heterocycle, phenyl and heteroaryl are optionally substituted with one or more R^{b} groups. In certain embodiments, R^{f} and R^{g} are independently selected from hydrogen and C₁-C₆ alkyl, wherein the alkyl is optionally substituted with halogen, CN or C₁-C₆ alkoxy. In certain embodiments, each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen. In certain embodiments, each R^{b} is independently selected from halogen, CN, C₁-C₆ alkyl, and C₁-C₆ alkoxy, wherein the alkyl and alkoxy are optionally substituted with halogen. In certain embodiments, R⁷ is selected from hydrogen and C₁-C₆ alkyl. In certain embodiments, R⁷ is selected from hydrogen and methyl. In certain embodiments, R⁷ is methyl.

In certain embodiments, R⁸ and R⁹ are independently selected from hydrogen, halogen, CN, C₁-C₆ alkyl, C₁-C₆ alkenyl, C₁-C₆ alkynyl, C₁-C₆ alkoxy, phenyl, a 5 to 6 membered heteroaryl and OR^{d}, wherein the alkyl, alkenyl, alkynyl, alkoxy, phenyl and heteroaryl are optionally substituted with halogen, or R⁸ and R⁹ taken together form an oxo group or C₁-C₆ alkenyl group, wherein the double bond of the alkenyl group is immediately attached to the carbon atom to which R⁸ and R⁹ are attached, or R⁸ and R⁹ together with the atom to which they are attached form a 3 to 6 membered heterocycle. In certain embodiments, R⁸ and R⁹ are independently selected from hydrogen, halogen, C₁-C₆ alkyl, C₁-C₆ alkenyl, C₁-C₆ alkynyl, and OR^{d}, or R⁸ and R⁹ taken together form an oxo group or C₁-C₆ alkenyl group, wherein the double bond of the alkenyl group is immediately attached to the carbon atom to which R⁸ and R⁹ are attached, or R⁸ and R⁹ together with the atom to which they are attached form a 3 to 6 membered heterocycle. In certain embodiments, each R^{d} is independently selected from hydrogen and C₁-C₆ alkyl, wherein the alkyl is optionally substituted with one or more R^{e} groups. In certain embodiments, R^{d} is selected from hydrogen, methyl, ethyl and cyclopropylmethyl. In certain embodiments, each R^{e} is independently selected from halogen and C₃-C₆ cycloalkyl. In certain embodiments, R^{e} is cyclopropyl. In certain embodiments, R⁸ and R⁹ are independently selected from hydrogen, F, OH, methyl, methoxy, ethoxy and cyclopropylmethoxy. In certain embodiments, R⁸ is selected from hydrogen, F and methyl, and R⁹ is selected from hydrogen, F, OH, methyl, methoxy, ethoxy and cyclopropylmethoxy. In certain embodiments, R⁸ and R⁹ taken together form an oxo group or C₁-C₆ alkenyl group, wherein the double bond of the alkenyl group is immediately attached to the carbon atom to which R⁸ and R⁹ are attached. In certain embodiments, R⁸ and R⁹ are taken together to form oxo or methylene. In certain embodiments, R⁸ and R⁹ are taken together to form an oxo group. In certain embodiments, R⁸ and R⁹ are taken together to form methylene. In certain embodiments, R⁸ and R⁹ together with the atom to which they are attached form a 3 to 6 membered heterocycle. In certain embodiments, R⁸ and R⁹ together with the atom to which they are attached form a 3 to 6 membered heterocycle, wherein the heterocycle contains one, two or three heteroatoms selected from N, O and S. In certain embodiments, R⁸ and R⁹ together with the atom to which they are attached form a 3 to 6 membered heterocycle, wherein the heterocycle contains two O heteroatoms. In certain embodiments, R⁸ and R⁹ together with the atom to which they are attached form a 3 to 6 membered heterocycle, wherein the heterocycle is 1,3-dioxolanyl. In certain embodiments, R⁸ and R⁹ together form oxo, methylene or 1,3-dioxolan-2-yl. In certain embodiments, R⁸ and R⁹ together form 1,3-dioxolan-2-yl.

In certain embodiments, R¹⁰ is selected from hydrogen, halogen and C₁-C₆ alkyl. In certain embodiments, R¹⁰ is hydrogen.

In certain embodiments, R¹¹ is selected from hydrogen, halogen and C₁-C₆ alkyl, wherein the alkyl is optionally substituted with one or more R^{b} groups. In certain embodiments, each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen. In certain embodiments, each R^{b} is independently selected from halogen, CN, C₁-C₆ alkyl, and C₁-C₆ alkoxy, wherein the alkyl and alkoxy are optionally substituted with halogen. In certain embodiments, R¹¹ is selected from hydrogen and halogen. In certain embodiments, R¹¹ is selected from hydrogen and F. In certain embodiments, R¹¹ is hydrogen. In certain embodiments, R¹¹ is F.

In certain embodiments, R¹¹ is selected from hydrogen, halogen and C₁-C₆ alkyl, wherein the alkyl is optionally substituted with one or more R^{b} groups. In certain embodiments, each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen. In certain embodiments, each R^{b} is independently selected from halogen, CN, C₁-C₆ alkyl, and C₁-C₆ alkoxy, wherein the alkyl and alkoxy are optionally substituted with halogen. In certain embodiments, R¹¹ is hydrogen.

One embodiment provides a compound of Formula I as named in any one of Examples 1 to 43 herein, or a stereoisomer, diastereomer, enantiomer, tautomer or pharmaceutically acceptable salt thereof.

One embodiment provides a compound of Formula I′ as named in any one of Examples 1 to 116 herein, or a stereoisomer, diastereomer, enantiomer, tautomer or pharmaceutically acceptable salt thereof.

It will be appreciated that certain compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present compounds.

In the structures shown herein, where the stereochemistry of any particular chiral atom is not specified, then all stereoisomers are contemplated and included as the compounds described herein. Where stereochemistry is specified by a solid wedge or dashed line representing a particular configuration, then that stereoisomer is so specified and defined.

It will also be appreciated that certain compounds of Formula I′may be used as intermediates for further compounds of Formula I′.

It will be further appreciated that the compounds described herein may exist in unsolvated as well as solvated forms with pharmaceutically acceptable solvents, such as water, ethanol, and the like, and it is intended that the compounds embrace both solvated and unsolvated forms.

Synthesis of Compounds

Compounds described herein may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

It will be appreciated that synthetic procedures employed in the preparation of compounds of the invention will depend on the particular substituents present in a compound. In preparing compounds of the invention, protection of remote functionalities (e.g., primary or secondary amines, etc.) of intermediates may be necessary but may not be illustrated in the following general Schemes. The need for such protection will vary depending on the nature of the remote functionality and the conditions of the preparation methods. The need for such protection is readily determined by one skilled in the art. For a general description of protecting groups and their use, see Greene's Protective Groups in Organic Synthesis, supra.

For illustrative purposes, Schemes 1-6 show general methods for preparing the compounds described herein, as well as key intermediates. For a more detailed description of the individual reaction steps, see the Examples section below. Those skilled in the art will appreciate that other synthetic routes may be used to synthesize the compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

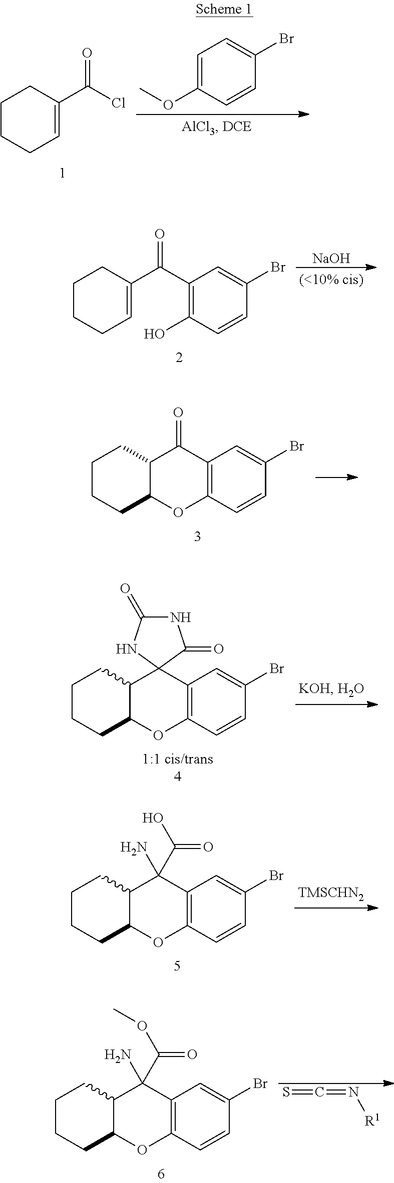

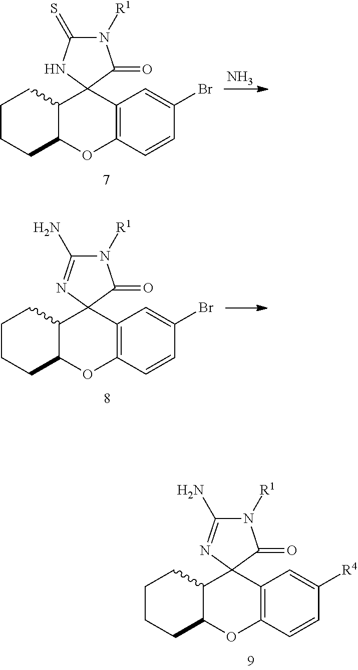

Scheme 1 shows a general scheme for the synthesis of compounds 8 and 9, wherein R¹ and R⁴ are as defined herein. Compound 1 may be reacted with 1-bromo-4-methoxybenzene to provide compound 2. Compound 2 may be reacted with a ring closing agent, such as NaOH, to provide compound 3. Compound 3 may be treated with a Bucherer-Bergs reaction, heated with cyanopotassium and ammonium carbonate, to provide compound 4. Compound 4 may be reacted with KOH to provide compound 5. Compound 5 may be reacted with TMSCHN₂ to provide compound 6. Compound 6 may be reacted with isothiocyanate-R¹ to provide compound 7. Compound 7 may be reacted with ammonia and an oxidant, such as tert-butyl hydroperoxide, to provide compound 8. When R⁴ is not bromine, a Suzuki, Negishi or Stille coupling installs the R⁴ group and provides compound 9.

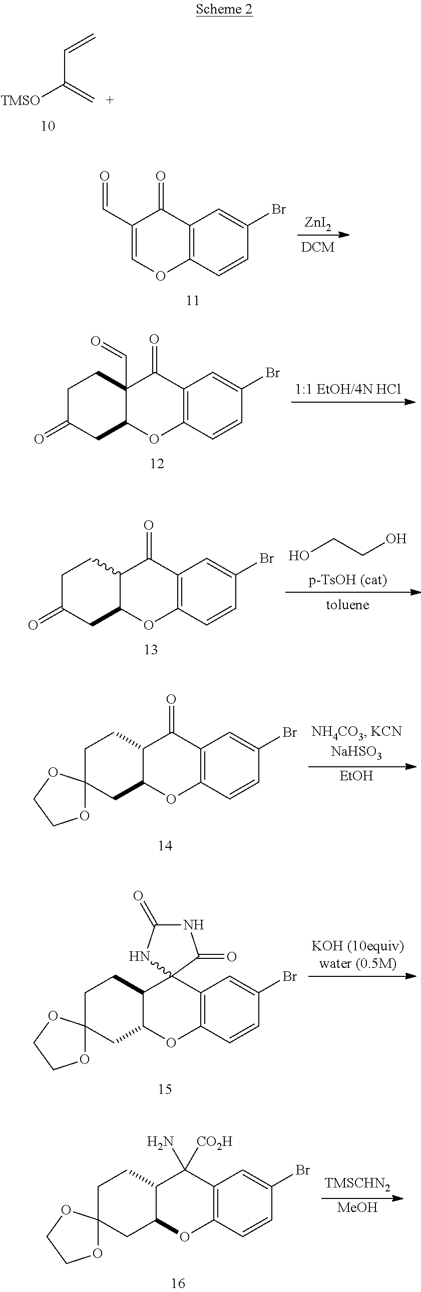

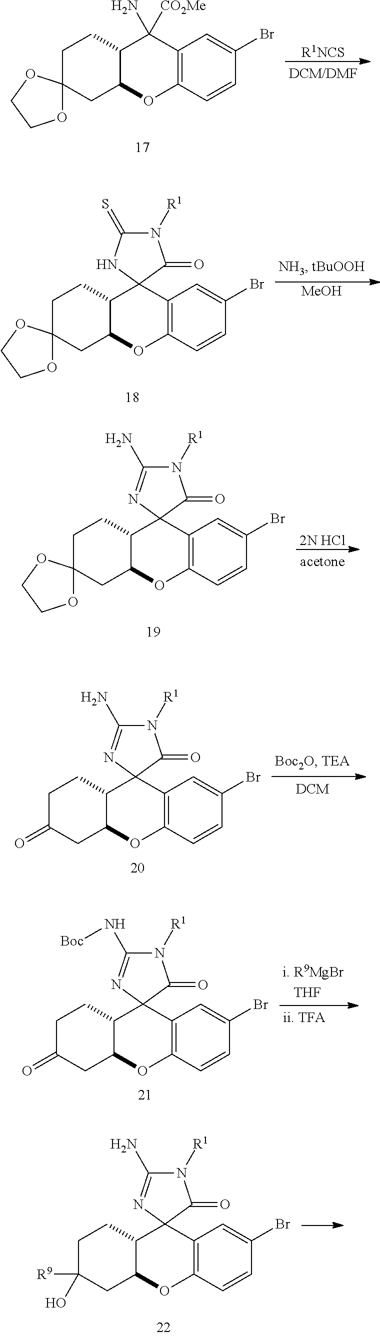

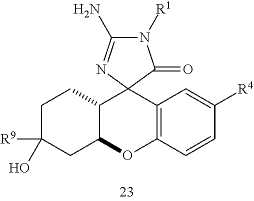

Scheme 2 shows a general scheme for the synthesis of compounds 22 and 23, wherein R¹, R⁴ and R⁹ are as defined herein. Compound 10 may be reacted with compound 11 to provide compound 12. Compound 12 may be reacted with EtOH/HCl to provide compound 13. Compound 13 may be reacted with ethane-1,2-diol and TsOH to provide compound 14. Compound 14 may be reacted with NH₄CO₃, KCN and NaHSO₃ to provide compound 15. Compound 15 may be reacted with KOH to provide compound 16. Compound 16 may be reacted with TMSCHN₂ to provide compound 17. Compound 17 may be reacted with isothiocyanate-R¹ to provide compound 18. Compound 18 may be reacted with ammonia and an oxidant, such as tert-butyl hydroperoxide, to provide compound 19. Compound 19 may be reacted with HCl to provide compound 20. Compound 20 may be protected with Boc₂O and triethylamine to provide compound 21. Compound 21 may be reacted with R⁹MgBr and then deprotected to provide compound 22. When R⁴ is not bromine, a Suzuki, Negishi or Stille coupling installs the R⁴ group and provides compound 23.

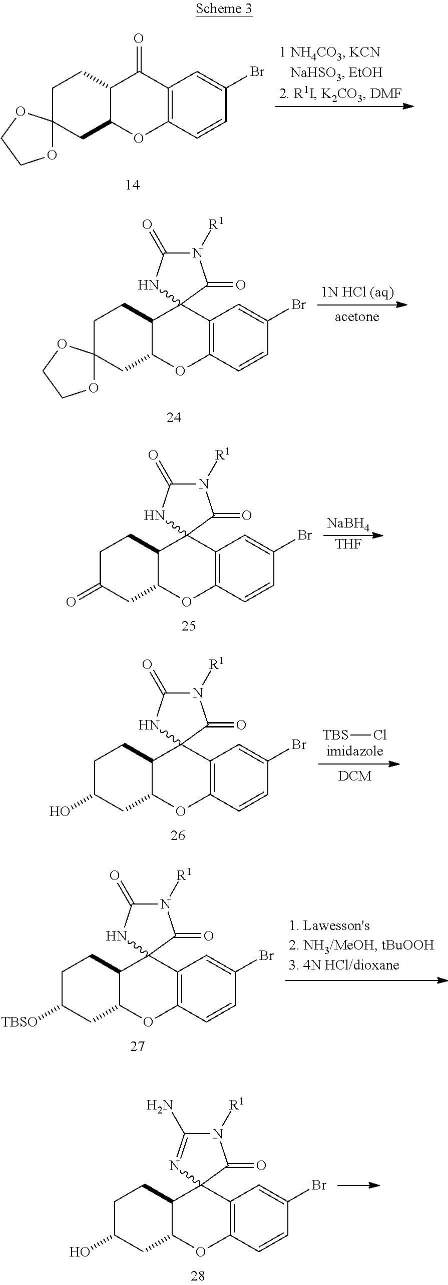

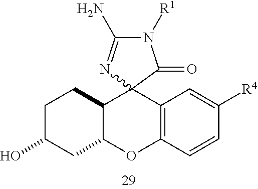

Scheme 3 shows a general scheme for the synthesis of compounds 28 and 29, wherein R¹ and R⁴ are as defined herein. Compound 14 may be reacted with NH₄CO₃, KCN and NaHSO₃, followed by R¹-iodide to provide compound 24. Compound 24 may be reacted with HCl to provide compound 25. Compound 25 may be reacted with NaBH₄ to provide compound 26. Compound 26 may be protected with TBS-Cl to provide compound 27. Compound 27 may be reacted with Lawesson's reagent, followed by ammonium hydroxide or ammonia in methanol and an oxidant, such as tert-butyl hydroperoxide, and then deprotected to provide compound 28. When R⁴ is not bromine, a Suzuki, Negishi or Stille coupling installs the R⁴ group and provides compound 29.

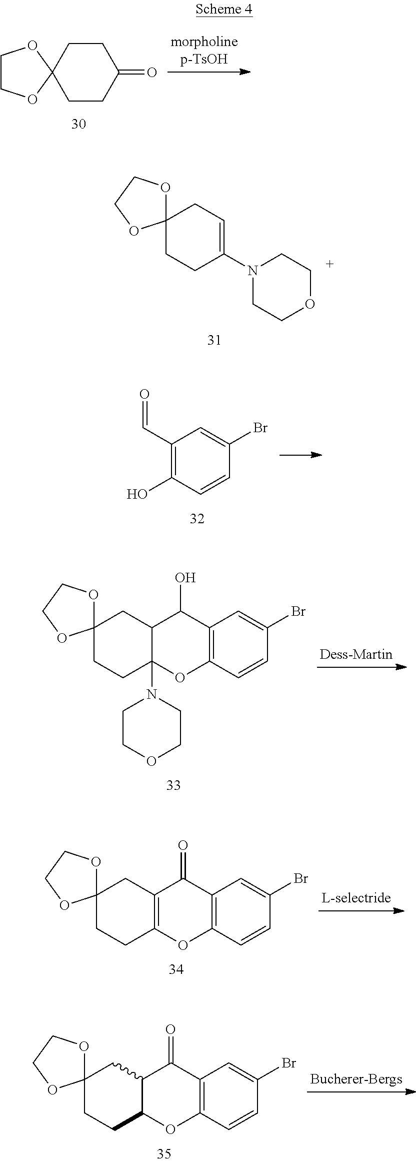

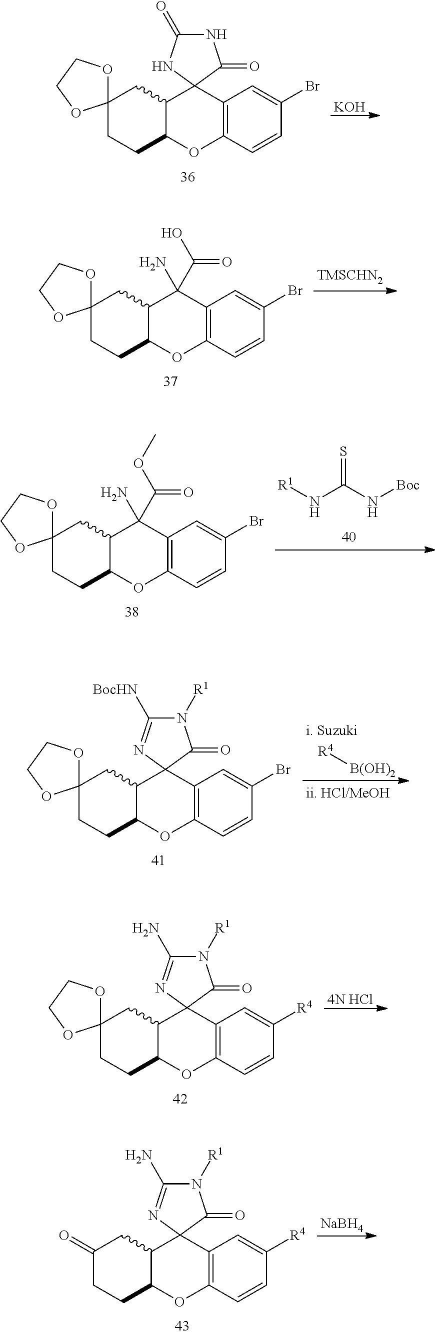

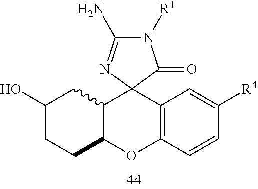

Scheme 4 shows a general scheme for the synthesis of compounds 42, 43 and 44, wherein R¹ and R⁴ are as defined herein. Compound 30 may be reacted with morpholine and p-TsOH in a solvent to provide compound 31. Compound 31 may be reacted with compound 32 to provide compound 33. Compound 33 may be oxidized with Dess-Martin Periodinane to provide compound 34. Compound 34 may be selectively reduced with L-selectride. Compound 35 may be subjected to a Bucherer-Bergs reaction to provide the hydantoin 36. Compound 36 may be reacted with KOH to provide compound 37. Compound 37 may be methylated with TMSCHN₂ in a solvent to provide compound 38. Compound 38 may be reacted with compound 40 to provide compound 41. When R⁴ is not bromine, a Suzuki, Negishi or Stille coupling installs the R⁴ group, followed by HCl in MeOH to deprotect and provide compound 42. Compound 42 may be reacted with HCl in a solvent to provide compound 43. Compound 43 may be reduced with sodium borohydride to provide compound 44.

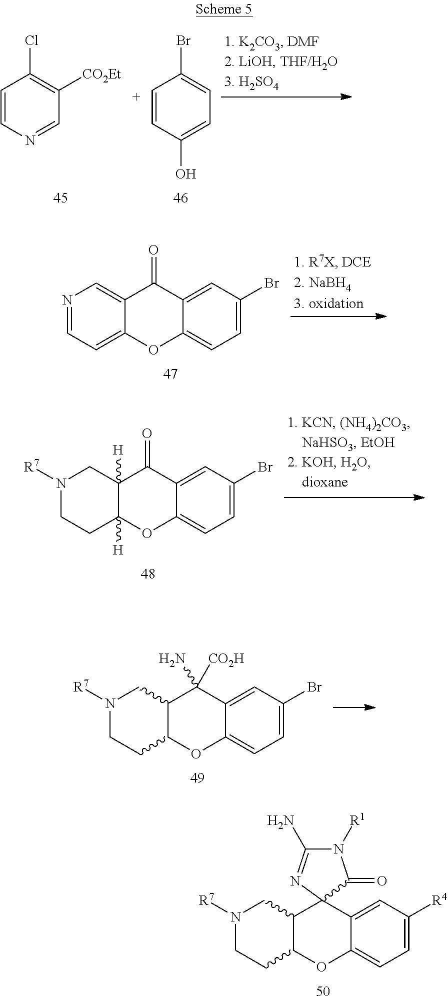

Scheme 5 shows a general scheme for the synthesis of compound 50, wherein R¹, R⁴ and R⁷ are as defined herein. Compound 45 may be reacted with compound 46 to provide compound 47. The R⁷ group may then be installed, followed by reduction with NaBH₄, and followed by oxidation to provide compound 48. Compound 49 may be prepared by first reacting compound 48 with potassium cyanide, ammonium carbonate, sodium bisulfite and ethanol, and then with potassium hydroxide, water and dioxane. Compound 50 is prepared in a similar manner to compounds 6-9 of Scheme 1.

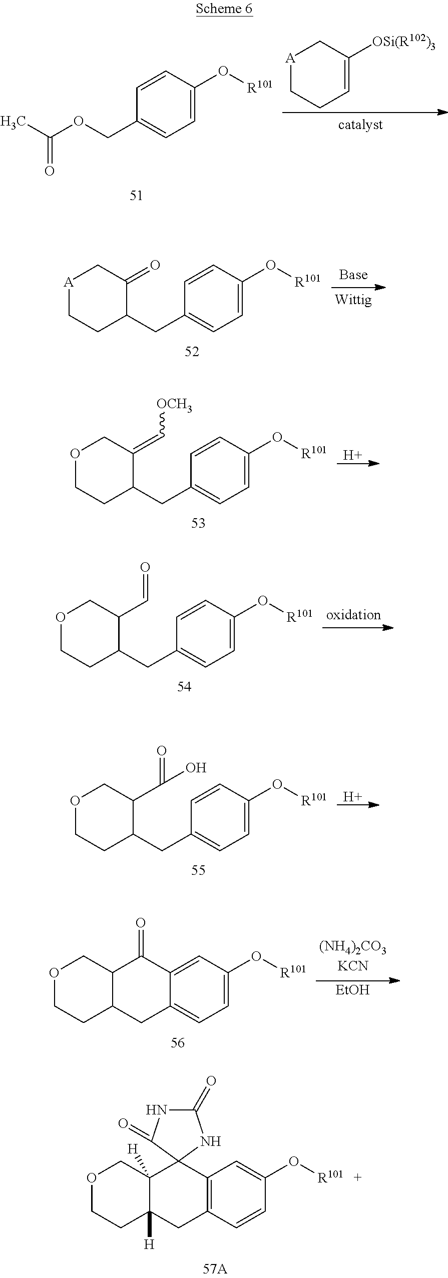

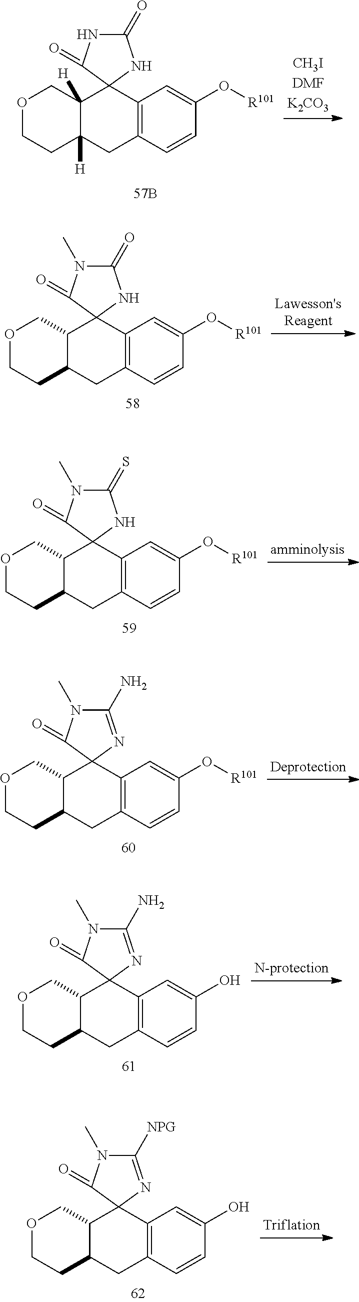

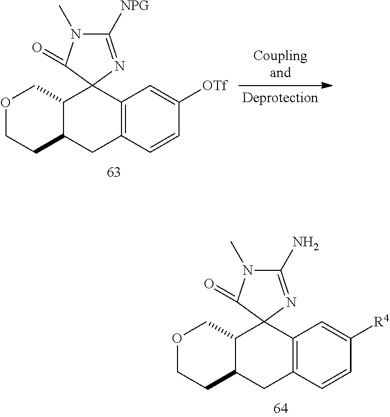

Scheme 6 shows a general scheme for the synthesis of compound 64, wherein R⁴ is as defined herein. The compound 52, wherein R¹⁰¹ may be alkyl, benzy or substituted benzyl, may be prepared from the reaction of an appropriate benzyl acetate derivative, wherein R¹⁰² may be alkyl and A may be oxygen or carbon, with a silyl vinyl ether in the presence of a catalyst, such as NH(SO₂CF₃)₂, as described in Mendoza, Oscar, et al. “Trialkylsilyl triflimides as easily tunable organocatalysts for allylation and benzylation of silyl carbon nucleophiles with non-genotoxc reagents.” Tetrahedron Letters. Vol. 51, No. 19 (2010): pp. 2571-2575. The ketone 52 may be subjected to Wittig reaction conditions as described in the literature (Anzalone, Luigi and Jerry A. Hirsch. “Syntheses and Equilibrations of 6- and 7-Carbomethoxy-trans-2-oxadcalins.” J. Org. Chem. Vol. 50, No. 15 (1985): pp. 2607-2613) to prepare vinyl ether 53. Hydrolysis of 53 with an aqueous acid, such as HCl, will furnish the aldehyde 54, which in turn may be oxidized to the corresponding carboxylic acid 55 with an oxidizing reagent, such as NaClO₂. The ring closure may be achieved by treatment of ketone 55 with a strong acid, such as TFA, MSA, PPA, concentrated H₂SO₄ or a mixture of these acids. The compound 56 may be reacted with KCN and (NH₄)₂CO₃ to provide a mixture of 57A and 57B, which may be separated by chromatography methods or by selective crystallization. Compound 57 may be reacted with an alkylating agent, such as CH₃I, in the presence of a base to provide compound 58. Compound 58 may be treated with Lawesson's reagent, followed by ammonium hydroxide or ammonia in the presence of an oxidant, such as tert-butyl hydroperoxide, to give compound 60. Deprotection of the R¹⁰¹ group can be achieved by treatment of compound 60 with HBr or BBR₃ when R¹⁰¹ is OCH₃. The protection of NH₂ group on compound 61 with an appropriate nitrogen protecting group followed by triflation of the phenol 62, wherein PG is a nitrogen protecting group such as Boc or CH═N(CH₃)₂, with a triflating agent, such as triflic anhydride or 1,1,1-trifluoro-N-phenyl-N-(trifluoromethylsulfonyl)methanesulfonamide, in the presence of a base will provide compound 63. Compound 64 can be prepared by subjecting compound 63 to various coupling reactions such as, but not limited to, Suzuki, Ullman, O-alkylation and Mitsunobu.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary, such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and S. Wilen. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds described herein may be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Indications

The compounds of the invention inhibit the cleavage of amyloid precursor protein by β-secretase which is implicated in diseases, in particular, neurodegenerative diseases such as Alzheimer's disease. In AD, processing of APP by β-secretase produces soluble N-APP, which activates extrinsic apoptotic pathways by binding to death receptor 6. Furthermore, APP that is processed by β-secretase is subsequently cleaved by γ-secretase, thereby producing amyloid beta peptides, such as Aβ 1-42 that form amyloid plaques, which contribute to nerve cell death. Compounds of the invention inhibit enzymatic cleavage of APP by β-secretase.

Accordingly, in an aspect of the invention, there is provided a method of inhibiting cleavage of APP by β-secretase in a mammal comprising administering to said mammal an effective amount of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j.

In another aspect of the invention, there is provided a method for treating a disease or condition mediated by the cleavage of APP by β-secretase in a mammal, comprising administering to said mammal an effective amount of a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j.

In another aspect, there is provided the use of a compound of Formula I′, I, I′a, Ia, I′b, Ib, I′c, Ic, I′d, Id, I′e, Ie, If, I′g, I″g, I′″g, Ig, I′h, I″h, I′″h, Ih, I′j, I″j or I′″j in the manufacture of a medicament for the treatment of a neurodegenerative disease. In one embodiment, the neurodegenerative disease is Alzheimer's disease.

In another aspect of the invention, there is provided a use of a compound of Formula I′, I, I′a, Ia, I′b, Ib, I′c, Ic, I′d, Id, I′e, Ie, If, I′g, I″g, I′″g, Ig, I′h, I″h, I′″h, Ih, I′j, I″j or I′″j in the treatment of neurodegenerative diseases. In one embodiment, the neurodegenerative disease is Alzheimer's disease.

Compounds of the invention may be administered prior to, concomitantly with, or following administration of other therapeutic compounds. Sequential administration of each agent may be close in time or remote in time. The other therapeutic agents may be anti-neurodegenerative with a mechanism of action that is the same as compounds of the invention, i.e., inhibit beta-secretase cleavage of APP, or a different mechanism of action, e.g., anti-Aβ antibodies. The compounds may be administered together in a unitary pharmaceutical composition or separately and, when administered separately this may occur simultaneously or sequentially in any order. Such sequential administration may be close in time or remote in time.

The invention also includes compositions containing the compounds of the invention and a carrier, diluent or excipient, as well as methods of using the compounds of the invention to prepare such compositions. In a particular embodiment, there is provided a pharmaceutical composition comprising a compound of Formula I′, I, I′a, Ia, I′b, Ib, I′c, Ic, I′d, Id, I′e, Ie, I′f, If, I′g, I″g, I′″g, Ig, I′h, I″h, I′″h, Ih, I′j, I″j or I′″j or Ih and a pharmaceutically acceptable carrier, diluent or excipient.

Typically, the compounds of the invention used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In one embodiment, formulations comprising compounds of the invention are sterile. The compounds ordinarily will be stored as a solid composition, although lyophilized formulations or aqueous solutions are acceptable.

Compositions comprising compounds of the invention will be formulated, dosed, and administered in a fashion consistent with good medical practice. Factors for consideration in this context include the particular disorder being treated, the particular mammal being treated, the clinical condition of the individual patient, the cause of the disorder, the site of administration, the method of administration, the scheduling of administration, and other factors known to medical practitioners.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

Generally, the initial pharmaceutically effective amount of the compound of the invention administered parenterally per dose will be in the range of about 0.01-100 mg/kg/day, for example about 0.1 to 20 mg/kg of patient body weight per day, with the typical initial range of compound used being 0.3 to 15 mg/kg/day. Oral unit dosage forms, such as tablets and capsules, may contain from about 25 to about 1000 mg of the compound of the invention.

The compound of the invention may be administered by any suitable means, including oral, sublingual, buccal, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

Another formulation may be prepared by mixing a compound described herein and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound described herein or pharmaceutical composition thereof) or aid in the manufacturing of the pharmaceutical product (i.e., medicament).

### EXAMPLES

The invention will be more fully understood by reference to the following examples. They should not, however, be construed as limiting the scope of the invention. For example, the synthesis of non-exemplified compounds may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds described herein. The identity and purity of compounds were checked by LCMS and ¹H NMR analysis.

Column chromatography was done on a Biotage system (Manufacturer: Dyax Corporation) having a silica gel column or on a silica SepPak cartridge (Waters) (unless otherwise stated). ¹H NMR spectra were recorded on a Varian instrument operating at 400 MHz. ¹H-NMR spectra were obtained as CDCl₃, CD₃OD, D₂O, (CD₃)₂SO, (CD₃)₂CO, C₆D₆, CD₃CN solutions (reported in ppm), using tetramethylsilane (0.00 ppm) or residual solvent (CDCl₃: 7.26 ppm; CD₃OD: 3.31 ppm; D₂O: 4.79 ppm; (CD₃)₂SO: 2.50 ppm; (CD₃)₂CO: 2.05 ppm; C₆D₆: 7.16 ppm; CD₃CN: 1.94 ppm) as the reference standard. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

### Biological Evaluation

### Cellular BACE1 Inhibition Assay

The BACE inhibition properties of the compounds of the invention may be determined by the following in vitro cellular Amyloidβ 1-40 production assay.

Inhibition of Amyloidβ 1-40 production was determined by incubating cells with compound for 48 hours and quantifying the level of Amyloidβ 1-40 using an homogeneous time-resolved fluorescence (“HTRF”) immunoassay.

Materials and Methods: HEK-293 cells stably transfected with a DNA construct containing the coding sequence for the wild type APP695 sequence were grown in Dulbecco's Modified Eagle Medium (“DMEM”) supplemented with 10% fetal bovine serum, penicillin/streptomycin and 150 μg/mL G418. Cells were plated in 96-well plates at 35,000 cells/well and allowed to attach for 8-12 hours. Media was changed to DMEM supplemented with 10% fetal bovine serum, penicillin/streptomycin 15 minutes prior to compound addition. Diluted compounds were then added at a final concentration of 0.5% DMSO. After 48 hours, 4 μL of media from each well was added to a corresponding well of a 384 well plate (Perkin Elmer Cat#6008280) containing the HTRF reagents. HTRF reagents were obtained from the CisBio Amyloidβ 1-40 peptide assay kit (Cat# 62B40PEC) and were prepared as follows anti-peptide β (1-40)-Cryptate and anti-peptide β (1-40)-XL655 were stored in 2 plate aliquots at −80° C. Diluent and Reconstitution buffer were stored at 4° C. Aliquots of the two antibodies were diluted 1:100 with Reconstitution buffer, and this mixture was diluted 1:2 with Diluent. 12 μL of the reagent mixture was added to the required wells of the 384 well assay plate. The assay plate was incubated at 4° C. for 17 hours and then analyzed for fluorescence at 665 and 620 nm. The reported IC₅₀ below may be from a single assay or the mean of multiple assays.

The following compounds were tested in the above assay:

| Example # | IC₅₀ (nM) |
|---|---|
| Example 1 | 2.0 |
| Example 2 | 1.5 |
| Example 3 | 1.3 |
| Example 7 | 18.3 |
| Example 5 | 6.1 |
| Example 6 | 36.2 |
| Example 7 | 35.9 |
| Example 8 | 5.5 |
| Example 10 | 168 |
| Example 11 | 46.6 |
| Example 12 | 11.4 |
| Example 14 | 48.3 |
| Example 15 | 8.6 |
| Example 17 | 74.1 |
| Example 18 | 125.0 |
| Example 19 | 21.4 |
| Example 20 | 172.2 |
| Example 28 | 51.2 |
| Example 29 | 554.1 |
| Example 30 | 22.3 |
| Example 32 | 426.2 |
| Example 33 | 32.2 |
| Example 34 | 1667 |
| Example 35 | 271.4 |
| Example 36 | 221.1 |
| Example 38 | 5.9 |
| Example 41 | 41.4 |
| Example 47 | 9.9 |
| Example 49 | 43.9 |
| Example 50 | 13.2 |
| Example 54 | 16.0 |
| Example 55 | 18.3 |
| Example 62 | 2.3 |
| Example 63 | 4.3 |
| Example 64 | 1.8 |
| Example 69 | 0.8 |
| Example 70 | 1.5 |
| Example 78 | 1.5 |
| Example 79 | 4.3 |
| Example 86 | 4.6 |
| Example 96 | 3.9 |
| Example 99 | 241.5 |
| Example 101 | 5.3 |
| Example 107 | 801 |

### Example 1

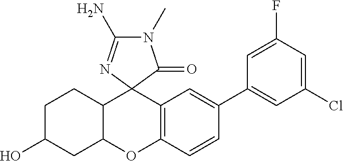

### 2-amino-7′-(3-chloro-5-fluorophenyl)-3′-hydroxy-1-methyl-1′,2′,3′,4′,4a′,9a′-hexahydro spiro[imidazole-4,9′-xanthen]-5(1H)-one

Step A: A solution of 6-bromo-4-oxo-4H-chromene-3-carbaldehyde (25.0 g, 98.8 mmol) in CH₂Cl₂ (988 mL) was stirred at room temperature until the solution was homogeneous (additional CH₂Cl₂ was added until completely dissolved). Zinc (II) iodide (4.73 g, 14.8 mmol) was added to this mixture and the mixture was cooled to 0° C. (Buta-1,3-dien-2-yloxy)trimethylsilane (21.1 g, 148 mmol) was added to this mixture, and the ice bath was removed. The reaction was stirred for 1.5 hours, or until complete by HPLC (if necessary, additional diene was added to drive reaction). Celite® (25 g) and 1 mL HCl (concentrated) were added to the reaction mixture, and the resulting mixture was stirred at room temperature for 15 minutes. The mixture was filtered through glass microfibre filter (“GF/F”) paper, and the filtrate was transferred to a separatory funnel and washed with water. The organic layer was dried and concentrated to give crude 7-bromo-3,9-dioxo-2,3,4,4a,9,9a-hexahydro-1H-xanthene-9a-carbaldehyde (28.0 g, 86.7 mmol, 88%) as a racemic mixture of diastereomers.

Step B: A mixture of 7-bromo-3,9-dioxo-2,3,4,4a,9,9a-hexahydro-1H-xanthene-9a-carbaldehyde (17.1 g, 52.9 mmol) and 4N HCl (132 mL) in ethanol (265 mL) was heated at 100° C. for 18 hours. The reaction mixture was concentrated to remove ethanol, dissolved in CH₂Cl₂, and then the layers were separated. The organic layer was washed with brine, dried and concentrated. The residue was dissolved with CH₂Cl₂ to load onto a silica chromatography column then eluted with 10-50% ethyl acetate/hexanes with 10% CH₂Cl₂ gradient. Both cis and trans 7-bromo-4,4-a-dihydro-1H-xanthene-3,9(2H, 9aH)-dione were collected.

Step C: A solution of 7-bromo-4,4-a-dihydro-1H-xanthene-3,9(2H, 9aH)-dione (5.00 g, 16.9 mmol), ethane-1,2-diol (1.04 mL, 18.6 mmol) and TsOH—H₂O (0.322 g, 1.69 mmol) in toluene (84.71 mL, 16.94 mmol) was heated to 130-135° C. (Dean-Stark apparatus) for 4 hours. Additional ethane-1,2-diol was added as necessary to drive the reaction to completion, because at 130-135° C., ethylene glycol was also collected in the Dean-Stark trap. Bis-ketal was formed in substantial amounts when the reaction was run at temperatures below 130° C. The reaction mixture was diluted with ethyl acetate and washed with water. The organic layer was washed with sodium carbonate, brine, dried and concentrated to give (4a′R,9a′R)-7′-bromo-1′,4′,4a′,9a′-tetrahydrospiro[[1,3]dioxolane-2,3′-xanthen]-9′(2′H)-one (4.95 g, 14.6 mmol, 86%). C is material epimerizes to trans under these reaction conditions.

Step D: Ammonium carbonate (5.80 g, 60.4 mmol), KCN (0.983 g, 15.1 mmol), and NaHSO₃ (0.314 g, 3.02 mmol) were added to a solution in a teflon-lined steel pressure reactor of 7′-bromo-1′,4′,4a′,9a′-tetrahydrospiro[[1,3]dioxolane-2,3′-xanthen]-9′(2′H)-one (2.56 g, 7.55 mmol) in EtOH (7.55 mL). The reactor was sealed and heated at 135° C. for 18 hours. The reactor was cooled to ambient temperature. The reaction mixture was transferred to an erlenmeyer flask and acidified with HCl (2N) and repeatedly washed with water/EtOAc to maximize transfer. The layers were separated, and the aqueous layer was extracted with EtOAc (3×). The combined organic layers were dried and concentrated to afford 7′-bromo-3′-(spiro[1,3]dioxolane)-1′,2′,3′,4′,4a′,9a′-hexahydro spiro[imidazolidine-4,9′-xanthene]-2,5-dione (3.00 g, 7.33 mmol, 97%).

Step E: A mixture of 7′-bromo-3′-(spiro[1,3]dioxolane)-1′,2′,3′,4′,4a′,9a′-hexahydrospiro[imidazolidine-4,9′-xanthene]-2,5-dione (3.0 g, 7.33 mmol), K₂CO₃ (3.04 g, 22.0 mmol) and MeI (0.457 mL, 7.33 mmol; d 2.275) in dimethylformamide (“DMF”, 36.7 mL, 7.33 mmol) was stirred at room temperature overnight. The reaction was diluted with water and extracted with ethyl acetate (3×). The combined organic layers were washed with brine (3×). The organic layer was dried and concentrated to afford 7′-bromo-3′-(spiro[1,3]dioxolane)-1-methyl-1′,2′,3′,4′,4a′,9a′-hexahydrospiro[imidazolidine-4,9′-xanthene]-2,5-dione (2.97 g, 7.02 mmol, 96%).

Step F: HCl (12 mL, 24 mmol) was added to a solution of 7′-bromo-3′-(spiro[1,3]dioxolane)-1-methyl-1′,2′,3′,4′,4a′,9a′-hexahydrospiro[imidazolidine-4,9′-xanthene]-2,5-dione (1.0 g, 2.4 mmol) in acetone (12 mL, 2.4 mmol), and the resulting solution was stirred at 60° C. for 24 hours. The mixture was extracted with EtOAc (3×) and the combined organic layers were dried and concentrated to afford 7′-bromo-1-methyl-1′,4′,4a′,9a′-tetrahydrospiro[imidazolidine-4,9′-xanthene]-2,3′,5(2′H)-trione (0.88 g, 2.3 mmol, 98%).

Step G: NaBH₄ (13.1 mg, 0.345 mmol) was added to a solution of 7′-bromo-1-methyl-1′,4′,4a′,9a′-tetrahydrospiro[imidazolidine-4,9′-xanthene]-2,3′,5(2′H)-trione (131 mg, 0.345 mmol) in tetrahydrofuran (“THF”, 1.73 mL, 0.345 mmol) at −78° C. The resulting mixture was slowly warmed to room temperature. After 1 hour, the reaction mixture was quenched with water, diluted with brine, and then extracted with ethyl acetate (3×). The combined organic layers were dried and concentrated to give 7′-bromo-3′-hydroxy-1-methyl-1′,2′,3′,4′,4a′,9a′-hexahydrospiro[imidazolidine-4,9′-xanthene]-2,5-dione (127 mg, 0.333 mmol, 96%).

Step H: tert-Butyldimethylsilyl chloride (“TBDMS-Cl”, 71.8 mg, 0.476 mmol) and imidazole (58.9 mg, 0.866 mmol) were added to a solution of 7′-bromo-3′-hydroxy-1-methyl-1′,2′,3′,4′,4a′,9a′-hexahydrospiro[imidazolidine-4,9′-xanthene]-2,5-dione (165 mg, 0.433 mmol) in CH₂Cl₂ (2.20 mL). The reaction mixture was stirred at room temperature for 24 hours. The reaction mixture was diluted with ethyl acetate and water, and the layers were separated. The aqueous layer was extracted with ethyl acetate (3×), and the combined organic layers were dried and concentrated to afford 7′-bromo-3′-(tert-butyldimethylsilyloxy)-1-methyl-1′,2′,3′,4′,4a′,9a′-hexahydrospiro[imidazolidine-4,9′-xanthene]-2,5-dione (117 mg, 0.236 mmol, 68%).

Step I: A solution of 7′-bromo-3′-(tert-butyldimethylsilyloxy)-1-methyl-1′,2′,3′,4′,4a′,9a′-hexahydrospiro[imidazolidine-4,9′-xanthene]-2,5-dione (115 mg, 0.232 mmol) and Lawesson's Reagent (56.3 mg, 0.139 mmol) in toluene (1.16 mL) was heated at 100° C. overnight. The reaction mixture was partitioned between ethyl acetate and water, and the aqueous layer was extracted with ethyl acetate (2×). The combined organic layers were dried and concentrated to afford 7′-bromo-3′-(tert-butyldimethylsilyloxy)-1-methyl-2-thioxo-1′,2′,3′,4′,4a′,9a′-hexahydrospiro[imidazolidine-4,9′-xanthen]-5-one (28 mg, 0.055 mmol, 97%).

Step J: A solution of 7′-bromo-3′-(tert-butyldimethylsilyloxy)-1-methyl-2-thioxo-1′,2′,3′,4′,4a′,9a′-hexahydrospiro[imidazolidine-4,9′-xanthen]-5-one (29 mg, 0.0567 mmol) in NH₃ (405 μL, 2.83 mmol, 7.0N in MeOH) and t-Butyl hydroperoxide (70% aqueous, 405 μL, 2.83 mmol) was stirred at room temperature for 18 hours. The reaction mixture was partitioned between ethyl acetate and water, and the aqueous was extracted with ethyl acetate (3×). The combined organic layers were dried and concentrated. The residue was purified by flash chromatography eluting with an ethyl acetate/hexanes gradient to afford 2-amino-7′-bromo-3′-(tert-butyldimethylsilyloxy)-1-methyl-1′,2′,3′,4′,4a′,9a′-hexahydrospiro[imidazole-4,9′-xanthen]-5(1H)-one (16.9 mg, 0.0342 mmol, 60%).

Step K: A solution of 2-amino-7′-bromo-3′-(tert-butyldimethylsilyloxy)-1-methyl-1′,2′,3′,4′,4a′,9a′-hexahydrospiro[imidazole-4,9′-xanthen]-5(1H)-one (115 mg, 0.233 mmol) in 4N HCl in dioxane (1.16 mL) was stirred for 6 hours at room temperature. The reaction mixture was concentrated, and the mixture was purified by flash chromatography, eluting with CH₂Cl₂/MeOH (15%) plus NH₄OH (1%) to afford 2-amino-7′-bromo-3′-hydroxy-1-methyl-1′,2′,3′,4′,4a′,9a′-hexahydrospiro[imidazole-4,9′-xanthen]-5(1H)-one (26 mg, 0.068 mmol, 29%).

Step L: A solution of 2-amino-7′-bromo-3′-hydroxy-1-methyl-1′,2′,3′,4′,4a′,9a′-hexahydro spiro[imidazole-4,9′-xanthen]-5(1H)-one (25.7 mg, 0.0676 mmol), 3-chloro-5-fluorophenylboronic acid (11.8 mg, 0.0676 mmol), Pd(PPh₃)₄ (3.91 mg, 0.00338 mmol), Na₂CO₃ (101 μL, 0.203 mmol, 2M) in dioxane (338 μL, 0.0676 mmol) was degassed with nitrogen for 5 minutes, sealed in a reaction vial, and stirred at 80° C. for 1 day. The reaction mixture was diluted with methanol (0.5 mL), filtered, and purified by semi-preparative C18 reversed-phase HPLC, eluting with acetonitrile/water (0.1% TFA). The product-containing fractions were concentrated to afford 2-amino-7′-(3-chloro-5-fluorophenyl)-3′-hydroxy-1-methyl-1′,2′,3′,4′,4a′,9a′-hexahydrospiro[imidazole-4,9′-xanthen]-5(1H)-one 2,2,2-trifluoroacetate (19.2 mg, 0.035 mmol, 52%). ¹H NMR (CD₃OD) δ 7.59 (m, 1H), 7.44 (m, 2H), 7.31 (m, 1H), 7.13 (dt, J=8.6, 2.3 Hz, 1H), 7.04 (d, J=9.0 Hz, 1H), 3.66 (m, 1H), 3.26 (s, 3H), 2.56 (m, 1H), 2.05 (m, 2H), 1.88 (m, 1H), 1.76-1.40 (m, 2H), 1.32 (m, 2H); m/z (APCI-pos) M+1=430.1 (100%), 431.1 (30%), 432.1 (40%).

### Example 2

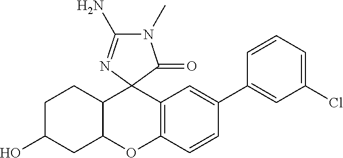

### 2-amino-7′-(3-chlorophenyl)-3′-hydroxy-1-methyl-1′,2′,3′,4′,4a′,9a′-hexahydrospiro[imidazole-4,9′-xanthen]-5(1H)-one

2-Amino-7′-(3-chlorophenyl)-3′-hydroxy-1-methyl-1′,2′,3′,4′,4a′,9a′-hexahydrospiro[imidazole-4,9′-xanthen]-5(1H)-one 2,2,2-trifluoroacetate was prepared according to Example 1, Step L, substituting 3-chlorophenyl boronic acid for 3-chloro-5-fluorophenylboronic acid. ¹H NMR (CD₃OD) δ 7.56 (m, 2H), 7.48 (m, 1H), 7.39 (m, 2H), 7.30 (m, 1H), 7.03 (d, 8.6 Hz, 1H), 3.66 (m, 1H), 3.25 (s, 3H), 2.57 (m, 1H), 2.16-2.00 (m, 2H), 1.90 (m, 1H), 1.71-1.56 (m, 1H), 1.52 (q, J=11 Hz, 1H), 1.32 (m, 1H), 1.01 (qd, 12, 3.0 Hz, 1H); m/z (APCI-pos) M+1=412.1 (100%), 414.1 (40%), 413.1 (20%).

### Example 3

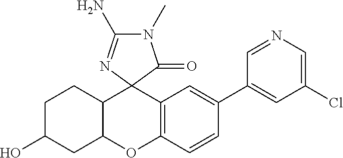

### 2-amino-7′-(5-chloropyridin-3-yl)-3′-hydroxy-1-methyl-1′,2′,3′,4′,4a′,9a′-hexahydrospiro[imidazole-4,9′-xanthen]-5(1H)-one

2-Amino-7′-(5-chloropyridin-3-yl)-3′-hydroxy-1-methyl-1′,2′,3′,4′,4a′,9a′-hexahydrospiro[imidazole-4,9′-xanthen]-5(1H)-one 2,2,2-trifluoroacetate was prepared according to Example 1, Step L, substituting 5-chloropyridin-3-yl boronic acid for 3-chloro-5-fluorophenylboronic acid. ¹H NMR (CD₃OD) δ 8.74 (br s, 1H), 8.55 (br s, 1H), 8.17 (s, 1H), 7.66 (m, 1H), 7.55 (m, 1H), 7.08 (d, J=6.0 Hz, 1H), 3.67 (m, 1H), 3.26 (s, 3H), 2.56 (m, 1H), 2.15-2.01 (m, 2H), 1.88 (m, 1H), 1.71-1.48 (m, 2H), 1.40-1.28 (m, 1H), 1.00 (m, 1H); m/z (APCI-pos) M+1=413.1 (100%), 415.1 (35%), 414.1 (20%).

### Example 4

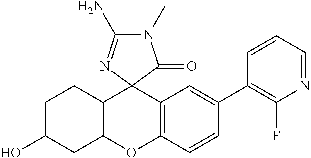

### 2-amino-7′-(2-fluoropyridin-3-yl)-3′-hydroxy-1-methyl-1′,2′,3′,4′,4a′,9a′-hexahydrospiro[imidazole-4,9′-xanthen]-5(1H)-one

Step A: A solution of 2-amino-7′-bromo-3′-(tert-butyldimethylsilyloxy)-1-methyl-1′,2′,3′,4′,4a′,9a′-hexahydrospiro[imidazole-4,9′-xanthen]-5(1H)-one (16.9 mg, 0.0342 mmol; Example 1, Step J), 2-fluoropyridin-3-ylboronic acid (6.02 mg, 0.0427 mmol), Pd(PPh₃)₄ (1.97 mg, 0.00171 mmol), Na₂CO₃ (51.3 μL, 0.103 mmol; 2M aqueous) in dioxane (171 μL, 0.0342 mmol) was degassed with nitrogen for 5 minutes, sealed in a vial and stirred at 80° C. for 1 day. The reaction mixture was purified by flash chromatography column (direct loading), and eluted with dichloromethane (“DCM”)/MeOH/1% NH₄OH gradient to afford 2-amino-3′(tert-butyldimethylsilyloxy)-7′-(2-fluoropyridin-3-yl)-1-methyl-1′,2′,3′,4′,4a′,9a′-hexahydrospiro[imidazole-4,9′-xanthen]-5(1H)-one (4.0 mg, 0.0078 mmol, 23%).

Step B: A solution of tetra-n-butylammonium fluoride (“TBAF”, 15.67 μL, 0.01567 mmol; 1.0M in THF) was added to a solution of 2-amino-3′-(tert-butyldimethylsilyloxy)-7′-(2-fluoropyridin-3-yl)-1-methyl-1′,2′,3′,4′,4a′,9a′-hexahydrospiro[imidazole-4,9′-xanthen]-5(1H)-one (4.0 mg, 0.0078 mmol) in DCM (78.3 μL, 0.0078 mmol). The resulting mixture was stirred at room temperature overnight. The reaction mixture was concentrated, diluted with methanol and purified by Gilson C18 to afford 2-amino-7′-(2-fluoropyridin-3-yl)-3′-hydroxy-1-methyl-1′,2′,3′,4′,4a′,9a′-hexahydrospiro[imidazole-4,9′-xanthen]-5(1H)-one 2,2,2-trifluoroacetate (2.1 mg, 0.0053 mmol, 67%). ¹H NMR (CD₃OD) δ 8.14 (br d, J=4.7 Hz, 1H), 7.98 (m, 1H), 7.55 (m, 7.42 (m, 1H), 7.37 (m, 1H), 7.07 (d, J=8.6 Hz, 1H), 3.66 (m, 1H), 3.23 (s, 3H), 2.56 (m, 1H), 2.13-2.00 (m, 2H), 1.87 (m, 1H), 1.62 (m, 1H), 1.42-1.24 (m, 2H), 1.07-0.93 (m, 1H); m/z (APCI-pos) M+1=397.1 (100%).

### Example 5

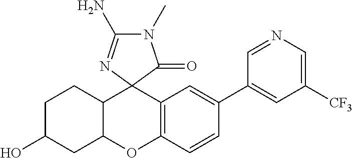

### 2-amino-3′-hydroxy-1-methyl-7′-(5-(trifluoromethyl)pyridin-3-yl)-1′,2′,3′,4′,4a′,9a′-hexahydrospiro[imidazole-4,9′-xanthen]-5(1H)-one

2-Amino-3′-hydroxy-1-methyl-7′-(5-(trifluoromethyl)pyridin-3-yl)-1′,2′,3′,4′,4a′, 9a′-hexahydrospiro[imidazole-4,9′-xanthen]-5(1H)-one 2,2,2-trifluoroacetate was prepared according to Example 1, Step L, substituting 5-(trifluoromethyl)pyridin-3-yl boronic acid for 3-chloro-5-fluorophenylboronic acid. ¹H NMR (CD₃OD) δ 9.07 (br s, 1H), 8.87 (br s, 1H), 8.33 (s, 1H), 7.70 (m, 1H), 7.60 (m, 1H), 7.11 (d, J=8.6 Hz, 1H), 3.67 (m, 1H), 3.26 (s, 3H), 2.57 (m, 1H), 2.09 (m, 2H), 1.72-1.49 (m, 2H), 1.32 (m, 2H), 1.01 (m, 1H); m/z (APCI-pos) M+1=447.1 (100%), 447.2 (20%).

### Example 6

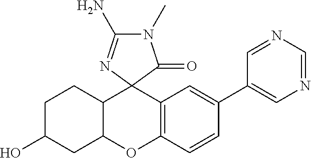

### 2-amino-3′-hydroxy-1-methyl-7′-(pyrimidin-5-yl)-1′,2′,3′,4′,4a′,9a′-hexahydrospiro[imidazole-4,9′-xanthen]-5(1H)-one

2-Amino-3′-hydroxy-1-methyl-7′-(pyrimidin-5-yl)-1′,2′,3′,4′,4a′,9a′-hexahydrospiro[imidazole-4,9′-xanthen]-5(1H)-one 2,2,2-trifluoroacetate was prepared according to Example 1, Step L, substituting pyrimidin-5-yl boronic acid for 3-chloro-5-fluorophenylboronic acid. m/z (APCI-pos) M+1=380.1 (100%).

### Example 7

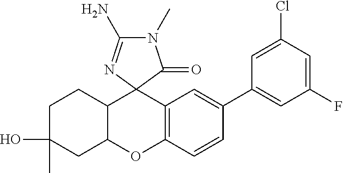

### 2-amino-7′-(3-chloro-5-fluorophenyl)-3′-hydroxy-1,3′-dimethyl-1′,2′,3′,4′,4a′,9a′-hexahydrospiro[imidazole-4,9′-xanthen]-5(1H)-one

Step A: In a teflon-lined metal pressure reactor, a mixture of 7′-bromo-3′-(spiro[1,3]dioxolane)-1′,2′,3′,4′,4a′,9a′-hexahydrospiro[imidazolidine-4,9′-xanthene]-2,5-dione (11.0 g, 26.8 mmol; Example 1, Step D) and KOH (15.04 g, 268 mmol) in water (53.6 mL) was heated at 195° C. (sand bath+metal bowl) overnight. The reaction mixture was cooled, transferred to a 1 L beaker, washed with a minimal amount of water, and neutraled (pH 7) with 4N HCl. The precipitate was collected by filtration, dried on the filter, and then high vacuum to afford 9′-amino-7′-bromo-1′,2′,4′,4a′,9′,9a′-hexahydrospiro[[1,3]dioxolane-2,3′-xanthene]-9′-carboxylic acid (7.10 g, 18.5 mmol, 69%).

Step B: A solution of 9′-amino-7′-bromo-1′,2′,4′,4a′,9′,9a′-hexahydrospiro[[1,3]dioxolane-2,3′-xanthene]-9′-carboxylic acid (7.00 g, 18.2 mmol) in MeOH (91.1 ml) was treated with trimethylsilyldiazomethane (“TMSCHN₂”, 45.5 mL, 91.1 mmol, 2.0M solution in hexanes). The reaction mixture was concentrated and diluted with ether, and 4N HCl/dioxane was added to this solution to precipitate the product. The solid was collected by filtration and dried on a high vacuum to afford methyl 9′-amino-7′-bromo-1′,2′,4′,4a′,9′,9a′-hexahydrospiro[[1,3]dioxolane-2,3′-xanthene]-9′-carboxylate hydrochloride (7.06 g, 17.7 mmol, 97%).

Step C: Methyl 9′-amino-7′-bromo-1′,2′,4′,4a′,9′,9a′-hexahydrospiro[[1,3]dioxolane-2,3′-xanthene]-9′-carboxylate hydrochloride (7.06 g, 16.2 mmol) was suspended in DMF (81.2 mL), and isothiocyanatomethane (2.22 mL, 32.5 mmol) was added. Triethylamine (“TEA”, 9.06 mL, 65.0 mmol) was added to this mixture, and the resulting solution was stirred at 65° C. for 24 hours. The reaction mixture was partitioned between ethyl acetate and water, and the aqueous layer was extracted with ethyl acetate (3×). The combined organic layers were dried and concentrated. The residue was purified by flash chromatography eluting with ethyl acetate/hexanes gradient to afford 7′-bromo-1-methyl-3′-(spiro[1,3]dioxolane)-2-thioxo-1′,2′,3′,4′,4a′,9a′-hexahydrospiro[imidazolidine-4,9′-xanthen]-5-one (3.40 g, 7.74 mmol, 48%).

Step D: A solution of 7′-bromo-1-methyl-3′-(spiro[1,3]dioxolane)-2-thioxo-1′,2′,3′,4′,4a′,9a′-hexahydrospiro[imidazolidine-4,9′-xanthen]-5-one (3.4 g, 7.7 mmol) in NH₃ (28 mL, 193 mmol, 7.0N in MeOH) and t-butyl hydroperoxide (70% aqueous, 28 mL, 193 mmol) was stirred at room temperature for 1 day. The reaction mixture was diluted with brine and ethyl acetate, and the aqueous layer was extracted with ethyl acetate (2×). The combined organic layers were dried and concentrated, and the residue was purified by flash chromatography, eluting with a DCM/MeOH/NH₄OH gradient to afford 2-amino-7′-bromo-3′-(spiro[1,3]dioxolane)-1-methyl-1′,2′,3′,4′,4a′,9a′-hexahydro spiro[imidazole-4,9′-xanthen]-5(1H)-one (2.20 g, 5.20 mmol, 67%).

Step E: A solution of 2-amino-7′-bromo-3′-(spiro[1,3]dioxolane)-1-methyl-1′,2′,3′,4′,4a′,9a′-hexahydrospiro[imidazole-4,9′-xanthen]-5(1H)-one (2.00 g, 4.76 mmol) and 4N HCl (23.7 mL, 47.4 mmol) in acetone (23.7 mL) was heated at 65° C. for 1 day. The solvent was removed, and the residue was azeotroped with toluene (3×) to afford 2-amino-7′-bromo-1-methyl-1′,4′,4a′,9a′-tetrahydrospiro[imidazole-4,9′-xanthene]-3′,5(1H, 2′H)-dione (1.80 g, 4.76 mmol, >99%).

Step F: Boc₂O (0.361 g, 1.65 mmol) was added to a solution of 2-amino-7′-bromo-1-methyl-1′,4′,4a′,9a′-tetrahydrospiro[imidazole-4,9′-xanthene]-3′,5(1H, 2′H)-dione (0.50 g, 1.32 mmol) and TEA (0.553 mL, 3.97 mmol) in DCM (6.61 mL), and the resulting solution was stirred at room temperature overnight. The reaction mixture was diluted with DCM and washed with brine. The organic layer was dried and concentrated to afford tert-butyl 7′-bromo-1-methyl-3′,5-dioxo-1,1′,2′,3′,4′,4a′,5,9a′-octahydrospiro[imidazole-4,9′-xanthene]-2-ylcarbamate (0.531 g, 1.11 mmol, 84%).

Step G: MeMgBr (176 tit, 0.528 mmol, 3.0M in ether) was added to a solution of tert-butyl 7′-bromo-1-methyl-3′,5-dioxo-1,1′,2′,3′,4′,4a′,5,9a′-octahydrospiro[imidazole-4,9′-xanthene]-2-ylcarbamate (101 mg, 0.211 mmol) in THF (1.06 mL) at −78° C. The solution was stirred at −78° C. for 15 minutes and then allowed to warm to room temperature. After 1 hour at room temperature, the reaction mixture was quenched with water and extracted with ethyl acetate (3×). The combined organic layers were dried and concentrated. The residue was dissolved with 4N HCl/dioxane, stirred at room temperature for 3 hours, and then concentrated at 50° C. by rotovap. The residue was purified by flash chromatography eluting with 0-10% MeOH/DCM+1% NH₄OH to afford 2-amino-7′-bromo-3′-hydroxy-1,3′-dimethyl-1′,2′,3′,4′,4a′,9a′-hexahydro spiro[imidazole-4,9′-xanthen]-5(1H)-one (52 mg, 0.132 mmol, 63%).

Step H: A solution of 2-amino-7′-bromo-3′-hydroxy-1,3′-dimethyl-1′,2′,3′,4′,4a′,9a′-hexahydrospiro[imidazole-4,9′-xanthen]-5(1H)-one (50 mg, 0.127 mmol), 3-chloro-5-fluorophenylboronic acid (23.2 mg, 0.133 mmol), Pd(PPh₃)₄ (7.33 mg, 0.00634 mmol), Na₂CO₃ (190 μL, 0.380 mmol, 2.0M) in dioxane (634 μL, 0.127 mmol) was degassed with nitrogen for 5 minutes, sealed in a vial and stirred at 80° C. for 1 day. The crude reaction mixture was filtered and then purified by semi-preparative C18 reversed-phase HPLC to afford 2-amino-7′-(3-chloro-5-fluorophenyl)-3′-hydroxy-1,3′-dimethyl-1′,2′,3′,4′,4a′,9a′-hexahydrospiro[imidazole-4,9′-xanthen]-5(1H)-one 2,2,2-trifluoroacetate (10.0 mg, 0.023 mmol, 18%). ¹H NMR (CD₃OD) δ 7.59 (m, 1H), 7.46 (m, 2H), 7.31 (d, J=9.0 Hz, 1H), 7.13 (m, 1H), 7.02 (t, J=8.2 Hz, 1H), 3.64 (m, 1H), 3.26 (s, 3H), 2.28 (m, 1H), 2.09 (m, 1H), 1.81-1.65 (m, 2H), 1.58 (t, J=12 Hz, 1H), 1.46 (d, J=11 Hz, 1H), 1.26 (s, 3H); m/z (APCI-pos) M+1=444.1 (100%), 446.1 (35%), 445.1 (20%).

### Example 8

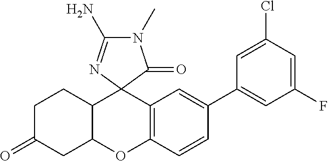

### 2-amino-7′-(3-chloro-5-fluorophenyl)-1-methyl-1′,4′,4a′,9a′-tetrahydrospiro[imidazole-4,9′-xanthene]-3′,5(1H, 2′H)-dione

2-Amino-7′-(3-chloro-5-fluorophenyl)-1-methyl-1′,4′,4a′,9a′-tetrahydrospiro[imidazole-4,9′-xanthene]-3′,5(1H, 2′H)-dione (0.140 g, 0.327 mmol, 48.5% yield) was made according to the procedure of Example 7, where after Step E, a mixture of 2-amino-7′-bromo-1-methyl-1′,4′,4a′,9a′-tetrahydrospiro[imidazole-4,9′-xanthene]-3′,5(1H, 2′H)-dione (0.255 g, 0.674 mmol), 3-chloro-5-fluorophenylboronic acid (0.153 g, 0.876 mmol), Pd(PPh₃)₄ (0.0390 g, 0.0337 mmol) and Na₂CO₃ (1.05 mL, 2.09 mmol) in dioxane (1.5 mL, 0.674 mmol) was heated to 90° C. overnight in a capped vial. LCMS showed that the reaction was complete. The mixture was then partitioned between DCM and water. The organics were extracted with DCM twice, washed with brine and dried with Na₂SO₄. This was then purified on the preparative HPLC to give the final product. ¹H NMR (CD₃OD) δ 7.37 (t, 1H), 7.23 (d, 1H), 7.07 (d, 2H), 7.01 (d, 1H), 6.97 (m, 1H), 5.00 (m, 1), 3.23 (s, 1), 3.15 (s, 1), 3.09 (s, 2), 3.05 (m, 1), 2.60 (m, 3), 2.40 (m, 2); MS m/z (APCI-pos) M+1=428.1.

### Example 9

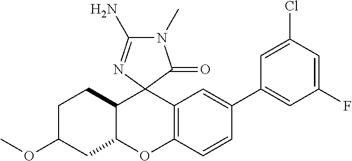

### (4a′R*,9a′S*)-2-amino-7′-(3-chloro-5-fluorophenyl)-3′-methoxy-1-methyl-1′,2′,3′,4′,4a′,9a′-hexahydrospiro[imidazole-4,9′-xanthen]-5(1H)-one

Step A: (4a′R*,9a′S*)-2-Amino-7′-(3-chloro-5-fluorophenyl)-3′-methoxy-1-methyl-1′,2′,3′,4′,4a′,9a′-hexahydrospiro[imidazole-4,9′-xanthen]-5(1H)-one was made according to the procedure of Example 7, where after Step E, NaBH₄ (0.0800 g, 2.12 mmol) was added to a mixture of 2-amino-7′-bromo-1-methyl-1′,4′,4a′,9a′-tetrahydrospiro[imidazole-4,9′-xanthene]-3′,5(1H, 2′H)-dione (0.400 g, 1.06 mmol) in THF (4 mL, 1.06 mmol) at 0° C. The reaction mixture was allowed to come to room temperature overnight. LCMS showed that the reaction was complete. The mixture was then partitioned between DCM and water. The organics were extracted with DCM twice, washed with brine and dried with Na₂SO₄. This was then concentrated down to give 14a′R*,9a′S*)-2-amino-7′-bromo-3′-hydroxy-1-methyl-1′,2′,3′,4′,4a′,9a′-hexahydrospiro[imidazole-4,9′-xanthen]-5(1H)-one (0.335 g, 0.881 mmol, 83.3% yield).

Step B: A mixture of (4a′R*,9a′S*)-2-amino-7′-bromo-3′-hydroxy-1-methyl-1′,2′,3′,4′,4a′,9a′-hexahydrospiro[imidazole-4,9′-xanthen]-5(1H)-one (0.135 g, 0.355 mmol), iodomethane (0.0221 mL, 0.355 mmol) and Cs₂CO₃ (0.139 g, 0.426 mmol) in DMF (1.5 mL, 0.355 mmol) was stirred overnight at 90° C. Mass spectrometry showed that the reaction was complete. The mixture was then partitioned between DCM and water. The organics were extracted with DCM twice, washed with brine and dried with Na₂SO₄. This was then concentrated down to give (4a′R*,9a′S*)-2-amino-7′-bromo-3′-methoxy-1-methyl-1′,2′,3′,4′,4a′,9a′-hexahydro spiro[imidazole-4,9′-xanthen]-5(1H)-one (0.139 g, 0.353 mmol, 99.3% yield).

Step C: A mixture of (4a′R*,9a′S*)-2-amino-7′-bromo-3′-methoxy-1-methyl-1′,2′,3′,4′,4a′,9a′-hexahydrospiro[imidazole-4,9′-xanthen]-5(1H)-one (0.140 g, 0.355 mmol), 3-chloro-5-fluorophenylboronic acid (0.0681 g, 0.391 mmol), Pd(PPh₃)₄ (0.0205 g, 0.0178 mmol) and Na₂CO₃ (0.373 mL, 0.746 mmol) in dioxane (1 mL, 0.355 mmol) was heated to 90° C. overnight in a capped vial. LCMS showed that the reaction was complete. The mixture was then partitioned between DCM and water. The organics were extracted with DCM twice, washed with brine and dried with Na₂SO₄. This was then purified on the preparative HPLC to give (4a′R*,9a′S*)-2-amino-7′-(3-chloro-5-fluorophenyl)-3′-methoxy-1-methyl-1′,2′,3′,4′,4a′,9a′-hexahydrospiro[imidazole-4,9′-xanthen]-5(1H)-one (0.0161 g, 0.0363 mmol, 10.2% yield). ¹H NMR (CD₃OD) δ 7.48 (dd, 1H), 7.21 (d, 1H), 7.04 (m, 3.5H), 6.85 (d, 0.5H), 5.08 (m, 1H), 4.37 (s, 1H), 3.52 (s, 0.5), 3.48 (s, 0.5), 3.40 (s, 2H), 3.31 (s, 1H), 3.10 (s, 2H), 2.50 (m, 1H), 1.90 (m, 1H), 1.60 (m, 5H); MS m/z (APCI-pos) M+1=444.1.

### Example 10

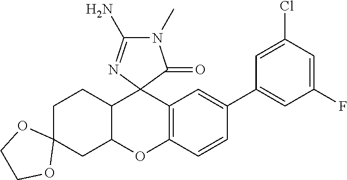

### 2″-amino-7′-(3-chloro-5-fluorophenyl)-1″-methyl-1″,2′,4′,4′a,5″,9′a-hexahydro-1′H-dispiro[1,3-dioxolane-2,3′-xanthene-9′,4″-imidazole]-5″-one

Example 10 was made according to Example 7, where after Step D, a mixture of 2-amino-7′-bromo-3′-(spiro[1,3]dioxolane)-1-methyl-1′,2′,3′,4′,4a′,9a′-hexahydrospiro[imidazole-4,9′-xanthen]-5(1H)-one (0.027 g, 0.064 mmol), 3-chloro-5-fluorophenylboronic acid (0.012 g, 0.070 mmol), Pd(PPh₃)₄ (0.0074 g, 0.0064 mmol) and Na₂CO₃ (0.070 mL, 0.14 mmol) in dioxane (0.8 mL, 0.064 mmol) was heated to 90° C. overnight in a capped vial. LCMS showed that the reaction was complete. The mixture was then partitioned between DCM and water. The organics were extracted with DCM twice, washed with brine and dried with Na₂SO₄. This was then purified on the preparative HPLC to give Example 10 (0.005 g, 0.011 mmol, 17% yield). ¹H NMR (CD₃OD) δ 7.42 (dd, 1H), 7.22 (s, 1H), 7.02 (m, 4H), 4.82 (m, 1H), 4.00 (m, 4H), 3.29 (s, 3H), 2.41 (d, 1H), 2.19 (dt, 1H), 1.83, (m, 3H), 1.66 (dt, 1H), 1.26 (m, 1H); MS m/z (APCI-pos) M+1=472.1.

### Example 11

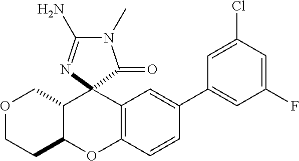

### (4R,4a′S,10a′S)-2-amino-8′-(3-chloro-5-fluorophenyl)-1-methyl-3′,4′,4a′,10a′-tetrahydro-1′H-spiro[imidazole-4,10′-pyrano[4,3-b]chromen]-5(1H)-one

Step A: A stainless steel bomb (50 mL capacity) containing a teflon-coated insert was charged with ethoxyethene (19 mL, 200 mmol) and 6-bromo-4-oxo-4H-chromene-3-carbaldehyde (2.5 g, 10 mmol). The bomb was sparged with N₂ for 3 minutes. The reaction mixture was heated to 100° C. with stirring for 18 hours. After cooling to room temperature, the reaction mixture was concentrated in vacuo to yield (3R,4aR)-8-bromo-3-ethoxy-4,4a-dihydropyrano[4,3-b]chromen-10(3H)-one (3.0 g, 90%). The product did not require purification. A 3:1 mixture of endo/exo isomers was obtained based on ¹H NMR.

Step B: A 25 mL round bottomed flask plus stir bar was charged with (3R,4aR)-8-bromo-3-ethoxy-4,4-a-dihydropyrano[4,3-b]chromen-10(3H)-one (2.8 g, 8.6 mmol), dioxane (35 mL), and PtO₂—H₂O, “Adam's catalyst,” (0.21 g, 0.86 mmol). The reaction mixture was stirred under an H₂ balloon at room temperature for 15 hours. The mixture was concentrated and purified by Biotage Flash 40 silica gel chromatography, eluting with a gradient of 10%-30% EtOAc/hexanes. The product yielded (3R,4aR,10aS)-8-bromo-3-ethoxy-1,4,4a,10a-tetrahydropyrano[4,3-b]chromen-10(3H)-one (375 mg, 11%).

Step C: A 10 mL round bottomed flask plus stir bar was charged with (3R,4aR,10aS)-8-bromo-3-ethoxy-1,4,4a,10a-tetrahydropyrano[4,3-b]chromen-10(3H)-one (325 mg, 0.993 mmol), DCM (2 mL), and triethylsilane (1.3 mL, 8.0 mmol). The mixture was cooled to 0° C. under N₂, and BF₃ Etherate (0.50 mL, 4.0 mmol) was added. The reaction mixture was stirred for 30 minutes. The reaction mixture was allowed to warm to room temperature while stirring for 3 hours. The mixture was quenched with saturated aqueous NaHCO₃ (2 mL) and stirred for 30 minutes. The phases were separated, and the aqueous phase was re-extracted with DCM (2×5 mL). The organic phases were combined, washed with brine (10 mL), dried (MgSO₄), filtered, and concentrated to yield (4aR,10aS)-8-bromo-1,4,4a,10a-tetrahydropyrano[4,3-b]chromen-10(3H)-one (260 mg, 81%). The mixture was carried forward without purification at this step.

Step D: A stainless steel bomb (20 mL capacity) containing a teflon insert was charged with EtOH (1 mL) and (4aR,10aS)-8-bromo-1,4,4a,10a-tetrahydropyrano[4,3-b]chromen-10(3H)-one (260 mg, 0.918 mmol). Next, ammonium carbonate (441 mg, 4.59 mmol), KCN (120 mg, 1.84 mmol) and sodium hydrogensulfite (24 mg, 0.23 mmol) were added. The reaction mixture was heated to 130° C. for 2 days with stirring. After cooling to room temperature, the reaction contents were transferred to an Erlenmeyer flask with EtOAc (10 mL) and water (5 mL). The mixture was carefully acidified with concentrated HCl, and then N₂ was bubbled through the mixture to sparge HCN (in back of hood with sashes closed to minimize exposure to HCN). The phases were separated, and the aqueous phase was re-extracted with EtOAc (2×10 mL). The organic phases were combined, washed with brine (20 mL), dried (MgSO₄), filtered, and concentrated to yield 8′-bromo-3′,4′,4a′,10a′-tetrahydro-1′H-spiro[imidazolidine-4,10′-pyrano[4,3-b]chromene]-2,5-dione (301 mg, 70%). A 1:1 mixture of cis/trans isomers was obtained as determined by ¹H NMR. The product was carried forward without purification.

Step E: A round bottomed flask plus stir bar was charged with potassium carbonate (117 mg, 0.849 mmol) and DMF (2 mL). 8′-Bromo-3′,4′,4a′,10a′-tetrahydro-1′H-spiro[imidazolidine-4,10′-pyrano[4,3-b]chromene]-2,5-dione (300 mg, 0.849 mmol) was added. Iodomethane (48 μL, 0.77 mmol) was added last. The mixture was stirred at room temperature for 18 hours. The reaction mixture was partioned between EtOAc (10 mL) and water (10 mL). The phases were separated, and the aqueous phase was re-extracted with EtOAc (10 mL) The combined organic phases were washed with water (10 mL), brine (10 mL), dried (MgSO₄), filtered, and concentrated. The cis/trans isomers were separated by Biotage Flash 40 silica gel chromatography, eluting with 20%-50% EtOAc/hexanes, then neat EtOAc to yield the “trans” isomer, (4a′R,10a′R)-8′-bromo-1-methyl-3′,4′,4a′,10a′-tetrahydro-1′H-spiro[imidazolidine-4,10′-pyrano[4,3-b]chromene]-2,5-dione (44 mg, 10%).

Step F: A 2 dram vial plus stir bar was charged with (4a′R,10a′R)-8′-bromo-1-methyl-3′,4′,4a′,10a′-tetrahydro-1′H-spiro[imidazolidine-4,10′-pyrano[4,3-b]chromene]-2,5-dione (44 mg, 0.12 mmol), Lawesson's Reagent (29 mg, 0.072 mmol), and toluene (0.5 mL) The reaction mixture was degassed with N₂. The mixture was then heated to 100° C. with stirring for 15 hours. The reaction mixture was partioned between EtOAc (5 mL) and saturated aqueous NaHCO₃ (5 mL). The phases were separated, and the aqueous phase was re-extracted with EtOAc (5 mL). The combined organic phases were washed with brine (10 mL), dried (MgSO₄), filtered, and concentrated to yield (4a′R,10a′R)-8′-bromo-1-methyl-2-thioxo-3′,4′,4a′,10a′-tetrahydro-1′H-spiro[imidazolidine-4,10′-pyrano[4,3-b]chromen]-5-one (56 mg, 109%; likely Lawesson's by-product accounts for extra mass). The product was carried forward without purification at this step.

Step G: A round bottomed flask plus stir bar was charged with (4a′R,10a′R)-8′-bromo-1-methyl-2-thioxo-3′,4′,4a′,10a′-tetrahydro-1′H-spiro[imidazolidine-4,10′-pyrano[4,3-b]chromen]-5-one (46 mg, 0.12 mmol), MeOH (1 mL), 70% aqueous t-butyl hydroperoxide (0.25 mL, 1.8 mmol), and 30% aqueous NH₄OH (0.47 mL, 3.6 mmol). The reaction mixture was stirred for 18 hours at room temperature. Water (1 mL) was added, and the mixture was concentrated in vacuo. The reaction mixture was pardoned between EtOAc (5 mL) and water (5 mL). The phases were separated. The aqueous phase was re-extracted with EtOAc (5 mL). The combined organic phases were washed with brine (10 mL), dried (MgSO₄), filtered, and concentrated. The product was purified by preparative TLC (0.5 mm plate thickness; Rf=0.15) eluting with 5% MeOH/DCM to yield (4a′R,10a′R)-2-amino-8′-bromo-1-methyl-3′,4′,4a′,10a′-tetrahydro-1′H-spiro[imidazole-4,10′-pyrano[4,3-b]chromen]-5(1H)-one (10 mg, 19%).

Step H: A 2 dram vial plus stir bar was charged with (4a′R,10a′R)-2-amino-8′-bromo-1-methyl-3′,4′,4a′,10a′-tetrahydro-1′H-spiro[imidazole-4,10′-pyrano[4,3-b]chromen]-5(1H)-one (10 mg, 0.027 mmol), dioxane (0.3 mL), 3-chloro-5-fluorophenylboronic acid (5.2 mg, 0.030 mmol), Pd(PPh₃)₄ (3.2 mg, 0.0027 mmol), and 2N aqueous Na₂CO₃ (34 μL, 0.068 mmol). The reaction mixture was sparged with N₂ for 30 seconds and then heated with stirring to 90° C. for 18 hours. After cooling to room temperature, the reaction mixture was loaded directly on to preparative TLC plate (0.5 mm plate thickness, Rf=0.65) and eluted with 10% MeOH (containing 7N NH₃) in DCM. The product required a second purification by preparative TLC (0.5 mm plate thickness) eluting with 5% MeOH/EtOAc to obtain a product 85% diastereomeric purity (trans/cis), (4R,4a′R,10a′R)-2-amino-8′-(3-chloro-5-fluorophenyl)-1-methyl-3′,4′,4a′,10a′-tetrahydro-1′H-spiro[imidazole-4,10′-pyrano[4,3-b]chromen]-5(1H)-one (2 mg, 18%).

It was later determined by crystallography that the final product was (4R,4a′S,10a′S)-2-amino-8′-(3-chloro-5-fluorophenyl)-1-methyl-3′,4′,4a′,10a′-tetrahydro-1′H-spiro[imidazole-4,10′-pyrano[4,3-b]chromen]-5(1H)-one. ¹H NMR (400 MHz, CDCl₃) δ 7.37 (dd, J=2, 8 Hz, 1H), 7.22 (s, 1H), 7.05 (m, 2H), 7.00 (m, 1H), 6.97 (d, J=9 Hz, 1H), 4.93 (td, J=5, 11 Hz, 1H), 4.07 (dd, J=5, 12 Hz, 1H), 3.99 (dd, J=4, 11 Hz, 1H), 3.48 (td, J=2, 13 Hz, 1H), 3.13 (s, 3H), 3.04 (t, J=11 Hz, 1H), 3.03 (br s, 2H), 2.27 (td, J=4, 11 Hz, 1H), 2.18 (m, 1H), 1.87 (m, 1H); m/z (APCI-pos) M+1=416.

### Example 12

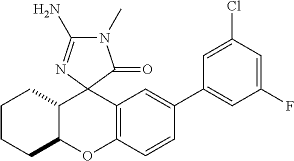

### trans 2-amino-7′43-chloro-5-fluorophenyl)-1-methyl-1′,2′,3′,4′,4a′,9a′-hexahydrospiro[imidazole-4,9′-xanthen]-5(1H)-one

Step A: Oxalyl chloride (8.64 mL, 99.1 mmol) was added to a solution of cyclohex-1-enecarboxylic acid (10 g, 79.3 mmol) in CH₂Cl₂ (159 mL). One drop of DMF was added to this solution, and the resulting solution was stirred at room temperature for 2 hours. The solvent was concentrated to give cyclohex-1-enecarbonyl chloride as an oil (11.5 g, 100%).

Step B: 1-Bromo-4-methoxybenzene (7.36 mL, 58.81 mmol) and aluminum chloride (15.68 g, 117.6 mmol) were added to a solution of cyclohex-1-enecarbonyl chloride (10.63 g, 73.52 mmol) in dichloroethane (“DCE”, 294.1 mL). The resulting solution was stirred at room temperature overnight. The mixture was poured into a beaker containing ice-Rochelle salt and filtered through GF/F paper. The organic layer was separated, and the aqueous layer was extracted with CH₂Cl₂. The combined organic extracts were dried (phase separator silicone treated filter paper), concentrated, and purified on silica gel (0-2% ether in hexanes) to provide (5-bromo-2-hydroxyphenyl)(cyclohexenyl)methanone as an oil (3.5 g, 21%).

Step C: A mixture of (5-bromo-2-hydroxyphenyl)(cyclohexenyl)methanone (3.5 g, 12.4 mmol) in 1N NaOH (62.2 mL, 62.2 mmol) was stirred at room temperature for 18 hours. A thick precipitate formed, and the reaction mixture was diluted with some water (20 mL) to help with stirring. The solution was cooled in an ice bath and acidified to pH 1 with concentrated HCl. The precipitate was collected by filtration, to give mostly the trans isomers of 7-bromo-2,3,4,4a-tetrahydro-1H-xanthen-9(9aH)-one (2.93 g, 84%) as a solid.

Step D: 7′-Bromo-1′,2′,3′,4′,4a′,9a′-hexahydro spiro[imidazolidine-4,9′-xanthene]-2,5-dione was prepared according to Example 1 Step D, substituting 7-bromo-2,3,4,4a-tetrahydro-1H-xanthen-9(9aH)-one for 7′-bromo-1′,4′,4a′,9a′-tetrahydrospiro[[1,3]dioxolane-2,3′-xanthen]-9′(2′H)-one.

Step E: A mixture of 7′-bromo-1′,2′,3′,4′,4a′,9a′-hexahydrospiro[imidazolidine-4,9′-xanthene]-2,5-dione (1.1 g, 3.13 mmol) in water (7.83 mL) was treated with potassium hydroxide (1.76 g, 31.3 mmol) and heated at 195° C. overnight in a Teflon-lined steel bomb. After cooling the mixture in an ice bath, it was transferred to a beaker, diluted with small volume of water, and the pH adjusted to 7 with 2N HCl. The precipitated solids were collected by filtration, to give 9-amino-7-bromo-2,3,4,4a,9,9a-hexahydro-1H-xanthene-9-carboxylic acid (0.950 g, 93%).

Step F: Trimethylsilyldiazomethane solution (10.2 mL, 20.4 mmol) was added to a cold (0° C.) crude suspension of 9-amino-7-bromo-2,3,4,4a,9,9a-hexahydro-1H-xanthene-9-carboxylic acid (0.95 g, 2.91 mmol) in MeOH (29 mL). After stirring at room temperature for 18 hours, the mixture was quenched with water and partitioned between ethyl acetate and water. The organic layer was dried (phase separator silicone treated filter paper), concentrated, purified on silica gel (10-40% ethyl acetate in hexanes) and first eluting the trans isomers of methyl 9-amino-7-bromo-2,3,4,4a,9,9a-hexahydro-1H-xanthene-9-carboxylate (0.27 g, 27%).

Step G: A solution of the trans isomers of methyl 9-amino-7-bromo-2,3,4,4a,9,9a-hexahydro-1H-xanthene-9-carboxylate (0.214 g, 0.6290 mmol), isothiocyanatomethane (0.1721 mL, 2.51 mmol) and triethylamine (0.35 mL, 2.51 mmol) in DMF (3.14 mL) was stirred at 60° C. overnight. The mixture was partitioned between ethyl acetate and water. The organic layer was washed with brine, dried (phase separator silicone treated filter paper), concentrated, and purified on silica gel (10-40% ethyl acetate in hexanes) to provide the trans isomers of 7′-bromo-1-methyl-2-thioxo-1′,2′,3′,4′,4a′,9a′-hexahydrospiro[imidazolidine-4,9′-xanthen]-5-one (0.155 g, 65%) as solids.

Step H: A solution of the trans isomers of 7′-bromo-1-methyl-2-thioxo-1′,2′,3′,4′,4a′,9a′-hexahydrospiro[imidazolidine-4,9′-xanthen]-5-one (0.057 g, 0.15 mmol) in ammonia (1.1 mL, 7.5 mmol, 7.0N in MeOH) and t-butyl hydroperoxide (70% aqueous, 1.1 mL, 7.5 mmol) was stirred at room temperature overnight. The mixture was concentrated. The residue was partitioned between DCM and water, the organic layer was dried (phase separator silicone treated filter paper), concentrated and purified on silica gel (1-5% MeOH in DCM) to provide the trans isomers of 2-amino-7′-bromo-1-methyl-1′,2′,3′,4′,4a′,9a′-hexahydrospiro[imidazole-4,9′-xanthen]-5(1H)-one (0.018 g, 33%).

Step I: The trans isomers of 2-amino-7′-(3-chloro-5-fluorophenyl)-1-methyl-1′,2′,3′,4′,4a′,9a′-hexahydrospiro[imidazole-4,9′-xanthen]-5(1H)-one 2,2,2-trifluoroacetate were prepared according to Example 1, Step L, substituting the trans isomers of 2-amino-7′-bromo-1-methyl-1′,2′,3′,4′,4a′,9a′-hexahydrospiro[imidazole-4,9′-xanthen]-5(1H)-one for 2-amino-7′-bromo-3′-hydroxy-1-methyl-1′,2′,3′,4′,4a′,9a′-hexahydrospiro[imidazole-4,9′-xanthen]-5(1H)-one. ¹H NMR (CDCl₃) δ 7.69-7.63 (m, 1H), 7.52-7.41 (m, 2H), 7.04-6.98 (m, 3H), 4.62-4.57 (m, 1H), 3.28 (s, 3H), 2.38-2.17 (m, 2H), 1.91-1.27 (m, 7H). MS m/z (APCI-pos) M+1=414.

### Example 13

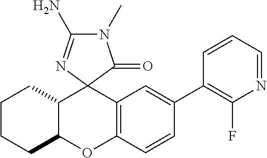

### trans 2-amino-7′-(2-fluoropyridin-3-yl)-1-methyl-1′,2′,3′,4′,4a′,9a′-hexahydro spiro[imidazole-4,9′-xanthen]-5(1H)-one

The trans isomers of 2-amino-7′-(2-fluoropyridin-3-yl)-1-methyl-1′,2′,3′,4′,4a′,9a′-hexahydrospiro[imidazole-4,9′-xanthen]-5(1H)-one 2,2,2-trifluoroacetate were prepared according to Example 1, Step L, substituting the trans isomers of 2-amino-7′-bromo-1-methyl-1′,2′,3′,4′,4a′,9a′-hexahydrospiro[imidazole-4,9′-xanthen]-5(1H)-one and 2-fluoropyridin-3-ylboronic acid for 2-amino-7′-bromo-3′-hydroxy-1-methyl-1′,2′,3′,4′,4a′,9a′-hexahydrospiro[imidazole-4,9′-xanthen]-5(1H)-one and 3-chloro-5-fluorophenylboronic acid.

The above mixture of isomers was purified by C18 chromatography, eluting with ACN/H2O+0.1% TFA to provide (4R,4a′R,9a′S)-2-amino-7′-(2-fluoropyridin-3-yl)-1-methyl-1′,2′,3′,4′,4a′,9a′-hexahydrospiro[imidazole-4,9′-xanthen]-5(1H)-one:

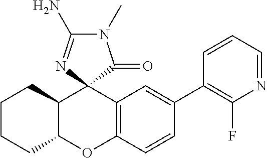

and (4R,4a′S,9a′R)-2-amino-7′-(2-fluoropyridin-3-yl)-1-methyl-1′,2′,3′,4′,4a′,9a′-hexahydrospiro[imidazole-4,9′-xanthen]-5(1H)-one. ¹H NMR (CDCl₃) δ 8.1-8.09 (m, 1H), 7.79-7.75 (m, 1H), 7.47-7.43 (m, 1H), 7.24-7.21 (m, 1H), 7.14-7.12 (m, 1H), 7.02-6.99 (m, 1H), 4.68-4.61 (m, 1H), 3.27 (s, 3H), 2.38-2.30 (m, 1H), 2.17-1.83 (m, 5H), 1.52-1.31 (m, 3H). MS m/z (APCI-pos) M+1=381.

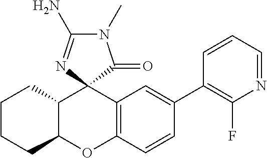

### Example 14

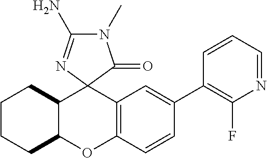

### cis 2-amino-7′-(2-fluoropyridin-3-yl)-1-methyl-1′,2′,3′,4′,4a′,9a′-hexahydrospiro[imidazole-4,9′-xanthen]-5(1H)-one

The cis isomers of 2-amino-7′-(2-fluoropyridin-3-yl)-1-methyl-1′,2′,3′,4′,4a′,9a′-hexahydrospiro[imidazole-4,9′-xanthen]-5 (1H)-one 2,2,2-trifluoroacetate were prepared according to Example 1, Step L, substituting the cis isomers of 2-amino-7′-bromo-1-methyl-1′,2′,3′,4′,4a′,9a′-hexahydrospiro[imidazole-4,9′-xanthen]-5(1H)-one and 2-fluoropyridin-3-ylboronic acid for 2-amino-7′-bromo-3′-hydroxy-1-methyl-1′,2′,3′,4′,4a′,9a′-hexahydrospiro[imidazole-4,9′-xanthen]-5(1H)-one and 3-chloro-5-fluorophenyl boronic acid. ¹H NMR (CDCl₃) δ 7.95-7.90 (m, 1H), 7.79-7.75 (m, 1H), 7.46-7.44 (m, 1H), 7.35-7.32 (m, 1H), 7.24-7.21 (m, 1H), 7.14 (br, 1H), 5.14 (br, 1H), 3.27 (s, 3H), 2.27-1.32 (m, 9H). MS m/z (APCI-pos) M+1=381.

### Example 15

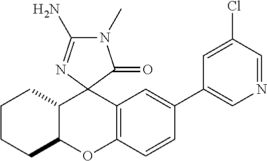

### trans 2-amino-7′-(5-chloropyridin-3-yl)-1-methyl-1′,2′,3′,4′,4a′,9a′-hexahydrospiro[imidazole-4,9′-xanthen]-5(1H)-one

The trans isomers of 2-amino-7′-(5-chloropyridin-3-yl)-1-methyl-1′,2′,3′,4′,4a′,9a′-hexahydrospiro[imidazole-4,9′-xanthen]-5(1H)-one 2,2,2-trifluoroacetate were prepared according to Example 1, Step L, substituting the trans isomers of 2-amino-7′-bromo-1-methyl-1′,2′,3′,4′,4a′,9a′-hexahydrospiro[imidazole-4,9′-xanthen]-5(1H)-one and 5-chloropyridin-3-yl boronic acid for 2-amino-7′-bromo-3′-hydroxy-1-methyl-1′,2′,3′,4′,4a′,9a′-hexahydrospiro[imidazole-4,9′-xanthen]-5(1H)-one and 3-chloro-5-fluorophenylboronic acid. ¹H NMR (CDCl₃) δ 8.51 (br, 1H), 7.89 (br, 1H), 7.49-7.45 (m, 1H), 7.17 (br, 1H), 7.10-7.08 (m, 2H), 5.14 (br, 1H), 3.27 (s, 3H), 2.27-2.21 (m, 1H), 1.98-1.25 (m, 8H). MS m/z (APCI-pos) M+1=397.

### Example 16

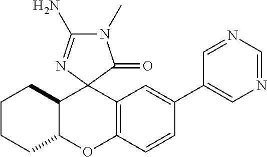

### trans 2-amino-1-methyl-7′-(pyrimidin-5-yl)-1′,2′,3′,4′,4a′,9a′-hexahydrospiro[imidazole-4,9′-xanthen]-5(1H)-one

The trans isomers of 2-amino-1-methyl-7′-(pyrimidin-5-yl)-1′,2′,3′,4′,4a′,9a′-hexahydrospiro[imidazole-4,9′-xanthen]-5(1H)-one 2,2,2-trifluoroacetate were prepared according to Example 1, Step L, substituting the trans isomers of 2-amino-7′-bromo-1-methyl-1′,2′,3′,4′,4a′,9a′-hexahydrospiro[imidazole-4,9′-xanthen]-5(1H)-one and pyrimidin-5-yl boronic acid for 2-amino-7′-bromo-3′-hydroxy-1-methyl-1′,2′,3′,4′,4a′,9a′-hexahydrospiro[imidazole-4,9′-xanthen]-5(1H)-one and 3-chloro-5-fluorophenylboronic acid. ¹H NMR (CDCl₃) δ 9.19 (br, 1H), 8.95 (br, 1H), 8.09 (br, 1H), 7.5 (dd, J=1.96, 8.6 Hz, 1H), 7.15 (d, J=7.96 Hz, 1H), 7.09 (d, J=8.61 Hz, 1H), 4.71-4.63 (m, 1H), 3.29 (s, 3H), 2.36-2.31 (m, 1H), 2.21-2.15 (m, 1H), 1.96-1.79 (m, 3H), 1.57-1.49 (m, 1H), 1.41-1.32 (m, 2H), 0.99-0.89 (m, 1H). MS m/z (APCI-pos) M+1=364.

### Example 17

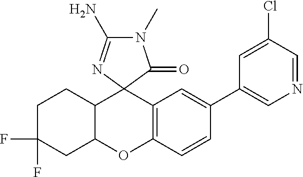

### 2-amino-7′-(5-chloropyridin-3-yl)-3′,3′-difluoro-1-methyl-1′,2′,3′,4′,4a′,9a′-hexahydrospiro[imidazole-4,9′-xanthen]-5(1H)-one

Step A: Bis(2-methoxyethyl)aminosulfur trifluoride (0.0731 mL, 0.397 mmol) was added to a mixture of 2-amino-7′-bromo-1-methyl-1′,4′,4a′,9a′-tetrahydrospiro[imidazole-4,9′-xanthene]-3′,5(1H, 2′H)-dione (0.050 g, 0.132 mmol) in DCE (0.5 mL, 0.132 mmol) at 0° C. The mixture was stirred at room temperature overnight. The mixture was partitioned between DCM and saturated NaHCO₃. The organics were extracted with DCM twice, washed with brine and dried with Na₂SO₄. This was then concentrated down and purified on preparative HPLC to give 2-amino-7′-bromo-3′,3′-difluoro-1-methyl-1′,2′,3′,4′,4a′,9a′-hexahydrospiro[imidazole-4,9′-xanthen]-5(1H)-one (0.184 g, 0.460 mmol, 18.5%).

Step B: A mixture of 2-amino-7′-bromo-3′,3′-difluoro-1-methyl-1′,2′,3′,4′,4a′,9a′-hexahydrospiro[imidazole-4,9′-xanthen]-5(1H)-one (0.030 g, 0.0750 mmol), 5-chloropyridin-3-ylboronic acid (0.0142 g, 0.0900 mmol), Pd(PPh₃)₄ (0.00433 g, 0.00375 mmol) and Na₂CO₃ (0.0825 mL, 0.165 mmol) in dioxane (0.5 mL, 0.0750 mmol) was heated to 90° C. overnight in a capped vial. The mixture was then partitioned between DCM and water. The organics were extracted with DCM twice, washed with brine and dried with Na₂SO₄. This was then purified on a column using DCM:MeOH:NH₄OH (90:10:1) to give 2-amino-7′-(5-chloropyridin-3-yl)-3′,3′-difluoro-1-methyl-1′,2′,3′,4′,4a′,9a′-hexahydro spiro[imidazole-4,9′-xanthen]-5(1H)-one (0.0136 g, 0.0314 mmol, 41.9% yield). ¹H NMR (CD₃OD) δ 8.59 (d, 1H), 8.48 (d, 1H), 7.74 (t, 1H), 7.39 (dd, 1H), 7.09 (s, 1H), 7.00 (d, 1H), 4.92 (m, 1H), 3.11 (s, 3H), 2.77 (m, 1H), 2.15 (m, 1H), 1.90 (m, 4H), 1.20 (m, 1H); MS m/z (APCI-pos) M+1=433.1.

### Example 18

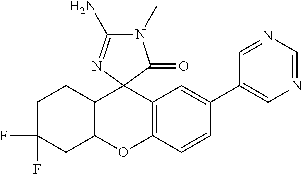

### 2-amino-3′,3′-difluoro-1-methyl-7′-(pyrimidin-5-yl)-1′,2′,3′,4′,4a′,9a′-hexahydrospiro[imidazole-4,9′-xanthen]-5(1H)-one

2-Amino-3′,3′-difluoro-1-methyl-7′-(pyrimidin-5-yl)-1′,2′,3′,4′,4a′,9a′-hexahydrospiro[imidazole-4,9′-xanthen]-5(1H)-one was made according to the procedure of Example 17, Step B, substituting pyrimidin-5-ylboronic acid for 5-chloropyridin-3-ylboronic acid. ¹H NMR (CD₃OD) δ 9.14 (s, 1H), 8.83 (s, 2H), 7.41 (dd, 1H), 7.10 (d, 1H), 7.03 (d, 1H), 4.95 (m, 1H), 3.11 (s, 3H), 2.78 (m, 1H), 2.14 (m, 1H), 1.95 (d, 1H), 1.83 (m, 2H), 1.68 (m, 1H), 1.20 (d, 1H); MS m/z (APCI-pos) M+1=400.1.

### Example 19

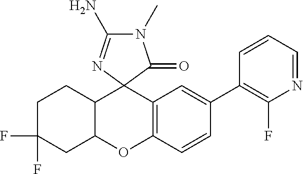

### 2-amino-3′,3′-difluoro-7′-(2-fluoropyridin-3-yl)-1-methyl-1′,2′,3′,4′,4a′,9a′-hexahydrospiro[imidazole-4,9′-xanthen]-5(1H)-one

2-Amino-3′,3′-difluoro-7′-(2-fluoropyridin-3-yl)-1-methyl-1′,2′,3′,4′,4a′,9a′-hexahydrospiro[imidazole-4,9′-xanthen]-5(1H)-one was made according to the procedure of Example 17, Step B, substituting 2-fluoropyridin-3-ylboronic acid for 5-chloropyridin-3-ylboronic acid. ¹H NMR (CD₃OD) δ 8.12 (d, 1H), 7.77 (m, 1H), 7.39 (d, 1H), 7.22 (m, 1H), 7.15 (s, 1H), 6.98 (d, 1H), 4.86 (m, 1H), 3.08 (s, 3H), 2.80 (m, 1H), 2.12, (m, 2H), 1.86 (m, 3H), 1.20 (m, 1H); MS m/z (APCI-pos) M+1=417.1.

### Example 20

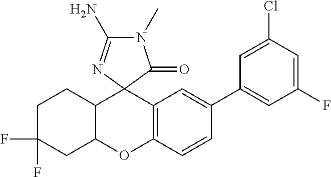

### 2-amino-7′-(3-chloro-5-fluorophenyl)-3′,3′-difluoro-1-methyl-1′,2′,3′,4′,4a′,9a′-hexahydrospiro[imidazole-4,9′-xanthen]-5(1H)-one

2-Amino-7′-(3-chloro-5-fluorophenyl)-3′,3′-difluoro-1-methyl-1′,2′,3′,4′,4a′,9a′-hexahydrospiro[imidazole-4,9′-xanthen]-5(1H)-one was made according to the procedure of Example 17, Step B, substituting 3-chloro-5-fluorophenylboronic acid for 5-chloropyridin-3-ylboronic acid. MS m/z (APCI-pos) M+1=450.1.

### Example 21

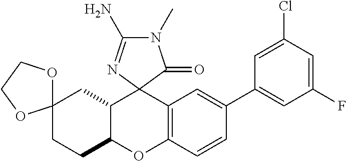

### (4a′S,9a′R)-2″-amino-7′-(3-chloro-5-fluorophenyl)-1″methyl-1″,3′,4′,4a′,5″,9′a-hexahydro-1′H-dispiro[1,3-dioxolane-2,2′-xanthene-9′,4″-imidazole]-5″-one

Step A: A solution of 1,4-cyclohexanedione monoethylene ketal (100 g, 640 mmol) and morpholine (83.7 mL, 960 mmol) in toluene (640 mL) was treated with p-toluenesulfonic acid hydrate (1.22 g, 6.40 mmol). The reaction was fitted with a Dean-Stark trap and a condenser and then heated at reflux for 24 hours. The reaction was cooled to ambient temperature and then concentrated in vacuo to provide 4-(1,4-dioxaspiro[4.5]dec-7-en-8-yl)morpholine (145 g, 515 mmol, 80%).

Step B: A solution of 5-bromo-2-hydroxybenzaldehyde (42.7 g, 213 mmol) and 4-(1,4-dioxaspiro[4.5]dec-7-en-8-yl)morpholine (68.4 g, 213 mmol) in toluene (106 mL) was stirred at room temperature for 24 hours. The precipitate was collected by filtration, and the solid was washed with cold toluene and then dried to afford 7′-bromo-4a′-morpholino-1′,3′,4′,4a′,9′,9a′-hexahydrospiro[[1,3]dioxolane-2,2′-xanthen]-9′-ol (58.0 g, 136 mmol, 64%).

Step C: A solution of 7′-bromo-4a′-morpholino-1′,3′,4′,4a′,9′,9a′-hexahydrospiro[[1,3]dioxolane-2,2′-xanthen]-9′-ol (50.0 g, 117 mmol) in DCM (586 mL) was cooled to 0° C., and Dess-Martin periodinane (59.7 g, 141 mmol) was slowly added. The mixture was stirred at room temperature for 2 hours, monitoring by TLC (50% ethyl acetate/hexanes). The reaction mixture was diluted with DCM and then slowly quenched with 2N NaOH. The mixture was poured into a separatory funnel, rinsing the flask with DCM and water. The organic layer was washed with 2N HCl, brine, dried and then concentrated to afford a residue. The residue was dissolved with a minimal amount of DCM, loaded onto a flash column and then eluted with a gradient of 40% DCM/hexanes to 40% DCM/ethyl acetate to afford T-bromo-3′,4′-dihydrospiro[[1,3]dioxolane-2,2′-xanthen]-9′(1′H)-one (38.0 g, 113 mmol, 96%).

Step D: A solution of 7′-bromo-3′,4′-dihydrospiro[[1,3]dioxolane-2,2′-xanthen]-9′(FH)-one (18.0 g, 53.4 mmol) in THF (267 mL) was cooled to −78° C., and L-selectride (1M in THF, 80.1 mL, 80.1 mmol) was added. The reaction was stirred at −78° C. for 1 hour and then quenched with NH₄Cl (saturated). The reaction mixture was warmed to room temperature and then partitioned between ethyl acetate and water. The aqueous layer was extracted with ethyl acetate (3×). The combined organic layers were dried and concentrated to give a residue that was purified by flash chromatography, eluting with 40% DCM/hexanes to 40% DCM/ethyl acetate gradient to afford (4a′S,9a′S)-7′-bromo-1′,4′,4a′,9a′-tetrahydrospiro[[1,3]dioxolane-2,2′-xanthen]-9′(3′H)-one (9.50 g, 28.0 mmol, 53% yield).

Step E: Ammonium carbonate (4.53 g, 47.2 mmol), KCN (0.768 g, 11.8 mmol), and NaHSO₃ (0.245 g, 2.36 mmol) were added to a teflon-lined steel pressure reactor containing a solution of (4a′S,9a′S)-7′-bromo-1′,4′,4a′,9a′-tetrahydrospiro[[1,3]dioxolane-2,2′-xanthen]-9′(3′H)-one (2.0 g, 5.90 mmol) in EtOH (5.90 mL). The reactor was sealed and heated at 130° C. for 18 hours. The reactor was cooled to ambient temperature. The reaction mixture was transferred to a 500 mL beaker and acidified with HCl (4N). The precipitate was collected by filtration and washed thoroughly with water to afford (4a′S,9′R,9a′R)-7′-bromo-2′,2′-spiro(1,3-dioxolane)-1′,2′,3′,4′,4a′,9a′-hexahydro spiro[imidazolidine-4,9′-xanthene]-2,5-dione (2.40 g, 5.86 mmol, 99%).

Step F: A mixture of (4a′S,9′R,9a′R)-7′-bromo-2′,2′-spiro(1,3-dioxolane)-1′,2′,3′,4′,4a′,9a′-hexahydrospiro[imidazolidine-4,9′-xanthene]-2,5-dione (1.06 g, 2.59 mmol) and KOH (1.45 g, 25.9 mmol) in water (5.18 mL) was heated at 195° C. (sand bath in metal bowl) overnight. The reactor was cooled to room temperature, and the reaction mixture was transferred to an erlenmeyer flask and neutralized with 4N HCl. (4a′S,9′R,9a′S)-9′-amino-7′-bromo-1′,3′,4′,4a′,9′,9a′-hexahydrospiro[[1,3]dioxolane-2,2′-xanthene]-9′-carboxylic acid (0.490 g, 1.28 mmol, 98%) precipitated at pH<7 and was collected by filtration. The filtrate was extracted with DCM (5×). The combined organic extracts were dried and concentrated to give (4a′S,9′R,9a′R)-9′-amino-7′-bromo-1′,3′,4′,4a′,9′,9a′-hexahydrospiro[[1,3]dioxolane-2,2′-xanthene]-9′-carboxylic acid (0.345 g, 0.898 mmol, 69%).

Step G: A solution of (4a′S,9′R,9a′R)-9′-amino-7′-bromo-1′,3′,4′,4a′,9′,9a′-hexahydrospiro[[1,3]dioxolane-2,2′-xanthene]-9′-carboxylic acid (0.345 g, 0.898 mmol) in MeOH (4.50 mL) was treated with TMSCHN₂ (2.24 mL, 4.50 mmol) as a 2.0M solution in hexanes. Within 30 seconds, a gentle bubbling initiated in the reaction mixture. Within 5 minutes, the bubbling stopped. The reaction mixture was concentrated to afford (4a′S,9′R,9a′R)-methyl 9′-amino-7′-bromo-1′,3′,4′,4a′,9′,9a′-hexahydrospiro[[1,3]dioxolane-2,2′-xanthene]-9′-carboxylate (0.280 g, 0.703 mmol, 78%).

Step H: EDCI (0.173 g, 0.904 mmol) was added to a solution of (4a′S,9′R,9a′R)-methyl 9′-amino-7′-bromo-1′,3′,4′,4a′,9′,9a′-hexahydrospiro[[1,3]dioxolane-2,2′-xanthene]-9′-carboxylate (0.200 g, 0.502 mmol), N-methyl-N′-tert-butyloxycarbonyl thiourea (0.143 g, 0.753 mmol) and DIEA (0.437 mL, 2.51 mmol) in DMF (2.51 mL), and the resulting mixture was heated at 55° C. for 6 hours. The reaction mixture was partitioned between ethyl acetate/water, and the aqueous layer was extracted with ethyl acetate (3×). The combined organic layers were dried and concentrated to give a residue that was purified by flash chromatography eluting with hexanes/ethyl acetate to afford (4a′S,9′R,9a′R)-2-amino-7′-bromo-2′-spiro[1,3]dioxolane-1-methyl-1′,2′,3′,4′,4a′,9a′-hexahydrospiro[imidazole-4,9′-xanthen]-5(1H)-one (0.203 g, 0.389 mmol, 77% yield).

Step I: A solution of (4a′S,9′R,9a′R)-2-amino-7′-bromo-2′-spiro[1,3]dioxolane-1-methyl-1′,2′,3′,4′,4a′,9a′-hexahydro spiro[imidazole-4,9′-xanthen]-5(1H)-one (50 mg, 0.0957 mmol), 3-chloro-5-fluorophenylboronic acid (17.5 mg, 0.100 mmol), Pd(PPh₃)₄ (5.53 mg, 0.00479 mmol), Na₂CO₃ (144 μL, 0.287 mmol, 2M aqueous) in dioxane (479 μL) was degassed with nitrogen for 5 minutes, and then sealed in a vial and stirred at 80° C. for 1 day. The reaction mixture was diluted with ethyl acetate and filtered through a syringe filter. The filtrate was concentrated, and the residue was treated with 4N HCl/dioxane in methanol (1 mL). After 5 minutes, the solvent was concentrated and the residue was purified by flash chromatography, eluting with DCM/MeOH+1% NH₄OH gradient to afford (4a′S,9a′R)-2″-amino-7′-(3-chloro-5-fluorophenyl)-1″methyl-1″,3′,4′,4a′,5″,9′a-hexahydro-1′H-dispiro[1,3-dioxolane-2,2′-xanthene-9′,4″-imidazole]-5″-one (21 mg, 0.045 mmol, 47%). m/z (APCI-pos) M+1=472 (100%), 473 (25%), 474 (50%).

### Example 22

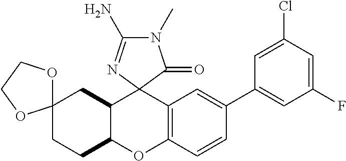

### (4R,4a′S,9a′S)-2″-amino-7′-(3-chloro-5-fluorophenyl)-1″methyl-1″,3′,4′,4a′,5″,9′a-hexahydro-1′H-dispiro[1,3-dioxolane-2,2′-xanthene-9′,4″-imidazole]-5″-one

Prepared in an analogous fashion as Example 21, carrying the solid from Example 21, Step F, (4a′S,9′R,9a′S)-9′-amino-7′-bromo-1′,3′,4′,4a′,9′,9a′-hexahydrospiro[[1,3]dioxolane-2,2′-xanthene]-9′-carboxylic acid, through step G-I, affording (4R,4a′S,9a′S)-2″-amino-7′-(3-chloro-5-fluorophenyl)-1″methyl-1″,3′,4′,4a′,5″,9′a-hexahydro-1′H-dispiro[1,3-dioxolane-2,2′-xanthene-9′,4″-imidazole]-5″-one. m/z (APCI-pos) M+1=472 (100%), 473 (30%), 474 (40%).

### Example 23

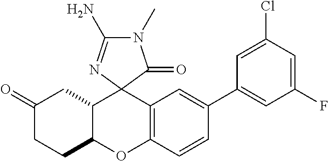

### (4R,4a′S,9a′R)-2-amino-7′-(3-chloro-5-fluorophenyl)-1-methyl-1′,4′,4a′,9a′-tetrahydrospiro[imidazole-4,9′-xanthene]-2′,5(1H, 3′H)-dione

The product from Example 21, Step I, (4a′S,9′R,9a′R)-2-amino-7′-(3-chloro-5-fluorophenyl)-2′,2′-spiro[1,3]-dioxolane-1-methyl-1′,2′,3′,4′,4a′,9a′-hexahydrospiro[imidazole-4,9′-xanthen]-5(1H)-one (21 mg, 0.045 mmol), was dissolved in HCl (223 μL, 0.45 mmol) and acetone (223 μL, 0.045 mmol), and the solution was heated at 55° C. for 1 day. The reaction mixture was diluted with ethyl acetate and washed with Na₂CO₃ (saturated). The aqueous layer was extracted with ethyl acetate (2×). The combined organic layers were dried and concentrated. The residue dried in vacuo for 1 hour to give a solid corresponding to (4R,4a′S,9a′R)-2-amino-7′-(3-chloro-5-fluorophenyl)-1-methyl-1′,4′,4a′,9a′-tetrahydrospiro[imidazole-4,9′-xanthene]-2′,5(1H, 3′H)-dione. ¹H NMR (CDCl₃) δ 7.37 (dd, J=9.0, 2.4 Hz, 1H), 7.21 (br s, 1H), 7.09 (d, J=2.0 Hz, 1H), 6.93-7.00 (m, 2H), 5.07 (td, J=11, 3.9 Hz, 1H), 3.13 (s, 3H), 2.57 (m, 1H), 2.48 (m, 2H), 2.33 (m, 2H), 1.80-2.00 m, 2H).

### Example 24

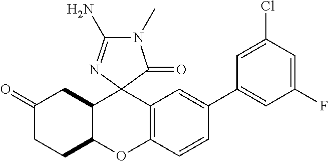

### (4R,4a′S,9a′S)-2-amino-7′-(3-chloro-5-fluorophenyl)-1-methyl-1′,4′,4a′,9a′-tetrahydro spiro[imidazole-4,9′-xanthene]-2′,5(1H, 3′H)-dione

Using an analogous route as Example 23, the product from Example 22 was hydrolyzed to afford (4R,4a′S,9a′S)-2-amino-7′-(3-chloro-5-fluorophenyl)-1-methyl-1′,4′,4a′,9a′-tetrahydrospiro[imidazole-4,9′-xanthene]-2′,5(1H, 3′H)-dione. ¹H NMR (CDCl₃) 7.42 (d, J=8.2 Hz, 1H), 7.25 (s, 1H), 7.16 (s, 1H), 7.05 (m, 2H), 6.98 (d, J=7.4 Hz, 1H), 5.3 (br s, 1H), 3.20 (s, 3H), 2.76 (m, 1H), 2.64-2.48 (m, 2H), 2.32 (t, J=13 Hz, 2H), 2.33 (m, 2H), 1.96 (m, 2H).

### Example 25

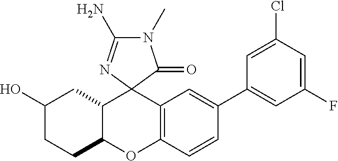

### (4R,4a′S,9a′R)-2-amino-7′-(3-chloro-5-fluorophenyl)-2′-hydroxy-1-methyl-1′,2′,3′,4′,4a′,9a′-hexahydrospiro[imidazole-4,9′-xanthen]-5(1H)-one

The product from Example 23, (4a′S,9a′R)-2-amino-7′-(3-chloro-5-fluorophenyl)-1-methyl-1′,4′,4a′,9a′-tetrahydro spiro[imidazole-4,9′-xanthene]-2′,5(1H, 3′H)-dione (10.6 mg, 0.0248 mmol), was dissolved in THF (248 μL, 0.0248 mmol), and the solution was cooled to −78° C. NaBH₄ (1.87 mg, 0.0495 mmol) and 2 drops of methanol were added to this solution, and the resulting mixture was stirred for 15 minutes. The mixture was filtered, and the material was purified by C18 chromatography, eluting with ACN/H₂O+0.1% TFA. The product-containing fractions were concentrated to afford (4R,4a′S,9a′R)-2-amino-7′-(3-chloro-5-fluorophenyl)-2′-hydroxy-1-methyl-1′,2′,3′,4′,4a′,9a′-hexahydrospiro[imidazole-4,9′-xanthen]-5(1H)-one as the TFA salt. ¹H NMR (CDCl₃) δ 7.40 (m, 1H), 7.26 (m, 1H), 7.12 (m, 2H), 7.05 (m, 1H), 6.95 (m, 1H), 4.25 (m, 1H), 3.65 (m, 1H), 3.10 (s, 3H), 2.28 (m, 1H), 2.10-1.95 (m, 2H), 1.60 (m, 1H), 1.35 (m, 1H), 0.90 (m, 2H).

### Example 26

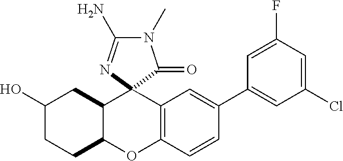

### (4R,4as′S,9a′S)-2-amino-7′-(3-chloro-5-fluorophenyl)-2′-hydroxy-1-methyl-1′,2′,3′,4′,4a′,9a′-hexahydrospiro[imidazole-4,9′-xanthen]-5(1H)-one

The product from Example 24, (4R,4a′S,9a′S)-2-amino-7′-(3-chloro-5-fluorophenyl)-1-methyl-1′,4′,4a′,9a′-tetrahydrospiro[imidazole-4,9′-xanthene]-2′,5(1H, 3′H)-dione (7.0 mg, 0.016 mmol), was dissolved in THF (164 μL, 0.016 mmol) was cooled to −78° C. NaBH₄ (1.2 mg, 0.033 mmol) and 2 drops of methanol were added to this solution, and the resulting mixture was stirred for 15 minutes. The mixture was filtered, and the material was purified by C18 chromatography, eluting with ACN/H₂O+0.1% TFA. The product-containing fractions were concentrated to afford (4R,4a′S,9a′S)-2-amino-7′-(3-chloro-5-fluorophenyl)-2′-hydroxy-1-methyl-1′,2′,3′,4′,4a′,9a′-hexahydrospiro[imidazole-4,9′-xanthen]-5(1H)-one (4.3 mg, 0.01 mmol, 61%). ¹H NMR (CDCl₃) δ 7.35 (m, 1H), 7.28 (m, 1H), 7.25 (m, 1H), 7.13 (m, 1H), 7.08 (m, 1H), 6.99 (m, 1H), 5.13 (m, 1H), 3.65 (m, 1H), 3.08 (s, 3H), 2.26 (m, 1H), 2.08 (m, 1H), 1.80 (m, 2H), 1.55 (m, 1H), 0.90 (m, 2H).

### Example 27

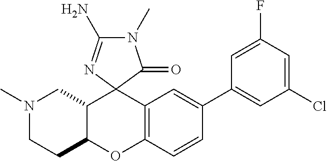

### rac-trans-(4a,10a)-2′-amino-8-(3-chloro-5-fluorophenyl)-1′,2-dimethyl-1,2,3,4,4a,10a-hexahydrospiro[chromeno[3,2-c]pyridine-10,4′-imidazol]-5′(1′H)-one

Step A: Ethyl 4-chloronicotinate was prepared from 4-chloronicotinic acid as described in WO 2008/024725.

Step B: Cs₂CO₃ (25.5 g, 78.2 mmol) was added to a solution of ethyl 4-chloronicotinate (12.1 g, 65.2 mmol) and 4-bromophenol (11.8 g, 68.5 mmol) in DMF (217 mL). The reaction mixture was heated in an 80° C. sand bath and stirred for 20 hours. The reaction mixture was concentrated in vacuo, and the residue was partitioned between water and ethyl acetate. The mixture was extracted with ethyl acetate (2×), and the combined extracts were washed with brine, dried (Na₂SO₄), filtered, and concentrated. The crude was purified on silica gel (5-40% ethyl acetate in dichloromethane gradient) to give ethyl 4-(4-bromophenoxy)nicotinate (19.2 g, 91.4%) as an oil that solidified on standing.

Step C: NaOH (3.58 g, 89.4 mmol) was added to a 0° C. solution of ethyl 4-(4-bromophenoxy)nicotinate (19.2 g, 59.6 mmol) in THF (300 mL) and H₂O (150 mL). The reaction mixture was warmed to room temperature and stirred for 7 hours. The THF was removed in vacuo, ice water (100 mL) was added, and the pH adjusted to about 3 by the addition of formic acid (3.60 mL, 95.4 mmol). Solid NaCl was added, and the mixture was extracted with ethyl acetate (2×). The combined extracts were dried (Na₂SO₄), filtered, and concentrated to give 4-(4-bromophenoxy)nicotinic acid (18.1 g, 103%) as a powder.

Step D: Concentrated sulfuric acid (123 mL, 2308 mmol) was added to a 1 L round-bottomed flask containing 4-(4-bromophenoxy)nicotinic acid (18.1 g, 61.5 mmol). The mixture was stirred until all of the solids dissolved, and the reaction mixture was heated in a 150° C. sand bath and stirred for 16 hours. The reaction mixture was then cooled to room temperature and poured slowly/portionwise into a 0° C. solution of NaOH (187 g, 4677 mmol) in 2 L of ice water (ice added periodically to maintain temp below 15° C.) causing precipitation. The solids were isolated by vacuum filtration through qualitative filter paper on a Buchner funnel, rinsed with water, and air dried. The filtrate was extracted with dichloromethane (2×), and the extracts were dried (Na₂SO₄), filtered, and concentrated. The resulting solids were combined with the solids above to give 8-bromo-10H-chromeno[3,2-c]pyridin-10-one (15.0 g, 88.3%) as a powder.

Step E: 8-Bromo-10H-chromeno[3,2-c]pyridin-10-one (3.00 g, 10.9 mmol) with 1,2-dichloroethane (54 mL) were combined into a 150 mL sealable reaction pressure tube, and neat MeI (4.07 mL, 65.2 mmol) was added. The reaction tube was capped tightly and heated in an 80° C. sand bath and stirred for 21 hours. The reaction mixture was then diluted with dichloromethane, and the solids were isolated by vacuum filtration through a 0.45 micron nylon filter membrane, rinsed with DCM and ether, and dried in vacuo to give 8-bromo-2-methyl-10-oxo-10H-chromeno[3,2-c]pyridin-2-ium iodide (4.50 g, 99.1%) as a powder.

Step F: NaBH₄ (3.26 g, 86.1 mmol) was added in portions to a 0° C. mixture of 8-bromo-2-methyl-10-oxo-10H-chromeno[3,2-c]pyridin-2-ium iodide (9.0 g, 21.5 mmol) in 1:1 EtOH:THF (172 mL). The reaction mixture was stirred at 0° C. for 1 hour, another 1 equivalent of NaBH₄ was added, and the reaction mixture continued to stir at 0° C. After 2 hours total, another 1 equivalent NaBH₄ was added, and the reaction mixture was stirred as the bath was allowed to slowly die. After 3 hours total, the reaction mixture was concentrated, and the resulting residue was combined with ethyl acetate, stirred, and ice saturated NH₄Cl was added. The mixture was diluted with brine and extracted with ethyl acetate (2×). The combined extracts were dried (Na₂SO₄), filtered, and concentrated to give a foam. The crude was purified on silica gel (5-50% ethyl acetate in hexanes gradient, then 10-40% ethyl acetate in dichloromethane gradient) to give rac-8-bromo-2-methyl-2,3,4,4a,10,10a-hexahydro-1H-chromeno[3,2-c]pyridin-10-ol (5.40 g, 84.1% yield) as a foam as a mixture of diastereomers.

Step G: A solution of DMSO (3.86 mL, 54.3 mmol) in dichloromethane (10 mL) was slowly added to a −78° C. solution of 2M oxalyl chloride in dichloromethane (13.6 ml, 27.2 mmol) in dichloromethane (100 mL) The reaction mixture was stirred for 10 minutes, and then a solution of rac-8-bromo-2-methyl-2,3,4,4a,10,10a-hexahydro-1H-chromeno[3,2-c]pyridin-10-ol (5.40 g, 18.1 mmol) in 2:1 dichloromethane:THF (30 mL) was added dropwise by syringe. The reaction mixture was stirred at −78° C. for 45 minutes, and then TEA (15.1 mL, 109 mmol) was added. The reaction mixture was allowed to warm to room temperature and stirred for 1 hour. Water (100 mL) was then added, the mixture was extracted with dichloromethane (2×), and the combined extracts were dried (Na₂SO₄), filtered, concentrated, and dried in vacuo to give crude rac-8-bromo-2-methyl-2,3,4,4a-tetrahydro-1H-chromeno[3,2-c]pyridin-10(10aH)-one (5.6 g, 104%) as a ca. 4:1 mixture of cis:trans diastereomers, which was used directly in the next step.

Step H: K₂CO₃ (0.261 g, 1.89 mmol) was added to a sonicated heterogeneous mixture ca. 4:1 cis:trans-rac-8-bromo-2-methyl-2,3,4,4a-tetrahydro-1H-chromeno[3,2-c]pyridin-10(10aH)-one (5.6 g, 18.9 mmol) in MeOH (160 mL), and the reaction mixture was stirred at room temperature. After 3 hours, the reaction mixture was poured into a mixture of saturated NH₄Cl (200 mL) and water (800 mL), and the resulting solids were isolated by vacuum filtration through qualitative filter paper on a Buchner funnel, rinsed with water, air dried, and dried in vacuo to give a powder. The crude was stirred in IPA (140 mL; 25 mL/g) in a 65° C. sand bath for 30 minutes, then cooled in an ice bath, and the solids were isolated by vacuum filtration through qualitative filter paper on a Buchner funnel, washed with IPA (2×40 mL), air dried, and dried in vacuo to give rac-(trans)-8-bromo-2-methyl-2,3,4,4a-tetrahydro-1H-chromeno[3,2-c]pyridin-10(10aH)-one (2.48 g, 8.37 mmol, 44.3% yield) as a powder.

Step I: A stainless steel Parr acid digestion bomb with Teflon insert was charged with rac-(trans)-8-bromo-2-methyl-2,3,4,4a-tetrahydro-1H-chromeno[3,2-c]pyridin-10(10aH)-one (1.0 g, 3.38 mmol), KCN (0.440 g, 6.75 mmol), ammonium carbonate (1.95 g, 20.3 mmol), NaHSO₃ (0.0878 g, 0.844 mmol) and absolute EtOH (4.8 mL), and the mixture was heated in a 100° C. oil bath and stirred. After 23 hours, the reaction mixture was diluted with ethyl acetate and IPA, and the mixture was vacuum filtered through GF/F paper and rinsed with ethyl acetate/IPA. The filtrate was dried (Na₂SO₄), filtered, concentrated, and dried in vacuo to give rac-8-bromo-2-methyl-1,2,3,4,4a,10a-hexahydrospiro-[chromeno[3,2-c]pyridine-10,4′-imidazolidine]-2′,5′-dione (1.19 g, 96%) as a solid as a mixture of diastereomers, which was taken forward without any further purification.

Step J: A 15 mL stainless steel Parr acid digestion bomb with Teflon insert was charged with rac-8-bromo-2-methyl-1,2,3,4,4a,10a-hexahydro-spiro-[chromeno[3,2-c]pyridine-10,4′-imidazolidine]-2′,5′-dione (0.500 g, 1.37 mmol), KOH (0.766 g, 13.7 mmol), and 1:1 water:dioxane (1.4 mL), and the bomb was sealed and heated in a 200° C. sand bath and stirred for 24 hours. Another 5 equivalents of KOH were added, and the reaction mixture was heated back to 200° C. and stirred another 3 days. The reaction mixture was then transferred to an Erlenmeyer flask, cooled to 0° C., and 6M HCl, followed by 1M HCl, were added until the pH was about 7. The reaction mixture was then added dropwise to vigorously stirring water (40 mL), causing a fine precipitate to form. The solids were removed by vacuum filtration through qualitative filter paper on a Buchner funnel, and rinsed with water. The filtrate was concentrated to dryness to give a residue. The resulting solids were sonicated with MeOH/THF, and the remaining solids were removed by vacuum filtration through a 0.2 micron nylon filter membrane, rinsed with MeOH/THF, and the filtrate was concentrated and dried in vacuo to give crude rac-10-amino-8-bromo-2-methyl-2,3,4,4a,10,10a-hexahydro-1H-chromeno[3,2-c]pyridine-10-carboxylic acid (553 mg, 119%) as a mixture of diastereomers as a residue, which contained some inorganic salts. The crude was used without further purification.

Step K: 2M TMSCHN₂ in hexanes (6.48 ml, 13.0 mmol) was added to a mixture of rac-10-amino-8-bromo-2-methyl-2,3,4,4a,10,10a-hexahydro-1H-chromeno[3,2-c]pyridine-10-carboxylic acid (0.553 g, 1.62 mmol) in 1:1 THF:MeOH (13 mL). The reaction mixture was stirred at room temperature for 12 hours. Another 8 equivalents of TMSCHN₂ were added, and the reaction mixture continued to stir at room temperature, and after 16 hours, another 5 equivalents TMSCHN₂ were added. After 19 hours total, the reaction mixture was quenched by the addition of ice with vigorous stirring until bubbling ceased. The organics were concentrated, then saturated NaHCO₃ and brine were added, and the mixture was extracted with 25% IPA/DCM (2×). The combined extracts were dried (Na₂SO₄), filtered, and concentrated, to give rac-methyl 10-amino-8-bromo-2-methyl-2,3,4,4a,10,10a-hexahydro-1H-chromeno[3,2-c]pyridine-10-carboxylate (162 mg, 28%) as a mixture of diastereomers as a residue, which was taken forward crude into the next step.

Step L: TEA (0.254 mL, 1.82 mmol) and a solution of isothiocyanatomethane (0.0667 g, 0.912 mmol) in THF (1 mL) were added to a solution of rac-methyl 10-amino-8-bromo-2-methyl-2,3,4,4a,10,10a-hexahydro-1H-chromeno[3,2-c]pyridine-10-carboxylate (0.162 g, 0.456 mmol) in THF (3.5 mL), and the reaction mixture was heated in a 60° C. reaction block and stirred for 6 hours. Another 4 equivalents of isothiocyanatomethane was added, and the reaction mixture continued to stir at 60° C. for 20 hours. The reaction mixture was concentrated and loaded directly onto a preparative TLC plate (2 mm plate, 9:1 DCM:MeOH) to give rac-trans-(4a,10a)-8-bromo-1′,2-dimethyl-2′-thioxo-1,2,3,4,4a,10a-hexahydrospiro[chromeno[3,2-c]pyridine-10,4′-imidazolidin]-5′-one (0.026 g, 0.0656 mmol, 14.4% yield) as a solid.

Step M: 7M NH₃ in MeOH (0.187 mL, 1.31 mmol) and t-butyl hydroperoxide (70% aqueous, 0.0938 mL, 0.656 mmol) were added to a solution of rac-trans-(4a,10a)-8-bromo-1′,2-dimethyl-2′-thioxo-1,2,3,4,4a,10a-hexahydrospiro[chromeno-[3,2-c]pyridine-10,4′-imidazolidin]-5′-one (0.026 g, 0.0656 mmol) in THF (0.3 mL), and the reaction mixture was capped and stirred at room temperature for 3 hours. Another 20 equivalents of 7M NH₃ in MeOH and 10 equivalents of 70% t-butyl hydroperoxide were added, and the reaction mixture was heated in a 35° C. reaction block and stirred for another 14 hours. The reaction mixture was concentrated, then diluted to 0.5 mL total volume with 1:1 ACN:H₂O, and purified by reverse phase HPLC (Phenomenex C18 column, 150×21.2 mm, 0-95% ACN in H₂O with 0.1% TFA) to give rac-trans-(4a,10a)-2′-amino-8-bromo-1′,2-dimethyl-1,2,3,4,4a,10a-hexahydro spiro[chromeno[3,2-c]pyridine-10,4′-imidazol]-5′(1′H)-one bis-TFA salt (0.011 g, 29%) as a residue.

Step N: Rac-trans-(4a,10a)-2′-amino-8-bromo-1′,2-dimethyl-1,2,3,4,4a,10a-hexahydrospiro[chromeno-[3,2-c]pyridine-10,4′-imidazol]-5′(1′H)-one (0.011 g, 0.0290 mmol), 3-chloro-5-fluoro-phenylboronic acid (0.00759 g, 0.0435 mmol), and Pd(PPh₃)₄ (0.00335 g, 0.00290 mmol) were combined with dioxane (0.3 mL) and 2M Na₂CO₃ (0.0725 mL, 0.145 mmol) (both degassed 20 minutes before use), and the reaction mixture was heated in a 90° C. reaction block and stirred for 15 hours. The reaction mixture was concentrated under nitrogen stream. The resulting residue was dissolved in 1:1 ACN:H₂O (0.4 mL) and MeOH (0.2 mL) plus several drops of TFA until acidic, and this solution was purified by reverse phase HPLC (Phenomenex C18 column, 150×21.2 mm, 0-95% ACN in H₂O with 0.1% TFA) to give rac-trans-(4a,10a)-2′-amino-8-(3-chloro-5-fluorophenyl)-1′,2-dimethyl-1,2,3,4,4a,10a-hexahydro spiro[chromeno[3,2-c]pyridine-10,4′-imidazol]-5′(1′H)-one bis-TFA salt (0.0041 g, 23%) as a powder. LC/MS APCI (+) m/z 429 (M+1) detected.

The following compounds in Table 1 were prepared according to the above procedures using appropriate intermediates.

TABLE 1

| Ex. | | | NMR/ |
|---|---|---|---|
| # | Structure | Name | MS |
| 28 | 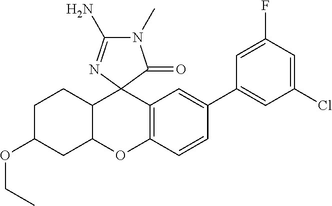 | 2-amino-7′-(3-chloro-5- fluorophenyl)-3′-ethoxy-1-methyl- l′,2′,3′,4′,4a′,9a′- hexahydrospiro[imidazole-4,9′- xanthen]-5(1H)-one | 458.1 |
| 29 | 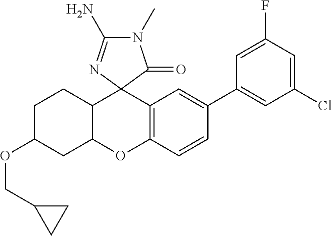 | 2-amino-7′-(3-chloro-5- fluorophenyl)-3′- (cyclopropylmethoxy)-1-methyl- 1′,2′,3′,4′,4a′,9a′- hexahydrospiro[imidazole-4,9′- xanthen]-5(1H)-one | 484.1 |
| 30 | 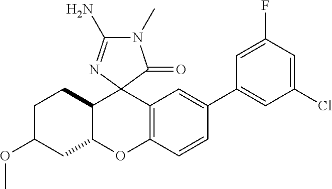 | (4a′R,9a′S)-2-amino-7′-(3-chloro- 5-fluorophenyl)-3′-methoxy-1- methyl-1′,2′,3′,4′,4a′,9a′- hexahydrospiro[imidazole-4,9′- xanthen]-5(1H)-one | 444.1 |
| 31 | 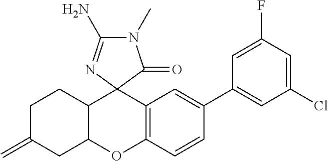 | 2-amino-7′-(3-chloro-5- fluorophenyl)-1-methyl-3′- methylene-1′,2′,3′,4′,4a′,9a′- hexahydrospiro[imidazole-4,9′- xanthen]-5(1H)-one | 426.1 |
| 32 | 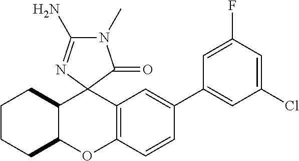 | cis 2-amino-7′-(3-chloro-5- fluorophenyl)-1-methyl- 1′,2′,3′,4′,4a′,9a′- hexahydrospiro[imidazole-4,9′- xanthen]-5(1H)-one | 414 |
| 33 | 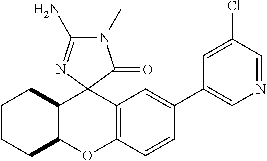 | cis 2-amino-7′-(5-chloropyridin-3- yl)-1-methyl-1′,2′,3′,4′,4a′,9a′- hexahydrospiro[imidazole-4,9′- xanthen]-5(1H)-one | 397 |
| 34 | 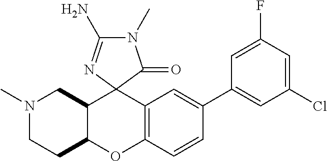 | (4aS,10aS)-2′-amino-8-(3-chloro- 5-fluorophenyl)-1′,2-dimethyl- 1,2,3,4,4a,10a- hexahydrospiro[chromeno[3,2- c]pyridine-10,4′-imidazol]-5′(1′H)- one | 429 |
| 35 | 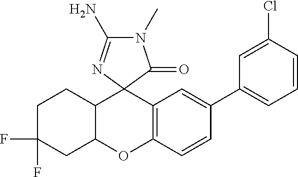 | 2-amino-7′-(3-chlorophenyl)-3′,3′- difluoro-1-methyl-1′,2′,3′,4′,4a′,9a′- hexahydrospiro[imidazole-4,9′- xanthen]-5(1H)-one | 432.1 |
| 36 | 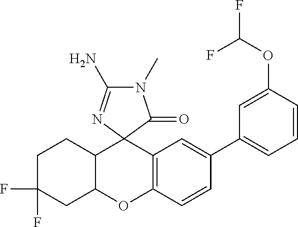 | 2-amino-7′-(3- (difluoromethoxy)phenyl)-3′,3′- difluoro-1-methyl-1′,2′,3′,4′,4a′,9a′- hexahydrospiro[imidazole-4,9′- xanthen]-5(1H)-one | 464.1 |
| 37 | 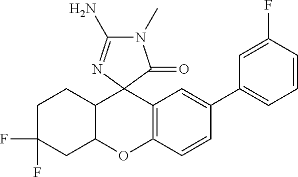 | 2-amino-3′,3′-difluoro-7′-(3- fluorophenyl)-1-methyl- 1′,2′,3′,4′,4a′,9a′- hexahydrospiro[imidazole-4,9′- xanthen]-5(1H)-one | 416.1 |
| 38 | 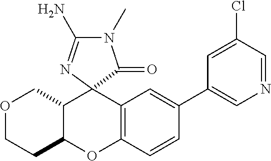 | rel-(4R,4a′S,10a′S)-2-amino-8′-(5- chloropyridin-3-yl)-1-methyl- 3′,4′,4a′,10a′-tetrahydro-1′H- spiro[imidazole-4,10′-pyrano[4,3- b]chromen]-5(1H)-one | 399 |
| 39 | 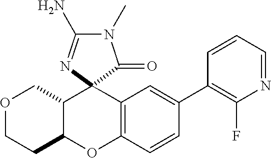 | rel-(4R,4a′S,10a′S)-2-amino-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 383 |

### Example 40

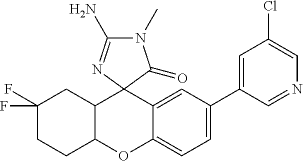

### 2-amino-7′-(5-chloropyridin-3-yl)-2′,2′-difluoro-1-methyl-1′,2′,3′,4′,4a′,9a′-hexahydrospiro[imidazole-4,9′-xanthen]-5(1H)-one

2-Amino-7′-(5-chloropyridin-3-yl)-2′,2′-difluoro-1-methyl-1′,2′,3′,4′,4a′,9a′-hexahydrospiro[imidazole-4,9′-xanthen]-5(1H)-one was made according to the procedure of Example 41, substituting (5-chloropyridin-3-yl)boronic acid for (2-fluoropyridin-3-yl)boronic acid. ¹H NMR (CD₃OD) δ 8.57 (m, 1H), 8.45 (d, 1H), 7.78 (m, 1H), 7.40 (m, 1H), 7.10 (dd, 1H), 7.02 (dd, 1H), 5.24 (s, 0.5H), 4.81 (m, 0.5H), 3.10 (d, 3H), 2.3 (m, 5H), 1.82 (m, 2H). MS m/z (APCI-pos) M+1=433.1.

### Example 41

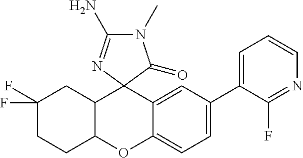

### 2-amino-2′,2′-difluoro-7′-(2-fluoropyridin-3-yl)-1-methyl-1′,2′,3′,4′,4a′,9a′-hexahydrospiro[imidazole-4,9′-xanthen]-5(1H)-one

Step A: Bis(2-methoxyethyl)aminosulfur trifluoride (0.585 mL, 3.17 mmol) was added to a mixture of 2-amino-7′-bromo-1-methyl-1′,4′,4a′,9a′-tetrahydrospiro[imidazole-4,9′-xanthene]-2′,5(1H, 3′H)-dione (0.40 g, 1.06 mmol) in DCE(bp83) (7 mL, 1.06 mmol) at 0° C. The mixture was stirred at room temperature overnight. The mixture was partitioned between DCM and saturated NaHCO₃. The organics were extracted with DCM twice, washed with brine and dried with Na₂SO₄. This was concentrated down and purified on a column using DCM:MeOH:NH₄OH (90:10:1) to give 2-amino-7′-bromo-2′,2′-difluoro-1-methyl -1′,2′,3′,4′,4a′,9a′-hexahydrospiro[imidazole-4,9′-xanthen]-5(1H)-one (0.243 g, 0.607 mmol, 57.4% yield).

Step B: A mixture of 2-amino-7′-bromo-2′,2′-difluoro-1-methyl-1′,2′,3′,4′,4a′,9a′-hexahydrospiro[imidazole-4,9′-xanthen]-5(1H)-one (0.060 g, 0.150 mmol), 2-fluoropyridin-3-ylboronic acid (0.0253 g, 0.180 mmol), Pd(PPh₃)₄ (0.00866 g, 0.00750 mmol) and Na₂CO₃ (0.165 mL, 0.330 mmol) in dioxane (1 mL, 0.150 mmol) was heated to 90° C. overnight in a capped vial. The mixture was then partitioned between DCM and water. The organics were extracted with DCM twice, washed with brine and dried with Na₂SO₄. This was then purified on a column using DCM:MeOH:NH₄OH (90:10:1) to give 2-amino-2′,2′-difluoro-7′-(2-fluoropyridin-3-yl)-1-methyl-1′,2′,3′,4′,4a′,9a′-hexahydro spiro[imidazole-4,9′-xanthen]-5(1H)-one (0.0279 g, 0.0670 mmol, 44.7% yield). ¹H NMR (CD₃OD) δ 8.15 (d, 1H), 7.75 (m, 1H), 7.36 (d, 1H), 7.18 (m, 2H), 6.99 (m, 1H), 5.27 (s, 0.5H), 4.83 (m, 0.5H), 3.04 (d, 3H), 2.2 (m, 5H), 1.8 (m, 2H); m/z (APCI-pos) M+1=417.1.

### Example 42

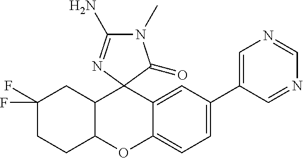

### 2-amino-2′,2′-difluoro-1-methyl-7′-(pyrimidin-5-yl)-1′,2′,3′,4′,4a′,9a′-hexahydrospiro[imidazole-4,9′-xanthen]-5(1H)-one

2-Amino-2′,2′-difluoro-1-methyl-7′-(pyrimidin-5-yl)-1′,2′,3′,4′,4a′,9a′-hexahydrospiro[imidazole-4,9′-xanthen]-5(1H)-one was made according to the procedure of Example 41, substituting pyrimidin-5-ylboronic acid for (2-fluoropyridin-3-yl)boronic acid. ¹H NMR (CD₃OD) δ 9.12 (d, 1H), 8.83 (d, 2H), 7.40 (m, 1H), 7.11 (dd, 1H), 7.04 (dd, 1H), 5.26 (s, 0.5H), 4.80 (m, 0.5H), 3.10 (d, 3H), 2.24 (m, 5H), 1.85 (m, 2H); m/z (APCI-pos) M+1=400.1.

### Example 43

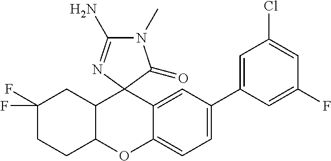

### 2-amino-7′-(3-chloro-5-fluorophenyl)-2′,2′-difluoro-1-methyl-1′,2′,3′,4′,4a′,9a′-hexahydrospiro[imidazole-4,9′-xanthen]-5(1H)-one

2-Amino-7′-(3-chloro-5-fluorophenyl)-2′,2′-difluoro-1-methyl-1′,2′,3′,4′,4a′,9a′-hexahydrospiro[imidazole-4,9′-xanthen]-5(1H)-one was made according to the procedure of Example 41, substituting (3-chloro-5-fluorophenyl)boronic acid for (2-fluoropyridin-3-yl)boronic acid. ¹H NMR (CD₃OD) δ 7.35 (m, 1H), 7.21 (m, 1H), 7.02 (m, 4H), 5.25 (s, 0.5H), 4.81 (m, 0.5H), 3.08 (d, 3H), 2.24 (m, 5H), 1.84 (m, 2H); m/z (APCI-pos) M+1=450.1.

### Example 44

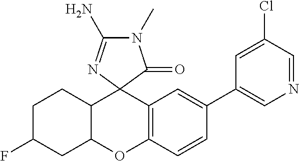

### 2-amino-7′-(5-chloropyridin-3-yl)-3′-fluoro-1-methyl-1′,2′,3′,4′,4a′,9a′-hexahydrospiro[imidazole-4,9′-xanthen]-5(1H)-one

Step A: A solution of (4R,4a′S,9aR)-2-amino-7′-bromo-3′-hydroxy-1-methyl-1′,2′,3′,4′,4a′,9a′-hexahydrospiro[imidazole-4,9′-xanthen]-5(1H)-one (322 mg, 0.847 mmol), Deoxo-Fluor® (749 mg, 3.39 mmol) in 1,2-dichloroethane (4.2 mL) in a plastic tube was stirred at room temperature overnight. The reaction mixture was poured into a separatory funnel containing NaHCO₃ (saturated), and the aqueous layer was extracted with CH₂Cl₂ (3×). The combined organic layers were dried and concentrated to give a residue that was purified by flash chromatography eluting with DCM/MeOH to afford (4R,4a′S,9a′R)-2-amino-7′-bromo-3′-fluoro-1-methyl-1′,2′,3′,4′,4a′,9a′-hexahydrospiro[imidazole-4,9′-xanthen]-5(1H)-one (270 mg, 0.706 mmol, 83%).

Step B: In a screw-top pressure vial, a suspension of 2-amino-7′-bromo-3′-fluoro-1-methyl-1′,2′,3′,4′,4a′,9a′-hexahydrospiro[imidazole-4,9′-xanthen]-5(1H)-one (115 mg, 0.301 mmol), 5-chloropyridin-3-ylboronic acid (49.7 mg, 0.316 mmol), Pd(PPh₃)₄ (17.4 mg, 0.0150 mmol) and Na₂CO₃ (2.0M, 0.5 mL, 0.903 mmol) in dioxane (1.5 mL) was degassed thoroughly with nitrogen, and the mixture was capped and heated at 90° C. overnight. The reaction mixture was diluted with MeOH and filtered through a syringe filter. The filtrate was purified by C18 prep HPLC to afford a residue that was further purified by flash chromatography eluting with a CH₂Cl₂/MeOH gradient to give 2-amino-7′-(5-chloropyridin-3-yl)-3′-fluoro-1-methyl-1′,2′,3′,4′,4a′,9a′-hexahydrospiro[imidazole-4,9′-xanthen]-5(1H)-one (15 mg, 0.0362 mmol, 12.0% yield). ¹H NMR (CD₃OD) δ 8.58 (d, J=1.6 Hz, 1H), 8.44 (d, J=2.4 Hz, 1H), 7.91 (t, J=2.0 Hz, 1H), 7.46 (s, 1H), 7.46 (dd, J=8.6, 2.3 Hz, 1H), 7.16 (d, J=2.0 Hz, 1H), 7.01 (d, J=8.6 Hz, 1H), 4.99 (td, J=11, 5 Hz, 1H), 3.11 (s, 3H), 2.64 (m, 1H), 2.09 (m, 1H), 1.98 (m, 1H), 1.69 (m, 3H), 1.28 (m, 1H); m/z (APCI-pos) M+1=415.1.

### Example 45

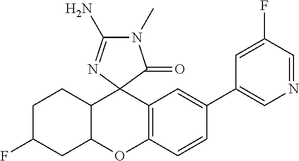

### 2-amino-3′-fluoro-7′-(5-fluoropyridin-3-yl)-1-methyl-1′,2′,3′,4′,4a′,9a′-hexahydrospiro[imidazole-4,9′-xanthen]-5(1H)-one

2-Amino-3′-fluoro-7′-(5-fluoropyridin-3-yl)-1-methyl-1′,2′,3′,4′,4a′,9a′-hexahydrospiro[imidazole-4,9′-xanthen]-5(1H)-one was prepared according to Example 44, substituting 5-fluoropyridin-3-ylboronic acid for 5-chloropyridin-3-ylboronic acid. ¹H NMR (CD₃OD) δ 8.68 (s, 1H), 8.45 (s, 1H), 7.94 (d, J=9.8 Hz, 1H), 7.69 (d, J=8.6 Hz, 1H), 7.56 (s, 1H), 7.10 (d, J=8.6 Hz, 1H), 4.92 (td, J=11.0, 4.7 Hz, 1H), 3.27 (s, 3H), 2.64 (m, 1H), 2.23 (m, 1H), 2.12 (m, 1H), 1.81 (m, 3H), 1.61 (m, 1H), 1.38 (m, 1H); m/z (APCI-pos) M+1=399.1.

### Example 46

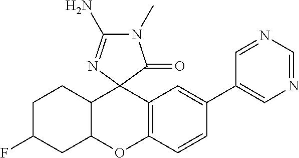

### 2-amino-3′-fluoro-1-methyl-7′-(pyrimidin-5-yl)-1′,2′,3′,4′,4a′,9a′-hexahydro spiro[imidazole-4,9′-xanthen]-5(1H)-one

2-Amino-3′-fluoro-1-methyl-7′-(pyrimidin-5-yl)-1′,2′,3′,4′,4a′,9a′-hexahydrospiro[imidazole-4,9′-xanthen]-5(1H)-one was prepared according to Example 44, substituting pyrimidin-5-ylboronic acid for 5-chloropyridin-3-ylboronic acid. ¹H NMR (CD₃OD) δ 9.10 (s, 1H), 9.03 (s, 2H), 7.70 (m, 1H), 7.59 (s, 1H), 7.12 (d, J=8.6 Hz, 1H), 4.92 (m, 1H), 3.27 (s, 3H), 2.64 (m, 1H), 2.21 (m, 1H), 2.11 (m, 1H), 1.75 (m, 3H), 1.61 (m, 1H), 1.39 (m, 1H); m/z (APCI-pos) M+1=382.1.

### Example 47

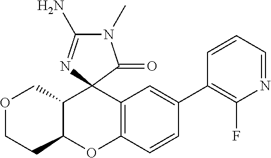

### (4R,4a′S,10a′S)-2-amino-8′-(2-fluoropyridin-3-yl)-1-methyl-3′,4′,4a′,10a′-tetrahydro-1′H-spiro[imidazole-4,10′-pyrano[4,3-b]chromen]-5(1H)-one

Step A: A mixture of dihydro-2H-pyran-4(3H)-one (100 g, 999 mmol) and morpholine (131 mL, 1498 mmol) in toluene (333 mL) was refluxed under Dean-Stark trap overnight. More than 1 equivalent of water was collected. This reaction mixture was then concentrated down to give 4-(3,6-dihydro-2H-pyran-4-yl)morpholine (169 g, 100% yield) as an oil.

Step B: A mixture of 4-(3,6-dihydro-2H-pyran-4-yl)morpholine (178.1 g, 1052 mmol) and 5-bromo-2-hydroxybenzaldehyde (211.6 g, 1052 mmol) in toluene (351 mL) was stirred overnight at room temperature. A solid crashed out and was filtered off. This was washed with toluene (50 mL) The solid product was collected and dried to give 8-bromo-4-a-morpholino-1,3,4,4a,10,10a-hexahydropyrano[4,3-b]chromen-10-ol (306.8 g, 79% yield).

Step C: DMSO (204 mL, 2878 mmol) was added dropwise to oxalyl chloride (470 mL, 939 mmol) in DCM (8 L) at −78° C. This was added such that the temperature did not rise above −65° C. This was then stirred for 40 minutes at −78° C. 8-Bromo-4-a-morpholino-1,3,4,4a,10,10a-hexahydropyrano[4,3-b]chromen-10-ol (533 g, 1439 mmol) was added as a solid (temperature did not rise) and this was stirred for 2 hours at −78° C. The solid did not fully go into solution. Triethylamine (602 mL, 4317 mmol) was added dropwise (some exotherm was seen, however the reaction temperature did not get above −65° C.). This was stirred for 30 minutes at −78° C. During the entire course of the reaction, the mixture was continually purged with N₂, which exited the flask via a line fed into a bleach trap. The mixture was then concentrated down. Glacial acetic acid (1000 mL) was added to the mixture. The material went into solution initially however after 5 minutes of stirring, product began to crash out. The material was stirred overnight at room temperature. A solid had crashed out and this was filtered. The solid was washed with glacial acetic acid (200 mL). This gave 8-bromo-3,4-dihydropyrano[4,3-b]chromen-10(1H)-one (340.8 g, 84% yield) as a solid.

Step D: 1-Selectride (587 mL, 587 mmol, 1M in THF) was added to a mixture of 8-bromo-3,4-dihydropyrano[4,3-b]chromen-10(1H)-one (150 g, 534 mmol) in DCM (2809 mL) at −78° C. This was stirred for 45 minutes. TLC showed that the reaction was complete. The mixture was placed in an ice bath. Aqueous Rochelle's salt (0.5M) was added to the mixture as it was warming to 0° C. This was then worked up with EtOAc/water. The organics were extracted twice, washed with brine, dried (Na₂SO₄), and concentrated. The crude was then triturated with hexanes to give (4aS*,10aS*)-8-bromo-1,4,4a,10a-tetrahydropyrano[4,3-b]chromen-10(3H)-one (100 g, 66% yield).

Step E: A mixture of (4aS*,10aS*)-8-bromo-1,4,4a,10a-tetrahydropyrano[4,3-b]chromen-10(3H)-one (75 g, 265 mmol), KCN (34.5 g, 530 mmol), ammonium carbonate (204 g, 2119 mmol) and NaHSO₃ (11.0 g, 106 mmol) in EtOH (265 mL) was heated to 130° C. overnight in a steel bomb with stirring. The mixture was poured into an Erlenmeyer flask with side arm in an ice bath. The flask was purged with N₂ and the outlet line was fed into a 2N NaOH solution to quench HCN. Concentrated HCl was carefully added to the flask until the pH was about 1. This was then stirred in an ice bath for 1 hour while purging with N₂. The resulting solid was filtered off and collected. This solid was dried and then taken up in IPA (500 mL) and heated at reflux for 30 minutes. This was then cooled to room temperature and then to 5° C. in an ice bath. The solid was filtered and washed with IPA (50 mL) to give (4S*,4a′S*,10a′S*)-8′-bromo-3′,4′,4a′,10a′-tetrahydro-1′H-spiro[imidazolidine-4,10′-pyrano[4,3-b]chromene]-2,5-dione (44.8 g, 43% yield).

Step F: A round bottomed flask plus stir bar was charged with DMF (100 mL) and (4R*,4a′S*,10a′S*)-8′-bromo-3′,4′,4a′,10a′-tetrahydro-1′H-spiro[imidazolidine-4,10′-pyrano[4,3-b]chromene]-2,5-dione (16.3 g, 46.2 mmol). The reaction mixture was cooled in an ice bath under N₂, and added K₂CO₃ (9.6 g, 69 mmol), followed by iodomethane (2.9 mL, 46 mmol). The reaction mixture was stirred in the ice bath for 10 minutes, the bath was removed, and the mixture was allowed to stir at room temperature for 2 hours. The reaction mixture was diluted with EtOAc (300 mL) and water (200 mL) The phases were separated, and re-extracted aqueous with EtOAc (150 mL). The organic phases were combined, washed with water (200 mL), brine (200 mL), dried (MgSO₄), filtered and concentrated to yield (4R*,4a′S*,10a′S*)-8′-bromo-1-methyl-3′,4′,4a′,10a′-tetrahydro-1′H-spiro[imidazolidine-4,10′-pyrano[4,3-b]chromene]-2,5-dione (15.9 g, 94% yield; the product was approximately 85% pure based on HPLC). The product was carried forward to the next step without purification.

Step G: A thick walled, glass pressure vessel was charged with (4R*,4a′S*,10a′S*)-8′-bromo-1-methyl-3′,4′,4a′,10a′-tetrahydro-1′H-spiro[imidazolidine-4,10′-pyrano[4,3-b]chromene]-2,5-dione (15.9 g, 43.3 mmol), Lawesson's reagent (10.5 g, 26.0 mmol), and toluene (150 mL). The reaction mixture was degassed with N₂ for several minutes and heated to 90° C. for 15 hours with stirring. The reaction had gone to approximately 50% conversion by HPLC and ¹H NMR. More Lawesson's Reagent (3.5 g, 0.2 equivalents) was added, and the reaction was heated to 100° C. for an additional 22 hours. HPLC indicated greater than 95% consumption of starting material. After cooling to room temperature, a solid had formed. The suspension was cooled in a freezer at 5° C. for 2 hours, and then the solids (14.9 g) were filtered, washing with toluene. The mother liquor was saved. This solid (14.9 g) was mostly desired product by ¹H NMR, and it was partially purified by silica gel plug, eluting with 10% Et₂O in DCM. However, after silica gel plug, the product (13 g) took on a deep green color, and was only slightly improved in purity by ¹H NMR analysis. The mother liquor that had been saved was partioned between EtOAc (200 mL) and saturated aqueous NaHCO₃ (200 mL). The phases were separated, and the aqueous phase was re-extracted with EtOAc (150 mL). Combined organic phases were washed with brine (200 mL), dried (MgSO₄), filtered, and concentrated to obtain crude material (15.3 g). By ¹H NMR, this crude contained approximately 20% desired product. To improve upon the product yield, it was chromatographed on a Biotage Flash 75 L system, eluting with 5% Et₂O/DCM isocratic (2 L to wet column, followed by 8 L to fully elute product). From this column, an additional product (2.1 g) was recovered, which was combined with the product (13 g) purified by silica gel plug, to obtain (4R*,4a′S*,10a′S*)-8′-bromo-1-methyl-2-thioxo-3′,4′,4a′,10a′-tetrahydro-1′H-spiro[imidazolidine-4,10′-pyrano[4,3-b]chromen]-5-one (15.1 g, 59% yield). The product purity was only 60-65% based on ¹H NMR and HPLC, but was carried forward to the next step without further purification.

Step H: A round bottomed flask plus stir bar was charged with (4R*,4a′S*,10a′S*)-8′-bromo-1-methyl-2-thioxo-3′,4′,4a′,10a′-tetrahydro-1′H-spiro[imidazolidine-4,10′-pyrano[4,3-b]chromen]-5-one (15.1 g, 39.4 mmol; 60-65% purity), MeOH (200 mL), 70% aqueous t-butyl hydroperoxide (38 mL, 276 mmol), and 30% aqueous NH₄OH (77 mL, 591 mmol). The mixture was heated to 50° C. for 2 hours with stirring. After cooling to room temperature, the mixture was diluted with water (20 mL) and concentrated (but not to dryness) in vacuo. The redisue was diluted with EtOAc (150 mL), and the phases were separated. The aqueous phase was re-extracted with EtOAc (2×75 mL) Combined organic phases were washed with brine (150 mL), dried (MgSO₄), filtered, and concentrated. Purified crude (15.6 g) by silica gel chromatography on a Biotage Flash 75 L system, eluting with 7% MeOH/DCM (4 L to wet column, followed by elution with 4 L) then with 10% MeOH/DCM (4 L) to fully elute desired product. (4R*,4a′S*,10a′S*)-2-Amino-8′-bromo-1-methyl-3′,4′,4a′,10a′-tetrahydro-1′H-spiro[imidazole-4,10′-pyrano[4,3-b]chromen]-5(1H)-one (7.4 g, 47%) was obtained as a powder. The product was greater than 95% pure trans diastereomer by ¹H NMR.

Step I: A thick walled, glass pressure vessel plus stir bar was charged with (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (2.2 g, 6.0 mmol), dioxane (30 mL), 2-fluoropyridin-3-ylboronic acid (1.3 g, 9.0 mmol), Pd(PPh₃)₄ (0.17 g, 0.15 mmol), and 2N aqueous Na₂CO₃ (9.0 mL, 18 mmol). The mixture was sparged with N₂ for 15 minutes and then heated to 90° C. for 1 hour with stirring. The starting material had been consumed by TLC analysis (elution with 10% MeOH/DCM allows for separation of starting material and product). The reaction mixture was partitioned between EtOAc (50 mL) and water (50 mL). The phases were separated, and the aqueous phase was re-extracted with EtOAc (2×30 mL) The combined organic phases were washed with brine (50 mL), dried (MgSO₄), filtered and concentrated. Combined the crude from this reaction with crude product from previous smaller scale reactions that totaled 1.5 g. The combined crude products were concentrated on the rotovap with DCM (2×30 mL) to remove residual solvents from the workup. Then the crude solid was triturated in DCM (10 mL) at room temperature. The solids were filtered, rinsing with DCM (3×5 mL). The resulting solid (2.8 g, 74% overall yield for the 1.5 g and 2.2 g scale reactions) was greater than 95% pure (4R*,4a′S*,10a′S*)-2-amino-8′-(2-fluoropyridin-3-yl)-1-methyl-3′,4′,4a′,10a′-tetrahydro-1′H-spiro[imidazole-4,10′-pyrano[4,3-b]chromen]-5(1H)-one. This racemic material was purified by chiral SFC chromatography to obtain enantiomerically pure (4R,4a′S,10a′S)-2-amino-8′-(2-fluoropyridin-3-yl)-1-methyl-3′,4′,4a′,10a′-tetrahydro-1′H-spiro[imidazole-4,10′-pyrano[4,3-b]chromen]-5(1H)-one. ¹H NMR (400 MHz, CDCl₃) δ 8.05 (m, 1H), 7.75 (m, 1H), 7.36 (m, 1H), 7.18 (m, 1H), 7.14 (m, 1H), 6.98 (d, J=9 Hz, 1H), 5.73 (br s, 2H), 4.95 (td, J=5, 11 Hz, 1H), 4.05 (dd, J=5, 12 Hz, 1H), 3.98 (dd, J=4, 11 Hz, 1H), 3.47 (m, 1H), 3.04 (s, 3H), 3.03 (m, 1H), 2.18 (m, 2H), 1.83 (m, 1H); m/z (APCI-pos) M+1=383.

### Example 48

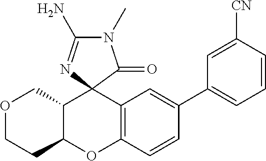

### 3-((4R,4a′S,10a′S)-2-amino-1-methyl-5-oxo-1,3′,4′,4a′,5,10a′-hexahydro-1′H-spiro[imidazole-4,10′-pyrano[4,3-b]chromen]-8′-yl)benzonitrile

The title compound (474 mg, 63%) was prepared according to Example 47, Step I, replacing racemic (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one with enantiopure (4R,4a′S,10a′S)-2-amino-8′-bromo-1-methyl-3′,4′,4a′,10a′-tetrahydro-1′H-spiro[imidazole-4,10′-pyrano[4,3-b]chromen]-5(1H)-one (700 mg, 1.91 mmol), that had been separated from its enantiomer by chiral SFC chromatography, and replacing 2-fluoropyridin-3-ylboronic acid with 3-cyanophenylboronic acid (421 mg, 2.87 mmol). NMR (400 MHz, CDCl₃+MeOD) δ 7.74 (s, 1H), 7.70 (m, 1H), 7.57 (m, 1H), 7.51 (m, 1H), 7.41 (m, 1H), 7.06 (m, 1H), 7.00 (d, J=9 Hz, 1H), 4.95 (td, J=5, 11 Hz, 1H), 4.07 (m, 1H), 3.96 (m, 1H), 3.51 (m, 1H), 3.10 (s, 3H), 3.05 (t, J=11 Hz, 1H), 2.24 (m, 2H), 1.91 (m, 1H); m/z (APCI-pos) M+1=389.

### Example 49

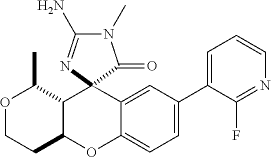

### (1′S*,4R*,4a′S*,10a′S*)-2-amino-8′-(2-fluoropyridin-3-yl)-1,1′-dimethyl-3′,4′,4a′,10a'-tetrahydro-1′H-spiro[imidazole-4,10′-pyrano[4,3-b]chromen]-5(1H)-one

Step A: Similar to a procedure described in Badawy, Doris S., et al. “Synthesis of Some New Naphthopyran, Pyrazole, Pyridine, and Thienobenzochromene Derivatives Using 1-(1-Hydroxy-2-naphthyl) Ethanone as a Versatile Starting Material.” Phosphorus, Sulfur, and Silicon. Vol. 184 (2009): pp. 179-196, a mixture of 1-(5-bromo-2-hydroxyphenyl)ethanone (50 g, 233 mmol) and DMF-dimethylacetal (42 g, 349 mmol) in dry toluene (250 mL) was refluxed for 3 hours. After cooling to room temperature, the mixture was concentrated to half volume, and the resulting suspension was cooled in an ice bath. Then the solids were filtered, washing with minimum amounts of toluene to yield (E)-1-(5-bromo-2-hydroxyphenyl)-3-(dimethylamino)prop-2-en-1-one (56 g, 87%).

Step B: Similar to a procedure described in Badawy, et al. (see above), acetic anhydride (196 mL) was added to a solution of (E)-1-(5-bromo-2-hydroxyphenyl)-3-(dimethylamino)prop-2-en-1-one (56 g, 207 mmol) in dry pyridine (84 mL), and the mixture was stirred at room temperature for 18 hours. The mixture was concentrated on the rotovap to one half volume at 80° C. The resulting suspension was cooled to room temperature, and then the solids were filtered. The solids were washed with hexanes and dried under high vacuum to yield 3-acetyl-6-bromo-4H-chromen-4-one (48 g, 85%).

Step C: A stainless steel bomb plus stir bar was charged with ethyl vinyl ether (169 mL, 1760 mmol) and 3-acetyl-6-bromo-4H-chromen-4-one (47 g, 176 mmol). The mixture was heated to 100° C. for 15 hours. After cooling to room temperature, the reaction mixture was filtered, washing the solids with a minimum amount of EtOAc to yield (3R*,4aR*)-8-bromo-3-ethoxy-1-methyl-4,4-a-dihydropyrano[4,3-b]chromen-10(3H)-one (44 g, 72%).

Step D: A round bottomed flask plus stir bar was charged with (3R*,4aR*)-8-bromo-3-ethoxy-1-methyl-4,4-a-dihydropyrano[4,3-b]chromen-10(3H)-one (43 g, 127 mmol), THF (500 mL), and cooled to −78° C. in a dry ice/acetone bath. DIBAL (1.5M in toluene, 101 mL, 152 mmol) was added dropwise and stirred at −78° C. for 1 hour. The reaction remained a suspension the entire time. The reaction mixture was quenched by inverse addition (via canula) to Rochelle's salt (500 mL) that was stirred at room temperature. The mixture was worked up by extraction with EtOAc (2×500 mL). The combined organics were washed with brine (500 mL), dried (MgSO₄), filtered, and concentrated. The crude was purified by Biotage Flash 75 silica gel chromatography, eluting with 5%-10% EtOAc/hexanes to yield (1R*,4aR*,10aR*)-8-bromo-3-ethoxy-1-methyl-1,4,4a,10a-tetrahydropyrano[4,3-b]chromen-10(3H)-one (22.4 g, 36%).

Step E: A round bottomed flask plus stir bar was charged with (1R*,4aR*,10aR*)-8-bromo-3-ethoxy-1-methyl-1,4,4a,10a-tetrahydropyrano[4,3-b]chromen-10(3H)-one (22.2 g, 65.1 mmol), DCM (200 mL), and triethylsilane (51.8 mL, 325 mmol). The mixture was cooled in an ice bath under N₂. Then BF₃-etherate (24.7 ml, 195 mmol) was added dropwise. The reaction mixture was stirred overnight at room temperature. The mixture was carefully quenched with saturated aqueous NaHCO₃ (200 mL) and stirred for 1 hour. The phases were separated, and the aqueous phase was re-extracted with DCM (2×75 mL). The combined organic phases were washed with brine (200 mL), dried (MgSO₄), filtered, and concentrated. The crude was purified by Biotage Flash 65 silica gel chromatography, eluting with 10%-20% EtOAc/hexanes to yield (1R*,4aR*,10aR*)-8-bromo-1-methyl-1,4,4a,10a-tetrahydropyrano[4,3-b]chromen-10(3H)-one (13.6 g, 60%).

Step F: A stainless steel bomb plust stir bar was charged with EtOH (10 mL) and (1R*,4aR*,10aR*)-8-bromo-1-methyl-1,4,4a,10a-tetrahydropyrano[4,3-b]chromen-10(3H)-one (3 g, 10 mmol). Next, ammonium carbonate (4.9 g, 50 mmol), KCN (1.3 g, 20 mmol) and sodium hydrogensulfite (0.26 g, 2.5 mmol) were added. The reaction was heated to 130° C. for 16 hours with stirring in an oil bath. After cooling to room temperature, the reaction contents were transferred to an Erlenmeyer flask using EtOAc (20 mL) and water (10 mL) to aid in transfer. The contents were chilled in an ice bath, carefully acidified with concentrated HCl, and then N₂ was bubbled through the mixture for 30 minutes to sparge HCN (near back of hood). The phases were separated, and the aqueous phase was re-extracted with EtOAc (2×10 mL). The combined organic phases were washed with brine (50 mL), dried (MgSO₄), filtered, and concentrated. The crude was purified by Biotage Flash silica gel chromatography, eluting with 5%-10% MeOH/DCM to yield (1′S*,4R*,4a′S*,10a′S*)-8′-bromo-1′-methyl-3′,4′,4a′,10a′-tetrahydro-1′H-spiro[imidazolidine-4,10′-pyrano[4,3-b]chromene]-2,5-dione and its diastereomer (1′R*,4R*,4a′R*,10a′R*)-8′-bromo-1′-methyl-3′,4′,4a′,10a′-tetrahydro-1′H-spiro[imidazolidine-4,10′-pyrano[4,3-b]chromene]-2,5-dione obtained in a 60:40 ratio (1.6 g, 35%).

Step G: A round bottomed flask plus stir bar was charged with DMF (10 mL) and the two diastereomers (1′S*,4R*,4a′S*,10a′S*)-8′-bromo-1′-methyl-3′,4′,4a′,10a′-tetrahydro-1′H-spiro[imidazolidine-4,10′-pyrano[4,3-b]chromene]-2,5-dione and (1′R*,4R*,4a′R*,10a′R*)-8′-bromo-1′-methyl-3′,4′,4a′,10a′-tetrahydro-1′H-spiro[imidazolidine-4,10′-pyrano[4,3-b]chromene]-2,5-dione (1.6 g, 4.36 mmol). The reaction mixture was cooled in an ice bath under N₂, and K₂CO₃ (0.903 g, 6.54 mmol) was added, followed by iodomethane (0.217 mL, 3.49 mmol). The mixture was stirred at room temperature overnight. The mixture was diluted with EtOAc (20 mL) and water (20 mL). The phases were separated, and the aqueous phase was re-extracted with EtOAc (20 mL) The combined organic phases were washed with water (20 mL), brine (20 mL), dried (MgSO₄), filtered, and concentrated. The diastereomers were separated by Biotage Flash 40 L silica gel chromatography, eluting with 20% EtOAc/hexanes-1:1 EtOAc/hexanes. (1′R*,4R*,4a′R*,10a′R*)-8′-Bromo-1,1′-dimethyl-3′,4′,4a′,10a′-tetrahydro-1′H-spiro[imidazolidine-4,10′-pyrano[4,3-b]chromene]-2,5-dione was pure enough to carry forward. The other diastereomer, (1′S*,4R*,4a′S*,10a′S*)-8′-bromo-1,1′-dimethyl-3′,4′,4a′,10a′-tetrahydro-1′H-spiro[imidazolidine-4,10′-pyrano[4,3-b]chromene]-2,5-dione, required a second purification by Biotage Flash 40 L silica gel chromatography, eluting with 1% MeOH/DCM. This yielded (1′S*,4R*,4a′S*,10a′S*)-8′-bromo-1,1′-dimethyl-3′,4′,4a′,10a′-tetrahydro-1′H-spiro[imidazolidine-4,10′-pyrano[4,3-b]chromene]-2,5-dione (133 mg, 6%) and (1′R*,4R*,4a′R*,10a′R*)-8′-bromo-1,1′-dimethyl-3′,4′,4a′,10a′-tetrahydro-1′H-spiro[imidazolidine-4,10′-pyrano[4,3-b]chromene]-2,5-dione (554 mg, 23%).

Step H: (1′S*,4R*,4a′S*,10a′S*)-2-Amino-8′-(2-fluoropyridin-3-yl)-1,1′-dimethyl-3′,4′,4a′,10a′-tetrahydro-1′H-spiro[imidazole-4,10′-pyrano[4,3-b]chromen]-5(1H)-one was prepared from (1′S*,4R*,4a′S*,10a′S*)-8′-bromo-1,1′-dimethyl-3′,4′,4a′,10a'-tetrahydro-1′H-spiro[imidazolidine-4,10′-pyrano[4,3-b]chromene]-2,5-dione according to the procedures described for Example 47, Steps G-I. The racemic product was purified by preparative TLC (0.5 mm thickness, Rf=0.43) eluting with 10% MeOH (containing 7N NH₃)/DCM. ¹H NMR (400 MHz, CDCl₃+MeOD) δ 8.11 (m, 1H), 7.81 (m, 1H), 7.39 (m, 1H), 7.27 (m, 1H), 7.07 (m, 1H), 6.97 (d, J=9 Hz, 1H), 4.97 (m, 1H), 4.04 (m, 1H), 3.59 (m, 1H), 3.34 (m, 1H), 3.12 (s, 3H), 2.19 (m, 1H), 1.98 (m, 2H), 1.19 (d, J=6 Hz, 3H); m/z (APCI-pos) M+1=397.

### Example 50

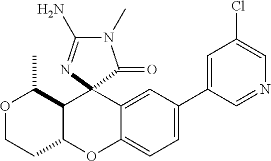

### (1′R*,4R*,4a′R*,10a′R*)-2-amino-8′45-chloropyridin-3-yl)-1,1′-dimethyl-3′,4′,4a′,10a'-tetrahydro-1′H-spiro[imidazole-4,10′-pyrano[4,3-b]chromen]-5(1H)-one

The title compound (15 mg, 50%) was prepared from (1′R*,4R*,4a′R*,10a′R*)-8′-bromo-1,1′-dimethyl-3′,4′,4a′,10a′-tetrahydro-1′H-spiro[imidazolidine-4,10′-pyrano[4,3-b]chromene]-2,5-dione (synthesized as described in Example 49, Step G), according to the procedures described for Example 47, Steps G-I, replacing 2-fluoropyridin-3-ylboronic acid with 5-chloropyridin-3-ylboronic acid in Step I. The racemic product was purified by preparative TLC (1 mm thickness, Rf=0.50) eluting with 10% MeOH (containing 7N NH₃)/DCM. ¹H NMR (400 MHz, CDCl₃+MeOD) δ 8.50 (m, 1H), 8.46 (m, 1H), 7.75 (m, 1H), 7.38 (m, 1H), 6.98 (m, 1H), 6.85 (m, Hi), 4.19 (m, 1H), 4.04 (m, 1H), 3.56 (m, Hi), 3.46 (m, 1H), 3.20 (s, 3H), 2.26 (m, 2H), 2.00 (m, 1H), 1.03 (d, J=6 Hz, 3H); m/z (APCI-pos) M+1=413.

### Example 51

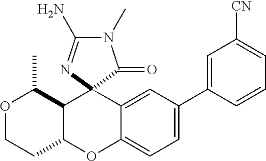

### 3-((1′R*,4R*,4a′R*,10a′R*)-2-amino-1,1′-dimethyl-5-oxo-1,3′,4′,4a′,5,10a′-hexahydro-1′H -spiro[imidazole-4,10′-pyrano[4,3-b]chromen]-8′-yl)benzonitrile

The title compound (12 mg, 41%) was prepared from (1′R*,4R*,4a′R*,10a′R*)-8′-bromo-1,1′-dimethyl-3′,4′,4a′,10a′-tetrahydro-1′H-spiro[imidazolidine-4,10′-pyrano[4,3-b]chromene]-2,5-dione (synthesized as described in Example 49, Step G), according to the procedures described for Example 47, Steps G-I, replacing 2-fluoropyridin-3-ylboronic acid with 3-cyanophenylboronic acid in Step I. Racemic product was purified by preparative TLC (1 mm thickness, Rf=0.50) eluting with 10% MeOH (containing 7N NH₃)/DCM. ¹H NMR (400 MHz, CDCl₃+MeOD) δ 7.68 (m, 1H), 7.66 (d, J=8 Hz, 1H), 7.59 (d, J=8 Hz, 1H), 7.51 (t, J=8 Hz, 1H), 7.38 (m, 1H), 6.97 (d, J=9 Hz, 1H), 6.84 (m, 1H), 4.19 (m, 1H), 4.03 (m, 1H), 3.71 (m, 1H), 3.56 (m, 1H), 3.21 (s, 3H), 2.25 (m, 2H), 2.01 (m, 1H), 1.03 (d, J=6 Hz, 3H); m/z (APCI-pos) M+1=403.

### Example 52

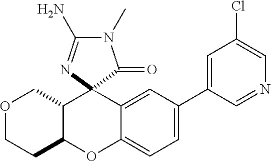

### (4R*,4a′S*,10a′S*)-2-amino-8′-(5-chloropyridin-3-yl)-1-methyl-3′,4′,4a′,10a′-tetrahydro-1′H-spiro[imidazole-4,10′-pyrano[4,3-b]chromen]-5(1H)-one

The title compound (42 mg, 38%) was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (synthesized as described in Example 47, Step H) according to the procedure described in Example 47, Step I, replacing 2-fluoropyridin-3-ylboronic acid with 5-chloropyridin-3-ylboronic acid. The racemic product was purified by preparative TLC (0.5 mm thickness, Rf=0.50) eluting with 10% MeOH (containing 7N NH₃)/DCM. ¹H NMR (400 MHz, CDCl₃) δ 8.56 (d, J=2 Hz, 1H), 8.45 (d, J=2 Hz, 1H), 7.70 (t, J=2 Hz, 1H), 7.37 (dd, J=2, 9H, 1H), 7.06 (d, J=2 Hz, 1H), 6.99 (d, J=9 Hz, 1H), 4.91 (td, J=5, 11 Hz, 1H), 4.77 (br s, 2H), 4.03 (dd, J=5, 11 Hz, 1H), 3.94 (dd, J=4, 11 Hz, 1H), 3.46 (m, 1H), 3.08 (s, 3H), 3.02 (m, 1H), 2.13 (m, 2H), 1.82 (m, 1H); m/z (APCI-pos) M+1=399.

### Example 53

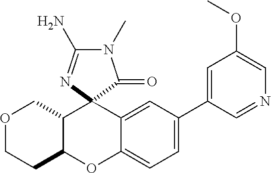

### (4R*,4a′S*,10a′S*)-2-amino-8′-(5-methoxypyridin-3-yl)-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (synthesized as described in Example 47, Step H) according to the procedure described in Example 47, Step I, replacing 2-fluoropyridin-3-ylboronic acid with 5-methoxypyridin-3-ylboronic acid. The racemic product was purified by preparative TLC (0.5 mm thickness, Rf=0.24) eluting with 10% MeOH (containing 7N NH₃)/DCM. ¹H NMR (400 MHz, CDCl₃+MeOD) δ 8.25 (d, J=2 Hz, 1H), 8.16 (d, J=3 Hz, 1H), 7.41 (m, 1H), 7.33 (m, 1H), 7.08 (d, J=2 Hz, 1H), 7.01 (d, J=9 Hz, 1H), 4.96 (td, J=5, 11 Hz, 1H), 4.09 (m, 1H), 3.94 (m, 1H), 3.92 (s, 3H), 3.51 (m, 1H), 3.10 (s, 3H), 3.05 (t, J=11 Hz, 1H), 2.27 (m, 2H), 1.88 (m, 1H); m/z (APCI-pos) M+1=395.

### Example 54

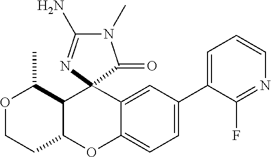

### (1′R*,4R*,4a′R*,10a′R*)-2-amino-8′-(2-fluoropyridin-3-yl)-1,1′-dimethyl-3′,4′,4a′,10a′-tetrahydro-1′H-spiro[imidazole-4,10′-pyrano[4,3-b)]chromen]-5(1H)-one

The title compound was prepared from (1′R*,4R*,4a′R*,10a′R*)-8′-bromo-1,1′-dimethyl-3′,4′,4a′,10a′-tetrahydro-1′H-spiro[imidazolidine-4,10′-pyrano[4,3-b]chromene]-2,5-dione (synthesized as described in Example 49, Step G), according to the procedures described for Example 47, Steps G-I. The racemic product was purified by preparative TLC (2 mm thickness, Rf=0.44) eluting with 10% MeOH (containing 7N NH₃)/DCM. Then the resulting product was triturated with a minimum amount of DCM and filtered. ¹H NMR (400 MHz, CDCl₃+MeOD) δ 8.11 (m, 1H), 7.78 (m, 1H), 7.37 (m, 1H), 7.25 (m, 1H), 6.97 (d, J=9 Hz, 1H), 6.93 (m, 1H), 4.18 (m, 1H), 4.02 (m, 1H), 3.55 (m, 1H), 3.46 (m, 1H), 3.18 (s, 3H), 2.25 (m, 2H), 2.00 (m, 1H), 1.03 (d, J=6 Hz, 3H); m/z (APCI-pos) M+1=397.

### Example 55

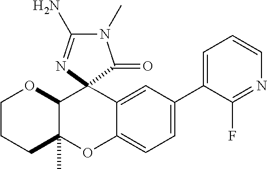

### (4S*,4a′S*,10a′S*)-2-amino-8′-(2-fluoropyridin-3-yl)-1,4a′-dimethyl-3′,4′,4a′,10a′-tetrahydro-2′H-spiro[imidazole-4,10′-pyrano[3,2-b]chromen]-5 (1H)-one

Step A: A thick walled glass pressure tube plus stir bar was charged with 5-hydroxypentan-2-one (15.3 g, 150 mmol), 1-(5-bromo-2-hydroxyphenyl)ethanone (21.5 g, 100 mmol), and toluene (100 mL). Then, pyrrolidine (8.21 mL, 100 mmol) was added, followed by acetic acid (5.72 mL, 100 mmol). The mixture was heated to 80° C. for 18 hours with stirring. After cooling to room temperature, the mixture was partitioned between EtOAc (100 mL) and aqueous 1N HCl (100 mL). The phases were separated. The aqueous phase was re-extracted with EtOAc (50 mL), and then carefully shook organic phases with aqueous saturated NaHCO₃ (100 mL; gas evolution, vent separatory funnel cautiously). The organic phases were washed with brine (100 mL), dried (MgSO₄), filtered, and concentrated. The crude was purified by Biotage Flash 65 silica gel chromatography, eluting with 25%-2:1 EtOAc/hexanes to yield 6-bromo-2-(3-hydroxypropyl)-2-methylchroman-4-one (15.8 g, 50%).

Step B: 6-Bromo-2-(3-hydroxypropyl)-2-methylchroman-4-one (9.8 g, 32.8 mmol) with TBDMS-Cl (5.43 g, 36.0 mmol) in DCM (50 mL) was stirred. The mixture was cooled in an ice bath, and imidazole (2.90 g, 42.6 mmol) was added. The mixture was stirred for 30 minutes in the ice bath and then stirred for 30 minutes more at room temperature after removal of the ice bath. The reaction mixture was worked up by washing with 1N HCl (30 mL), saturated aqueous NaHCO₃ (30 mL), then drying (MgSO₄), filtration, and concentration to yield 6-bromo-2-(3-((tert-butyldimethylsilyl)oxy)propyl)-2-methylchroman-4-one (13.1 g, 92%).

Step C: Ethyl formate (14.0 mL, 174 mmol) was added to a stirred slurry of sodium methoxide powder (7.53 g, 139 mmol) in toluene (150 mL) under nitrogen. The mixture was stirred for 10 minutes at room temperature and then cooled in an ice bath under N₂. Next, 6-bromo-2-(3-((tert-butyldimethylsilyl)oxy)propyl)-2-methylchroman-4-one (14.4 g, 34.8 mmol, material from Step B combined with a second batch of 6-bromo-2-(3-((tert-butyldimethylsilyl)oxy)propyl)-2-methylchroman-4-one) in toluene (50 mL) was added dropwise, and the mixture stirred in the ice bath as ice melted for 2 hours. The reaction mixture was quenched with saturated NH₄Cl (200 mL) and diluted with EtOAc (100 mL) The phases were separated, and the aqueous phase was re-extracted with EtOAc (100 mL) The combined organic phases were washed with brine (100 mL), dried (MgSO₄), filtered, and concentrated. The crude was purified by Biotage Flash 65 silica gel chromatography, eluting with 5%-20% EtOAc/hexanes to yield of 6-bromo-2-(3-((tert-butyldimethylsilyl)oxy)propyl)-2-methyl-4-oxochroman-3-carbaldehyde (4.5 g, 21%).

Step D: Diethylamine (1.5 g, 20 mmol) was added to a solution of 6-bromo-2-(34(tert-butyldimethylsilyl)oxy)propyl)-2-methyl-4-oxochroman-3-carbaldehyde (4.5 g, 10 mmol) and 4-methylbenzenesulfonyl azide (2.4 g, 12 mmol; prepared as described in WO 2010/011147, but replacing DCM with EtOAc during the workup) in Et₂O (20 mL) in an ice bath. The reaction mixture was stirred at room temperature for 18 hours. The reaction mixture was concentrated, and then the crude was purified by Biotage Flash 65 silica gel chromatography eluting with 5%-10% EtOAc/hexanes to yield 6-bromo-2-(3-((tert-butyldimethylsilyl)oxy)propyl)-3-diazo-2-methylchroman-4-one (3.4 g, 38%).

Step E: A round bottomed flask plus stir bar was charged with 6-bromo-2-(3-((tert-butyldimethylsilyl)oxy)propyl)-3-diazo-2-methylchroman-4-one (3.4 g, 7.8 mmol), THF (20 mL), acetic acid (20 mL), and water (10 mL) The reaction mixture was stirred at room temperature for 18 hours. The mixture was concentrated to approximately half volume in vacuo. The crude product was pardoned between EtOAC (30 mL) and water (30 mL). The phases were separated, and the aqueous phase was re-extracted with EtOAc (30 mL) The combined organic phases were washed with brine (50 mL), dried (MgSO₄), filtered, and concentrated. The product was purified by Biotage Flash 40 silica gel chromatography, eluting with 25′,1:1 EtOAc/hexanes to yield 6-bromo-3-diazo-2-(3-hydroxypropyl)-2-methylchroman-4-one (2.1 g, 52%).

Step F: A round bottomed flask plus stir bar containing 6-bromo-3-diazo-2-(3-hydroxypropyl)-2-methylchroman-4-one (2 g, 6.15 mmol) and toluene (20 mL) was charged with Rh₂(OAc)₄ (0.136 g, 0.308 mmol). The reaction mixture was heated to 70° C. There was gas evolution noted as temperature rose above 55° C., and the mixture was vented adequately. The mixture was stirred for 20 minutes at 70° C. The mixture was then concentrated to half volume in vacuo. The crude was purified by Biotage Flash 40 silica gel chromatography, eluting with 10%-1:1 EtOAc/hexanes to yield (4aR*,10aR*)-8-bromo-4-a-methyl-2,3,4,4a-tetrahydropyrano[3,2-b]chromen-10(10aH)-one (450 mg, 23%).

Step G: A stainless steel bomb plus stir bar was charged with EtOH (1 mL) and (4aR*,10aR*)-8-bromo-4-a-methyl-2,3,4,4a-tetrahydropyrano[3,2-b]chromen-10(10aH)-one (200 mg, 0.673 mmol). Next, ammonium carbonate (323 mg, 3.37 mmol), KCN (87.7 mg, 1.35 mmol) and sodium hydrogensulfite (17.5 mg, 0.168 mmol) were added. The reaction mixture was heated to 130° C. for 16 hours with stirring in an oil bath. The mixture was transferred to an Erlenmeyer flask with EtOAc (10 mL) and water (10 mL). The mixture was acidified with concentrated HCl, and sparged with N₂ (in back of hood, sashes closed) for 15 minutes to purge excess HCN. The phases were separated, and the aqueous phase was re-extracted with EtOAc (10 mL) The combined organic phases were washed with brine (20 mL), dried (MgSO₄), filtered, and concentrated. The diastereomers were separated by preparative TLC (2 mm thickness) eluting with 5% MeOH/DCM. Diastereomer A (Rf=0.43, 63 mg, 14% yield) was (4S*,4a′S*,10a′S*)-8′-bromo-4a′-methyl-3′,4′,4a′,10a′-tetrahydro-2′H-spiro[imidazolidine-4,10′-pyrano[3,2-b]chromene]-2,5-dione. Diastereomer B (Rf=0.34, 79 mg, 19% yield) was (4S*,4a′R*,10a′S*)-8′-bromo-4a′-methyl-3′,4′,4a′,10a′-tetrahydro-2′H-spiro[imidazolidine-4,10′-pyrano[3,2-b]chromene]-2,5-dione.

Step H: (4S*,4a′S*,10a′S*)-2-Amino-8′-(2-fluoropyridin-3-yl)-1,4a′-dimethyl-3′,4′,4a′,10a′-tetrahydro-2′H-spiro[imidazole-4,10′-pyrano[3,2-b]chromen]-5(1H)-one was prepared from (4S*,4a′S*,10a′S*)-8′-bromo-4a′-methyl-3′,4′,4a′,10a′-tetrahydro-2′H-spiro[imidazolidine-4,10′-pyrano[3,2-b]chromene]-2,5-dione according to the procedures described for Example 47, Steps F-I. The racemic product was purified by preparative TLC (0.5 mm thickness, Rf=0.65) eluting with 10% MeOH (containing 7N NH₃)/DCM. ¹H NMR (400 MHz, CDCl₃) δ 8.14 (d, J=4 Hz, 1H), 7.78 (t, J=9 Hz, 1H), 7.45 (d, J=9 Hz, 1H), 7.28 (m, 1H), 7.23 (m, 1H), 6.97 (d, J=9 Hz, 1H), 4.10 (m, 1H), 3.53 (s, 1H), 3.47 (m, 1H), 3.16 (s, 3H), 2.12 (m, 2H), 1.67 (m, 1H), 1.54 (s, 3H), 1.49 (m, 1H); m/z (APCI-pos) M+1=397.

### Example 56

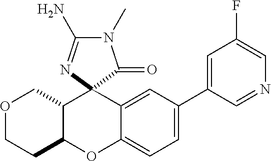

### (4R*,4a′S*,10a′S*)-2-amino-8′-(5-fluoropyridin-3-yl)-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (Example 47, Step H) according to the procedure described in Example 47, Step I, replacing 2-fluoropyridin-3-ylboronic acid with 5-fluoropyridin-3-ylboronic acid. The racemic product was purified by preparative TLC (0.5 mm thickness, Rf=0.29) eluting with 10% MeOH (containing 7N NH₃)/DCM. ¹H NMR (400 MHz, CDCl₃+MeOD) δ 8.50 (m, 1H), 8.34 (d, J=3 Hz, 1H), 7.56 (m, 1H), 7.43 (dd, J=2, 9 Hz, 1H), 7.09 (d, J=2 Hz, 1H), 7.02 (d, J=9 Hz, 1H), 4.97 (td, J=td, 1H), 4.09 (m, 1H), 3.96 (m, 1H), 3.51 (m, 1H), 3.10 (s, 3H), 3.05 (t, J=11 Hz, 1H), 2.27 (m, 2H), 1.90 (m, 1H); m/z (APCI-pos) M+1=383.

### Example 57

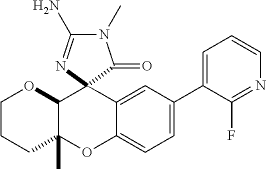

### (4S*,4a′R*,10a′S*)-2-amino-8′-(2-fluoropyridin-3-yl)-1,4a′-dimethyl-3′,4′,4a′,10a′-tetrahydro-2′H-spiro[imidazole-4,10′-pyrano[3,2-b]chromen]-5(1H)-one

The title compound was prepared from (4S*,4a′R*,10a′S*)-8′-bromo-4-a′-methyl-3′,4′,4a′,10a′-tetrahydro-2′H-spiro[imidazolidine-4,10′-pyrano[3,2-b]chromene]-2,5-dione (Example 55, Step G) according to the procedures described for Example 47, Steps F-I. The racemic product was purified by preparative TLC (0.5 mm thickness, Rf=0.50) eluting with 10% MeOH (containing 7N NH₃)/DCM. ¹H NMR (400 MHz, CDCl₃) δ 8.14 (d, J=5 Hz, 1H), 7.75 (m, 1H), 7.41 (d, J=9 Hz, 1H), 7.22 (m, 1H), 6.99 (br s, 1H), 6.94 (d, J=9 Hz, 1H), 4.43 (br s, 2H), 4.08 (s, 1H), 4.04 (m, 1H), 3.56 (m, 1H), 3.19 (s, 3H), 2.09 (m, 1H), 1.92 (m, 2H), 1.73 (m, 1H), 1.45 (s, 3H); m/z (APCI-pos) M+1=397.

### Example 58

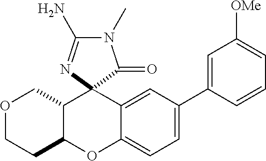

### (4R*,4a′S*,10a′S*)-2-amino-8′-(3-methoxyphenyl)-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (Example 47, Step H) according to the procedure described in Example 47, Step I, replacing 2-fluoropyridin-3-ylboronic acid with 3-methoxyphenylboronic acid. The racemic product was purified by preparative TLC (0.5 mm thickness, Rf=0.49) eluting with 10% MeOH (containing 7N NH₃)/DCM. ¹H NMR (400 MHz, CDCl₃+MeOD) δ 7.42 (m, 1H), 7.31 (t, J=8 Hz, 1H), 7.08 (m, 1H), 7.06 (m, 1H), 6.99 (m, 1H), 6.96 (d, J=9 Hz, 1H), 6.85 (m, 1H), 4.94 (td, J=5, 11 Hz, 1H), 4.08 (m, 1H), 3.95 (m, 1H), 3.84 (s, 3H), 3.51 (m, 1H), 3.08 (s, 3H), 3.06 (t, J=11 Hz, 1H), 2.25 (m, 2H), 1.89 (m, 1H); m/z (APCI-pos) M+1=394.

### Example 59

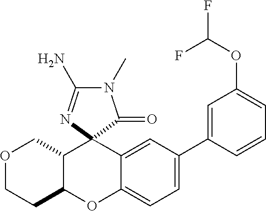

### (4R*,4a′S*,10a′S*)-2-amino-8′-(3-(difluoromethoxy)phenyl)-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (Example 47, Step H) according to the procedure described in Example 47, Step I, replacing 2-fluoropyridin-3-ylboronic acid with 3-(difluoromethoxy)phenylboronic acid. The racemic product was purified by preparative TLC (0.5 mm thickness, Rf=0.49) eluting with 10% MeOH (containing 7N NH₃)/DCM. ¹H NMR (400 MHz, CDCl₃+MeOD) δ 7.39 (dd, J=2, 8 Hz, 1H), 7.37 (t, J=8 Hz, 1H), 7.31 (m, 1H), 7.20 (m, 1H), 7.06 (d, J=2 Hz, 1H), 7.05 (m, 1H), 6.97 (d, J=9 Hz, 1H), 6.56 (t, J=74 Hz, 1H), 4.94 (td, J=5, 11 Hz, 1H), 4.06 (m, 1H), 3.96 (m, 1H), 3.50 (m, 1H), 3.08 (s, 3H), 3.05 (t, J=12 Hz, 1H), 2.27 (m, 2H), 1.89 (m, 1H); m/z (APCI-pos) M+1=430.

### Example 60

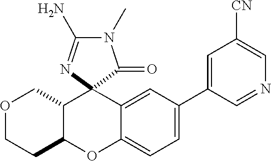

### 5-((4R*,4a′S*,10a′S*)-2-amino-1-methyl-5-oxo-1,3′,4′,4a′,5,10a′-hexahydro-1′H-spiro[imidazole-4,10′-pyrano[4,3-b]chromen]-8′-yl)nicotinonitrile

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (Example 47, Step H) according to the procedure described in Example 47, Step I, replacing 2-fluoropyridin-3-ylboronic acid with 5-(4,4,5,5-tetramethyl-1,3,2-dioxaborolan-2-yl)nicotinonitrile. The racemic product was purified by preparative TLC (2 mm thickness, Rf=0.56) eluting with 10% MeOH (containing 7N NH₃)/DCM. ¹H NMR (CDCl₃, 400 MHz) δ 8.90 (m, 1H), 8.76 (m, 1H), 8.06 (m, 1H), 7.42 (m, 1H), 7.08 (m, 1H), 7.03 (d, J=9 Hz, 1H), 4.97 (m, 1H), 4.08 (m, 1H), 3.95 (m, 1H), 3.47 (d, J=12 Hz, 1H), 3.09 (s, 3H), 3.03 (t, J=12 Hz, 1H), 2.20 (m, 2H), 1.88 (m, 1H); m/z (APCI-pos) M+1=390.

### Example 61

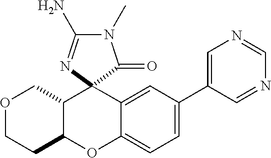

### (4R*,4a′S*,10a′S*)-2-amino-1-methyl-8′-(pyrimidin-5-yl)-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-amino-8′-bromo-1-methyl-3′,4′,4a′,10a′-tetrahydro-1′H-spiro[imidazole-4,10′-pyrano[4,3-b]chromen]-5(1H)-one (Example 47, Step H) according to the procedure described in Example 47, Step I, replacing 2-fluoropyridin-3-ylboronic acid with pyrimidin-5-ylboronic acid. The racemic product was purified by preparative TLC (0.5 mm thickness, Rf=0.33) eluting with 10% MeOH (containing 7N NH₃)/DCM. ¹H NMR (400 MHz, CDCl₃+MeOD) δ 9.10 (s, 1H), 8.87 (s, 2H), 7.46 (m, 1H), 7.12 (s, 1H), 7.06 (d, J=9 Hz, 1H), 4.96 (td, J=5, 11 Hz, 1H), 4.10 (m, 1H), 3.97 (m, 1H), 3.51 (m, 1H), 3.12 (s, 3H), 3.05 (t, J=11 Hz, 1H), 2.27 (m, 2H), 1.91 (m, 1H); m/z (APCI-pos) M+1=366.

### Example 62

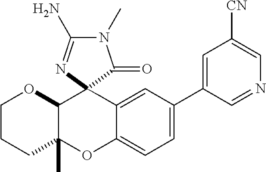

### (4S*,4a′R*,10a′S*)-2-amino-8′-(5-chloropyridin-3-yl)-1,4a′-dimethyl-3′,4′,4a′,10a′-tetrahydro-2′H-spiro[imidazole-4,10′-pyrano[3,2-b]chromen]-5(1H)-one

Step A: A solution of 5-hydroxypentan-2-one (65.7 mL, 644 mmol) and imidazole (65.7 g, 965 mmol) in DCM (600 mL) was cooled in an ice bath and treated dropwise (by addition funnel) with a solution of TBDMS-Cl (97 g, 644 mmol) in DCM (500 mL) over a 1 hour time period. The ice bath was removed, and the reaction was allowed to come to room temperature and stirring continued for 1 hour. The reaction was washed with 1N aqueous HCl (1 L), water (1 L), then saturated aqueous NaHCO₃ (1 L) and dried over Na₂SO₄ to yield 5-((tert-butyldimethylsilyl)oxy)pentan-2-one (116.7 g, 67%).

Step B: A round bottomed flask plus stir bar was charged with 1-(2-hydroxy-5-methoxyphenyl)ethanone (72.9 g, 439 mmol), 5-((tert-butyldimethylsilyl)oxy)pentan-2-one (86.3 g, 399 mmol), EtOH (500 mL) and pyrrolidine (31.2 g, 439 mmol) and was heated to 80° C. for 18 hours with stirring and an attached water reflux condenser. After cooling to room temperature, the reaction mixture was transferred to a separatory funnel with diethyl ether (500 mL). The mixture was washed with 1N aqueous NaOH (500 mL). The aqueous phase was re-extracted with diethyl ether (150 mL) The combined organic phases were washed with 1N aqueous HCl (500 mL), re-extracting the aqueous phase with diethyl ether (150 mL). Then, the combined organic phases were washed with saturated aqueous NaHCO₃ (500 mL), dried (MgSO₄), filtered, and concentrated to yield 2-(3-((tert-butyldimethylsilyl)oxy)propyl)-6-methoxy-2-methylchroman-4-one (117 g, 65%).

Step C: A round bottomed flask plus stir bar was charged with ethyl formate (155 mL, 1926 mmol), diethyl ether (600 mL) and sodium methoxide (86.7 g, 1605 mmol) at 0° C. The reaction mixture was stirred for 20 minutes. Next, 2-(3-((tert-butyldimethylsilyl)oxy)propyl)-6-methoxy-2-methylchroman-4-one (117 g, 321 mmol) dissolved in diethyl ether (200 mL) was added by canula over a 30 minute period with vigorous stirring. The reaction mixture was removed from bath and stirred at room temperature. The reaction mixture was stirred at room temperature for 3 hours and then worked up by cooling to 0° C., and carefully adding saturated aqueous NH₄Cl (500 mL) in small portions maintaining internal temperature below 15° C. The reaction mixture was transferred to a separatory funnel, rinsing with diethyl ether. The phases were separated, and the aqueous phase was re-extracted with diethyl ether (200 mL). The combined organic phases were dried (MgSO₄), filtered, and concentrated to yield 2-(3-((tert-butyldimethylsilyl)oxy)propyl)-6-methoxy-2-methyl-4-oxochroman-3-carbaldehyde (130 g, 62%).

Step D: Diethylamine (45.1 g, 616 mmol) was added to a solution of crude 2-(3-((tert-butyldimethylsilyl)oxy)propyl)-6-methoxy-2-methyl-4-oxochroman-3-carbaldehyde (121 g, 308 mmol) and naphthalene-2-sulfonyl azide (79.1 g, 339 mmol, prepared according to the procedure described for 4-methylbenzenesulfonyl azide in WO 2010/011147, but replacing 4-methylbenzenesulfonyl chloride with naphthalene-2-sulfonyl chloride, and replacing DCM with EtOAc during the workup) in Et₂O (600 mL) while cooled in an ice bath. The reaction mixture was left in the ice bath to warm up slowly, while stirring under N₂. The reaction mixture was stirred at room temperature for 18 hours. The reaction mixture was filtered to remove most of the sulfonamide by-product and concentrated in vacuo. The crude was purified by Biotage Flash 75 silica gel chromatography (split material over 2 columns), eluting with DCM, then 2% MeOH/DCM. The product fractions were pooled, and the mixed fractions were pooled separately and re-chromatographed with the same conditions to yield 2-(3-((tert-butyldimethylsilyl)oxy)propyl)-3-diazo-6-methoxy-2-methylchroman-4-one (58 g, 29%).

Step E: A round bottomed flask plus stir bar was charged with 2-(3-((tert-butyldimethylsilyl)oxy)propyl)-3-diazo-6-methoxy-2-methylchroman-4-one (58 g, 149 mmol), THF (150 mL) and TBAF (1M in THF, 223 mL, 223 mmol). The reaction mixture was cooled in an ice bath during addition of the TBAF and stirred at room temperature for 3 hours. As TLC indicated, there was still unreacted starting material, and more TBAF (1M in THF, 75 mL) was added and continued stirring for 2 hours. The reaction mixture was worked up by pardoning between EtOAc (250 mL) and water (250 mL). The phasese were separated. The aqueous phase was re-extracted with EtOAc (250 mL). The combined organic phases were washed again with water (250 mL), brine (250 mL), dried (MgSO₄), filtered, and concentrated. The crude was purified by Biotage Flash 75 silica gel chromatography eluting with DCM, 2% MeOH/DCM, then 3% MeOH/DCM to fully elute products to yield 3-diazo-2-(3-hydroxypropyl)-6-methoxy-2-methylchroman-4-one (33.3 g, 61%).

Step F: A round bottomed flask plus stir bar was charged with 3-diazo-2-(3-hydroxypropyl)-6-methoxy-2-methylchroman-4-one (17.7 g, 64.1 mmol) and anhydrous toluene (180 mL). The reaction mixture was degassed with N₂ for 10 minutes, and then rhodium(II) acetate dimer (1.02 g, 2.31 mmol) was added. Immediately submerged the container into a pre-heated oil bath at 90° C. with stirring under a stream of N₂. The container was removed from the oil bath after gas evolution ceased (approximately 5-10 minutes). This reaction crude was combined with previous reactions performed similarly on smaller scale to yield a crude material (23.9 g). The combined crudes were filtered through Celite®, rinsing with DCM. The filtrate was concentrated, and the crude was purified by Biotage Flash 75 silica gel chromatography eluting with 30%-1:1 EtOAc/hexanes to yield (4aR*,10aR*)-8-methoxy-4-a-methyl-2,3,4,4a-tetrahydropyrano[3,2-b]chromen-10(10aH)-one (15.2 g, 32%).

Step G: Three diastereomers, (4S*,4a′R*,10a′S*)-, (4S*,4a′S*,10a′S*)- and (4S*,4a′S*,10a′R*)-, of 2-amino-8′-methoxy-1,4a′-dimethyl-3′,4′,4a′,10a′-tetrahydro-2′H-Spiro[imidazole-4,10′-pyrano[3,2-b]chromen]-5(1H)-one were synthesized from (4aR*,10aR*)-8-methoxy-4-a-methyl-2,3,4,4a-tetrahydropyrano[3,2-b]chromen-10(10aH)-one according to the procedures described for Example 47, Steps E-H. The diastereomer (4S*,4a′S*,10a′R*)-2-amino-8′-methoxy-1,4a′-dimethyl-3′,4′,4a′,10a′-tetrahydro-2′H-spiro[imidazole-4,10′-pyrano[3,2-b]chromen]-5(1H)-one was separated from the other two (which were not separated at this step) by Biotage Flash 65 silica gel chromatography, eluting with 2%-7% MeOH (containing 7N NH₃)/DCM.

Step H: A round bottomed flask plus stir bar was charged with a 1:1 mixture of (4S*,4a′R*,10a′S*)-2-amino-8′-methoxy-1,4a′-dimethyl-3′,4′,4a′,10a′-tetrahydro-2′H-spiro[imidazole-4,10′-pyrano[3,2-b]chromen]-5(1H)-one, and its diastereomer (4S*,4a′S*,10a′S*)-2-amino-8′-methoxy-1,4a′-dimethyl-3′,4′,4a′,10a′-tetrahydro-2′H-spiro[imidazole-4,10′-pyrano[3,2-b]chromen]-5(1H)-one (506 mg of the mixture, 1.53 mmol) and DCM (5 mL). The reaction mixture was chilled in an acetone/dry ice bath, that was chilled to −20° C. with addition of dry ice under N₂. BBr₃ (3.0 mL, 3.05 mmol, 1M in DCM) was added dropwise. The contents of the reaction vessel were transferred to a 0° C. ice water bath and stirred for 3 hours. The reaction mixture was quenched with ice chips. The reaction mixture was poured into saturated aqueous NaHCO₃ (20 mL). The solution was saturated with NaCl powder and then extracted with EtOAc/MeOH cosolvent (4×10 mL). The combined organics were dried (MgSO₄), filtered, and concentrated to yield of mixture of diastereomers (4S*,4a′R*,10a′S*)-2-amino-8′-hydroxy-1,4a′-dimethyl-3′,4′,4a′,10a'-tetrahydro-2′H-spiro[imidazole-4,10′-pyrano[3,2-b]chromen]-5(1H)-one and (4S*,4a′S*,10a′S*)-2-amino-8′-hydroxy-1,4a′-dimethyl-3′,4′,4a′,10a′-tetrahydro-2′H-spiro[imidazole-4,10′-pyrano[3,2-b]chromen]-5(1H)-one (433 mg, 89%).

Step I: The diastereomeric mixture (4S*,4a′R*,10a′S*)-2-amino-8′-hydroxy-1,4a′-dimethyl-3′,4′,4a′,10a′-tetrahydro-2′H-spiro[imidazole-4,10′-pyrano[3,2-b]chromen]-5(1H)-one and (4S*,4a′S*,10a′S*)-2-amino-8′-hydroxy-1,4a′-dimethyl-3′,4′,4a′,10a′-tetrahydro-2′H-spiro[imidazole-4,10′-pyrano[3,2-b]chromen]-5(1H)-one (433 mg, 1.36 mmol) in DMF (9 mL) with DMF-DMA (0.8 mL, 6.82 mmol) was stirred overnight at room temperature. The reaction mixture was concentrated in vacuo at 70° C. and then dried under high vacuum until a solid was obtained. The crude mixture of diastereomers, (E)-N′-((4S*,4a′R*,10a′S*)-8′-hydroxy-1,4a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide and (E)-N′-((4S*,4a′S*,10a′S*)-8′-hydroxy-1,4a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide were carried forward to the next step without purification.

Step J: A solution of (E)-N′-((4S*,4a′R*,10a′S*)-8′-hydroxy-1,4a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide and (E)-N′-((4S*,4a′S*,10a′S*)-8′-hydroxy-1,4a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide (508 mg, 1.36 mmol) in DCM (5 mL) was treated with triethylamine (380 μL, 2.73 mmol) and 1,1,1-trifluoro-N-phenyl-N-(trifluoromethylsulfonyl) methanesulfonamide (731 mg, 2.05 mmol). The reaction was sealed in a round bottomed flask plus stir bar and stirred for 4 hours at room temperature. The reaction mixture was washed with water (10 mL), and the aqueous phase was re-extracted with DCM (10 mL). The combined organics were washed with brine (10 mL), dried (MgSO₄), filtered, and concentrated. The two diastereomers were separated by Biotage Flash 65 silica gel chromatography, eluting with 2%-3% MeOH/DCM to yield (4S*,4a′R*,10a′S*)-2-((E)-((dimethylamino)methylene)amino)-1,4a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate (240 mg, 24%).

Step K: A vial plus stir bar was charged with (4S*,4a′R*,10a′S*)-2-((E)-((dimethylamino)methylene)amino)-1,4a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate (60 mg, 0.12 mmol), dioxane (1 mL), 5-chloropyridin-3-ylboronic acid (28 mg, 0.18 mmol), Pd(PPh₃)₄ (14 mg, 0.012 mmol), and 2N aqueous Na₂CO₃ (178 μL, 0.36 mmol). The reaction mixture was sparged with N₂ for 2 minutes and then heated to 90° C. for 2 hours with stirring. The reaction mixture was loaded directly onto a preparative TLC plate (1 mm thickness, Rf=0.51) and eluted with 10% MeOH (containing 7N NH₃)/DCM. The product required a second purification by preparative TLC, eluting with 10% MeOH/DCM to remove by-products and yield (4S*,4a′R*,10a′S*)-2-amino-8′-(5-chloropyridin-3-yl)-1,4a′-dimethyl-3′,4′,4a′,10a′-tetrahydro-2′H-spiro[imidazole-4,10′-pyrano[3,2-b]chromen]-5(1H)-one (5 mg, 10%). ¹H NMR (1:1 MeOD/CDCl₃) δ 8.53 (d, J=2 Hz, 1H), 8.46 (d, J=2 Hz, 1H), 7.78 (t, J=2 Hz, 1H), 7.41 (dd, J=2, 9 Hz, 1H), 6.98 (d, J=9 Hz, 1H), 6.91 (d, J=2 Hz, 1H), 4.03 (s, 1H), 4.02 (m, 1H), 3.54 (m, 1H), 3.20 (s, 3H), 2.09 (m, 1H), 1.91 (m, 2H), 1.74 (m, 1H), 1.46 (s, 3H); m/z (APCI-pos) M+1=413.

### Example 63

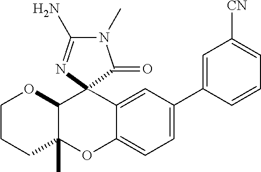

### 3-((4S*,4a′R*,10a′S*)-2-amino-1,4a′-dimethyl-5-oxo-1,3′,4′,4a′,5,10a′-hexahydro-2′H-spiro[imidazole-4,10′-pyrano[3,2-b]chromen]-8′-yl)benzonitrile

The title compound was prepared from (4S*,4a′R*,10a′S*)-2-((E)-((dimethylamino)methylene)amino)-1,4a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate according to the procedure for Example 62, Step K, replacing 5-chloropyridin-3-ylboronic acid with 3-cyanophenylboronic acid. ¹H NMR (1:1 MeOD/CDCl₃) δ 7.72 (m, 1H), 7.68 (m, 1H), 7.58 (m, 1H), 7.51 (t, J=8 Hz, 1H), 7.40 (dd, J=2, 9 Hz, 1H), 6.96 (d, J=9 Hz, 1H), 6.90 (d, J=2 Hz, 1H), 4.03 (s, 1H), 4.02 (m, 1H), 3.54 (m, 1H), 3.21 (s, 3H), 2.09 (m, 1H), 1.94 (m, 2H), 1.74 (m, 1H), 1.46 (s, 3H); m/z (APCI-pos) M+1=403.

### Example 64

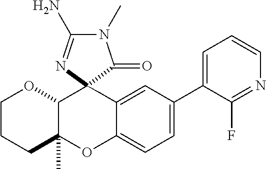

### (4S*,4a′S*,10a′R*)-2-amino-8′-(2-fluoropyridin-3-yl)-1,4a′-dimethyl-3′,4′,4a′,10a′-tetrahydro-2′H-spiro[imidazole-4,10′-pyrano[3,2-b]chromen]-5(1H)-one

The title compound was prepared from (4S*,4a′S*,10a′R*)-2-amino-8′-methoxy-1,4a′-dimethyl-3′,4′,4a′,10a′-tetrahydro-2′H-spiro[imidazole-4,10′-pyrano[3,2-b]chromen]-5(1H)-one (Example 62, Step G) according to the procedures described for Example 62, Steps H-K, replacing 5-chloropyridin-3-ylboronic acid with 2-fluoropyridin-3-ylboronic acid in Step K. ¹H NMR (1:1 MeOD/CDCl₃) δ 8.11 (m, 1H), 7.81 (m, 1H), 7.38 (m, 1H), 7.27 (m, 1H), 7.01 (m, 1H), 6.96 (d, J=9 Hz, 1H), 4.06 (m, 1H), 3.83 (s, 1H), 3.53 (m, 1H), 3.12 (s, 3H), 2.05 (m, 1H), 1.90 (m, 2H), 1.72 (m, 1H), 1.62 (s, 3H); m/z (APCI-pos) M+1=397.

### Example 65

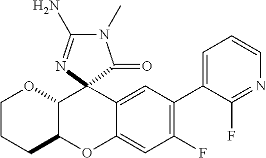

### (4R*,4a′S*,10a′S*)-2-amino-7′-fluoro-8′-(2-fluoropyridin-3-yl)-1-methyl-3′,4′,4a′,10a'-tetrahydro-1′H-spiro[imidazole-4,10′-pyrano[4,3-b]chromen]-5(1H)-one

Step A: A stirred solution of 1-(3-fluoro-4-methoxyphenyl)ethanone (50 g, 297 mmol) in DCM (1.2 L) was treated with m-CPBA (83.3 g, 372 mmol). The suspension was heated to 40° C. with stirring, and the suspension became a solution. The reaction was stirred for 72 hours at 40° C., and TLC suggested only partial conversion. The reaction was cooled to room temperature, and an additional m-CPBA (80 g) was added in a single portion. The reaction was returned to 40° C., and the reaction stirred for an additional 48 hours. TLC confirmed conversion of starting material. The reaction was cooled to room temperature and washed with aqueous 1N NaOH, repeating until the organic phase was clear. The organic phase was then washed with brine, dried (Na2SO₄) and concentrated to an oil to yield of 3-fluoro-4-methoxyphenyl acetate (47.7 g, 87%).

Step B: Neat trifluoromethanesulfonic acid (194 g, 1295 mmol) was added dropwise by addition funnel into 3-fluoro-4-methoxyphenyl acetate (47.7 g, 259 mmol) stirring at 0° C. The reaction was heated to 60° C. for 1 hour and cooled to room temperature. The reaction was poured carefully into an ice slurry (1 L). The resulting suspension was filtered, and the solid was partitioned between Et₂O and saturated aqueous NaHCO₃. The organic phase was washed with brine, dried (Na₂SO₄) and concentrated under vacuum to yield 1-(4-fluoro-2-hydroxy-5-methoxyphenyl)ethanone (44.2 g, 93%) as an oil.

Step C: A solution of bis(trichloromethyl) carbonate (71.2 g, 240 mmol) in DCE (160 mL) was added dropwise to a flask containing a mixture of N,N-dimethylformamide (254 mL, 2880 mmol) and DCE (300 mL) that was stirred in an ice bath. The reaction temperature was maintained below 25° C. After addition, the reaction was cooled to 0° C. and treated with a solution of 1-(4-fluoro-2-hydroxy-5-methoxyphenyl)ethanone (44.2 g, 240 mmol) in DCE (160 mL). The ice bath was removed and the reaction was allowed to warm to room temperature while monitoring by HPLC. After 5 hours of stirring at room temperature, the reaction was poured into a 2 L ice slurry and stirred for an additional 2 hours. The aqueous phase was extracted multiple times with DCE. The combined organic extracts were washed with saturated aqueous NaHCO₃, brine, and then the organic phase was concentrated under vacuum. The resulting solid was placed in a vacuum oven and heated to 60° C. over night to yield 7-fluoro-6-methoxy-4-oxo-4H-chromene-3-carbaldehyde (28 g, 53%) as a powder.

Step D: A stirred suspension of 7-fluoro-6-methoxy-4-oxo-4H-chromene-3-carbaldehyde (31.4 g, 141 mmol) and ethyl vinyl ether (67.9 mL, 707 mmol) was heated at 100° C. in a teflon lined stainless steel reaction “bomb” for 8 hours. The heat was removed and the reaction continued to stir an additional 7 hours at room temperature. The resulting residue was crystallized from hot EtOH, and the solids were filtered to yield (3S*,4aS*)-3-ethoxy-7-fluoro-8-methoxy-4,4-a-dihydropyrano[4,3-b]chromen-10(3H)-one (16.6 g, 40%).

Step E: A suspension of (3S*,4aS*)-3-ethoxy-7-fluoro-8-methoxy-4,4-a-dihydropyrano[4,3-b]chromen-10(3H)-one (13.3 g, 45.3 mmol) in EtOH (100 mL) was treated with Pd/C (10 wt %, 0.8 g) and shaken in a Parr shaker under H₂ (50 psi) for 3 hours. The reaction was filtered through GF/F paper, and the filtrate concentrated. The solid was resuspended in DCM (100 mL) and stirred with MnO₂ (7.9 g, 90.5 mmol) overnight. The mixture was filtered and concentrated to provide 3-ethoxy-7-fluoro-8-methoxy-1,4,4a,10a-tetrahydropyrano[4,3-b]chromen-10(3H)-one (12.1 g, 90% yield) as a solid.

Step F: A solution of 3-ethoxy-7-fluoro-8-methoxy-1,4,4a,10a-tetrahydropyrano[4,3-b]chromen-10(3H)-one (11.1 g, 37 mmol) in DCM (3 mL) was cooled to 0° C. and treated with triethylsilane (18 mL, 112 mmol), then BF₃ Etherate (9.2 mL, 75 mmol). The reaction was allowed to stir at room temperature overnight. The reaction was incomplete, so an additional 3 equivalents of triethylsilane and 2 equivalents of BF₃ etherate were added. The reaction continued to stir at room temperature. After 40 hours, the reaction mixture was dissolved in EtOAc (30 mL) and MeOH (5 mL) and quenched with aqueous saturated NaHCO₃ and stirred for 4 h. The yellow organic layer became colorless, was separated and washed with brine. The organic was dried (Na₂SO₄) and concentrated under vacuum to provide 7-fluoro-8-methoxy-1,4,4a,10a-tetrahydropyrano[4,3-b]chromen-10(3H)-one (8.58 g, 91% yield) as an oil, which formed crystals upon standing.

Step G: 2-Amino-7′-fluoro-8′-methoxy-1-methyl-3′,4′,4a′,10a′-tetrahydro-1′H-spiro[imidazole-4,10′-pyrano[4,3-b]chromen]-5(1H)-one (mixture of cis/trans diastereomers, 0.500 g, 1.49 mmol; synthesized from 7-fluoro-8-methoxy-1,4,4a,10a-tetrahydropyrano[4,3-b]chromen-10(3H)-one, according to the procedures described for Example 47, Steps E-H) was treated with 48% aqueous HBr (7.5 mL, 1.49 mmol) and heated to 100° C. in a sealed vial for 3 hours. The reaction was cooled to room temperature and treated with DCM (50 mL) and saturated aqueous NaHCO₃ until slightly basic. The pH was brought to about 5 by careful addition of aqueous 1N HCl. The product remained completely dissolved in the aqueous layer in the pH range 5-8. The entire biphasic mixture was then concentrated under vacuum, and the residue was triturated with 10% MeOH/DCM. The resulting suspension was filtered and the filtrate was concentrated to provide 2-amino-7′-fluoro-8′-hydroxy-1-methyl-3′,4′,4a′,10a′-tetrahydro-1′H-spiro[imidazole-4,10′-pyrano[4,3b]chromen]-5(1H)-one (mixture of cis/trans diastereomers) as a crude oil.

Step H: (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate was synthesized from a mixture of cis/trans diastereomers 2-amino-7′-fluoro-8′-hydroxy-1-methyl-3′,4′,4a′,10a′-tetrahydro-1′H-spiro[imidazole-4,10′-pyrano[4,3-b]chromen]-5(1H)-one according to the procedures described for Example 47, Steps I-J. Cis/trans diastereomers of (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate were separated by silica gel chromatography to provide (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate and its diastereomer (4R*,4a′S*,10a′R*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chrome]-8′-yl trifluoromethanesulfonate (90 mg, 12%).

Step I: (4R*,4a′S*,10a′S*)-2-Amino-7′-fluoro-8′-(2-fluoropyridin-3-yl)-1-methyl-3′,4′,4a′,10a′-tetrahydro-1′H-spiro[imidazole-4,10′-pyrano[4,3-b]chromen]-5(1H)-one (2.5 mg, 11%) was synthesized from (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate according to the procedure for Example 47, Step K, replacing 5-chloropyridin-3-ylboronic acid with 2-fluoropyridin-3-ylboronic acid. ¹H NMR (400 MHz, CDCl₃): δ 8.16 (s, 1H), 7.77 (m, 1H), 7.22 (m, 1H), 6.97 (m, 1H), 6.73 (m, 1H), 4.98 (m, 1H), 4.10-3.92 (m, 2H), 3.48 (m, 1H), 3.14-3.02 (m, 1H), 3.07 (s, 3H), 2.28-2.10 (m, 2H), 1.92-1.77 (m, 1H); m/z (APCI-pos) M+1=401.

### Example 66

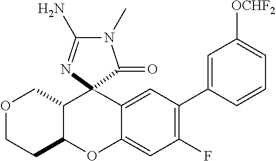

### (4R*,4a′S*,10a′S*)-2-amino-8′-(3-(difluoromethoxy)phenyl)-7′-fluoro-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate (Example 65, Step H) according to the procedure for Example 62, Step K, replacing 5-chloropyridin-3-ylboronic acid with 3-(difluoromethoxy)phenylboronic acid. ¹H NMR (400 MHz, CDCl₃) δ 7.38 (m, 1H), 7.31-7.25 (m, 1H), 7.19 (m, 1H), 7.09 (m, 1H), 6.94 (m, 1H), 6.72 (m, 1H), 6.54 (t, J=74 Hz, 1H), 4.96 (m, 1H), 4.10-3.91 (m, 2H), 3.48 (m, 1H), 3.08 (s, 3H), 3.04 (m, 1H), 2.23-2.11 (m, 2H), 1.91-1.78 (m, 1H); m/z (APCI-pos) M+1=448.

### Example 67

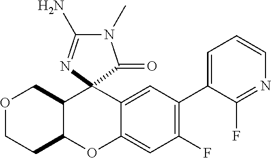

### (4R*,4a′S*,10a′R*)-2-amino-7′-fluoro-8′-(2-fluoropyridin-3-yl)-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′R*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate (Example 65, Step H) according to the procedure for Example 62, Step K, replacing 5-chloropyridin-3-ylboronic acid with 2-fluoropyridin-3-ylboronic acid. m/z (APCI-pos) M+1=401.

### Example 68

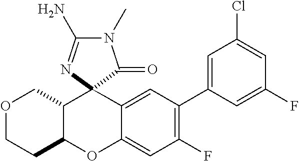

### (4R*,4a′S*,10a′S*)-2-amino-8′43-chloro-5-fluorophenyl)-7′-fluoro-1-methyl-3′,4′,4a′,10a′-tetrahydro-1′H-spiro[imidazole-4,10′-pyrano[4,3-b]chromen]-5(1H)-one

The title compound was prepared from (4R*,4a′S*,10a′S*)-2-((E)-((dimethylamino)methylene)amino)-7′-fluoro-1-methyl-5-oxo-1,3′,4′,4a′,5,10a′-hexahydro-1′H-spiro[imidazole-4,10′-pyrano[4,3-b]chromen]-8′-yl trifluoromethanesulfonate (Example 65, Step H) according to the procedure for Example 62, Step K, replacing 5-chloropyridin-3-ylboronic acid with 3-chloro-5-fluorophenylboronic acid. m/z (APCI-pos) M+1=434.

### Example 69

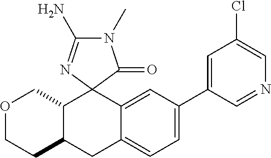

### (4aS,10aS)-2′-amino-8-(5-chloropyridin-3-yl)-1′-methyl-1,3,4,4a,5,10a-hexahydrospiro[benzo[g] isochromene-10,4′-imidazol]-5′(1′H)-one

Step A: A solution of 1,1,1-trifluoro-N-(trifluoromethylsulfonyl)methanesulfonamide (0.304 g, 1.08 mmol) in DCM (5 mL) was added dropwise to a solution of (5,6-dihydro-2H-pyran-3-yloxy)trimethylsilane (19.5 g, 110 mmol, prepared according to the method described in WO 2009/043883) and 4-methoxybenzyl acetate (19.5 g, 108 mmol) in dichloromethane (216 mL, 108 mmol) at 0° C. under N₂ atmosphere. The mixture was stirred at 0° C. for 10 minutes and quenched with ice water (30 mL). The organic layer was separated, washed with brine (50 mL), dried (MgSO₄) and concentrated in vacuo. The crude isolated was purified by flash chromatography on silica gel (Ready Sep 220 g) eluting with 10% EtOAc/hexane to provide 4-(4-methoxybenzyl)dihydro-2H-pyran-3(4H)-one (21 g, 88% yield) as a solid. ¹H NMR (400 MHz, CDCl₃) δ 7.07 (d, J=8.61 Hz, 2H), 6.83 (d, J=8.61 Hz, 2H), 4.06 (d, J=15.65 Hz, 1H), 3.98-3.94 (m, 2H), 3.79 (s, 3H), 3.76-3.70 (m, 1H), 3.29 (dd, J1=4.30 Hz, J2=14.08 Hz, 1H), 2.71-2.63 (m, 1H), 2.06-1.98 (m, 1H), 1.79-1.69 (m, 1H).

Step B: A solution of n-butyllithium 2.5 in hexanes (48.3 mL, 121 mmol) was added dropwise to a stirred suspension of (methoxymethyl)triphenylphosphonium chloride (43.6 g, 127 mmol) in tetrahydrofuran (254 mL, 63.6 mmol) at 0° C. under N₂ atmosphere. Once the addition was complete, the ice bath was removed, and the mixture was stirred at ambient temperature for 15 minutes. The mixture was then cooled to −78° C. and treated dropwise with a solution of 4-(4-methoxybenzyl)dihydro-2H-pyran-3(4H)-one (14 g, 63.6 mmol) in THF (60 mL) over 30 minutes. After 2 hours at −78° C., the mixture was poured into saturated aqueous NaHCO₃ solution (100 mL) and partitioned with EtOAc (4×100 mL). The organic layers were combined, washed with brine (2×60 mL), dried (MgSO₄) and concentrated in vacuo. The residue obtained was purified by flash chromatography on silica gel (Ready Sep 330 g) eluting with 10% EtOAc/hexane to provide (Z)-4-(4-methoxybenzyl)-3-(methoxymethylene)tetrahydro-2H-pyran (8.2 g, 52% yield) and (E)-4-(4-methoxybenzyl)-3-(methoxymethylene)tetrahydro-2H-pyran (2.1 g, 13.5% yield) as oils. Major isomer: ¹H NMR (400 MHz, CDCl₃) δ 7.056 (d, J=7.043 Hz, 2H), 6.83 (d, J=6.65 Hz, 2H), 5.73 (s, 1H), 4.48 (d, J=12.52 Hz, 1H), 4.00 (d, J=12.52 Hz, 1H), 3.91-3.83 (m, 1H), 3.79 (s, 3H), 3.59-3.54 (m, 1H), 3.54 (s, 3H), 2.90 (dd, J1=5.869 Hz, J2=13.30 Hz, 1H), 2.55-2.48 (m, 1H), 2.40-3.32 (m, 1H), 1.69-1.62 (m, 1H), 1.43-1.345 (m, 1H). Minor isomer: ¹H NMR (400 MHz, CDCl₃) δ 7.11 (d, J=7.43 Hz, 2H), 6.816 (d, J=7.43, Hz, 2H), 5.88 (s, 1H), 4.14 (d, J=12.52 Hz, 1H), 3.85 (d, 12.52 Hz, 1H), 3.78-3.76 (m, 5H), 3.41 (s, 3H), 3.18-3.08 (m, 1H), 2.86-2.73 (m, 2H), 1.84-1.73 (m, 1H), 1.43 (d, J=13.694 Hz, 1H).

Step C: A solution of (Z)-4-(4-methoxybenzyl)-3-(methoxymethylene)tetrahydro-2H-pyran (8.21 g, 33.1 mmol) in THF:2N HCl (1:1, 40 mL) and concentrated HCl (4 mL) was stirred at ambient temperature. After 18 hours, the mixture was diluted with water (70 mL) and extracted with EtOAc (2×100 mL). The organic layers were combined, dried (MgSO₄), concentrated in vacuo and purified by flash chromatography on silica gel (Ready Sep 220 g) eluting with 10% EtOAc/hexane to provide a mixture of cis and trans 4-(4-methoxybenzyl)tetrahydro-2H-pyran-3-carbaldehyde (6.8 g, 88% yield) as an oil. ¹H NMR (400 MHz, CDCl₃) 6 Major isomer: 9.96 (s, 1H), 7.09-7.07 (m, 2H), 6.85-6.82 (m, 2H), 4.23 (d, J=11.74 Hz, 1H), 4.07-4.0 (m, 1H), 3.78 (s, 3H), 3.56-3.51 (m, 1H), 3.49-3.40 (m, 1H), 2.87-2.79 (m, 1H), 2.73-2.65 (m, 1H), 2.34-2.28 (m, 1H), 2.14-2.05 (m, 1H), 1.89-1.769 (m, 1H), 1.61-1.54 (m, 1H). Minor isomer: 9.66 (s, 0.2H), 7.09-7.07 (m, 1H), 6.85-6.82 (m, 1H), 4.23 (d, J=11.74 Hz, 0.2H), 4.07-4.0 (m, 0.2H), 3.79 (s, 1.5H), 3.56-3.51 (m, 0.3H), 3.49-3.40 (m, 0.3H), 2.87-2.79 (m, 0.3H), 2.49-2.43 (m, 0.3H), 1.67-1.61 (m, 1H), 1.61-1.54 (m, 0.4H).

Step D: A solution of 4-(4-methoxybenzyl)tetrahydro-2H-pyran-3-carbaldehyde (6.85 g, 29.2 mmol) in tert-BuOH (112 mL, 29.2 mmol), tetrahydrofuran (112 mL, 29.2 mmol) and water (112 mL, 29.2 mmol) was cooled to 0° C. and sequentially added 2-methylbut-2-ene 2M in THF (87.7 mL, 87.7 mmol) and NaH₂PO₄ (42 g, 35 mmol). Then NaClO₂ (3.3 g, 29.2 mmol) was added in small portions. The mixture was stirred at 0° C. for 3 hours and allowed to warm to ambient temperature slowly over 15 hours. The reaction mixture was quenched with saturated NH₄Cl (30 mL) and extracted into EtOAc (250 mL). The organic layers were combined, dried (MgSO₄) and concentrated in vacuo to provide 4-(4-methoxybenzyl)tetrahydro-2H-pyran-3-carboxylic acid (8.1 g, 99.6% yield) as an oil. LCMS (APCI−) m/z 249 (M−H)⁻.

Step E: A mixture of 90% pure 4-(4-methoxybenzyl)tetrahydro-2H-pyran-3-carboxylic acid (8.1 g, 29.1 mmol) and PPA (5 mL) was heated at 80° C. for 1 hour. The mixture was cooled to ambient temperature and quenched with ice water. The resulting mixture was partitioned with EtOAc (300 mL). The organic layers were combined, washed sequentially with saturated NaHCO₃ (2×50 mL), brine (50 mL) then dried (MgSO₄) and concentrated in vacuo. The crude isolated was purified by flash chromatography on silica gel (Ready Sep 220 g) eluting with a gradient of 10%-30% EtOAc/hexanes (Biotage SP1, 10CV) to provide (4aR,10aS)-8-methoxy-3,4,4a,5-tetrahydro-1H-benzo[g]isochromen-10(10aH)-one (1.5 g 22% yield). ¹H NMR (400 MHz, CDCl₃) δ 7.47 (d, J=2.73 Hz, 1H), 7.15 (d, J=8.216 Hz, 1H), 7.07 (dd, J1=2.739 Hz, J2=8.216 Hz, 1H), 4.52 (dd, J1=4.695 Hz, J2=12.129 Hz, 1H), 4.023 (dd, J1=4.695 Hz, J2=11.738 Hz, 1H), 3.83 (s, 3H), 3.46-3.88 (m, 2H), 2.89 (dd, J1=4.304 Hz, J2=16.041 Hz, 1H), 2.77 (dd, J=11.738 Hz, J2=16.04 Hz, 1H), 2.54-2.46 (m, 1H), 2.19-2.09 (m, 1H), 1.81-1.75 (m, 1H), 1.72-1.65 (m, 1H).

Step F: A metal bomb was charged with a mixture of (4aR,10aS)-8-methoxy-3,4,4a,5-tetrahydro-1H-benzo[g]isochromen-10(10aH)-one (1.4 g, 6 mmol), ammonium carbonate (6.37 g, 66.3 mmol), potassium cyanide (0.98 g, 15 mmol) and 200 proof ethanol (6 mL, 6 mmol). The bomb was sealed and stirred at 130° C. for 24 hours and allowed to cool to room temperature. The contents were suspended in EtOH/H₂O (1:1, 3×10 mL) and transferred to a 500 mL Erlenmeyer flask. The suspension was diluted with additional water (100 mL) and slowly acidified to a pH of about 2 to 3 with 6M HCl. During this time, the mixture was sparged with N₂. The mixture was allowed to stir at room temperature for 30 minutes. The solid formed was filtered, washed with water (3×10 mL) and evaporated from CH₃CN to provide a mixture of cis and trans 8-methoxy-1,3,4,4a,5,10a-hexahydrospiro[benzo[g]isochromene-10,4′-imidazolidine]-2′,5′-dione (1.51 g, 83% yield) as a solid. LCMS: (APCI−) m/z 301 (M−H)⁻.

Step G: K₂CO₃ (0.823 g, 5.95 mmol) and iodomethane (0.3 mL, 4.7 mmol) were added to a solution of 8-methoxy-1,3,4,4a,5,10a-hexahydrospiro[benzo[g]isochromene-10,4′-imidazolidine]-2′,5′-dione (1.5 g, 4.96 mmol) in N,N-dimethylformamide (10 mL, 5 mmol). The mixture was stirred at ambient temperature overnight and poured into ice water. The mixture was then partitioned with EtOAc. The combined organic layers were dried (MgSO₄), filtered and concentrated in vacuo. The crude isolated was crystallized from IPA to provide (4aS,10aS)-8-methoxy-1′-methyl-1,3,4,4a,5,10a-hexahydrospiro[benzo[g] isochromene-10,4′-imidazolidine]-2′,5′-dione (735 mg, 47% yield) as a solid. ¹N NMR (400 MHz, CDCl₃) δ 7.01 (d, J=8.216 Hz, 1H), 6.83 (dd, J1=2.348 Hz, J2=8.216 Hz, 1H), 6.61 (d, J=2.348 Hz, 1H), 5.51 (s, 1H), 4.07 (dd, J1=4.304 Hz, J2=10.955 Hz, 1H), 3.99 (dd, J1=4.695 Hz, J2=11.346 Hz, 1H), 3.75 (s, 3H), 3.45 (t, J=11.346 Hz, 1H), 3.15 (t, J=11.346 Hz, 1H), 3.04 (s, 3H), 2.93 (dd, J1=4.695 Hz, J2=16.041 Hz, 1H), 2.80-2.69 (m, 1H), 2.47 (dd, J1=11.346 Hz, J2=16.433 Hz, 1H), 1.97 (dt, J1=4.304 Hz, J2=11.346 Hz, 1H), 1.87-1.78 (m, 1H), 1.53-1.43 (m, 1H).

Step H: A resealable glass pressure tube was charged with a suspension of (4aS,10aS)-8-methoxy-1′-methyl-1,3,4,4a,5,10a-hexahydrospiro[benzo[g] isochromene-10,4′-imidazolidine]-2′,5′-dione (810 mg, 2.56 mmol) in toluene (5121 μL, 2.56 mmol), and the mixture was stirred at reflux for 5 minutes. Once the starting material was in solution, Lawesson's Reagent (570 mg, 1.41 mmol) was added in one portion. The tube was capped with a Teflon screw cap and heated at 110° C. with stirring. After 18 hours, the reaction mixture was concentrated in vacuo, and the solid residue obtained was crystallized from IPA to provide (4aS,10aS)-8-methoxy-1′-methyl-2′-thioxo-1,3,4,4a,5,10a-hexahydrospiro[benzo[g]isochromene-10,4′-imidazolidin]-5′-one (715 mg, 84% yield) as a solid. LCMS (APCI−) m/z 331 (M−H)⁻.

Step I: Ammonia 7M in methanol (9 mL, 63.6 mmol) was sequentially added to a stirred solution of (4aS,10aS)-8-methoxy-1′-methyl-2′-thioxo-1,3,4,4a,5,10a-hexahydro spiro[benzo[g] isochromene-10,4′-imidazolidin]-5′-one (705 mg, 2.12 mmol) in methanol (8.5 mL, 2.12 mmol), followed by ter-butylhydroperoxide 70% in water (4.4 mL, 32 mmol). The mixture was stirred at room temperature overnight. Water (20 mL) was added to the mixture, and the resulting suspension was extracted into EtOAc (4×50 mL) The organic layers were combined, dried (MgSO₄) and concentrated in vacuo. The crude isolated was purified by flash chromatography (Ready Sep 80 g silica gel, Biotage SP1 unit) eluting with a gradient of 5-35% IPA/DCM+2% NH₃ (15 CV) to provide (4aS,10aS)-2′-amino-8-methoxy-1′-methyl-1,3,4,4a,5,10a-hexahydrospiro[benzo[g]isochromene-10,4′-imidazol]-5′(1H)-one (440 mg, 65.8% yield) as a solid. LCMS (APCI+) m/z 316 (M+H)+.

Step J: A mixture of (4aS,10aS)-2′-amino-8-methoxy-1′-methyl-1,3,4,4a,5,10a-hexahydro spiro[benzo[g]isochromene-10,4′-imidazol]-5′(1′H)-one (435 mg, 1.38 mmol) in 48% HBr was heated at 80° C. with stirring. After 6 hours, the mixture was cooled to 0° C. and poured slowly to an ice cold solution of saturated NaHCO₃. The resulting mixture was stirred for 1 hour and adjusted to a pH of about 8 with formic acid. The mixture was partitioned with 5% MeOH/DCM and 5% MeOH/EtOAc several times. The organic layers were combined, dried and concentrated in vacuo to provide the first batch of crude phenol (218 mg) as a solid. The aqueous phase was concentrated, and the inorganic salts were precipitated out with 5% IPA/DCM. The filtrate containing the product was concentrated in vacuo to provide the rest of the product. The combined batches gave crude (87% pure) (4aS,10aS)-2′-amino-8-hydroxy-1′-methyl-1,3,4,4a,5,10a-hexahydrospiro[benzo[g]isochromene-10,4′-imidazol]-5′(PH)-one (450 mg, 94% yield) as a solid. LCMS (APCI+) m/z 302 (M+H)+.

Step K: DMF dimethylacetal (429.7 μL, 3.567 mmol) was added to a solution of crude (4aS,10aS)-2′-amino-8-hydroxy-1′-methyl-1,3,4,4a,5,10a-hexahydrospiro[benzo[g]isochromene-10,4′-imidazol]-5′(1′H)-one (215 mg, 0.7135 mmol) in N,N-dimethylformamide (4757 μL, 0.7135 mmol). The mixture was stirred at ambient temperature overnight. The mixture was poured into ice water (10 mL) and partitioned with EtOAc (5×30 mL). Most of the product remained in the aqueous phase. The organic and the aqueous phases were combined and concentrated in vacuo to provide 82% pure (E)-N′-((4aS,10aS)-8-hydroxy-1′-methyl-5′-oxo-1,1′,3,4,4a,5,5′,10a-octahydrospiro[benzo[g] isochromene-10,4′-imidazol]-2′-yl)-N,N-dimethylformimidamide (310 mg, 97.5% yield) as a gum. LCMS (APCI+) m/z 357 (M+H)+.

Step L: Triethylamine (202 μL, 1.45 mmol) was sequentially added to a solution of (E)-N′-((4aS,10aS)-8-hydroxy-1′-methyl-5′-oxo-1,1′,3,4,4a,5,5′,10a-octahydro spiro[benzo[g]isochromene-10,4′-imidazole]-2′-yl)-N,N-dimethylformimidamide (258 mg, 0.724 mmol) in DCM, followed by 1,1,1-trifluoro-N-phenyl-N-(trifluoromethylsulfonyl)methanesulfonamide (388 mg, 1.1 mmol). The resulting mixture was stirred at ambient temperature for 24 hours. The mixture was poured into brine and extracted with DCM (4×30 mL). The organic layers were combined, washed with brine (20 mL) and dried (MgSO₄) and concentrated in vacuo. The crude isolated was purified by flash chromatography on silica gel (Ready Sep 40 g) eluting with a gradient of 1-25% IPA/DCM+2% aqueous NH₄OH (15CV, on Biotage SP1 unit) to provide (4aS,10aS)-2′-((E)-(dimethylamino)methyleneamino)-1′-methyl-5′-oxo-1,1′,3,4,4a,5,5′,10a-octahydrospiro[benzo[g] isochromene-10,4′-imidazole]-8-yl trifluoromethanesulfonate (198 mg, 56% yield). LCMS: (APCI)+ m/z 489 (M+H)+.

Step M: A resealable glass pressure tube was charged with (4aS,10aS)-2′-((E)-(dimethylamino)methyleneamino)-1′-methyl-5′-oxo-1,1′,3,4,4a,5,5′,10a-octahydrospiro[benzo[g]isochromene-10,4′-imidazole]-8-yl trifluoromethanesulfonate (40 mg, 0.082 mmol), 5-chloropyridin-3-ylboronic acid (19 mg, 0.12 mmol), PdCl₂(dppf) dichloromethane adduct (6.7 mg, 0.0082 mmol), 20% aqueous Na₂CO₃ (152 μL, 0.29 mmol), and 1,4-dioxane (328 μL, 0.082 mmol). The reaction mixture was sparged with N₂ for 5 minutes, capped, and stirred at 85° C. for 18 hours and allowed to cool to ambient temperature. The reaction mixture was diluted with THF (6 mL), filtered (45 micron filter) and concentrated in vacuo. The residue obtained was redissolved in THF (1 mL) and purified by C-18 reverse phase flash chromatography (Biotage 12M+) eluting with a gradient of 5-60% water/CH₃CN+0.1% TFA. The product isolated was repurified by reverse phase C-18 Prep HPLC (Gilson Unipoint) using a gradient of 5-95% CH₃CN/water+0.1% TFA to provide (4aS,10aS)-2′-amino-8-(5-chloropyridin-3-yl)-1′-methyl-1,3,4,4a,5,10a-hexahydrospiro[benzo[g]isochromene-10,4′-imidazol]-5′(1′H)-one 2,2,2-trifluoroacetate (29 mg, 69% yield) as a solid. ¹H NMR (400 MHz, CDCl₃) δ 8.754 (brs, 1H), 8.591 (brs, 1H), 8.04 (s, 1H), 7.49 (dd, J1=1.956 Hz, J2=8.216 Hz, 1H), 7.366 (d, J=7.825 Hz, 1H), 7.196 (d, J=1.565 Hz, 1H), 4.048 (dt, J1-3.913 Hz, J2=10.564 Hz, 2H), 3.49-3.99 (m 1H), 3.27 (s, 3H), 3.14-3.07 (m, 2H), 2.80-2.71 (m, 1H), 2.70-2.62 (m, 1H), 2.39-2.31 (m, 1H), 1.91-1.84 (m, 1H), 1.62-1.49 (m, 1H); LCMS (APCI+) m/z 397 (M+H)+.

### Example 70

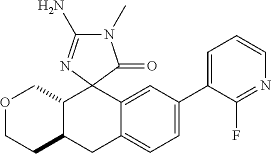

### (4aS,10aS)-2′-amino-8-(2-fluoropyridin-3-yl)-1′-methyl-1,3,4,4a,5,10a-hexahydrospiro[benzo[g]isochromene-10,4′-imidazol]-5′(1′H)-one

(4aS,10aS)-2′4E)-(Dimethylamino)methyleneamino)-1′-methyl-5′-oxo-1,1′,3,4,4a,5,5′,10a-octahydrospiro[benzo[g] isochromene-10,4′-imidazole]-8-yl trifluoromethanesulfonate (40 mg, 0.082 mmol) was processed as described for Example 69, Step M, except substituting 5-chloropyridin-3-ylboronic acid with 2-fluoropyridin-3-ylboronic acid to provide (4aS,10aS)-2′-amino-8-(2-fluoropyridin-3-yl)-1′-methyl-1,3,4,4a,5,10a-hexahydro spiro[benzo[g] isochromene-10,4′-imidazol]-5′(1′H)-one 2,2,2-trifluoroacetate (17 mg, 42% yield) as a solid. ¹H NMR (400 MHz, CDCl₃) δ 8.18 (m, 1H), 7.86-7.81 (m, 1H), 7.48-7.46 (m, 1H), 7.32 (s, 1H), 7.29-2.82 (m, 1H), 7.11 (s, 1H), 4.05-3.99 (m, 2H), 3.44-3.39 (m, 1H), 3.22 (s, 3H), 3.11-3.04 (m, 2H), 2.76-2.70 (m, 1H), 2.69-2.62 (m, 1H), 2.33 (dt, J1=4.304 Hz, J2=11.346 Hz, 1H), 1.91-1.84 (m, 1H), 1.62-1.50 (m, 1H); LCMS (APCI+) m/z 381 (M+H)+.

### Example 71

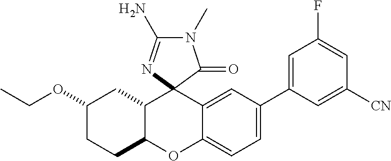

### 3-((2′S,4R,4a′S,9a′R)-2-amino-2′-ethoxy-1-methyl-5-oxo-1,1′,2′,3′,4′,4a′,5,9a′-octahydro spiro[imidazole-4,9′-xanthen]-7′-yl)-5-fluorobenzonitrile

Step A: A solution of (4a′S,9′R,9a′R)-7′-bromo-2′,2′-spiro(1,3-dioxolane)-1′,2′,3′,4′,4a′,9a′-hexahydrospiro[imidazolidine-4,9′-xanthene]-2,5-dione (12.0 g, 28.4 mmol) in 2N HCl (71 mL) and acetone (142 mL) was heated at reflux for 1 day. The reaction mixture was diluted with ethyl acetate, and the aqueous layer was extracted with ethyl acetate (3×). The combined organic layers were dried and concentrated to afford a residue that was purified by flash chromatography eluting with a 0-10% gradient of DCM and MeOH+1% NH₄OH to afford (4a′S,9a′R)-7′-bromo-1-methyl-1′,4′,4a′,9a′-tetrahydrospiro[imidazolidine-4,9′-xanthene]-2,2′,5(3′H)-trione (9.0 g, 23.7 mmol, 84%).

Step B: to a solution of ethoxytrimethylsilane (1.56 g, 13.2 mmol) and 7′-bromo-1-methyl-1′,4′,4a′,9a′-tetrahydrospiro[imidazolidine-4,9′-xanthene]-2,2′,5(3′H)-trione (1.0 g, 2.64 mmol) in DCM (25 mL) at 0° C. was added TMSOTf (2.34 mL, 13.2 mmol). The reaction mixture was stirred at 0° C. for 2 hours. Triethylsilane (2.11 ml, 13.2 mmol) was added to this mixture, and the resulting mixture was stirred at room temperature overnight. The reaction mixture was concentrated, and the residue was purified by C18 preparative HPLC to afford (2′S,4R,4a′S,9a′R)-7′-bromo-2′-ethoxy-1-methyl-1′,2′,3′,4′,4a′,9a′-hexahydrospiro[imidazolidine-4,9′-xanthene]-2,5-dione (first eluting peak, 340 mg, 0.83 mmol, 32%).

Step C: A mixture of (2′S,4R,4a′S,9a′R)-7′-bromo-2′-ethoxy-1-methyl-1′,2′,3′,4′,4a′,9a′-hexahydrospiro[imidazolidine-4,9′-xanthene]-2,5-dione (320 mg, 0.78 mmol) and Lawesson's Reagent (190 mg, 0.47 mmol) in toluene (4.0 mL) was heated at 100° C. for 24 hours. The reaction mixture was cooled to ambient temperature, diluted with ethyl acetate then washed with NaHCO₃ and brine. The organic layer was dried and concentrated to give a residue that was dissolved in methanol (5.5 mL). t-Butyl hydroperoxide (70% aqueous, 1.7 mL, 16.5 mmol) and ammonium hydroxide (1.2 mL, 33 mmol) were added to this solution, and the resulting mixture was stirred at room temperature overnight. The reaction mixture was concentrated and then extracted with ethyl acetate. The aqueous layer was extracted with ethyl acetate (3×), and the combined organic layers were dried and concentrated to give a residue that was purified by C18 preparative HPLC to afford (2′S,4R,4a′S,9a′R)-2-amino-7′-bromo-2′-ethoxy-1-methyl-1′,2′,3′,4′,4a′,9a′-hexahydrospiro[imidazole-4,9′-xanthen]-5(1H)-one (293 mg, 0.72 mmol, 92%).

Step D: A suspension of (2′S,4R,4a′S,9a′R)-2-amino-7′-bromo-2′-ethoxy-1-methyl-1′,2′,3′,4′,4a′,9a′-hexahydrospiro[imidazole-4,9′-xanthen]-5(1H)-one (37 mg, 0.089 mmol), 3-cyano-5-fluorophenylboronic acid (16 mg, 0.094 mmol), Pd(PPh₃)₄ (5.2 mg, 0.0045 mmol) and Na₂CO₃ (134 μL) in dioxane (447 μL) was degassed thoroughly with nitrogen, and the mixture was capped and heated at 95° C. overnight. The reaction mixture was diluted with MeOH, and the suspension was filtered. The filtrate was purified by C18 preparative HPLC to afford 34(2′S,4R,4a′S,9a′R)-2-amino-2′-ethoxy-1-methyl-5-oxo-1,1′,2′,3′,4′,4a′,5,9a′-octahydro spiro[imidazole-4,9′-xanthene]-7′-yl)-5-fluorobenzonitrile trifluoroacetic acid salt (10 mg, 0.022 mmol, 25% yield). ¹H NMR (CD₃OD) δ 7.84 (s, 1H), 7.72 (d, J=7.8 Hz, 1H), 7.66 (d, J=7.8 Hz, 1H), 7.54 (s, 1H), 7.49 (m, 1H), 7.05 (d, J=7.8 Hz, 1H), 4.59 (m, 1H), 3.56 (m, 2H), 3.43 (m, 1H), 3.28 (s, 3H), 2.34 (m, 1H), 2.20 (m, 2H), 2.06 (m, 1H), 1.62 (m, 1H), 1.30 (m, 1H), 1.19 (m, 3H), 0.95 (m, 1H); MS m/z (APCI-pos) M+1=448.8.

### Example 72

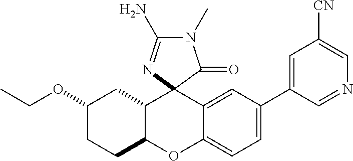

### 5-((2′S,4R,4a′S,9a′R)-2-amino-2′-ethoxy-1-methyl-5-oxo-1,1′,2′,3′,4′,4a′,5,9a′-octahydrospiro[imidazole-4,9′-xanthen]-7′-yl)nicotinonitrile

5-((2′S,4R,4a′S,9a′R)-2-Amino-2′-ethoxy-1-methyl-5-oxo-1,1′,2′,3′,4′,4a′,5,9a′-octahydrospiro[imidazole-4,9′-xanthene]-7′-yl)nicotinonitrile trifluoroacetic acid salt (14 mg, 0.032 mmol, 52% yield) was prepared according to Example 71, substituting 5-(4,4,5,5-tetramethyl-1,3,2-dioxaborolan-2-yl)nicotinonitrile for 3-cyano-5-fluorophenylboronic acid. ¹H NMR (CD₃OD) δ 9.03 (m, 1H), 8.83 (d, J=7.8 Hz, 1H), 8.43 (d, J=7.8 Hz, 1H), 7.69 (dd, J=8.2, 2.0 Hz, 1H), 7.59 (d, J=2.3 Hz, 1H), 7.08 (d, J=8.6 Hz, 1H), 4.59 (td, J=10.2, 4.3 Hz, 1H), 3.56 (m, 2H), 3.43 (m, 1H), 3.27 (s, 3H), 2.34 (m, 1H), 2.23 (m, 2H), 2.06 (m, 1H), 1.62 (m, 1H), 1.30 (m, 1H), 1.19 (t, J=7.0 Hz, 3H), 0.95 (m, 1H); MS m/z (APCI-pos) M+1=431.8.

### Example 73

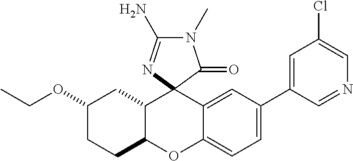

(2′S,4R,4a′S,9a′R)-2-amino-7′-(5-chloropyridin-3-yl)-2′-ethoxy-1-methyl-1′,2′,3′,4′,4a′,9a′-hexahydrospiro[imidazole-4,9′-xanthen]-5(1H)-one

(2′S,4R,4a′S,9a′R)-2-Amino-7′-(5-chloropyridin-3-yl)-2′-ethoxy-1-methyl-1′,2′,3′,4′,4a′,9a′-hexahydrospiro[imidazole-4,9′-xanthen]-5(1H)-one trifluoroacetic acid salt (19 mg, 0.043 mmol, 61% yield) was prepared according to Example 71, substituting 5-chloropyridin-3-ylboronic acid for 3-cyano-5-fluorophenylboronic acid. ¹H NMR (CD₃OD) δ 8.72 (m, 1H), 8.52 (s, 1H), 8.16 (d, J=2.0 Hz, 1H), 7.65 (dd, J=8.6, 2.3 Hz, 1H), 7.55 (d, J=2.3 Hz, 1H), 7.06 (d, J=8.6 Hz, 1H), 4.59 (td, J=11.0, 4.7 Hz, 1H), 3.56 (m, 2H), 3.43 (m, 1H), 3.27 (s, 3H), 2.34 (m, 1H), 2.23 (m, 2H), 2.06 (m, 1H), 1.62 (m, 1H), 1.30 (m, 1H), 1.17 (t, J=7.0 Hz, 3H), 0.93 (q, J=11.3 Hz, 1H); MS m/z (APCI-pos) M+1=440.8.

### Example 74

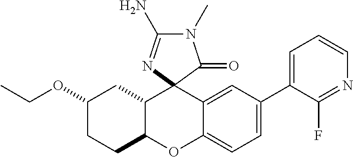

### (2′S,4R,4a′S,9a′R)-2-amino-2′-ethoxy-7′-(2-fluoropyridin-3-yl)-1-methyl-1′,2′,3′,4′,4a′,9a′-hexahydrospiro[imidazole-4,9′-xanthen]-5(1H)-one

(2′S,4R,4a′S,9a′R)-2-Amino-2′-ethoxy-7′-(2-fluoropyridin-3-yl)-1-methyl-1′,2′,3′,4′,4a′,9a′-hexahydrospiro[imidazole-4,9′-xanthen]-5(1H)-one (22 mg, 0.052 mmol, 53% yield) was prepared according to Example 71, substituting 2-fluoropyridin-3-ylboronic acid for 3-cyano-5-fluorophenylboronic acid. ¹H NMR (CD₃OD) δ 8.13 (d, J=4.7 Hz, 1H), 8.00 (m, 1H), 7.53 (m, 1H), 7.43 (m, 1H), 7.37 (m, 1H), 7.04 (d, J=8.6 Hz, 1H), 4.60 (td, J=11.0, 4.7 Hz, 1H), 3.58 (m, 2H), 3.43 (m, 1H), 3.25 (s, 3H), 2.34 (m, 1H), 2.23 (m, 2H), 2.06 (m, 1H), 1.62 (m, 1H), 1.30 (m, 1H), 1.17 (t, J=7.0 Hz, 3H), 0.94 (q, J=11.3 Hz, 1H); MS m/z (APCI-pos) M+1=424.8.

### Example 75

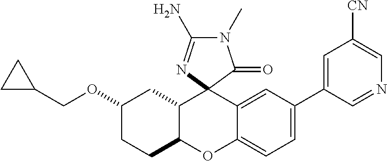

### 5-((2′S,4R,4a′S,9a′R)-2-amino-2′-(cyclopropylmethoxy)-1-methyl-5-oxo-1,1′,2′,3′,4′,4a′,5,9a′-octahydrospiro[imidazole-4,9′-xanthen]-7′-yl)nicotinonitrile

Step A: TMSOTf (2.39 ml, 13.2 mmol) was added to a solution of cyclopropylmethanol (0.953 g, 13.2 mmol) and 2,6-lutidine (1.54 mL, 13.2 mmol) in DCM (26 mL) at 0° C. The reaction mixture was stirred at 0° C. for 2 hours. 2-Amino-7′-bromo-1-methyl-1′,4′,4a′,9a′-tetrahydrospiro[imidazole-4,9′-xanthene]-2′,5(1H, 3′H)-dione (Example 71, Step A, 1.00 g, 2.644 mmol) and triethylsilane (2.111 mL, 13.22 mmol) were added to this mixture, and the resulting mixture was stirred at room temperature for 1 day. The reaction mixture was concentrated, and the residue was purified by C18 preparative HPLC to obtain (2′S,4R,4a′S,9a′R)-2-amino-7′-bromo-2′-(cyclopropylmethoxy)-1-methyl-1′,2′,3′,4′,4a′,9a′-hexahydrospiro[imidazole-4,9′-xanthen]-5(1H)-one (200 mg, 0.461 mmol, 17%).

Step B: (2′S,4R,4a′S,9a′R)-2-Amino-7′-bromo-2′-(cyclopropylmethoxy)-1-methyl-1′,2′,3′,4′,4a′,9a′-hexahydrospiro[imidazole-4,9′-xanthen]-5(1H)-one was prepared according to Example 71, Step C, substituting (2′S,4R,4a′S,9a′R)-2-amino-7′-bromo-2′-(cyclopropylmethoxy)-1-methyl-1′,2′,3′,4′,4a′,9a′-hexahydrospiro[imidazole-4,9′-xanthen]-5(1H)-one for (2′S,4R,4a′S,9a′R)-2-amino-7′-bromo-2′-ethoxy-1-methyl-1′,2′,3′,4′,4a′,9a′-hexahydrospiro[imidazole-4,9′-xanthen]-5(1H)-one (210 mg, 0.484 mmol, 48%).

Step C: 5-((2′S,4R,4a′S,9a′R)-2-Amino-2′-(cyclopropylmethoxy)-1-methyl-5-oxo-1,1′,2′,3′,4′,4a′,5,9a′-octahydrospiro[imidazole-4,9′-xanthen]-7′-yl)nicotinonitrile (6 mg, 0.013 mmol, 31% yield) was prepared according to Example 71, substituting (2′S,4R,4a′S,9a′R)-2-Amino-7′-bromo-2′-(cyclopropylmethoxy)-1-methyl-1′,2′,3′,4′,4a′,9a′-hexahydro spiro[imidazole-4,9′-xanthen]-5 (1H)-one for (2′S,4R,4a′S,9a′R)-2-amino-7′-bromo-2′-ethoxy-1-methyl-1′,2′,3′,4′,4a′,9a′-hexahydrospiro[imidazole-4,9′-xanthen]-5(1H)-one and 5-(4,4,5,5-tetramethyl-1,3,2-dioxaborolan-2-yl)nicotinonitrile for 3-cyano-5-fluorophenylboronic acid. ¹H NMR (CD₃OD) δ 9.03 (s, 1H), 8.84 (s, 1H), 8.43 (s, 1H), 7.69 (d, J=8.6 Hz, 1H), 7.59 (s, 1H), 7.08 (d, J=8.6 Hz, 1H), 4.58 (m, 1H), 3.30 (m, 2H), 3.27 (s, 3H), 2.33 (m, 1H), 2.18 (m, 4H), 2.05 (m, 1H), 1.90 (m, 214), 1.62 (m, 3H), 1.30 (m, 1H), 0.95 (m, 1H); MS m/z (APCI-pos) M+1=457.8.

### Example 76

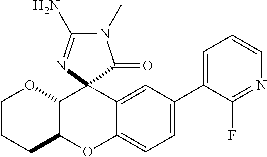

### (4S,4a′S,10a′R)-2-amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one

Step A: A 1-liter round bottom flask was charged with dihydro-2H-pyran-3(4H)-one (25.1 g, 251 mmol) and morpholine (32.8 g, 376 mmol) in toluene (500 mL). The mixture was heated to reflux with azeotropic removal of water for 4 hours and then concentrated under reduced pressure to give a quantitative yield of 4-(3,4-dihydro-2H-pyran-5-yl)morpholine and 4-(5,6-dihydro-2H-pyran-3-yl)morpholine (˜7.5:1, based on ¹H NMR analysis). m/z (APCI-pos) M+1=170.

Step B: 5-Bromo-2-hydroxybenzaldehyde (50.4 g, 251 mmol) was added to a round bottom flask containing 4-(3,4-dihydro-2H-pyran-5-yl)morpholine (42.4 g, 251 mmol) in toluene (500 mL). This mixture was stirred at room temperature for 16 hours and then concentrated under reduced pressure to yield 8-bromo-4-a-morpholino-2,3,4,4a,10,10a-hexahydropyrano[3,2-b]chromen-10-ol as an oil. This material was used as is in the next step. m/z (APCI-pos) M+1=369.9 and 371.9.

Step C: Dess-Martin periodinane (138 g, 326 mmol) was added to a round bottom flask containing 8-bromo-4-a-morpholino-2,3,4,4a,10,10a-hexahydropyrano[3,2-b]chromen-10-ol (92.8 g, 251 mmol) in dichloromethane (1 L) chilled to 0° C. This mixture was allowed to gradually warm to room temperature over a 16 hour period. 2M Aqueous sodium carbonate (1.5 liters) and 25% IPA/DCM (500 mL) were then added to the reaction mixture and stirred vigorously for 20 minutes. The resulting solids were removed by filtration, and the organics were isolated from the filtrate. The filtrate was extracted once more with 25% IPA/DCM, and the organics were combined, dried over sodium sulfate and concentrated under reduced pressure. The crude product was purified by flash chromatography (eluting with DCM to 10% ethyl acetate:DCM) to give 8-bromo-3,4-dihydropyrano[3,2-b]chromen-10(2H)-one (22.4 g, 32% yield).

Step D: A round bottom flask was charged with 8-bromo-3,4-dihydropyrano[3,2-b]chromen-10(2H)-one (17 g, 60.5 mmol) and dry THF (600 mL). This mixture was chilled to −78° C., and L-Selectride (72.6 mL, 72.6 mmol, 1M in THF) was then added slowly by syringe. After 1 hour at −78° C., additional L-Selectride (20 mL) was added to the reaction mixture, and it was stirred for 1 hour. Saturated ammonium chloride solution (250 mL) was then added to the reaction mixture, which was then allowed to warm to room temperature. The mixture was then extracted with EtOAc (2×), and the extracts were dried over sodium sulfate and concentrated under reduced pressure. The crude product was purified by flash chromatography (30% ethyl acetate:hexanes) to give (4aR,10aR)-8-bromo-2,3,4,4a-tetrahydropyrano[3,2-b]chromen-10(10aH)-one (8.1 g, 47%) as a pure trans diastereomer.

Step E: A stainless steel bomb containing a teflon insert was charged with EtOH (17 mL) and (4aR,10aR)-8-bromo-2,3,4,4a-tetrahydropyrano[3,2-b]chromen-10(10aH)-one (7.4 g, 26.1 mmol). Next, ammonium carbonate (25.1 g, 261 mmol), KCN (2.3 g, 35.3 mmol) and sodium hydrogensulfite (680 mg, 6.53 mmol) were added. The reaction mixture was heated to 150° C. for 16 hours with stirring. The contents of the teflon insert were then removed by rinsing with water (about 200 mL). The contents were extracted with EtOAc (2×), and the extracts were dried over sodium sulfate and concentrated under reduced pressure. The crude material was purified by flash chromatography to give 8′-bromo-3′,4′,4a′,10a′-tetrahydro-2′H-spiro[imidazolidine-4,10′-pyrano[3,2-b]chromene]-2,5-dione, as the trans (ring junction) diastereomers (2.5 g), a mixture of trans and cis diastereomers (2.0 g), and cis diastereomers (2.15 g), an overall 71% yield.

Step F: A round bottom flask was charged with a mixture of (4S,4a′S,10a′R)-8′-bromo-3′,4′,4a′,10a′-tetrahydro-2′H-spiro[imidazolidine-4,10′-pyrano[3,2-b]chromene]-2,5-dione and (4R,4a′S,10a′R)-8′-bromo-3′,4′,4a′,10a′-tetrahydro-2′H-spiro[imidazolidine-4,10′-pyrano[3,2-b]chromene]-2,5-dione (2.48 g, 7.02 mmol) and dry DMF (70 mL). Powdered potassium carbonate (1.46 g, 10.5 mmol) and MeI (0.997 g, 7.02 mmol) were added to the reaction mixture, and the mixture was stirred at ambient temperature for one hour. The mixture was then diluted with brine (200 mL) and then extracted with EtOAc (2×). The extracts were washed with brine, dried over sodium sulfate and concentrated under reduced pressure. The crude product was triturated with diethyl ether and filtered to provide a solid (500 mg) that by ¹H NMR is consistent with (4R,4a′S,10aR)-8′-bromo-1-methyl-3′,4′,4a′,10a′-tetrahydro-2′H-spiro[imidazolidine-4,10′-pyrano[3,2-b]chromene]-2,5-dione. The mother liquor from the filtration was purified by flash chromatography to give a foam (1.03 g, 40%) that is consistent with (4S,4a′S,10a′R)-8′-bromo-1-methyl-3′,4′,4a′,10a′-tetrahydro-2′H-spiro[imidazolidine-4,10′-pyrano[3,2-b]chromene]-2,5-dione (by ¹H NMR).

Step G: (4S,4a′S,10a′R)-8′-Bromo-1-methyl-3′,4′,4a′,10a′-tetrahydro-2′H-spiro[imidazolidine-4,10′-pyrano[3,2-b]chromene]-2,5-dione (750 mg, 2.04 mmol), dry toluene (20 mL), and Lawesson's reagent (578 mg, 1.43 mmol) were added to a heavy walled pressure tube. The tube was sealed and heated to 115° C. for 16 hours. After cooling to room temperature, the mixture was diluted with EtOAc, washed with saturated sodium bicarbonate solution (2×), dried over sodium sulfate and concentrated under reduced pressure to a quantitative recovery of crude (4S,4a′S,10a′R)-8′-bromo-1-methyl-2-thioxo-3′,4′,4a′,10a′-tetrahydro-2′H-spiro[imidazolidine-4,10′-pyrano[3,2-b]chromen]-5-one. This was carried onto the next step as is.

Step H: A pressure tube was charged with (4S,4a′S,10a′R)-8′-bromo-1-methyl-2-thioxo-3′,4′,4a′,10a′-tetrahydro-2′H-spiro[imidazolidine-4,10′-pyrano[3,2-b]chromen]-5-one (783 mg, 2.04 mmol), methanol (20 mL), 70% aqueous t-butyl hydroperoxide (2.83 mL, 20.4 mmol), and 30% ammonium hydroxide (5.3 mL, 40.9 mmol). The tube was sealed and warmed to 50° C. for 16 hours. The mixture was then diluted with EtOAc (100 mL), washed with 10% aqueous sodium thiosulfate, brine, dried over sodium sulfate and concentrated under reduced pressure. The crude product was purified by flash chromatography to give (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (277 mg, 37%). m/z (APCI-pos) M+1=366.1, 368.1.

Step I: A heavy walled pressure tube was charged with (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (250 mg, 0.683 mmol), 2-fluoropyridin-3-ylboronic acid (125 mg, 0.887 mmol), Pd(PPh₃)₄ (0.079 mg, 0.0683 mmol), 2M aqueous potassium carbonate (0.853 mL, 1.71 mmol) in dioxane (7 mL). This mixture was purged with argon for 5 minutes, the tube was sealed and heated to 100° C. for 16 hours. The mixture was diluted with EtOAc and washed with water, and the organics were dried over sodium sulfate and concentrated under reduced pressure. The crude product was purified by preparative thin layer chromatography to give (4S,4a′S,10a′R)-2-amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one (20 mg, 7.7%). ¹H NMR (400 MHz, CD₃OD) δ 8.13-8.10 (m, 1H), 7.98-7.92 (m, 1H), 7.46-7.42 (m, 1H), 7.38-7.33 (m, 1H), 7.17-7.14 (m, 1H), 7.00-6.96 (m, 1H), 4.78-4.69 (m, 1H), 3.94-3.88 (m, 1H), 3.59-3.55 (m, 1H), 3.48-3.38 (m, 1H), 3.06 (s, 3H), 2.39-2.30 (m, 1H), 1.81-1.64 (m, 3H); m/z (APCI-pos) M+1=383.1.

### Example 77

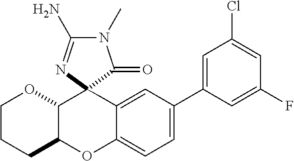

### (4S,4a′S,10a′R)-2-amino-8′-(3-chloro-5-fluorophenyl)-1-methyl-3′,4′,4a′,10a′-tetrahydro-2′H-spiro[imidazole-4,10′-pyrano[3,2-b]chromen]-5(1H)-one

A reaction vial was charged with (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (30 mg, 0.082 mmol), 3-chloro-5-fluorophenylboronic acid (57 mg, 0.328 mmol), Pd(PPh₃)₄ (9 mg, 0.008 mmol), 2M aqueous potassium carbonate (0.123 mL, 0.246 mmol) in dioxane (1 mL). This mixture was purged with argon for 5 minutes, and the vial was sealed and heated to 100° C. for 16 hours. The mixture was diluted with EtOAc and washed with water, and the organics were dried over sodium sulfate and concentrated under reduced pressure. The crude product was purified by preparative thin layer chromatography to give (4S,4a′S,10a′R)-2-amino-8′-(3-chloro-5-fluorophenyl)-1-methyl-3′,4′,4a′,10a′-tetrahydro-2′H-spiro[imidazole-4,10′-pyrano[3,2-b]chromen]-5(1H)-one (10 mg, 29%). ¹H NMR (400 MHz, CDCl₃) δ 7.38-7.32 (m, 1H), 7.28-7.22 (m, 2H), 7.16-6.92 (m, 3H), 4.81-4.73 (m, 1H), 3.96-3.86 (m, 1H), 3.46-3.40 (m, 1H), 3.35-3.24 (m, 1H), 3.11 (s, 3H), 2.38-2.30 (m, 1H), 1.83-1.53 (m, 3H); m/z (APCI-pos) M+1=416.2.

### Example 78

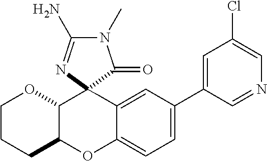

### (4S,4a′S,10a′R)-2-amino-8′-(5-chloropyridin-3-yl)-1-methyl-3′,4′,4a′,10a′-tetrahydro-2′H-spiro[imidazole-4,10′-pyrano[3,2-b]chromen]-5(1H)-one

A reaction vial was charged with (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (25 mg, 0.068 mmol), 5-chloropyridin-3-ylboronic acid (22 mg, 0.137 mmol), Pd(PPh₃)₄ (8 mg, 0.007 mmol), 2M aqueous potassium carbonate (0.102 mL, 0.205 mmol) in dioxane (1 mL). This mixture was purged with argon for 5 minutes, and the vial was sealed and heated to 100° C. for 16 hours. The mixture was diluted with EtOAc and washed with water, and the organics were dried over sodium sulfate and concentrated under reduced pressure. The crude product was purified by preparative thin layer chromatography to give (4S,4a′S,10a′R)-2-amino-8′-(5-chloropyridin-3-yl)-1-methyl-3′,4′,4a′,10a′-tetrahydro-2′H-spiro[imidazole-4,10′-pyrano[3,2-b]chromen]-5(1H)-one (8 mg, 29%). ¹H NMR (400 MHz, CDCl₃) δ 8.62-8.58 (m, 1H), 8.50-8.46 (m, 1H), 7.77-7.73 (m, 1H), 7.42-7.36 (m, 1H), 7.17-7.12 (m, 1H), 7.01-6.96 (m, 1H), 4.84-4.73 (m, 1H), 3.97-3.88 (m, 1H), 3.53-3.47 (m, 1H), 3.38-3.28 (m, 1H), 3.12 (s, 3H), 2.39-2.30 (m, 1H), 1.83-1.53 (m, 3H); m/z (APCI-pos) M+1=399.1, 401.1.

### Example 79

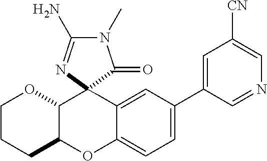

### 5-((4S,4a′S,10a′R)-2-amino-1-methyl-5-oxo-1,3′,4′,4a′,5,10a′-hexahydro-2′H-spiro[imidazole-4,10′-pyrano[3,2-b]chromen]-8′-yl)nicotinonitrile

A reaction vial was charged with (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (30 mg, 0.082 mmol), 5-(4,4,5,5-tetramethyl-1,3,2-dioxaborolan-2-yl)nicotinonitrile (38 mg, 0.164 mmol), Pd(PPh₃)₄ (9.5 mg, 0.008 mmol), 2M aqueous potassium carbonate (0.123 mL, 0.246 mmol) in dioxane (1 mL). This mixture was purged with argon for 5 minutes, and the vial was sealed and heated to 100° C. for 16 hours. The mixture was diluted with EtOAc and washed with water, and the organics were dried over sodium sulfate and concentrated under reduced pressure. The crude product was purified by preparative thin layer chromatography to give 5-04S,4a′S,10a′R)-2-amino-1-methyl-5-oxo-1,3′,4′,4a′,5,10a′-hexahydro-2′H-spiro[imidazole-4,10′-pyrano[3,2-b]chromen]-8′-yl)nicotinonitrile (13 mg, 41%). ¹H NMR (400 MHz, CDCl₃) δ 8.92-8.90 (m, 1H), 8.79-8.77 (m, 1H), 8.03-8.00 (m, 1H), 7.42-7.37 (m, 1H), 7.16-7.12 (m, 1H), 7.04-6.99 (m, 1H), 4.86-4.77 (m, 1H), 3.96-3.90 (m, 1H), 3.55-3.50 (m, 1H), 3.40-3.31 (m, 1H), 3.13 (s, 3H), 2.41-2.33 (m, 1H), 1.86-1.55 (m, 3H); m/z (APCI-pos) M+1=390.1.

### Example 80

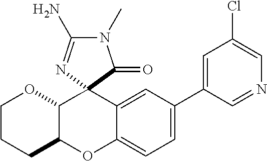

### (4R,4a′S,10a′R)-2-amino-8′-(5-chloropyridin-3-yl)-1-methyl-3′,4′,4a′,10a′-tetrahydro-2′H-spiro[imidazole-4,10′-pyrano[3,2-b]chromen]-5(1H)-one

(4R,4a′S,10a′R)-2-Amino-8′-(5-chloropyridin-3-yl)-1-methyl-3′,4′,4a′,10a′-tetrahydro-2′H-spiro[imidazole-4,10′-pyrano[3,2-b]chromen]-5(1H)-one (2.7 mg, 12%) was prepared according to Example 76, Step I, substituting (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one for (4R,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (from Example 76, Step E) and 5-chloropyridin-3-ylboronic acid for 2-fluoropyridin-3-ylboronic acid. m/z (APCI-pos) M+1=399.1, 401.1.

### Example 81

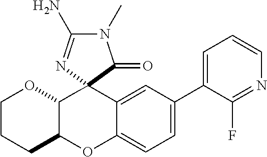

### (4R,4a′S,10a′R)-2-amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one

(4R,4a′S,10a′R)-2-Amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one (4 mg, 19%) was prepared according to Example 76, Step I, substituting (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one for (4R,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one (from Example 76, Step E). m/z (APCI-pos) M+1=383.2.

### Example 82

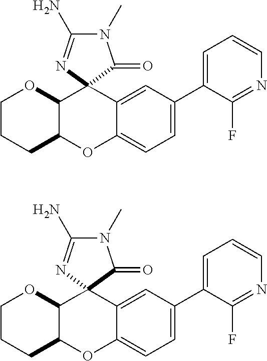

### (4S,4a′S,10a′S)-2-amino-8′-(2-fluoropyridin-3-yl)-1-methyl-3′,4′,4a′,10a′-tetrahydro-2′H-spiro[imidazole-4,10′-pyrano[3,2-b]chromen]-5 (1H)-one

### (4R,4a′S,10a′S)-2-amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one

Step A: A round bottom flask was charged with a mixture of (4S,4a′S,10a′S)-8′-bromo-3′,4′,4a′,10a′-tetrahydro-2′H-spiro[imidazolidine-4,10′-pyrano[3,2-b]chromene]-2,5-dione and (4R,4a′S,10a′S)-8′-bromo-3′,4′,4a′,10a′-tetrahydro-2′H-spiro[imidazolidine-4,10′-pyrano[3,2-b]chromene]-2,5-dione (200 mg, 0.566 mmol) and dry DMF (6 mL). Powdered potassium carbonate (117 mg, 0.849 mmol) and MeI (80 mg, 0.566 mmol) were added to the reaction mixture. This mixture was stirred at ambient temperature for 48 hours, then diluted with brine and extracted with EtOAc (2×). The extracts were washed once with brine, dried over sodium sulfate and concentrated under reduced pressure to give (4S,4a′S,10a′S)-8′-bromo-1-methyl-3′,4′,4a′,10a′-tetrahydro-2′H-spiro[imidazolidine-4,10′-pyrano[3,2-b]chromene]-2,5-dione and (4R,4a′S,10a′S)-8′-bromo-1-methyl-3′,4′,4a′,10a′-tetrahydro-2′H-spiro[imidazolidine-4,10′-pyrano[3,2-b]chromene]-2,5-dione (195 mg, 94%) as a mixture of diastereomers. This material was carried onto the next step as is.

Step B: A pressure tube was charged with (4S,4a′S,10a′S)-8′-bromo-1-methyl-3′,4′,4a′,10a′-tetrahydro-2′H-spiro[imidazolidine-4,10′-pyrano[3,2-b]chromene]-2,5-dione and (4R,4a′S,10a′S)-8′-bromo-1-methyl-3′,4′,4a′,10a′-tetrahydro-2′H-spiro[imidazolidine-4,10′-pyrano[3,2-b]chromene]-2,5-dione (195 mg, 0.531 mmol), Lawesson's reagent (129 mg, 0.319 mmol) and dry toluene (5 mL). The tube was sealed and heated to 90° C. for 16 hours, and then allowed to cool to ambient temperature. The mixture was concentrated under reduced pressure to give a quantitative recovery of crude (4S,4a′S,10a′S)-8′-bromo-1-methyl-2-thioxo-3′,4′,4a′,10a′-tetrahydro-2′H-spiro[imidazolidine-4,10′-pyrano[3,2-b]chromen]-5-one and (4R,4a′S,10a′S)-8′-bromo-1-methyl-2-thioxo-3′,4′,4a′,10a′-tetrahydro-2′H-spiro[imidazolidine-4,10′-pyrano[3,2-b]chromen]-5-one, which was taken onto the next step as is.

Step C: A pressure tube was charged with (4S,4a′S,10a′S)-8′-bromo-1-methyl-2-thioxo-3′,4′,4a′,10a′-tetrahydro-2′H-spiro[imidazolidine-4,10′-pyrano[3,2-b]chromen]-5-one and (4R,4a′S,10a′S)-8′-bromo-1-methyl-2-thioxo-3′,4′,4a′,10a′-tetrahydro-2′H-spiro[imidazolidine-4,10′-pyrano[3,2-b]chromen]-5-one (200 mg, 0.522 mmol), methanol (5 mL), 70% aqueous t-butyl hydroperoxide (0.722 mL, 5.22 mmol), and 30% ammonium hydroxide (1.35 mL, 35.1 mmol). The tube was sealed and warmed to 50° C. for 16 hours. The mixture was then diluted with EtOAc, washed with 10% aqueous sodium thiosulfate, brine, dried over sodium sulfate and concentrated under reduced pressure. The crude product was purified by preparative thin layer chromatography to give (4S,4a′S,10a′S)-2-amino-8′-bromo-1-methyl-3′,4′,4a′,10a′-tetrahydro-2′H-spiro[imidazole-4,10′-pyrano[3,2-b]chromen]-5(1H)-one and (4R,4a′S,10a′S)-2-amino-8′-bromo-1-methyl-3′,4′,4a′,10a′-tetrahydro-2′H-spiro[imidazole-4,10′-pyrano[3,2-b]chromen]-5(1H)-one (25 mg, 13%), as a mixture of diastereomers. m/z (APCI-pos) M+1=366.2, 368.2.

Step D: A pressure tube was charged with (4S,4a′S,10a′S)-2-amino-8′-bromo-1-methyl-3′,4′,4a′,10a′-tetrahydro-2′H-spiro[imidazole-4,10′-pyrano[3,2-b]chromen]-5(1H)-one and (4R,4a′S,10a′S)-2-amino-8′-bromo-1-methyl-3′,4′,4a′,10a′-tetrahydro-2′H-spiro[imidazole-4,10′-pyrano[3,2-b]chromen]-5(1H)-one (25 mg, 0.068 mmole), 2-fluoropyridin-3-ylboronic acid (29 mg, 0.205 mmol), Pd(PPh₃)₄ (8 mg, 0.007 mmol), 2M aqueous potassium carbonate (0.102 mL, 0.205 mmol) in dioxane (1 mL). This mixture was purged with argon for 5 minutes, and the vial was sealed and heated to 100° C. for 16 hours. The mixture was diluted with EtOAc and washed with water, and the organics were dried over sodium sulfate and concentrated under reduced pressure. The crude product was purified by preparative thin layer chromatography to give a less polar diastereomer ((4S,4a′S,10a′S)-2-amino-8′-(2-fluoropyridin-3-yl)-1-methyl-3′,4′,4a′,10a′-tetrahydro-2′H-spiro[imidazole-4,10′-pyrano[3,2-b]chromen]-5(1H)-one) (3 mg) and a more polar diastereomer ((4R,4a′S,10a′S)-2-amino-8′42-fluoropyridin-3-yl)-1-methyl-3′,4′,4a′,10a′-tetrahydro-2′H-spiro[imidazole-4,10′-pyrano[3,2-b]chromen]-5(1H)-one (2.8 mg).

Less polar diastereomer: ¹H NMR (400 MHz, CDCl₃) δ 8.15-8.12 (m, 1H), 7.80-7.74 (m, 1H), 7.48-7.43 (m, 1H), 7.39-7.36 (m, 1H), 7.25-7.21 (m, 1H), 7.05-7.02 (m, 1H), 4.86-4.83 (m, 1H), 4.09-4.03 (m, 1H), 3.62-3.52 (m, 2H), 3.12 (s, 3H), 2.33-2.25 (m, 1H), 2.16-2.05 (m, 1H), 1.81-1.71 (m, 1H), 1.50-1.44 (m, 1H); m/z (APCI-pos) M+1=383.2.

More polar diastereomer: ¹H NMR (400 MHz, CDCl₃) δ 8.18-8.12 (m, 1H), 7.82-7.75 (m, 1H), 7.50-7.43 (m, 1H), 7.40-7.35 (m, 1H), 7.26-7.21 (m, 1H), 7.07-7.01 (m, 1H), 4.86-4.82 (m, 1H), 4.09-4.02 (m, 1H), 3.63-3.52 (m, 2H), 3.13 (s, 3H), 2.34-2.26 (m, 1H), 2.17-2.03 (m, 1H), 1.81-1.72 (m, 1H), 1.53-1.43 (m, 1H); m/z (APCI-pos) M+1=383.2.

### Example 83

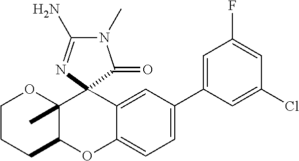

### (4S,4a′S,10a′R)-2-amino-8′-(3-chloro-5-fluorophenyl)-1,10a′-dimethyl-3′,4′,4a′,10a′-tetrahydro-2′H-spiro[imidazole-4,10′-pyrano[3,2-b]chromen]-5(1H)-one

Step A: 8-Methoxy-4-a-morpholino-2,3,4,4a,10,10a-hexahydropyrano[3,2-b]chromen-10-ol (100%) was prepared according to Example 76, Step B, substituting 2-hydroxy-5-methoxybenzaldehyde for 5-bromo-2-hydroxybenzaldehyde.

Step B: 8-Methoxy-3,4-dihydropyrano[3,2-b]chromen-10(2H)-one (41%) was prepared according to Example 76, Step C, substituting 8-methoxy-4-a-morpholino-2,3,4,4a,10,10a-hexahydropyrano[3,2-b]chromen-10-ol for 8-bromo-4-a-morpholino-2,3,4,4a,10,10a-hexahydropyrano[3,2-b]chromen-10-ol.

Step C: (4aS,10aS)-8-Methoxy-2,3,4,4a-tetrahydropyrano[3,2-b]chromen-10(10aH)-one (62%) was prepared according to Example 76, Step D, substituting 8-methoxy-3,4-dihydropyrano[3,2-b]chromen-10(2H)-one for 8-bromo-3,4-dihydropyrano[3,2-b]chromen-10(2H)-one.

Step D: A round bottom flask was charged with (4aS,10aS)-8-methoxy-2,3,4,4a-tetrahydropyrano[3,2-b]chromen-10(10aH)-one (1.03 g, 4.397 mmol) and MeI (2.5 g, 17.59 mmol) in dry THF (45 mL). This mixture was chilled to −78° C., and potassium t-butoxide (11 mL, 11 mmol, 1M in THF) was then added by syringe over a 5 minute period. Once the addition was complete, the mixture was stirred at −78° C. for 1 hour, then allowed to warm to −20° C. and stirred for 1 hour. The mixture was then quenched with saturated ammonium chloride solution (100 mL) and allowed to warm to ambient temperature. Water (100 mL) was then added, and the mixture was extracted with EtOAc (2×), dried over sodium sulfate and concentrated under reduced pressure. The crude product was purified by flash chromatography to give (4aS,10aS)-8-methoxy-10a-methyl-2,3,4,4a-tetrahydropyrano[3,2-b]chromen-10(10aH)-one (679 mg, 62%) as one diastereomer.

Step E: (4S,4a′S,10a′R)-8′-Methoxy-10a′-methyl-3′,4′,4a′,10a′-tetrahydro-2′H-spiro[imidazolidine-4,10′-pyrano[3,2-b]chromene]-2,5-dione (76%), as one predominantly one distereomer, was prepared according to Example 76, Step E, substituting (4aS,10aS)-8-methoxy-10a-methyl-2,3,4,4a-tetrahydropyrano[3,2-b]chromen-10(10aH)-one for (4aR,10aR)-8-bromo-2,3,4,4a-tetrahydropyrano[3,2-b]chromen-10(10aH)-one. This was taken onto the next step as is.

Step F: (4S,4a′S,10a′R)-8′-Methoxy-1,10a′-dimethyl-3′,4′,4a′,10a′-tetrahydro-2′H-spiro[imidazolidine-4,10′-pyrano[3,2-b]chromene]-2,5-dione (91%) was prepared according to Example 76, Step F, substituting (4S,4a′S,10a′R)-8′-methoxy-10a′-methyl-3′,4′,4a′,10a′-tetrahydro-2′H-spiro[imidazolidine-4,10′-pyrano[3,2-b]chromene]-2,5-dione for (4S,4a′S,10a′R)-8′-bromo-3′,4′,4a′,10a′-tetrahydro-2′H-spiro[imidazolidine-4,10′-pyrano[3,2-b]chromene]-2,5-dione and (4R,4a′S,10a′R)-8′-bromo-3′,4′,4a′,10a′-tetrahydro-2′H-spiro[imidazolidine-4,10′-pyrano[3,2-b]chromene]-2,5-dione. This was taken onto the next step as is.

Step G: (4S,4a′S,10a′R)-8′-Methoxy-1,10a′-dimethyl-2-thioxo-3′,4′,4a′,10a′-tetrahydro-2′H-spiro[imidazolidine-4,10′-pyrano[3,2-b]chromen]-5-one (51%) was prepared according to Example 76, Step G, substituting (4S,4a′S,10a′R)-8′-methoxy-1,10a′-dimethyl-3′,4′,4a′,10a′-tetrahydro-2′H-spiro[imidazolidine-4,10′-pyrano[3,2-b]chromene]-2,5-dione for (4S,4a′S,10a′R)-8′-bromo-1-methyl-3′,4′,4a′,10a′-tetrahydro-2′H-spiro[imidazolidine-4,10′-pyrano[3,2-b]chromene]-2,5-dione.

Step H: (4S,4a′S,10a′R)-2-Amino-8′-methoxy-1,10a′-dimethyl-3′,4′,4a′,10a'-tetrahydro-2′H-spiro[imidazole-4,10′-pyrano[3,2-b]chromen]-5(1H)-one (99%) was prepared according to Example 76, Step H, substituting (4S,4a′S,10a′R)-8′-methoxy-1,10a′-dimethyl-2-thioxo-3′,4′,4a′,10a′-tetrahydro-2′H-spiro[imidazolidine-4,10′-pyrano[3,2-b]chromen]-5-one for (4S,4a′S,10a′R)-8′-bromo-1-methyl-2-thioxo-3′,4′,4a′,10a′-tetrahydro-2′H-spiro[imidazolidine-4,10′-pyrano[3,2-b]chromen]-5-one.

Step I: A round bottom flask was charged with (4S,4a′S,10a′R)-2-amino-8′-methoxy-1,10a′-dimethyl-3′,4′,4a′,10a′-tetrahydro-2′H-spiro[imidazole-4,10′-pyrano[3,2-b]chromen]-5(1H)-one (125 mg, 0.377 mmol) and dry DCM (4 mL) This mixture was chilled to 0° C., and boron tribromide (0.754 mL, 0.754 mmol, 1M in DCM) was then added by syringe. This mixture was stirred at 0° C. for 15 minutes, then allowed to warm to room temperature and stirred for 1 hour. The reaction was then quenched with ice and then diluted with 25% IPA/DCM (50 mL). This was washed twice with saturated sodium bicarbonate solution, dried over sodium sulfate and concentrated under reduced pressure to give crude (4S,4a′S,10a′R)-2-amino-8′-hydroxy-1,10a′-dimethyl-3′,4′,4a′,10a′-tetrahydro-2′H-spiro[imidazole-4,10′-pyrano[3,2-b]chromen]-5(1H)-one (114 mg, 95%).

Step J: A round bottom flask containing (4S,4a′S,10a′R)-2-amino-8′-hydroxy-1,10a′-dimethyl-3′,4′,4a′,10a′-tetrahydro-2′H-spiro[imidazole-4,10′-pyrano[3,2-b]chromen]-5(1H)-one (114 mg, 0.359 mmol) and dimethyl formamide dimethylacetal (214 mg, 1.80 mmol) in dry DMF (4 mL) was stirred at room temperature for 16 hours. This mixture was then concentrated under reduced pressure to give crude (E)-N′-((4S,4a′S,10a′R)-8′-hydroxy-1,10a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide (134 mg, 100%).

Step K: A round bottom flask was charged with (E)-N′-((4S,4a′S,10a′R)-8′-hydroxy-1,10a′-dimethyl-5-oxo-1,3′,4′,4a′,5,10a′-hexahydro-2′H-spiro[imidazole-4,10′-pyrano[3,2-b]chromen]-2-yl)-N,N-dimethylformimidamide (134 mg, 0.360 mmol), TEA (0.100 mL, 0.720 mmol) in dry DCM (4 mL). 1,1,1-Trifluoro-N-phenyl-N-(trifluoromethylsulfonyl)methanesulfonamide was added to the reaction mixture, and the mixture was stirred at room temperature for 4 hours. The mixture was then diluted with 10% aqueous potassium carbonate (20 mL), extracted with DCM (2×), dried over sodium sulfate and concentrated under reduced pressure. The crude product was purified by preparative thin layer chromatography to give (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate (90 mg, 50%).

Step L: (4S,4a′S,10a′R)-2-Amino-8′-(3-chloro-5-fluorophenyl)-1,10a'-dimethyl-3′,4′,4a′,10a′-tetrahydro-2′H-spiro[imidazole-4,10′-pyrano[3,2-b]chromen]-5(1H)-one (10%) was prepared according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one and 3-chloro-5-fluorophenylboronic acid for 2-fluoropyridin-3-ylboronic acid. ¹H NMR (400 MHz, CDCl₃) δ 7.43-7.38 (m, 1H), 7.25-7.21 (m, 1H), 7.13-7.09 (m, 1H), 7.09-7.04 (m, 1H), 7.01-6.98 (m, 1H), 6.97-6.94 (m, 1H), 5.26-5.20 (m, 1H), 3.73-3.61 (m, 2H), 3.12 (s, 3H), 2.14-1.62 (m, 4H), 1.34 (s, 3H); m/z (APCI-pos) M+1=430.2.

### Example 84

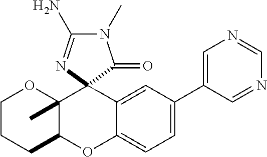

### (4S,4a′S,10a′R)-2-amino-1,10a′-dimethyl-8′-(pyrimidin-5-yl)-3′,4′,4a′,10a′-tetrahydro-2′H-spiro[imidazole-4,10′-pyrano[3,2-b]chromen]-5(1H)-one

(4S,4a′S,10a′R)-2-Amino-1,10a′-dimethyl-8′-(pyrimidin-5-yl)-3′,4′,4a′,10a′-tetrahydro-2′H-spiro[imidazole-4,10′-pyrano[3,2-b]chromen]-5(1H)-one (12%) was prepared according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one and pyrimidin-5-ylboronic acid for 2-fluoropyridin-3-ylboronic acid. ¹H NMR (400 MHz, CDCl₃) δ 9.13 (s, 1H), 8.84 (s, 2H), 7.43-7.38 (m, 1H), 7.17-7.11 (m, 1H), 7.05-7.01 (m, 1H), 5.28-5.21 (m, 1H), 3.73-3.58 (m, 2H), 3.09 (s, 3H), 2.13-1.69 (m, 4H), 1.32 (s, 3H); m/z (APCI-pos) M+1=380.2.

### Example 85

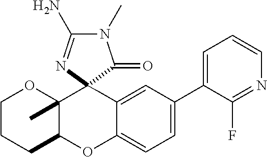

### (4S,4a′S,10a′R)-2-amino-8′-(2-fluoropyridin-3-yl)-1,10a′-dimethyl-3′,4′,4a′,10a′-tetrahydro-2′H-spiro[imidazole-4,10′-pyrano[3,2-b]chromen]-5(1H)-one

(4S,4a′S,10a′R)-2-Amino-8′-(2-fluoropyridin-3-yl)-1,10a′-dimethyl-3′,4′,4a′,10a′-tetrahydro-2′H-spiro[imidazole-4,10′-pyrano[3,2-b]chromen]-5(1H)-one (20%) was prepared according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one. ¹H NMR (400 MHz, CDCl₃) δ 8.13 (s, 1H), 7.80-7.74 (m, 1H), 7.42-7.37 (m, 1H), 7.23-7.15 (m, 2H), 6.99-6.95 (m, 1H), 5.28-5.21 (m, 1H), 3.72-3.60 (m, 2H), 3.09 (s, 3H), 2.16-1.69 (m, 4H), 1.32 (s, 3H); m/z (APCI-pos) M+1=397.2.

### Example 86

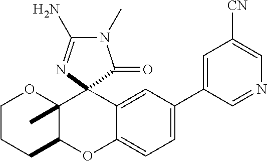

### 5-((4S,4a′S,10a′R)-2-amino-1,10a′-dimethyl-5-oxo-1,3′,4′,4a′,5,10a′-hexahydro-2′H-spiro[imidazole-4,10′-pyrano[3,2-b]chromen]-8′-yl)nicotinonitrile

5-(4S,4a′S,10a′R)-2-Amino-1,10a′-dimethyl-5-oxo-1,3′,4′,4a′,5,10a'-hexahydro-2′H-spiro[imidazole-4,10′-pyrano[3,2-b]chromen]-8′-yl)nicotinonitrile (29%) was prepared according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one and 5-(4,4,5,5-tetramethyl-1,3,2-dioxaborolan-2-yl)nicotinonitrile for 2-fluoropyridin-3-ylboronic acid. ¹H NMR (400 MHz, CDCl₃) δ 8.94-8.91 (m, 1H), 8.79-8.77 (m, 1H), 8.03-8.00 (m, 1H), 7.42-7.37 (m, 1H), 7.15-7.10 (m, 1H), 7.05-7.01 (m, 1H), 5.27-5.22 (m, 1H), 3.70-3.60 (m, 2H), 3.12 (s, 3H), 2.17-2.07 (m, 1H), 1.96-1.68 (m, 3H), 1.32 (s, 3H); m/z (APCI-pos) M+1=404.2.

### Example 87

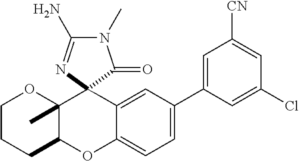

### 3-((4S,4a′S,10a′R)-2-amino-1,10a′-dimethyl-5-oxo-1,3′,4′,4a′,5,10a′-hexahydro-2′H-spiro[imidazole-4,10′-pyrano[3,2-b]chromen]-8′-yl)-5-chlorobenzonitrile

3-((4S,4a′S,10a′R)-2-Amino-1,10a′-dimethyl-5-oxo-1,3′,4′,4a′,5,10a'-hexahydro-2′H-spiro[imidazole-4,10′-pyrano[3,2-b]chromen]-8′-yl)-5-chlorobenzonitrile (19%) was prepared, as a mono TFA salt after reverse phase HPLC purification, according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one and 3-((4S,4a′S,10a′R)-2-amino-1,10a′-dimethyl-5-oxo-1,3′,4′,4a′,5,10a′-hexahydro-2′H-spiro[imidazole-4,10′-pyrano[3,2-b]chromen]-8′-yl)-5-chlorobenzonitrile for 2-fluoropyridin-3-ylboronic acid. ¹H NMR (400 MHz, CD₃OD) δ 7.98-7.91 (m, 2H), 7.75-7.63 (m, 2H), 7.58-7.54 (m, 1H), 7.10-7.04 (m, 1H), 3.72-3.59 (m, 2H), 3.20 (s, 3H), 2.15-1.85 (m, 2H), 1.81-1.64 (m, 2H), 1.36-1.23 (m, 4H); m/z (APCI-pos) M+1=437.2, 439.2.

### Example 88

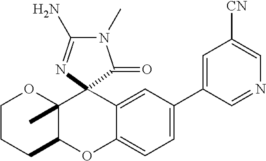

### (4S,4a′S,10a′R)-2-amino-8′-(5-chloropyridin-3-yl)-1,10a′-dimethyl-3′,4′,4a′,10a′-tetrahydro-2′H-spiro[imidazole-4,10′-pyrano[3,2-b]chromen]-5(1H)-one

(4S,4a′S,10a′R)-2-Amino-8′-(5-chloropyridin-3-yl)-1,10a′-dimethyl-3′,4′,4a′,10a′-tetrahydro-2′H-spiro[imidazole-4,10′-pyrano[3,2-b]chromen]-5(1H)-one (19%) was prepared according to Example 76, Step I, substituting (4S,4a′S,10a′R)-2-((E)-((dimethylamino)methylene)amino)-1,10a′-dimethyl-5-oxo-1,3′,4′,4a′,5,10a′-hexahydro-2′H-Spiro[imidazole-4,10′-pyrano[3,2-b]chromen]-8′-yl trifluoromethanesulfonate for (4S,4a′S,10a′R)-2-amino-8′-bromo-1-methyl-3′,4′,4a′,10a′-tetrahydro-2′H-spiro[imidazole-4,10′-pyrano[3,2-b]chromen]-5(1H)-one and 5-chloropyridin-3-ylboronic acid for 2-fluoropyridin-3-ylboronic acid. ¹H NMR (400 MHz, CDCl₃) δ 8.65-8.56 (m, 1H), 8.54-8.46 (m, 1H), 7.80-7.72 (m, 1H), 7.46-7.35 (m, 1H), 7.17-7.10 (m, 1H), 5.31-5.16 (m, 1H), 3.83-3.53 (m, 2H), 3.13 (s, 3H), 2.19-2.03 (m, 1H), 1.96-1.68 (m, 3H), 1.32 (s, 3H); m/z (APCI-pos) M+1=413.1, 415.1.

### Example 89

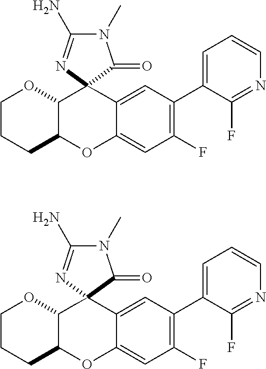

### (4S,4a′S,10a′R)-2-amino-7′-fluoro-8′-(2-fluoropyridin-3-yl)-1-methyl-3′,4′,4a′,10a′-tetrahydro-2′H-spiro[imidazole-4,10′-pyrano[3,2-b]chromen]-5(1H)-one

### (4R,4a′S,10a′R)-2-amino-7′-fluoro-8′-(2-fluoropyridin-3-yl)-1-methyl-3′,4′,4a′,10a′-tetrahydro-2′H-spiro[imidazole-4,10′-pyrano[3,2-b]chromen]-5(1H)-one

Step A: Bromine (14.1 g, 88.3 mmol) in chloroform (25 mL) was added over a 10 minute period to a round bottom flask containing 4-fluoro-2-hydroxybenzaldehyde (13.6 g, 97.1 mmol) in chloroform (68 mL). This mixture was stirred at room temperature for 16 hours, and additional bromine (14.1 g) was added. The mixture was stirred for an additional 24 hours at room temperature. The mixture was then washed with 30% aqueous sodium thiosulfate solution (2×) to discharge the color, water, and then dried over sodium sulfate. The organics were concentrated under reduced pressure to give 5-bromo-4-fluoro-2-hydroxybenzaldehyde (21.3 g, 100%).

Step B: 8-Bromo-7-fluoro-4-a-morpholino-2,3,4,4a,10,10a-hexahydropyrano[3,2-b]chromen-10-ol (100%) was prepared according to Example 76, Step B, substituting 5-bromo-4-fluoro-2-hydroxybenzaldehyde for 5-bromo-2-hydroxybenzaldehyde.

Step C: 8-Bromo-7-fluoro-3,4-dihydropyrano[3,2-b]chromen-10(2H)-one (46%) was prepared according to Example 76, Step C, substituting 8-bromo-7-fluoro-4-a-morpholino-2,3,4,4a,10,10a-hexahydropyrano[3,2-b]chromen-10-ol for 8-bromo-4-a-morpholino-2,3,4,4a,10,10a-hexahydropyrano[3,2-b]chromen-10-ol.

Step D: (4aS,10aS)-8-Bromo-7-fluoro-2,3,4,4a-tetrahydropyrano[3,2-b]chromen-10(10aH)-one (33%) was prepared according to Example 76, Step D, substituting 8-bromo-7-fluoro-3,4-dihydropyrano[3,2-b]chromen-10(2H)-one for 8-bromo-3,4-dihydropyrano[3,2-b]chromen-10(2H)-one.

Step E: (4S,4a′S,10a′R)-8′-Bromo-7′-fluoro-3′,4′,4a′,10a′-tetrahydro-2′H-spiro[imidazolidine-4,10′-pyrano[3,2-b]chromene]-2,5-dione and (4R,4a′S,10a′R)-(4R,4a′S,10a′R)-8′-bromo-7′-fluoro-3′,4′,4a′,10a′-tetrahydro-2′H-spiro[imidazolidine-4,10′-pyrano[3,2-b]chromene]-2,5-dione (38%), as a mixture of trans ring junction diastereomers, were prepared according to Example 76, Step E, substituting (4aS,10aS)-8-bromo-7-fluoro-2,3,4,4a-tetrahydropyrano[3,2-b]chromen-10(10aH)-one for (4aR,10aR)-8-bromo-2,3,4,4a-tetrahydropyrano[3,2-b]chromen-10(10aH)-one.

Step F: (4S,4a′S,10a′R)-8′-Bromo-7′-fluoro-1-methyl-3′,4′,4a′,10a′-tetrahydro-2′H-spiro[imidazolidine-4,10′-pyrano[3,2-b]chromene]-2,5-dione and (4R,4a′S,10a′R)-8′-bromo-7′-fluoro-1-methyl-3′,4′,4a′,10a′-tetrahydro-2′H-spiro[imidazolidine-4,10′-pyrano[3,2-b]chromene]-2,5-dione (22%) were prepared according to Example 76, Step F, substituting (4S,4a′S,10a′R)-8′-bromo-7′-fluoro-3′,4′,4a′,10a′-tetrahydro-2′H-spiro[imidazolidine-4,10′-pyrano[3,2-b]chromene]-2,5-dione and (4R,4a′S,10a′R)-8′-bromo-7′-fluoro-3′,4′,4a′,10a′-tetrahydro-2′H-spiro[imidazolidine-4,10′-pyrano[3,2-b]chromene]-2,5-dione for (4S,4a′S,10a′R)-8′-bromo-3′,4′,4a′,10a′-tetrahydro-2′H-spiro[imidazolidine-4,10′-pyrano[3,2-b]chromene]-2,5-dione and (4R,4a′S,10a′R)-8′-bromo-3′,4′,4a′,10a′-tetrahydro-2′H-spiro[imidazolidine-4,10′-pyrano[3,2-b]chromene]-2,5-dione.

Step G: (4S,4a′S,10a′R)-8′-Bromo-7′-fluoro-1-methyl-2-thioxo-3′,4′,4a′,10a'-tetrahydro-2′H-spiro[imidazolidine-4,10′-pyrano[3,2-b]chromen]-5-one and (4R,4a′S,10a′R)-8′-bromo-7′-fluoro-1-methyl-2-thioxo-3′,4′,4a′,10a′-tetrahydro-2′H-spiro[imidazolidine-4,10′-pyrano[3,2-b]chromen]-5-one (84%) were prepared according to Example 76, Step G, substituting (4S,4a′S,10a′R)-8′-bromo-7′-fluoro-1-methyl-3′,4′,4a′,10a′-tetrahydro-2′H-spiro[imidazolidine-4,10′-pyrano[3,2-b]chromene]-2,5-dione and (4R,4a′S,10a′R)-8′-bromo-7′-fluoro-1-methyl-3′,4′,4a′,10a′-tetrahydro-2′H-spiro[imidazolidine-4,10′-pyrano[3,2b]-chromene]-2,5-dione for (4S,4a′S,10a′R)-8′-bromo-1-methyl-3′,4′,4a′,10a′-tetrahydro-2′H-spiro[imidazolidine-4,10′-pyrano[3,2-b]chromene]-2,5-dione.

Step H: (4S,4a′S,10a′R)-2-Amino-8′-bromo-7′-fluoro-1-methyl-3′,4′,4a′,10a'-tetrahydro-2′H-spiro[imidazole-4,10′-pyrano[3,2-b]chromen]-5(1H)-one and (4R,4a′S,10a′R)-2-amino-8′-bromo-7′-fluoro-1-methyl-3′,4′,4a′,10a′-tetrahydro-2′H-spiro[imidazole-4,10′-pyrano[3,2-b]chromen]-5(1H)-one (68%) were prepared according to Example 76, Step H, substituting (4S,4a′S,10a′R)-8′-bromo-7′-fluoro-1-methyl-2-thioxo-3′,4′,4a′,10a′-tetrahydro-2′H-spiro[imidazolidine-4,10′-pyrano[3,2-b]chromen]-5-one and (4R,4a′S,10a′R)-8′-bromo-7′-fluoro-1-methyl-2-thioxo-3′,4′,4a′,10a′-tetrahydro-2′H-spiro[imidazolidine-4,10′-pyrano[3,2-b]chromen]-5-one for (4S,4a′S,10a′R)-8′-bromo-1-methyl-2-thioxo-3′,4′,4a′,10a′-tetrahydro-2′H-spiro[imidazolidine-4,10′-pyrano[3,2-b]chromen]-5-one.

Step I: (4S,4a′S,10a′R)-2-Amino-7′-fluoro-8′-(2-fluoropyridin-3-yl)-1-methyl-3′,4′,4a′,10a′-tetrahydro-2′H-spiro[imidazole-4,10′-pyrano[3,2-b]chromen]-5(1H)-one and (4R,4a′S,10a′R)-2-amino-7′-fluoro-8′-(2-fluoropyridin-3-yl)-1-methyl-3′,4′,4a′,10a′-tetrahydro-2′H-spiro[imidazole-4,10′-pyrano[3,2-b]chromen]-5(1H)-one (20%), as a 75:25 mixture of diastereomers, were prepared according to Example 76, Step I, substituting (4S,4a′S,10M-2-amino-8′-bromo-7′-fluoro-1-methyl-3′,4′,4a′,10a′-tetrahydro-2′H-spiro[imidazole-4,10′-pyrano[3,2-b]chromen]-5(1H)-one and (4R,4a′S,10a′R)-2-amino-8′-bromo-7′-fluoro-1-methyl-3′,4′,4a′,10a′-tetrahydro-2′H-spiro[imidazole-4,10′-pyrano[3,2-b]chromen]-5(1H)-one for (4S,4a′S,10a′R)-8′-bromo-1-methyl-2-thioxo-3′,4′,4a′,10a′-tetrahydro-2′H-spiro[imidazolidine-4,10′-pyrano[3,2-b]chromen]-5-one. m/z (APCI-pos) M+1=401.1.

### Example 90

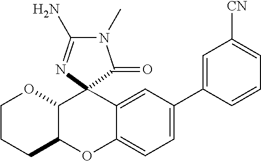

### 3-((4S,4a′S,10a′R)-2-amino-1-methyl-5-oxo-1,3′,4′,4a′,5,10a′-hexahydro-2′H-spiro[imidazole-4,10′-pyrano[3,2-b]chromen]-8′-yl)benzonitrile

3-((4S,4a′S,10a′R)-2-Amino-1-methyl-5-oxo-1,3′,4′,4a′,5,10a′-hexahydro-2′H-spiro[imidazole-4,10′-pyrano[3,2-b]chromen]-8′-yl)benzonitrile (9%) was prepared according to Example 76, Step I, substituting 3-cyanophenylboronic acid for 2-fluoropyridin-3-ylboronic acid. ¹H NMR (400 MHz, CDCl₃) δ 7.76-7.60 (m, 2H), 7.59-7.32 (m, 3H), 7.00-6.90 (m, 2H), 4.88-4.65 (m, 1H), 4.06-3.76 (m, 1H), 3.53-3.37 (m, 1H), 3.34-3.26 (m, 1H), 3.13 (s, 3H), 2.37-2.23 (m, 1H), 1.82-1.49 (m, 3H); m/z (APCI-pos) M+1=389.1.

### Example 91

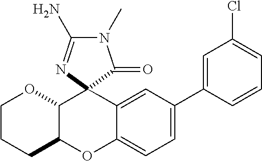

### (4S,4a′S,10a′R)-2-amino-8′-(3-chlorophenyl)-1-methyl-3′,4′,4a′,10a′-tetrahydro-2′H-spiro[imidazole-4,10′-pyrano[3,2-b)]chromen]-5(1H)-one

(4S,4a′S,10a′R)-2-Amino-8′-(3-chlorophenyl)-1-methyl-3′,4′,4a′,10a′-tetrahydro-2′H-spiro[imidazole-4,10′-pyrano[3,2-b]chromen]-5 (1H)-one (37%) was prepared according to Example 76, Step I, substituting 3-chlorophenylboronic acid for 2-fluoropyridin-3-ylboronic acid. ¹H NMR (400 MHz, CDCl₃) δ 7.47-7.21 (m, 5H), 7.16-7.12 (m, 1H), 6.98-6.92 (m, 1H), 4.86-4.67 (m, 1H), 3.93-3.78 (m, 2H), 3.44-3.33 (m, 1H), 3.09 (s, 3H), 2.36-2.28 (m, 1H), 1.82-1.48 (m, 3H); m/z (APCI-pos) M+1=398.1, 400.1.

The following compounds in Table 2 were prepared according to the above procedures using appropriate intermediates.

TABLE 2

| | | | NMR/ |
|---|---|---|---|
| Ex. # | Structure | Name | MS |
| 92 | 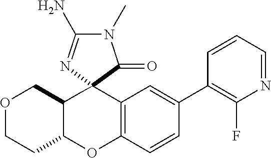 | (4S,4a′R,10a′R)-2-amino-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 383 |
| 93 | 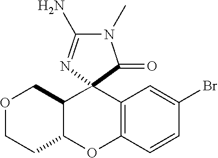 | (4S,4a′R,10a′R)-2-amino-8′-bromo-1- methyl-3′,4′,4a′,10a′-tetrahydro-l′H- spiro[imidazole-4,10′-pyrano[4,3- b]chromen]-5(1H)-one | 366, 368 |
| 94 | 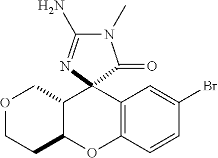 | (4R,4a′S,10a′S)-2-amino-8′-bromo-1- methyl-3′,4′,4a′,10a′-tetrahydro-l′H- spiro[imidazole-4,10′-pyrano[4,3- b]chromen]-5(1H)-one | 366, 368 |
| 95 | 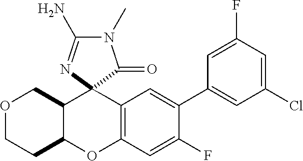 | (4R*,4a′S*,10a′R*)-2-amino-8′-(3- chloro-5-fluorophenyl)-7′-fluoro-1- methyl-3′,4′,4a′,10a′-tetrahydro-1′H- spiro[imidazole-4,10′-pyrano[4,3- b]chromen]-5(1H)-one | 434 |
| 96 | 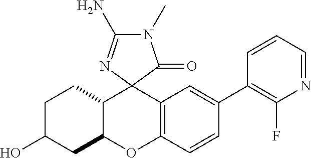 | (4a′S,9a′R)-2-amino-7′-(2- fluoropyridin-3-yl)-3′-hydroxy-1- methyl-1′,2′,3′,4′,4a′,9a′- hexahydrospiro[imidazole-4,9′- xanthen]-5(1H)-one | 397.1 |
| 97 | 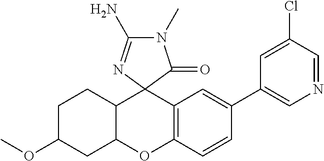 | 2-amino-7′-(5-chloropyridin-3-yl)-3′- methoxy-1-methyl-1′,2′,3′,4′,4a′,9a′- hexahydrospiro[imidazole-4,9′- xanthen]-5(1H)-one | 427.1 |
| 98 | 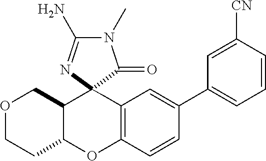 | 3-((4R,4a′R,10a′R)-2-amino-1-methyl- 5-oxo-1,3′,4′,4a′,5,10a′-hexahydro- 1′H-spiro[imidazole-4,10′-pyrano[4,3- b]chromen]-8′-yl)benzonitrile | 389 |
| 99 | 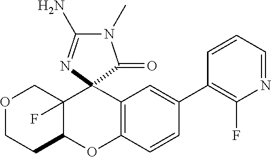 | (4R,4a′S)-2-amino-10a′-fluoro-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 401 |
| 100 | 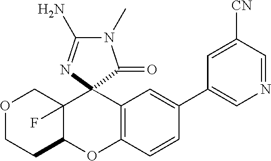 | 5-((4R,4a′S)-2-amino-10a′-fluoro-1- methyl-5-oxo-1,3′,4′,4a′,5,10a′- hexahydro-1′H-spiro[imidazole-4,10′- pyrano[4,3-b]chromen]-8′- yl)nicotinonitrile | 408 |
| 101 | 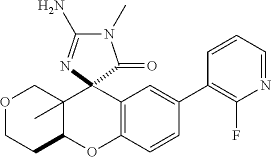 | (4R,4a′S)-2-amino-8′-(2- fluoropyridin-3-yl)-1,10a′-dimethyl- 3′,4′,4a′,10a′-tetrahydro-1′H- spiro[imidazole-4,10′-pyrano[4,3- b]chromen]-5(1H)-one | 397 |
| 102 | 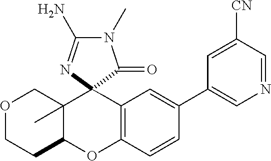 | 5-((4R,4a′S)-2-amino-1,10a′-dimethyl- 5-oxo-1,3′,4′,4a′,5,10a′-hexahydro- 1′H-spiro[imidazole-4,10′-pyrano[4,3- b]chromen]-8′-yl)nicotinonitrile | 404 |
| 103 | 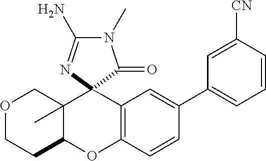 | 3-((4R,4a′S)-2-amino-1,10a′-dimethyl- 5-oxo-1,3′,4′,4a′,5,10a′-hexahydro- 1′H-spiro[imidazole-4,10′-pyrano[4,3- b]chromen]-8′-yl)benzonitrile | 403 |
| 104 | 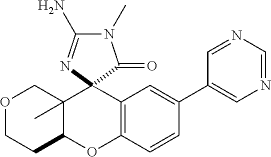 | (4R,4a′S)-2-amino-1,10a′-dimethyl-8′- (pyrimidin-5-yl)-3′,4′,4a′,10a′- tetrahydro-1′H-spiro[imidazole-4,10′- pyrano[4,3-b]chromen]-5(1H)-one | 380 |
| 105 | 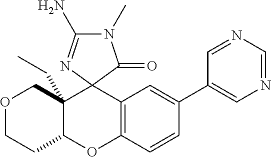 | (4a′R,10a′R)-2-amino-10a′-ethyl-1- methyl-8′-(pyrimidin-5-yl)- 3′,4′,4a′,10a′-tetrahydro-1′H- spiro[imidazole-4,10′-pyrano[4,3- b]chromen]-5(1H)-one | 394 |
| 106 | 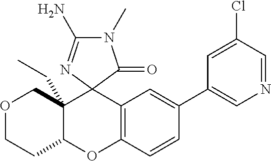 | (4a′R,10a′R)-2-amino-8′-(5- chloropyridin-3-yl)-10a′-ethyl-1- methyl-3′,4′,4a′,10a′-tetrahydro-l′H- spiro[imidazole-4,10′-pyrano[4,3- b]chromen]-5(1H)-one | 427, 429 |
| 107 | 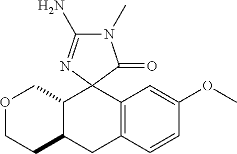 | (4aS,10aS)-2′-amino-8-methoxy-1′- methyl-1,3,4,4a,5,10a- hexahydrospiro[benzo[g]isochromene- 10,4′-imidazol]-5′(1′H)-one | 316 |
| 108 | 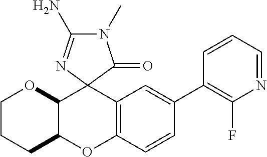 | (4a′S,10a′S)-2-amino-8′-(2- fluoropyridin-3-yl)-1-methyl- 3′,4′,4a′,10a′-tetrahydro-2′H- spiro[imidazole-4,10′-pyrano[3,2- b]chromen]-5(1H)-one | 383 |
| 109 | 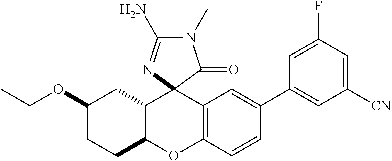 | 3-((2′R,4R,4a′S,9a′R)-2-amino-2′- ethoxy-1-methyl-5-oxo- 1,1′,2′,3′,4′,4a′,5,9a′- octahydrospiro[imidazole-4,9′- xanthen]-7′-yl)-5-fluorobenzonitrile | 448.8 |
| 110 | 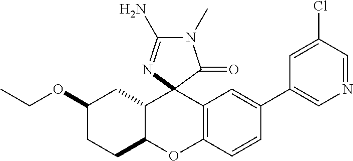 | (2′R,4R,4a′S,9a′R)-2-amino-7′-(5- chloropyridin-3-yl)-2′-ethoxy-1- methyl-1′,2′,3′,4′,4a′,9a′- hexahydrospiro[imidazole-4,9′- xanthen]-5(1H)-one | 440.8 |
| 111 | 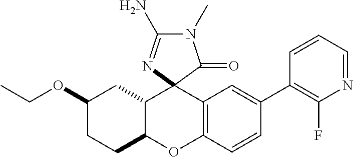 | (2′R,4R,4a′S,9a′R)-2-amino-2′-ethoxy- 7′-(2-fluoropyridin-3-yl)-1-methyl- 1′,2′,3′,4′,4a′,9a′- hexahydrospiro[imidazole-4,9′- xanthen]-5(1H)-one | 424.8 |
| 112 | 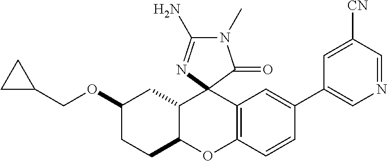 | 5-((2′R,4R,4a′S,9a′R)-2-amino-2′- (cyclopropylmethoxy)-1-methyl-5- oxo-1,1′,2′,3′,4′,4a′,5,9a′- octahydrospiro[imidazole-4,9′- xanthen]-7′-yl)nicotinonitrile | 457.8 |
| 113 | 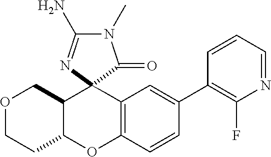 | (4R,4a′R,10a′R)-2-amino-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 383 |
| 114 | 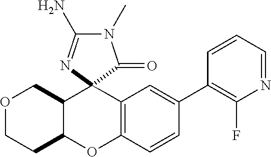 | (4R,4a′S,10a′R)-2-amino-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 383 |
| 115 | 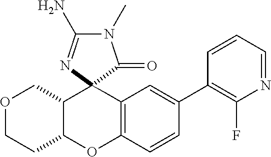 | (4R,4a′R,10a′S)-2-amino-8′-(2- fluoropyridin-3-yl)-1-methyl- 3′,4′,4a′,10a′-tetrahydro-1′H- spiro[imidazole-4,10′-pyrano[4,3- b]chromen]-5(1H)-one | 383 |
| 116 | 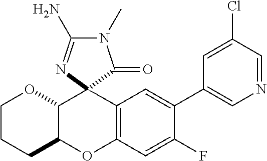 | (4S,4a′S,10a′R)-2-amino-8′-(5- chloropyridin-3-yl)-7′-fluoro-1- methyl-3′,4′,4a′,10a′-tetrahydro-2′H- spiro[imidazole-4,10′-pyrano[3,2- b]chromen]-5(1H)-one | 417, 419 |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

1. A compound selected from Formula I′: 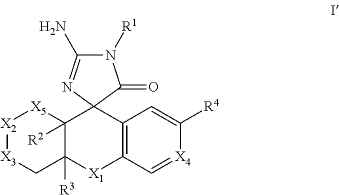
and stereoisomers, diastereomers, enantiomers, tautomers and pharmaceutically acceptable salts thereof, wherein:
X₁ is selected from O, S, S(O), SO₂, NR¹⁰ and CHR¹⁰;
X₂ is selected from CR⁵R⁶, NR⁷ and O;
X₃ is selected from CR⁸R⁹ and O;
X₄ is selected from CR¹¹ and N;
X₅ is selected from CR¹²R¹³ and O, wherein two of X₂, X₃ and X₅ must contain C;
R¹ is selected from hydrogen, alkyl, aralkyl, heteroaryl and heteroaralkyl;
R² and R³ are independently selected from hydrogen, halogen and alkyl;
R⁴ is selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;
R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or
R⁵ and R⁶ taken together form an oxo group, or
R⁵ and R⁶ together with the atom to which they are attached form a carbocycle or heterocycle;
R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;
R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or
R⁸ and R⁹ taken together form an oxo or alkenyl group, wherein the double bond of the alkenyl group is immediately attached to the carbon atom to which R⁸ and R⁹ are attached, or
R⁸ and R⁹ together with the atom to which they are attached form a carbocycle or heterocycle;
R¹⁰ is selected from hydrogen, halogen and alkyl;
R¹¹ is selected from hydrogen, halogen and alkyl; and
R¹² and R¹³ are independently selected from hydrogen and alkyl.

2. A compound as claimed in claim 1, comprising:
X₁ is selected from O, S, S(O), SO₂, NR¹⁰ and CHR¹⁰;
X₂ is selected from CR⁵R⁶, NR⁷ and O;
X₃ is selected from CR⁸R⁹ and O;
X₄ is selected from CR¹¹ and N;
X₅ is selected from CR¹²R¹³ and O, wherein two of X₂, X₃ and X₅ must contain C;
R¹ is selected from hydrogen, benzyl and C₁-C₃ alkyl, wherein the alkyl is optionally substituted with one or more R^{a} groups;
R² and R³ are independently selected from hydrogen, halogen and C₁-C₆ alkyl;
R⁴ is selected from hydrogen, halogen, C₁-C₆ alkyl, C₁-C₆ alkenyl, C₁-C₆ alkynyl, C₁-C₆ alkoxy, —NHC(═O)(C₁-C₆ alkyl), —C(═O)NH(C₁-C₆ alkyl), a 3 to 6 membered carbocycle, a 3 to 6 membered heterocycle, phenyl, and a 5 to 6 membered heteroaryl, wherein the alkyl, alkenyl, alkynyl, alkoxy, carbocycle, heterocycle, phenyl and heteroaryl are optionally substituted with one or more R^{b} groups;
R⁵ and R⁶ are independently selected from hydrogen, halogen, hydroxyl, CN, C₁-C₆ alkyl, C₁-C₆ alkoxy, phenyl, and a 5 to 6 membered heteroaryl, wherein the alkyl, alkoxy, phenyl and heteroaryl are optionally substituted with halogen or a 3 to 6 membered carbocycle, or
R⁵ and R⁶ taken together form an oxo group, or
R⁵ and R⁶ together with the atom to which they are attached form a 3 to 6 membered carbocycle or heterocycle;
R⁷ is selected from hydrogen, C₁-C₆ alkyl, C₁-C₆ alkoxycarbonyl, —C(═O)NR^{f}R^{g}, —SO₂(C₁-C₆ alkyl), a 3 to 6 membered carbocycle, a 3 to 6 membered heterocycle, phenyl, and a 5 to 6 membered heteroaryl, wherein the alkyl, alkoxycarbonyl, carbocycle, heterocycle, phenyl and heteroaryl are optionally substituted with one or more R^{b} groups;
R⁸ and R⁹ are independently selected from hydrogen, halogen, CN, C₁-C₆ alkyl, C₁-C₆ alkenyl, C₁-C₆ alkynyl, C₁-C₆ alkoxy, phenyl, a 5 to 6 membered heteroaryl and OR^{d}, wherein the alkyl, alkenyl, alkynyl, alkoxy, phenyl and heteroaryl are optionally substituted with halogen, or
R⁸ and R⁹ taken together form an oxo group or C₁-C₆ alkenyl group wherein the double bond of the alkenyl group is immediately attached to the carbon atom to which R⁸ and R⁹ are attached, or
R⁸ and R⁹ together with the atom to which they are attached form a 3 to 6 membered carbocycle or heterocycle;
R¹⁰ is selected from hydrogen, halogen and C₁-C₆ alkyl;
R¹¹ is selected from hydrogen, halogen and C₁-C₆ alkyl, wherein the alkyl is optionally substituted with one or more R^{b} groups;
R¹² and R¹³ are independently selected from hydrogen and C₁-C₆ alkyl;
each R^{a} is independently selected from OH, OCH₃, halogen, a 5 to 6 membered heteroaryl, and a 3 to 6 membered heterocyclyl, wherein the heterocyclyl is optionally substituted with C₁-C₃ alkyl optionally substituted with oxo;
each R^{b} is independently selected from halogen, CN, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen;
each R^{d} is independently selected from hydrogen and C₁-C₆ alkyl, wherein the alkyl is optionally substituted with one or more R^{e} groups;
each R^{e} is independently selected from halogen and C₃-C₆ cycloalkyl; and
R^{f} and R^{g} are independently selected from hydrogen and C₁-C₆ alkyl, wherein the alkyl is optionally substituted with halogen, CN or C₁-C₆ alkoxy.

3. A compound as claimed in claim 2, comprising:
X₁ is selected from O and CH₂;
X₂ is selected from CR⁵R⁶, NR⁷ or O;
X₃ is CR⁸R⁹;
X₄ is CR¹¹;
X₅ is selected from CHR¹² and O, wherein one of X₂ and X₅ must contain C;
R¹ is C₁-C₃ alkyl;
R² and R³ are independently selected from hydrogen, halogen and C₁-C₆ alkyl;
R⁴ is selected from halogen, C₁-C₆ alkoxy, phenyl and 5 to 6 membered heteroaryl, wherein the phenyl and heteroaryl are optionally substituted with one or two R^{b} groups;
R⁵ and R⁶ are independently selected from hydrogen, halogen hydroxyl and C₁-C₆ alkoxy optionally substituted with a 3 to 6 membered carbocycle, or
R⁵ and R⁶ taken together form an oxo group, or
R⁵ and R⁶ together with the atom to which they are attached form a 3 to 6 membered heterocycle;
R⁷ is selected from hydrogen and C₁-C₆ alkyl;
R⁸ and R⁹ are independently selected from hydrogen, halogen, C₁-C₆ alkyl, C₁-C₆ alkenyl, C₁-C₆ alkynyl, and OR^{d}, or
R⁸ and R⁹ taken together form an oxo group or C₁-C₆ alkenyl group wherein the double bond of the alkenyl group is immediately attached to the carbon atom to which R⁸ and R⁹ are attached, or
R⁸ and R⁹ together with the atom to which they are attached form a 3 to 6 membered heterocycle;
R¹¹ is selected from hydrogen and halogen;
R¹² is selected from hydrogen and C₁-C₆ alkyl;
each R^{b} is independently selected from halogen, CN, C₁-C₆ alkyl and C₁-C₆ alkoxy, wherein the alkyl and alkoxy are optionally substituted with halogen;
each R^{d} is independently selected from hydrogen and C₁-C₆ alkyl, wherein the alkyl is optionally substituted with one or more R^{e} groups; and
each R^{e} is independently selected from halogen and C₃-C₆ cycloalkyl.

4. A compound as claimed in claim 1, having the Formula I′a:

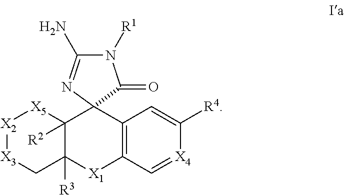

5. A compound as claimed in claim 1, having the Formula I′b:

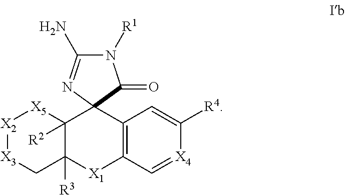

6. A compound as claimed in claim 1, having the Formula I′c:

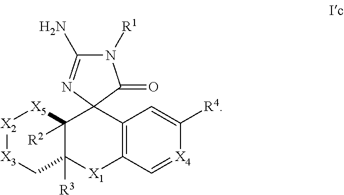

7. A compound as claimed in claim 1, having the Formula I′d:

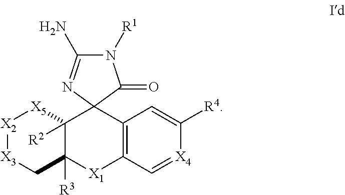

8. A compound as claimed in claim 1, having the Formula I′e:

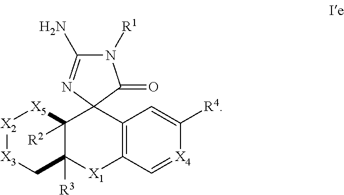

9. A compound as claimed in claim 1, having the Formula I′f:

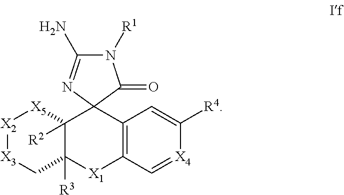

10. A compound as claimed in claim 1, having the Formula I′g:

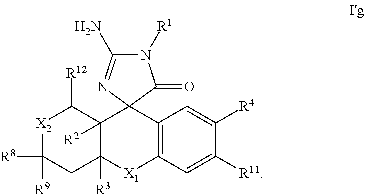

11. A compound as claimed in claim 1, having the Formula I′h:

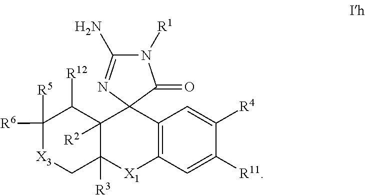

12. A compound as claimed in claim 1, having the Formula I′j:

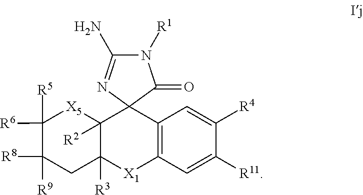

13. A compound as claimed in claim 1, wherein X₁ is O.

14. A compound as claimed in claim 1, wherein X₁ is CH₂.

15. A compound as claimed in claim 1, wherein X₂ is CR⁵R⁶.

16. A compound as claimed in claim 1, wherein X₂ is NR⁷.

17. A compound as claimed in claim 1, wherein X₂ is O.

18. A compound as claimed in claim 1, wherein X₃ is CR⁸R⁹.

19. A compound as claimed in claim 1, wherein X₅ is CHR¹².

20. A compound as claimed in claim 1, wherein X₅ is O.

21. A compound as claimed in claim 1, wherein X₄ is CR¹¹ and R¹¹ is selected from H and F.

22. A compound as claimed in claim 21, wherein R¹¹ is H.

23. A compound as claimed in claim 21, wherein R¹¹ is F.

24. A compound as claimed in claim 1, wherein R¹ is C₁-C₃ alkyl.

25. A compound as claimed in claim 24, wherein R¹ is methyl.

26. A compound as claimed in claim 1, wherein R⁵ and R⁶ are independently selected from hydrogen, OH, F, ethoxy and cyclopropylmethoxy.

27. A compound as claimed in claim 26, wherein R⁵ is hydrogen and R⁶ is selected from hydrogen, OH, ethoxy and cyclopropylmethoxy.

28. A compound as claimed in claim 26, wherein R⁵ and R⁶ are F.

29. A compound as claimed in claim 1, wherein R⁵ and R⁶ are taken together and form an oxo group.

30. A compound as claimed in claim 1, wherein R⁵ and R⁶ together form 1,3-dioxolan-2-yl.

31. A compound as claimed in claim 1, wherein R⁷ is methyl.

32. A compound as claimed in claim 1, wherein R⁸ and R⁹ are independently selected from hydrogen, F, OH, methyl, methoxy, ethoxy and cyclopropylmethoxy.

33. A compound as claimed in claim 32, wherein R⁸ is selected from hydrogen, F and methyl, and R⁹ is selected from hydrogen, F, OH, methyl, methoxy, ethoxy and cyclopropylmethoxy.

34. A compound as claimed in claim 1, wherein R⁸ and R⁹ together form oxo, methylene or 1,3-dioxolan-2-yl.

35. A compound as claimed in claim 1, wherein R⁸ and R⁹ taken together form oxo or methylene.

36. A compound as claimed in claim 1, wherein R⁸ and R⁹ together form 1,3-dioxolan-2-yl.

37. A compound as claimed in claim 1, wherein R⁴ is selected from Br, methoxy, 3-chloro-5-fluorophenyl, 3-chlorophenyl, 5-chloropyridin-3-yl, 2-fluoropyridin-3-yl, 5-(trifluoromethyl)pyridin-3-yl, pyrimidin-5-yl, 3-(difluoromethoxy)phenyl, 3-fluorophenyl, 5-fluoropyridin-3-yl, 3-cyanophenyl, 5-methoxypyridin-3-yl, 3-methoxyphenyl, 5-cyanopyridin-3-yl, 3-cyano-5-fluorophenyl and 3-cyano-5-chlorophenyl.

38. A compound as claimed in claim 37, wherein R⁴ is selected from 3-chloro-5-fluorophenyl, 3-chlorophenyl, 5-chloropyridin-3-yl, 2-fluoropyridin-3-yl, 5-(trifluoromethyl)pyridin-3-yl, pyrimidin-5-yl, 3-(difluoromethoxy)phenyl, 3-fluorophenyl, 5-fluoropyridin-3-yl, 3-cyanophenyl, 5-methoxypyridin-3-yl, 3-methoxyphenyl, 5-cyanopyridin-3-yl, 3-cyano-5-fluorophenyl and 3-cyano-5-chlorophenyl.

39. A compound of Formula I as defined in claim 1 and having the structure: 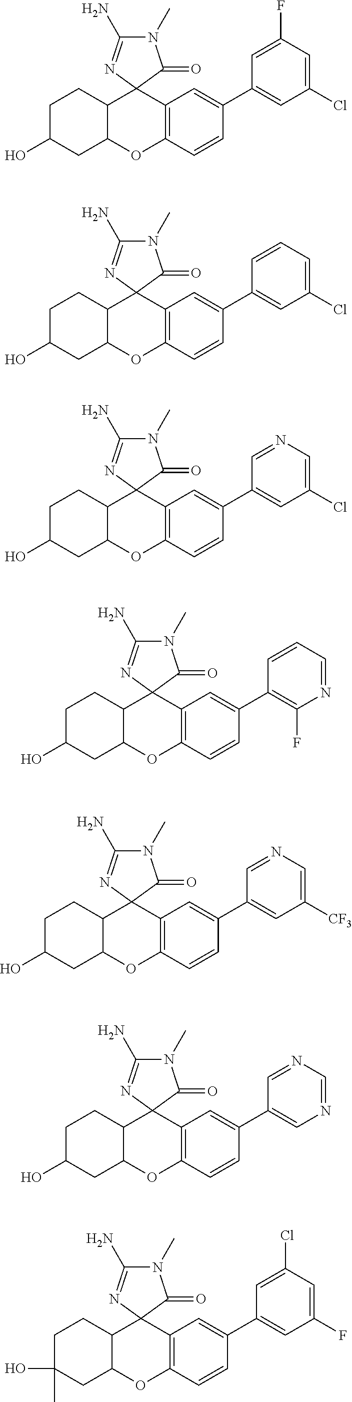 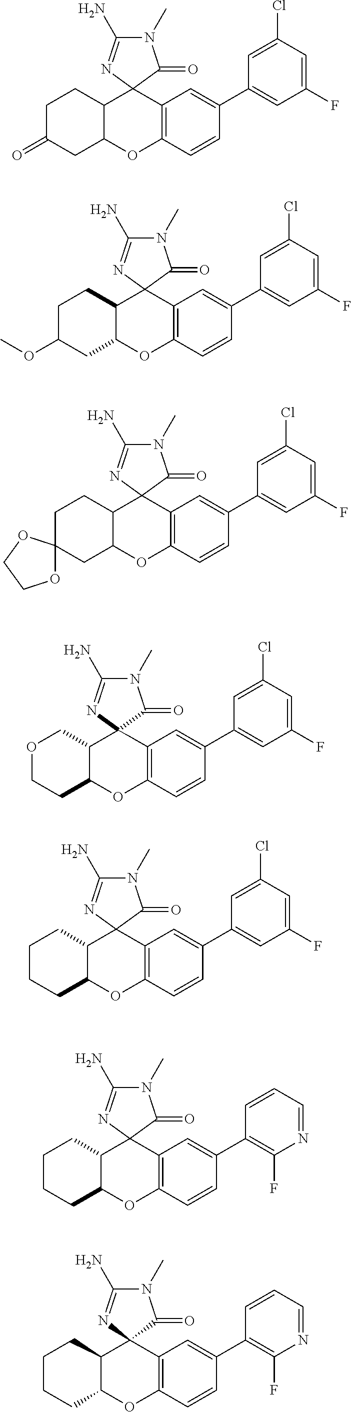 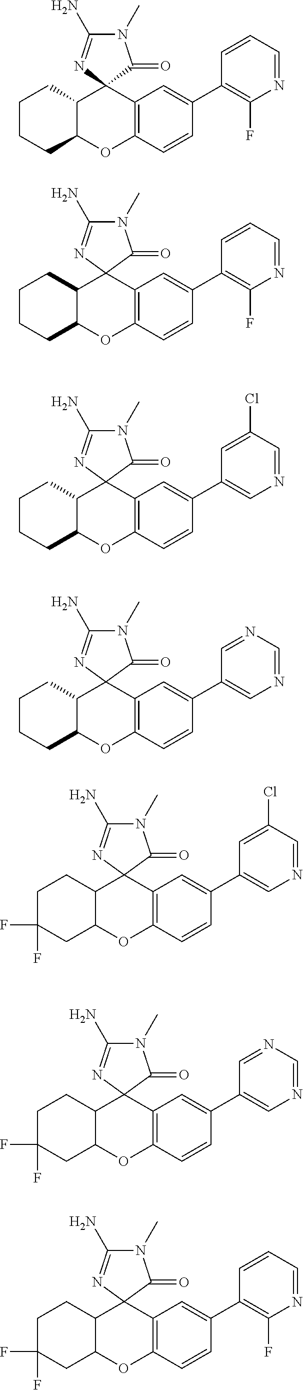 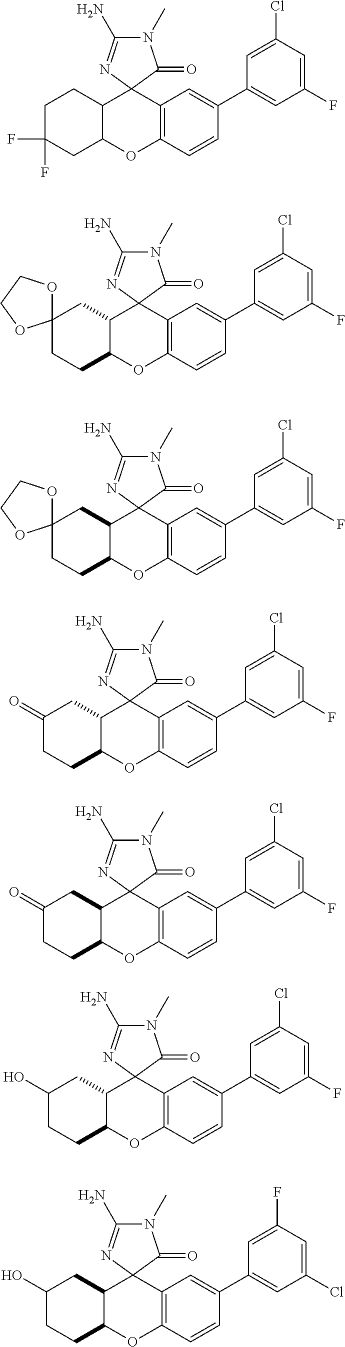 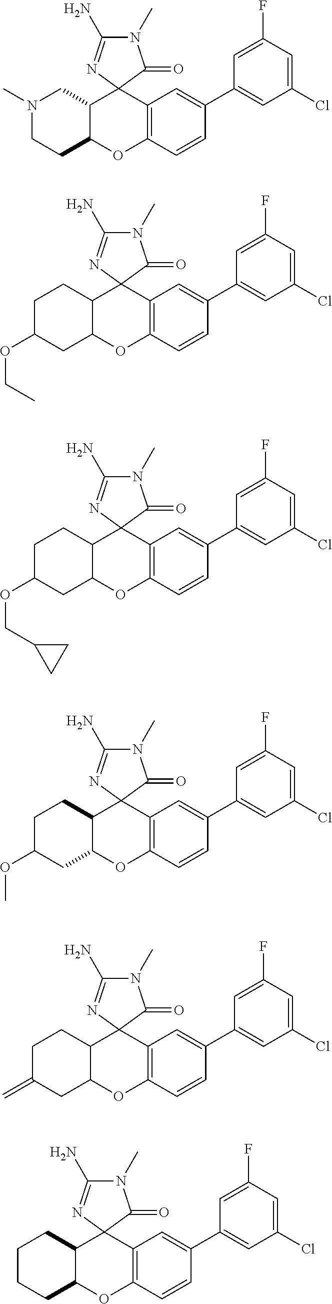 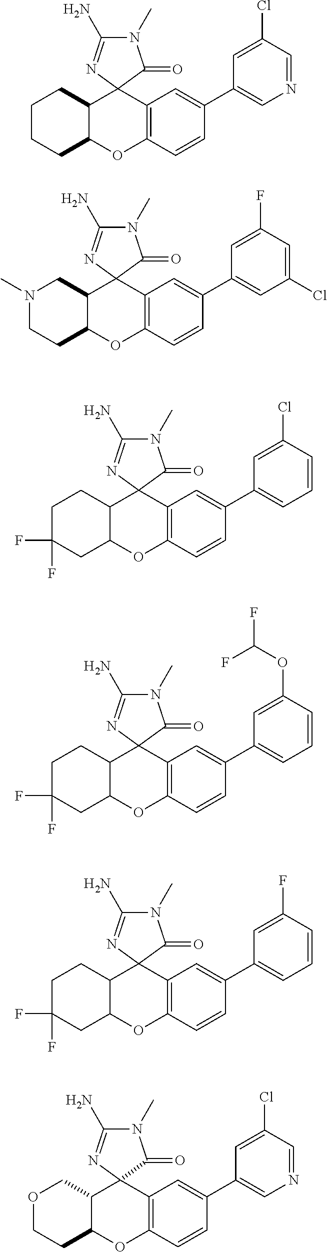 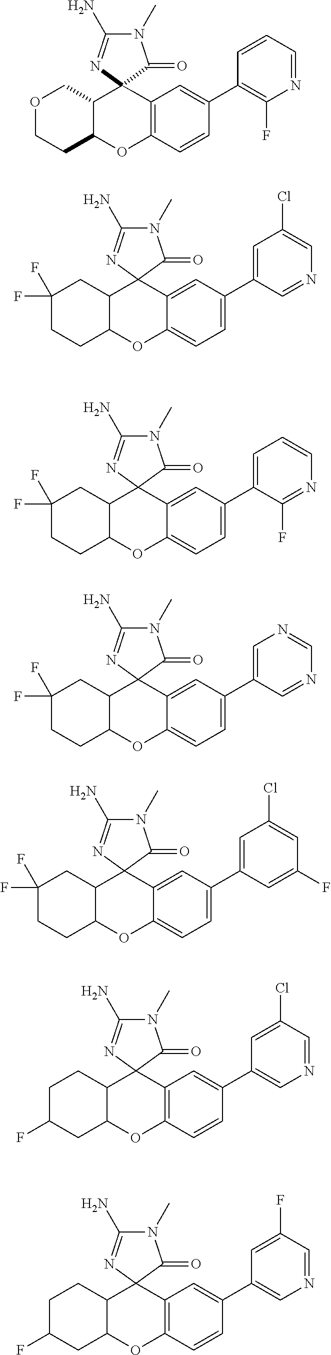 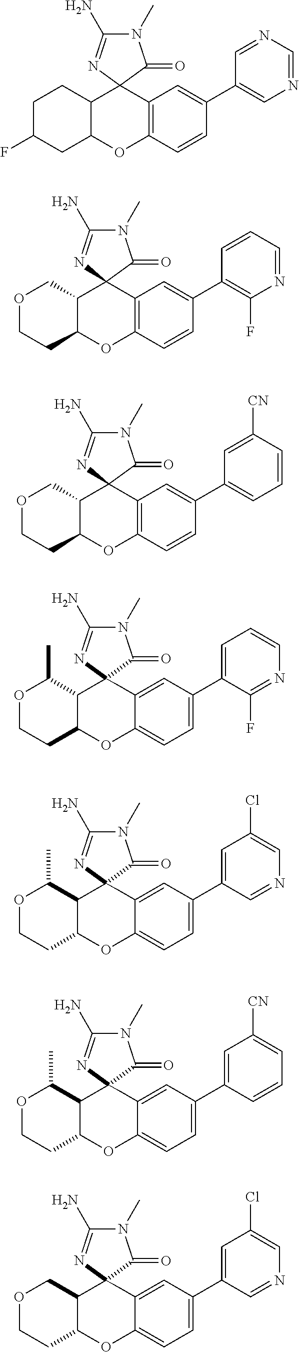 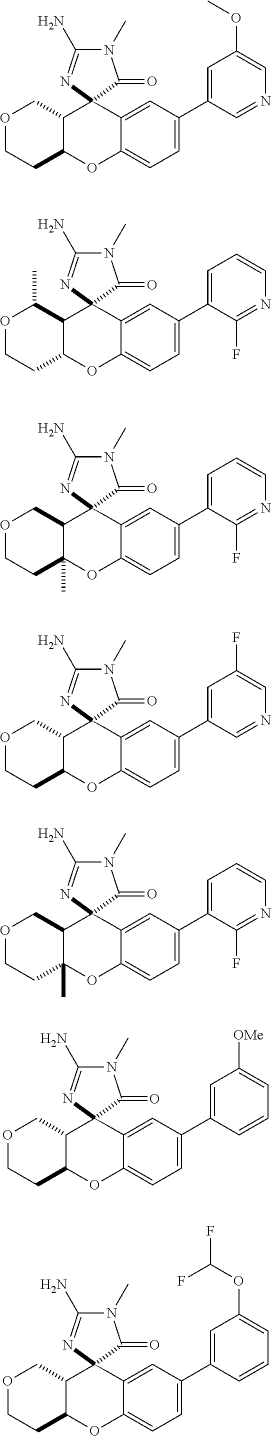 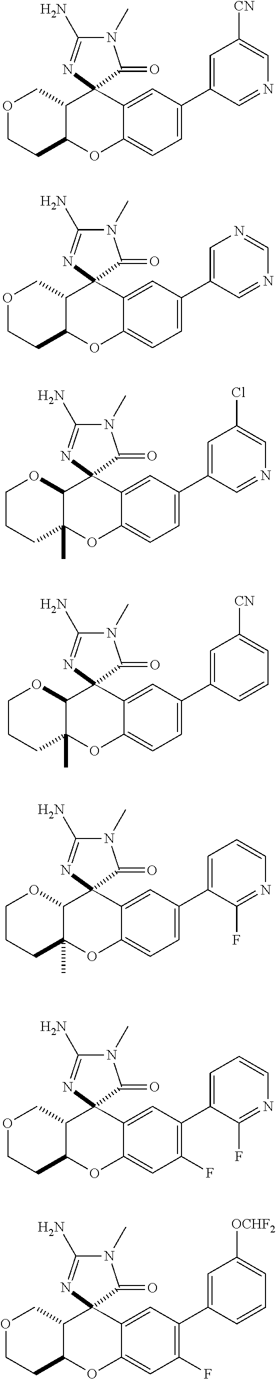 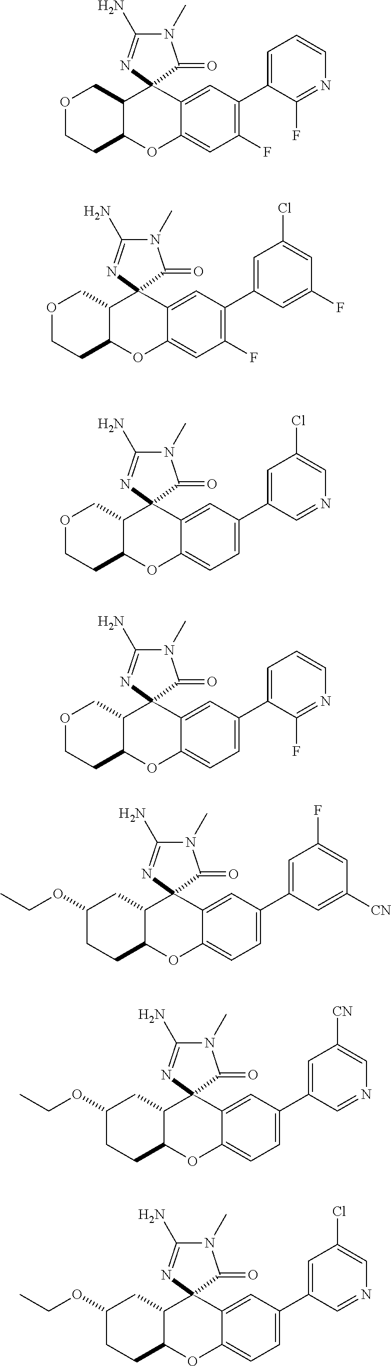 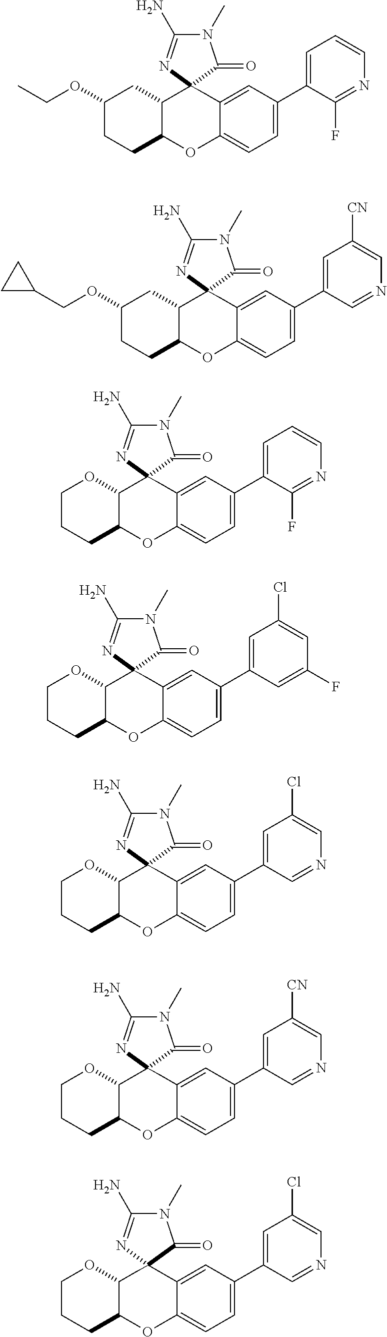 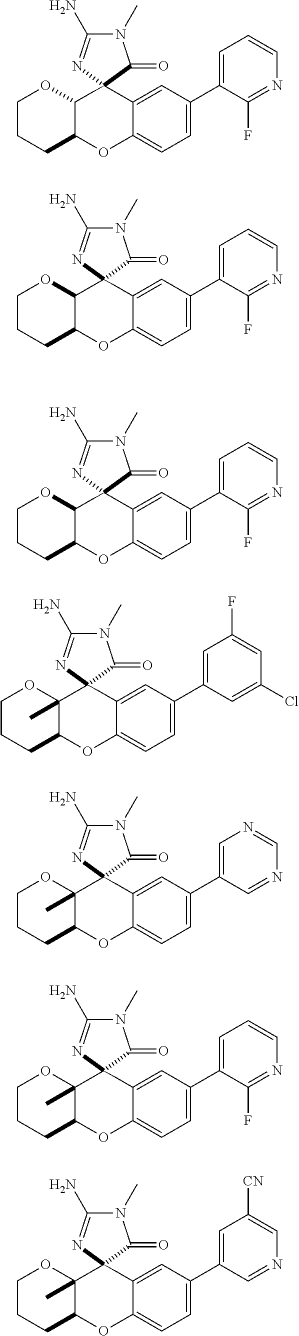 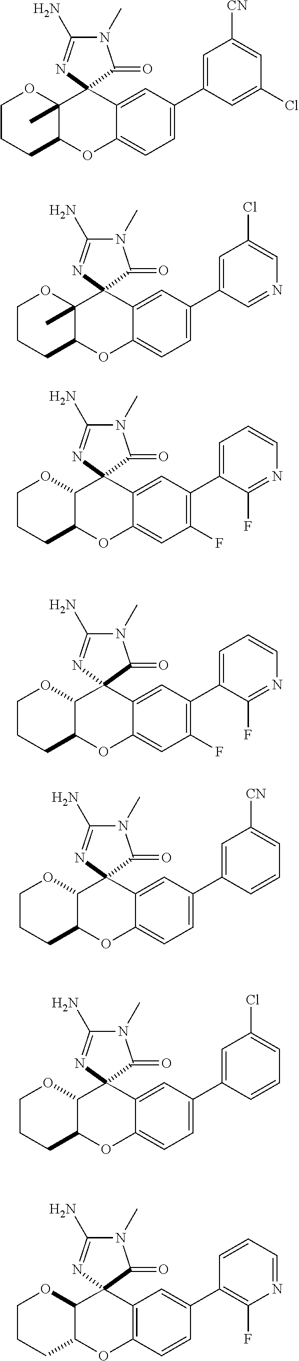 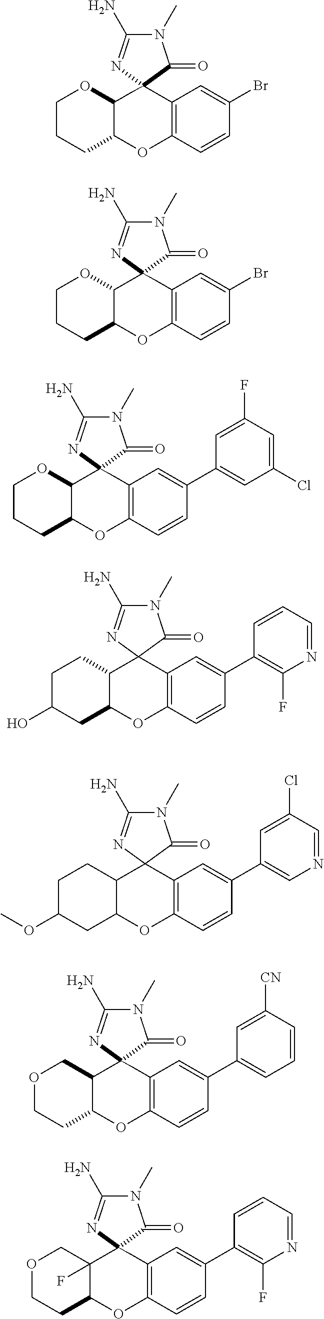 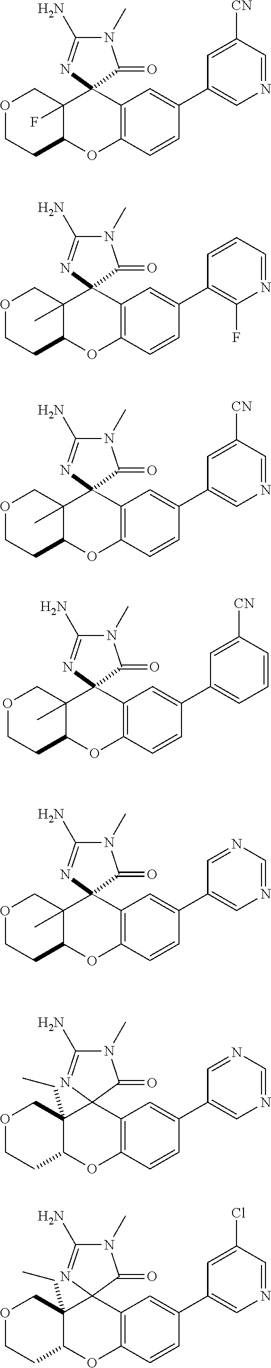 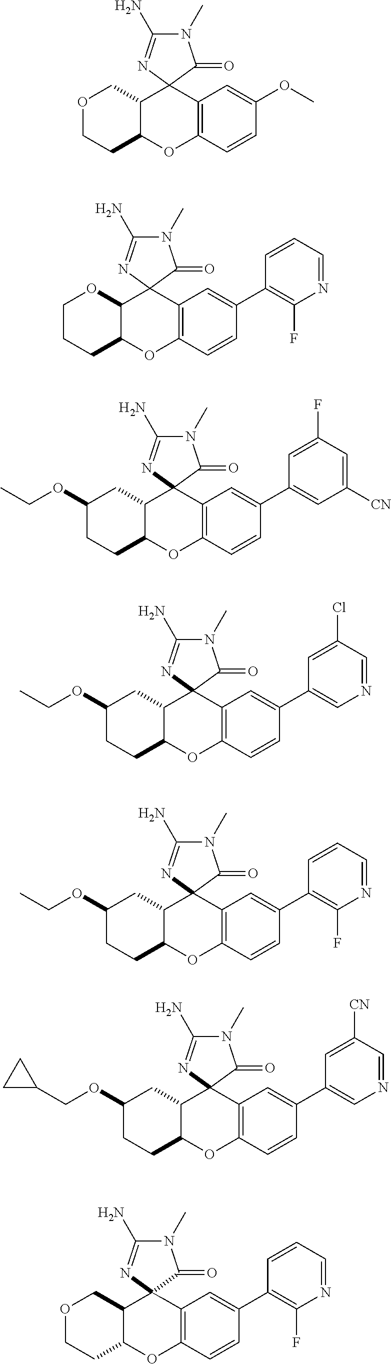 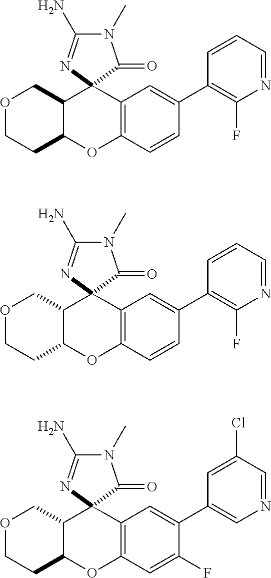
or a stereoisomer, diastereomer, enantiomer, tautomer or pharmaceutically acceptable salt thereof.

40. A method of inhibiting cleavage of APP by β-secretase in a mammal comprising administering to said mammal an effective amount of a compound of claim 1.

41. A method for treating a disease or condition mediated by the cleavage of APP by β-secretase in a mammal, comprising administering to said mammal an effective amount of a compound of claim 1.

42. The method of claim 41, wherein the disease is Alzheimer's disease.

43. A pharmaceutical composition comprising a compound of claim 1 and a pharmaceutically acceptable carrier, diluent or excipient.

44. (canceled)

45. (canceled)

46. (canceled)

47. (canceled)
